SEQUENCE LISTING (19 sequences)

SEQ ID NO: 1 - DNA, 2999 bp, Artificial Sequence
  4qV3_a
  misc_feature (1)..(2999): 4qV3_a
ttcattccataccgagttttcaaagagttttttataattaaagaatattaagttttatcaaataattttttagcataattgaaatgattatatggattttgtccttttatttatttatttatttttgagatgaagtctccctctgtcacccaggctggagtgcattggtgtgatctcagctcactgcaacctctgcctcctgggttcaagtgattttccttctttagcctcccgagtagctgggattacaggcacccaccaccgtgcctgcttaatttttgtatttttagtagagacggggttttgccatgttggccagactggtttcaaactcctaaacccaggtgatctgccagcctcagcttcccaaagtgctgggattacaggcatgagccactgtgcctgaccccttcattctgtttatgtgatgtatcacattgattaatttgaatgttgaaccatctttgcatccctgggataaatcccacttggtcattatgacttacctatttatgtattgtttaattgagtttgctaggtgttttgcaaatttttgcaccaatattctcagatatgggcctgtagtttgcctttttaatgtgtctttgtctggttttgatatcagggtaatactagcctcaaagaatgagtttggaaatgttctttccttctctatttttcagtctggtcctactgacttttttattaaggctttaattttgttaattgttattggtctgttcaggttttagattttttcctagttcaatctgggtaagctgtgtgtatctaagaattaatttcctctaggttttctaatttgttggcatacaattgctgatagtagccactaatgaccttttgattttctgaagcgttacttgtaatgtctcctttttcacctctgattttactaatttctatcttctctgttttttagttagcctgtctaaatatttgtcaactgttttacttttcaaaaaatcaactttttgtttcattaatcttttgcattgttttcttcattttatctttatttacttccactgtaatctttattatttctttccttctaattttgggtttggtttggccttttcattctagttaattaagatgtattgttaggttatttatttgaagcttttctgttttttatgtaggcacttacagttataaattttccttttataatagtacctcttttactatattccataggttttgctatgctatgtttccattatcatttgtttcaagaaatttttctgtgttcttcttaacttttttatcgacctactaatcattcaggatcatattgtttaatgtccacgtgtgtgtatagtttctgaaattcctttttaaattgatttctagttttattccctgtcgtcagagaaaatgcttgatattacttcaattttttggaatgtttttagacttgttacttaacatatggtctatccttgagaataacccatgtgctgaggagaagaatgtttattctgcagctgttgcatgaaattttctgtaaatatctattaggttcatttgttctatagtgcagattgtctgatgtttctttgttgattttctgtctgcaaggtctatccaatgcctaaagtggggtattgaagtgtccagccattattgtattgaggtctctttctttagctcttataatatttgcttcatttatctaggtgctttagtgttgagtgcatatatattttcaattattatatcctcttgatgaattgatctgtttattattatataatgacctttttaatctctttctacagtcttctgttgaaatctatttttgtctggtataagtatagctatttctgcaaagaactcaaacaaatttacaagaaaaaaacaaacaaccccagtgggtgaaggatatgaacagacacttctcaaaagaagacatttatgcagccaacagacacatgaaaaaatgctcatcatcactgaccatcagagaaatgcaaatcaaaaccacaatgagataccatctcacaccagttagaatggtgatcattaaaaagtcaggaaacaacaggtgctggagaggatgtggagaaataggaacacttttacattgttggtgggactgtaaactagttcaaccactgtggaagacagtgtggctattcctcaaggatctagaactagaaatagcatttgacccagccatcccattactgggtatatatccaaaggattataaatcatgctgctataaaaaaatggaggctttctaaatagatcttcattaatgttgaggttttaagtcttggtctcagccaaaaggaattcctcgcatacccaggagggatgaaacacttgcattgtcgcgttagatattcttagagagaaagaggaccacgcgtaatggaaaattgctaactttgcctcagggagaccaggttgattcactgagaaagtttgaattggtttagaaagcatagttctgagttttctaggagaaggagaaaagttagggtagggcataagaagtaatgcccacatcaaaaagttagaaagatctcaaattaacaacctaacatcacaactgaaagaattagagaagcaagaagaattcaaccacaaagtttatagaagacaagaaataactaaaatcagagcagaactgaaagaaatagagacatgaaaaactattcaaaaaatcacttaatcaaagttttttgaaaaaattagtaagatagatagggtactagctagatgaataaagaagaaaagagagaagattcaaataaacaaaattagaaatgatgaagggaatgttaccactgaccccagagaaataaaaataacaaccagcaactactatgaacacatctacacacacaaactagaaaccctagaaaagatggataaattcctggccacatacaccatctcaaggctgaaccaggaagaaactgagtccctgaacagaccaataatgagctccaaaaattcaatca

SEQ ID NO: 2 - DNA, 1950 bp, Artificial Sequence
  4qV3_b
  misc_feature (1)..(1950): 4qV3_b
gcccaggacttgatagattaatagtaaaattttaacagatatacgaagaagagccagtaccattcctactaaaactatttcaagaaatagaggaggagggactcttccccaacttgttctacaaggccagcatcatcctgataccaaagcctggcagtgacacaacaaaaaaagaaagcttcaggccagtatccttgatgaacatccattcaataatccacaacaaaatccttgcaaaccgaatccagcaacacatcaaaagtctaattcaccacagtgaagtaggattcttccctggaatggaaggttggtccatcatgggcaaatcaataaaatgtgatttataacataaataaaactaaagacaagaaccacgtaattgtctcaatagacgcagaaaagccttcagtaaaattcagcaatgcttcatgttaaaaagtctcaataaatgagatattgaaggaacatacctcaaaataataaaggccacctatgacaaactcacagccagcattatactaaatgggcaaagtctggaagcattctccttgaaaactggcacaagacaaagatgccctctctcaccactcctattcaacacagtattagaagtccttgctggagcaatcagacaagagaaagaaataaagtgtatttaaatagaaagagaagtccaactacctctgtttgcagacaacataattctctatctgaaccctatagttgtgacccaaaagctccttaagctgatacaacttccacacagtttcaggatacaaaatcaatgtacaaaatttgctaacattcctacacaccaagaagagccaaactaagagccaaatcagagaggcaattttattcacaatttccacaaaaagaataaaatacataggaatacagctaaccagggtggtgaaaaatctctataatgaaaattacaaaacactgctcaaagaagtcagaaaaaacacaaacaaatgaaaaatcattccatgttaatggagagaaagaagtaatatcatttaaatggttgtactgcccaaagctatattaaactaccaatgacaggcttcacggaagtagaaaaagctatttaaaaattcgcatagaaccaagaaagaacctaagtagccaaggcaatctgcaaagcttgaggcatcacattacttgacttcaaactatactacagcactacagtaaccaaaacagcatggtactggtataaaaacagacacatacaccaatggaacagaatagagagcccagaaataagaccacagacctacaattatctaatctttgacaaagctggcaaaaacaagcaatggggaaaagatttcctattcaataaatggtgctggaataacttgctagccatatgcagaagattgaagttggtcttcttccttataccacacacaaaaatcaacccaagatggattaaatacttaaatgtaaaacccaaaactataaaagccctggaagacaacctaggcaataccatcctggacatagaaacaggcaaagatttcatgataaagacaccaaaaacaattacaacaaatgcaactattgacaagtgggatctaattaaacttaaaagcttctgctcagcaaaagaaattatcaatagagtgaacagacaacctataaaatgagaaaaagtatttacaaactatgtctctgacaaggatctaatattcaatataaggaacttaaatttacaagagaaaaacaaacaaccctattaaaaagtgtccaaagaattgtctattcatgtccttagcctactttttgatgggattgtttgttttttttcttgctaatttgtttgagctcattgtatattctggatattagttctttgtcagatatacagattgtgaagattttctcccattctgtggattgtctgtttactctgctgactgttccttttgccgtgaaaaa

SEQ ID NO: 3 - DNA, 1957 bp, Artificial Sequence
  4qV3_c
tttgcttttgggttcttggtcatgaaatctttgcctaagccaatgtctagaagggtttttttgatgtcatcatctagaatttttatactttcaggtcctagatttaagttcttgatccatcttgagttgatttttgtataaggtgagagataaagatccaatttcattctcctacttgtggcttgccaattatcccagtaccatttgttgggtagggtgttctttccccactttgtttttgtttgctttgtcaaagatcagttggctgtaaatatttgggttaatttctgggttcttcattatgttctatttgtctatgtgcctgtttttatacaagaaccatgctgttttggtgactatggtcttatgatatagtttgaaatcaggtaatgtgatgccttctgatttgttctgtttgtttaatattgctttggctatgcgggctcctttttggttccatatgaattttaggattgttttttctagttctgtgaagaatgatgatggtattttgatggcaattgcattgaatttgtagattgcttttggcactacggtcatagacaattctcagaagatatacaaatggccaacaaacatatgaaaaaatgctcaacataactaatgatcagggaaatgcaaattacaaccacaatgcaatgccaccctactcccacaagaatggtcataataaaaaaatagtagatgttggtgtgaatgtggtgaaaagggaacgcttctgcactgctggtgggaatgcaagctagtacaatcactatggacaatagtgtggcgattccttaaagaactaaaagtagaagtaccatttgatccaccaatcccactactggctatgtacccagaggaaaaaaaagtcattataagaaaaaagatacttgcacacacatatttatggcagcacaatttgcaattgcaaaaatgtagaaccaatccaagtgcccatcaatcaacgagtggatgaagaaactgtggtacatacgtacgatggaataccactcagccacaaaaaggaatgaattaatggcagttgcagcaacctggatgggattgaagactattattctaagtgaagtaactcaggaatggaaaaccaaacatcatatattctcacttataattgggagctaagctataaggatgcaaaagcataagaatgacacaatggactttggggactcagggagaaaggggagaaggtggtgagggataaaaggctacagattgggttcagtgtatacagatgaggtgatggatgcaccaaaatcttacaaatcaatcaataacttaccaatgtaaccaaatgccccctgttgtcctaaaacctatggaaataaaaaaatgtttaaaaagtgggcaaaggacatgaacgcttttcaaaagaatatatacatgtgaccaacaaccatagaatacaaagctcaatatcactgatcattagagaaatgcaaatcaaaggcacagtgagacaccatctcacaccagtcaggatggctactatgaaaaagtcaaaaaataacagatgctggcaaggttgtggagaaagagaacacatacactgttggtgggagtgtacattggtcccaccattgtggaaagcacaacagtgctttcctcgaaagagataaaagcagaactatcacttgacccagcaatctcactactgggtatatacccagaggtatataaattgttctgtcataaggacacaagcacgtaaatgtttgttgcagcactattcacaatagtaaagacatggaatcaacctaaatgcccaacgatgacagattggatgcagataatatggcacactgtggaggaaaagttaaatattaaatttgaactcaattgaacatggacacaaacaatggtcactaagtcctggaatgagttgtgtgagccccttgaggcatccatccagtgctgcttcggagaaaca

SEQ ID NO: 4 - DNA, 1618 bp, Artificial Sequence
  4qV3_d
catccatccagtgctgcttcggagaaacagttattgaaaaacaacagttattgaaaaacagttattgaaaaacaacaggcaattgcaaaaacaaattgacctttttgtgttccttgagctcagttgcgaagggccctcatgactgggcctcatgacaaacaacttgttacaaaaagagctcggtttccagatcgcaccgaagcttcctgggacctctcctcatctgtgcacggactagtggccaattctgaatcccaggctgttgtttcccagtctggtgatgaatcctccacagtctggtgagtgtaaatgtatataaatgtatgtatatactttttcccttctccccttcccattaaaatttgtttgttgtatcatttgcttattatatctatattgccatatactcggggtaaagtctgtttacctttaaaagtattgtgtgtttcttttctttcctcacacgtttcccacacagaacacacatatgcaccatggaatactatgcagctacgtgttctcacttgtaaatgggagctaaataatgagaacttatgaacacaagggaacaacagacaacagacactgggggtctacctgagcggggaaggtgggagaagggagaggaacagaaaagataactgtggggtactgggcttaatacttgtgttatgaaataatctgtataacaagcccctgtgttatgagtttatctatgtaacaaaccttcacatgtacccccaaacctaaaataaaacattttttaaatcctttatgaaagctgtaagatctgctcctgtgtgtttgtatgtctatatgtgttacatgtatgtaataatattttgtaaataaagctcattcttaaatcgttaaatagaaatggctttacaattatccattaaaaataattagatacttgcttgatttaactgtgagcttacatcttttgttgagagtttcttgattcatgggtcttgataggtgaacatgaagaagtatggagacacattctcagtgcctagaccagcagctacaagccagaatcaagcccaatcggccccttctttctatgctttccctgttttgcctcctggctattttaggcgggggtggatcctccagttatagccttcacagttctgtctttagtcctaatggactcaggcaggccctgatcttcatagttctcctgggtgccatgtggctacttggaaccgaggattactgagggaagacattacggatgccacctgtgtcatagtttcaaaattctgttcattaatttaaaatcttaaaatcacattaaattaagtaatacataaccataaaatatcttgagtcatttgtaagctaaaatattgaaatactaaccattaaaaattagtttaggtctatataccatgacacgttacttgtatatggtatacaaaacctaaatatctttagttctgttaataaacagtaatttgaagaattatatttcttaaaagttataaaatggtttttatctaaaaatactgatacaagacagttgaaaatcactttttagggttttcactgaaaattggggctcctaagacttaattactagatatgagagaaacaattctgcatacag

SEQ ID NO: 5 - DNA, 4097 bp, Artificial Sequence
  4qV3_e
tgtgaacatacgcttttgatgataaaacttataaagacataaaaatgtgttttaatttttttttggtttgaagttacttaaagatttcaaattgaagaagtaaaaaaaaactagataaaactagataaatatagaaagttgggggaaaatgcaaagcatacgttcacaaaaatctgggattaaaagatgacaacatttgataaatttatttataaagttttattaaattaactttagaggctgggcatggtggctcacgcctgtaaataataaaattatcttgccaattatgtctaactatgatagtttaaagtcatttccactgtaaattgcttaattctgaggcagtttctgaaaactttacaagcttgcaaaatcctggaatattgtttctttaaggaggttcatgaaaggatggagaaggccctgagaagtactcttgaatataggtttttgagaactttagaattatattatttgaactgggtaagaatttccagaactttaatgaagagactgactggttaataaaattgctaacccaagcagaataaaaatcaattgaataccaagaaaatactttgccagattttcatgccaaatcagctagtacttaaattgtctagataaagaatttgaatgaactgcatagtccaagtcaaattatctatgataatccctgcttagtcagtgctatgcacctaagttgaagaaacagtccaatgttaagcatggagaaccagaatgtcttccttgtccttcctgagttctgaaagcttttcttattaaaagttctgcattctgattcttgggtgtgagagtaaagcaagacggcagaaagaaggctccactgattgtccctcctgcaaggacaccagatgaataactattaatatccacatagaaaatacattcataagaaccaaaaatcaggtgagtactcaaagtatctggttttaacctcagatcactgaaagaggcactgaagagacagaaaaaacagtcctgaaatcaccatttccttatccccagcagtgatggcatggtgcagacaacaactctgggttctgcaggagggagaacacagcaattgtgacgcactgcactcagtgctgtcctgttagagcagaaaggaaaaccagaccaaatgcagttaatgcccatccacagagggatcaattaaaccagccctagctagaggggaatcagattccagtggttggaacttcagtgtttggaaacctggcaactaagggctcccgcactgtgtgtctaagaaaacttgaaaggcagcctaggccatcaggactgcaactcttaggtgaggcctagggctgaaccgggcccagggacagtggactggggtggggagggcatgcaacacactgagacaccagctggggcagccaaaggagttctggcattatcactcccttaacaccagactgcacagctcatggctccaaaagggacaccatccttctgcttgaggagaggagggaaaagagtagggagtactttgtctttcatcctggacaccagctcagtcacagcaagatagtgcactggtcagaatcctgaggcctcctttccagtctctggctcctagacattcctagacacatcctgggccagaaggaaacctgctgccttgaagaaaagaaccatgtgctggcaggatttatcgcctgcaaacttgaaaacctttgggctctgaataaccagcagccatacccagtactacatcaagggccttggatgaccctcatagacttgctagatttaggtgaactcagcacattaccagctgtggtagctaaaaggcaaaactccttcttcttgggaaaagtagagggaaaattaaaggggactttgtcttgccccttaggtaccagcaaggccagaggtgggtagagcaccaagcaaaatttcataatccctgattctaggatttgattcttggctggcatttatgaagctgcccagggccagtagggagcccattgccctgaaaacggcaagtcccaggccaggcagcattcatcgcaagttgacaagaggttttgggccttaagggaacattggtggtagcctggcagtactcattgtggcctgaggtggtggtggctactctgcctttaggaaaggaagggaggaatgggaaggactatgtcttgtggtttgagtgtcaggtcagctgcaatgcaatagaatgtcaggtggacttctaaaacttttgactctactccctgacttctgaatggcacttctcgacccagccagggactgagggcactcactgccctaaaggaaagaacacaggcctggctggctttgccacctgctaattgtagagaccaaaggccttgagtgaacataggcagtcatcagaaagtgcatgcagcaggacttgagcaagacccagtgctgtgctagcttcaggtctgatccagggcagtcatagtggtggtggccaggggtgcttgtgtctttcttctcccagctttaggtggcttagaacagagagaaagactctgtatctttgagagaaaataatggtagagaacaagagtctctggctagtaatctagaaaattctcctggatcttgttgaaggctgtcaaggtggtacttctctgagtctggaagaattacagcattattgggtacaaggtacccataaagcagatatggcttagatcacaacacccaagtcttttaaaatatgtgaaaagtcttcccaagaaagacagctacaaataagcccagacagtgaagactacaataaatactcaccttttctcttttttaattttattttttaaacctctcatatggtgctgcatgcccaagaatttaatgtccagacactgaataacatctactagcatcaacaccatccaggaaaacatgacctcaccaagtgaactaaataaggcaccggggacaaatcctggagaaaaagagatatgtgacctttcagacagagaattcaacatagctgtattaaaaaaaaactgaaagaaatttaagataacaaaataaagaaattccaaattttgtcagctaaactcaacaaagagattgaaaaatttacaacaaattaagcagaaattctggagctgaaaaatgcaattggcatgctgaagaatgcattagagccatttaatagcagaatggatcaagcagaagaaatagtgagcttgaagacaggctatttgaaaatacgtagtcagaagagacaaaagaaaaagaataaaaacaacaaatcatgcctacaggatctagaaaaatagcctcacaaagacagatctaagagttattggttttaaagaagatgtagagaaagaggcaggggtagaaaaatttattcaaagggataataacagaaaactttccaaacctagagaaagatatccatatccaactacaaaaatgttataggacattaagcagatttaacccaaaaaaagactacttcaaagcattcaatagatcttccaaaagtcaaagataaagaaaagttctaaaggaagaaagagaagagaaacaaagaacataacagtgagctccaacacctctggcagcagactttacggtggaaaccctaccggccaggagagagaagcaataaatatttaaagtactaaaggaaaaaaaaccttttaccttagaatagcatatacagtgaaaatattcttcaaataaaaagaagaaatactgacttttccagacaaaagctgaagtattttatgaataccatgtctgtcctaaaggaatgctaaagagagtacttcaatcaggaagaaaaggacattaatgagcaataaatgatcacctaaaggtaaaaaaaatcctcagtgctaataggatacaaaacaaaacagaatattataacgctgtagctgtgggatgtgcaaactactcttattcaaagtaggaatactaaataatatccaatcaaaagtaatagctacaacatttccagacatagcacaataaaatataaatagaaacaacaaaaagttaaaaagtgaagggatgagcacacccgtcctgagccggccgatgtggtggagctcggagctcgggagccg

SEQ ID NO: 6 - DNA, 1065 bp, Artificial Sequence
  4qv3_f
tcgggcgtagagggagacagctgcccgggggcacggacgagcggctctgggggtcccggatccgagccccgctgccccggggtggcggtgacgcctggagccgggcggacgtggctgggccgggctgtcggggcgggcaggcgccgctgccggtgtctaggccacaccaccctgagcgcgccccagctcctctgagctcgtgaagctaagcagggtccggcctggttagtacttggatggattccgcctgggaatagcgggtaccgtaggcttttggcttcccgctccctccctctttccccgttttgtctccgtgcttcccaaccgccccctccctccaccctgtccgcccctgcgccccagccgaagcccaggacctccccctgaagatccgccactgcagcaccgccaggcagcagcatcccaccttttccgactcgccgcagccccaccaggagccgggctccaacccaggcggactggaccgaccccgagggcgcaggcgcgggttccctgaggtcccgggtgtctttcacgctccccggaagcccaggcaattcattcatccagcgccgctgcgacggtccaaaccgggggaaggggcgggcaggggcagcgggtcccacagacgccagccgagacctccgctccggaacccgtgggctgctttacccgggggaaggacattgccctcgccagccgtgaggaaatccgtccctgtgcactttgccaccgacttcgtgtaaattccagtcccgaggacacgagagagacccaggcctggccccgtggacaggctcagagccagggctctcgccagacggatgggcgcatctacaaatctggggggccaccgcatcgagaagacagaaagaagcaaacaaaggaaggaccctatgaaacgcacccccaaagcaaacaaccaatccaagaaaaaacacgtctcagggctccgttggttttcccgcatggggggccctgaccccctgttctagcccggcctaagcaccctccaccccaccccggcctgctcaaagggggcctgtctacctgagcagagcctcc

SEQ ID NO: 7 - DNA, 1232 bp, Artificial Sequence
  4qv3_h
caacggggcgagtcctcgttgggacaagcgacaatggtgtgggcgttgtgagaaaaagggccctcagggctgggccggctgtttgcccctggacagccctgacggctctgggtgtgtggggcaagagggggccttgcaggaggggcggcgagggatccaaaacaatttttctgcggcaagacgaaggaccggaggggatcccaggacagttggccctgggccctgacgcctcggagcacaccccgtcctgagccggccagatgtgttggaagcttgggagctcccaagccggggaaggcctggagcgtctgcgaaaagggaggccacctggtgtgcctggagcctgggcaggggactgaggtctctggctctcccgcgctgcagcagggcggtgccacggcgcgttctctggccgcaggcgacgggggtggctctgggaggccggcggaaggcgcagcgcggcggccggcggtgctgggaccttgatggcgagagcagcccggccgcgggggagcggttctggggccccggatctgagccccgcggcccaggggtggcggtgacgccaggagtcgagcagccgccactgccggcctttacggccacaccaccctgaacgcacgggatctcgactgaccttgaaagctaagcagagtcgggcctggttagtacttgggatgcgagaccccctgggaatacagggtgctgaaggcttttggctccccgctccctccctctttcccccttttgtcgccgtgcttcccaacggccccctgactctactcccacttttccgcacgcctgcgctccacaacgcatgggctggtttccaccgcggaaggacattgccttcgtcagccaccaagaaaaccgtccctgtgcacttggattttcattgccaccgacttcgtgtaaaatccagtctcggggacacgagagagacccaggtcttgagccctgtaagcacgctcggcgttatggctccggtcagatggacaggcgcactctacaaatctcggagcctactgcatcaagaagacagaaaggagcaaacaaaggaaggaccctaccaaacgcacccctgaagtaactaaccaatccaagagtgaacacgtctcagggctcagttagtcctctcccttggacggccctgcccccctgttctggcccagcccaagcaccctccaccccaccccggcctgctcaaaggggccctgtctgccagagcagag

SEQ ID NO: 8 - DNA, 1152 bp, Artificial Sequence
  4qv3_i
gctgccggggctctagtgccagcgcgggacaaagcagggccttcctgccccgttggccacgggccggtcttcctcgggacaagcgacaatggtgtgggcgttgtgagaaaaagggcccgcggggctgggccggctgttcgcccccgggcagccctggcttctctgggagcgtggggcaagagggggccttgcaggaggggcggcgaggaatccaaaataatttttccgcggcaaggcggaggaccagaggggatcccaggaccgtgggccctgggccctgacacctgggatcacaccccgtcctgagcaggccccaagtgttggaagctccggagctggagagccaggggaaggcctggagtgtcagctaaagggaggccgcctggagagtccagagccctggcagggaatggaggcctcgggcgcctcagcggtcctgcgcgtggtctggccgccggcgatagcgggacgctctgcgaggccggcggaaaacgcagcgcggcgactggtgcttgggcgtatagagggggagagcagcccggccgcgggcgagcggctccgggggtgcctgatcccagcctcgcggccccgggttggtggtgacgcctggaatcagacgcgcgtagctggaccgggctcttggaccagccaggcgccaccgccattgtctacggccataccattctaaacacgagagaaacccaagccggggcccgtgggcacgctcggggccactgctcccaccacagggacgggcgcactcgacaactttcaggacccacagcaccaagaggacagggaggagccagcaaaggaatgacgctacgaaacgcacccccaaagccaccaaccaatccaagtgaaaacacgtctcagggctcccttggttttccctcgtgggcggccctgcccccctgttctagcccggcccaggcacgctccaccctaccctggccaaaggggcccctgtctaccagagcagagcctccttctccaaggctctgttgctctccttctctagctccctcgcccgctccctttctctacttccctctccaccttgcccgctctcctccctctctctctctctctctctctcgctgtttctctctctccttccgtttctatctttccatccctctgtcctttgct

SEQ ID NO: 9 - DNA, 3289 bp, Artificial Sequence
  D4Z4
agggcacgtgcggtgcgggaagccccgttccccactcgccggtgtgggcgaaggcgacccacgagggagcagggtgacccccgccgggggccgcgctgcacaggccgcctgcctgcgcgggcgccctgccaccctgtcccgggtgcctggcccttcgattctgaaaccagatctgaatcctggactccgggaggcccgtctctctggccagctcctcccgggcggcgatgcctggaaagcgatccttctcaaaggctcggaggagcagggcggtctgggatccggtgacggcggtccgctttcgccggccttctggcgggccgcgtctcccgggccagggccgagattcccgccggtgctgcctcagctggcgtgacctctcattctgaaaccaaatctggaccctgggctccggaatgccgatggcctgggccagccgttctctggtggcgatgcccgggtacgggttccgctcaaagcaggctcgcagggcctcgctttggctcggggtccaaacgagtctccgtcgccgtcctcgtccccgggcttccgcggggagggtgctgtccgagggtgtcgggagggccatcgcggtgagccccggccggaatttcacggacggacgcgggcagagagaggccggcgggctcccgtgcacctcagccggactgtgcactgcggcaggtgcagccaggaggcctgcccggacagccagccagccagccagccgcccttgtaaaggcccacaggcaggcaggctccaccccttcatgaatggcggtgagcccccctgggacagcccgccccaccccggaagggacccagggcgtcgaggcctggggccggccggcggggtggtggtggtgggggggggggggggggggggagggcgtggtggcggtggtggtggtggggccggagagacgaagaggaagggggagaggggggaggggggaggggggcgcgtttcgggggccggctctccggacctctccagggatcccgcgggaacgggaagccgctctctgggctcccacgcgtcggcagcagggagaaaccagcctgggagggtggaggggagtgtggaactgaacctccgtgggagtcttgagtgtgccaggccctctctccgtgaaggaggcaatgcctgtgggcgtcgccgttgccgggacggtctcgcacacgcaggcgtgtggctctcgttcatttccacgtagaagaccagagcgagaccccagagaggagatgcctccccggcgtgatggcctgacgatggattcccgcgtgcggcaacgtgggggagtctgcagtgtggccggtttggaacctggcaaggagagcgaaggcaccatgccgggcttgcacccttccctgcatgtttccgggtgcccgcagagctccgggagcaaacagtcggcatggccagcctttcgggggccggagagacgtgagcaacaggccgccttgcggagggcaaagccacgcggaaaccaaaatcacgcctccgtcgtcctgcgtgtggctcctccgtggccgggtctgtcggcctcgcgccgcgttgcagggctcagcctggggatgtgcggtctgtgaaccgcgcgggtgaaaacccgacggcaacccgaattccggtcttttgtcccggaggaaaccgcccactccctgggccccggaaccggggcgaatgggtggtgccccgccggccggcgcggccgctgtgggcccagccctcagcccgcgccggacgctgaccgttttcccggagggcgggggtcccgctactcccggaggccgaggaccgcttttcctccctgccttcctccccccgtccgtccccggctccctcccgcccgcccccagtccctgcgtcgctctgtctctccctccgttcctccctgcctccctgcctccctgcctccctcctaacgtccctccgcccatccttccgcccctctaggtctcccgttcctctctccatctctgcccgccttccctcccgcctggaacgctcagcgtcccggtgtgcgccgggcctggggtctgcgttccgccgccaggcgctccgtgctggcagctgggcggctgcaggggcccgggcggcgggcgacggtggcccgggggcgacagggaggaggcgagccgccggagcggtgtcaggcccggacgctgcgcggggcccggtgtttcgcgggacgggggtctccacccagcccaggggacgacgcgttttccgggggtggggggtggggggtggggatggggcggtcaggcggcggggtgggctggtggagaggcaggagagctctgcccgggctgctcccacagcccaggcggctgcccgcaaacccgcgcgtgcgcagtaggcggcccacctgctggtacctgggccggctctgggatccccgggatgcccaggaaagaatggcagttctccgcggtgtggagtctctcaccggcctggacctagaaggcaggaatcccaggccggtcagcccggtggagggggcggggcggagacacgcccctccgtagccagccaggtgttccccgcgaaagagaggccaccgccctgccccgaaccacccgaccccgtcccaaccccgcgtcctaaagctcctccagcagagcccggtattcttcctcgctgaggggtgcttccagcgaggcgcctcttccgaggcctccagctcccccggggcctccgtttctaggagaggttgcgcctgctgcagaaactccgggctcgccaggagctcatccagcagcaggccgcaggggagtgcagaccagggcgccggctcctggagcgcctgggagggcgccgggatgccttgcatctgcccctgccgcgcggaggcggaggcgtccggggggcgcgggctggggaggtggagctgccccggcttggggttcccacgccgccccggcgacctggggaccccggccccagccccaccacggactcccctgggacgtgggtggcgcaagcaccccttggccctgcggccccgcttgagcgggcccaggctgtgccaccgcgcaggggcccggcaggccgtcgcgctgcgggtcccggtcctcccggcttttgcccgggtgcggaggccaccgaggagcctgagggtgggagagcgccccggctccggaggagccggggcggcgtaggcgaaatccccgcgcgccggggcaggttgggagatcccctctgccggcgcggcctggctgggctgcagcgcgggggcggccctcgctgcctggctcacgaaagccccctgtgggagagccccag

SEQ ID NO: 10 - DNA, 2408 bp, Artificial Sequence
  10qV3_a
ctgcctcctgagagcagagaacacaaattatttggaattcaaaagatttgtcttttctccccatttatttatatttatttgttcattcaatcatttacttagattacatggctcacaaatatttatttcatatttttggttataatccagtactattttattattttgttgctcaaattaattcaattttttgccacttggaaatctttcagttacctttgacatatccccatcacttttttttttttttttgagatggagtttcgctcttgttgcccaggctggagtgcaatggtgcgatcttggctcactgcaatctctgcctcccaggtacaagcgagcctgagatatggcctcatgtgaagggaaagaccttactgtcccccagcccaacacccgtaaagggtctgtgctgaggaggattagtaaaagaggaaggcctctttgcggttgagataagaggaaggcctctgtttcctgcatgtccctgggaatggaatgtctcagtgtaaagccgaacattcattctattctgaaataggagaaaaccgccctgtggctggaggtgagatatgccagcggtgatactgctctgttactctttgctacactgagatatttggataaagagaaacataaatctagcccatgtgcacatccgagcacagtagctttccttgaacttatttatgacgcagattcctttgctcacatgtttttctgctgaccttctccccgccatcaccctgttctcctgccgcactccccttgttgagatagtgaaaagagtaatcaataaatactgagggaactcagagaccagctctggtgcaggtcctcgcatgctgagtgtgccggtcccctgggcccactgttctttctctgtactttgtctctgtgtcttatttttctgtctctcatctccacctgatgagaaatacccacaggtgtggagggggaggcccccttcacctatattaagctaaacatgagttcatactgattctccaacttgaatccattagcacttggatcatactaacctcctcctgttcgttatctataaatgctcaccccaacagtgagaccctggactcagaagcattttaattaccaaatttcattctcttactagctattcaggttatccgtttcactttggctgagctttggtagtttgtggctttcaaggaattggtccatttcttcgaaattgttgaatttatgagtttaaaattgttaatagtattgtcttattatcattttaatggcttaagggtttcttatgatgtcacttttttgttcttgatattggtattccgtgtgtgcttttttttaatgtttgtcagtcttgattgagctttattattattttttatttttttcaaagtattggctttttgtttaattttctgtttttctattttcaatttcattgatttataacttaatcttaatttttccttccttatgcatactgtgactttttgtagatatttattttttagtgctttttgagataggaatttggatgattgatttgaggtctttcctcttaggtaaacttctagtgctataaatttttctctcagtgtttgagagtttttctctcagtgtttttctcagcacaaattttggtgtgctgtattttcattttcattctgttctgtgatttttaaagtttcatttgagaccatggattatttcctttgacctacagattatttagaagtgtgtttaatttccaagtgtttggagatttttcttttgtgtttctgttattgatttcttgtttcattcccttataatcagaaaacacactctgtatgacttctcttcttctaaatgtgttttatgacctaggatatagagagtcattttgatgaatgttccgtgtgtacatgaaatgaatgtgtattatgcttttgttgtatggagagttttataaacctcagttaggtcttactggctgaagatggtgtgtttaatccttctaaatccttgctgactttttgtctagtaggtctatcaattactgtgagtgggatgttgaaatctccaactaaaattgcgtatatgtctatttctccctttagttctctcagtttttgcttcatttacgttaatgctctgtggtttggtgcatatacctttaggattgctctacatttttggtggattgcaacttttatcattgtgtaatgtctgttttttctctctactgcttttcttctgtctgaaatcttttctttgacattaatatacccacttgtgctttttgggttaatatttacttttttttttgcatccttttactttcaacctacatatgtcatgttggcagagagtttcttgtaaacaacacatattttgctgttttaaaaattcatgctgccaatatctgcctt

SEQ ID NO: 11 - DNA, 3047 bp, Artificial Sequence
  10qV3_w
ctccagactgggcaacagagtgagaacctgtctcaaaaaaaagtaatcattgatatgttagggcttaagactgccattttactttttgttttctatctgtttgcaccattttttatgcctctttttcttttttctcttatcagcctgtgcgttatgtggacatagagttccattttgatgcatttttgacatatttgaggatactatttgcaaaattttcttattgcttgctctaagtattactatatacaaatataactttcacagcttacagctattgatgttttaccagtttgaattaagtatagaaacattaatcacatttaggttctgtgatggttaaaatgaggtgtcaacttgattgggttgaaggatgcctagatagcttgtataatattgtttttgggtgtgtctgtgagggctttgccagaggaggttgacattgcagtcagtggactggaagaggaagacccaccctccatttgggtgggtaccttccaatcggctgctagtgtggctagaacaaagtaggaagtagaaggtaggataagctggcttgctgagtcttcttgctttcatctttctctcatgctggattcttccttctattcctcctgcccttggacatcagactctaggttctttggaatttagactcttggacttaaacaccagtggtttgcaggagcctcttgggcctttgggcacagactgaaggctgcactgtcagcttccctgcttttgaggcttttggactcgaactgagcgactgctggcttcttatttccttagcttgcagagggcctatcatgggacttggccttttgatcatgtgagccaattttccctaataaactgcctttcatatatacatagatcctatcagttctatccctgtggagaacactgactaatacagcttcctttaccttcccacttaaaaaagtataattgtcctgggtatttcctctgtatacattgaataccacatgaggtagtattataacttttacttcaaacataaaacatgattaaagaaacacataaagtgaagaatattctatcatgtatacttctccttttaccaatttcattattttcctttatgaaaattctgtcttgctgttttcatgttctttctgttttcattttatttctgtttggagggatttctttagccattcttgaagggtaagtctgctagcaacaaattctcttagttctctttcatctgagaatatctttatttttcccttcactccagcagcatggtgttggtagaagagccgagccaggactagcttgtctgtcataatgtaaaaaagtcttggaacatgtcctgggtccagggtctaaacccccttgtggcctttggaacaccaagctctgtgccaaagggtggaaggctgccctgtggcaccacagtctaagcccagggcataaaacccctcatggcttgaatggaatccagggctcagggcgtcaaaaccctcatagcctctggaatgtgtccagacttgctggctccttgcttcttgctctcccaggatcataaattgattgtatcttgagttggaagaacatgttctctattatctcaagtagcagagcatgttccatgtgcttcaaaggaaatgctaaaccgtcacagctatgcttgatgcagcgctacctttctacccccacatctgcacatcctcaccacctgcttctttgtttgatcaccaataaatagtatgggatcccagagctcagggcctttgcagcctccataccagcattggccccctggacccatcttatgcactcttaacctgtcttttctcattcctttgactctgctggacttcgtagcccccacagcctggtattgggtctgatcatgccagtattcctagctcccaacatggtgctatgaataccctggtgaaggaacactagagcgtgtggaggtggaggatgcatcatcagaggacacctgaggacgactgaaagaagctagaataaatgttacaggagaaaaatatccttctttcagttctgtccaacaaggacctaaagaaccatatactgattttattgctcggctccaagagactatgcataaagctgtaactgacagaacagctcaagatgttgtaatacatctccttgcatatgataatgccaatatagagtgtcaagctgctattaaacctatgagacggaaggtcatttggctgaatatattaaggcttgtgatgcattgggggtaacttacataagggctactcttctagctcaagctatgactggactaaaggtgggaaaaatatgccctgtttctcaggctcttgttttaattgtgggcaatttggacacacaaaaaaaagtaatataaaaaggaaatcaacaggcgagggatactaccagtaagcaacaaagaagtcctggtatcttcccctgatgcaagaagggcaatcactggggaaatcagtgtcattctaaatttagcaaagatgggcaaccttttttgggaaatgggaagaggggcccgcctcaggtccctcaacaaaccgaggtgaatccggcacagtcagtgccgttacaaatgtacaataattgtcccctgccacagcaggctgtgccgctgcagatctttgcagcacagttcccatttccttacatcctggggagccaccaaagaaggtccccacaggagttaggggccctttaccctcgggaacagttgggctattgcttggaaggtctagtttaaatttaagaggtgtcactgtacatacaggaataattgattctgattataccagagaaattcaattagttattagttcctcaactccgtgtctgcttcctcaggagaaggaattgctcagttattactattaccttacaccaagctaggaagtagtacagtaaaaagaacaggaggctttggtagtacagatccagcaggaaaggctgtctattgggttaatcaagtgtctgacaaaagacctatttgtacaataaccattcaaggtaaagactttgaatgattagtagac

SEQ ID NO: 12 - DNA, 3036 bp, Artificial Sequence
  10qV3_x
caatggccccgaaatggcccaaagaaaaggcttcagtgggtattgttggcatagagactgcctcagaagtttttcaaagttctttaattttgccatgccaagggttggatggccaagaagggacaattcaacccatttacacctattcctgtcaatctatagggtagagtcttattacaatgatgggttgctgaaatatctattcctgtggatcagtatagtaataacagtaaagaaatgatgagaaaaatgggatatctcctggggaaagtactaggaaaaaatgaaaatggccaaccagaacctttagaattaaaagggcacacagatcgaactggattagggtatcatttttaggagaggccattgctgagcctctggctcccattcctcttgtttggttagctgccaaaccagtttgggtagagcagtggctgctgaaacaggaaaaactggaggctttaaaagaattggtgcaggaacaattgcaaaagggacacatagagcctactttctctccttgtaattctcttgtgcttgttattaagaaaaaatcaggtaaatggaaaatgttaacagctttaagggctattaatgctgtaattcaacccacgggcatgctgcaaccagagctggcttccccaactatgattcctagatactggcctctcatagtgatagatttaaaagattgcttctttactattcctttagctacccaagattatgaaaaatttgcttttactgttcctgctgtaaataacaaagaactagtggacagataccattggaaagtattaccagaaagcatgttaagtagcccaactctttgtcaaacttatgtcagacaagctattaagccagttagagaacaatttttaaaatgtgatagtatccattacatggatattttatgtgcagctgaaactagggaggaattaatgttatgctacaaacaattagaaaaggctgtaatggcaatagggttaatcatagcccctgataaaatccaaacttctaccccctttcaatatttagtagtgaaggtagaataaagttatattaagcctcaaaaggttcaaattcgaagagataatttaaaaactctaaatggttttcaaaaatttttaggagatattaattggattcatcccacattaggcatttctacttatggtatgtctcacctctttgctaccttacaaggtgattctgagcttaatagtaaacgctctgtatccaaagaagcattagaagaacttcaattaattgaagaaaaagcacaggtgaaacgaattgaccctacacagccattacagtttttagtttttcccactaaacgttcacctacaggagttattgttcaacagaatgatctggttgagtggctttttctacttcacaatgcaactaaaacgctcaccctgtacttagatcaaattgctttactagtaggacaagcaaggctgcacacaacagagttaatgggatatgatccaaatcggattatagtttcattaaacaaacaacaaattcagcaagcttatattaattcccaggaatggcaagttaatttggctggttttattggtgttcttgatagacattatcctaaattcaaaatattccagtttcttaagttaacatcatggatattgccttccattactcaaaaagcccctattgaaggggccattactgtttttactgacgggtttggtaatggaaaagactcatttgtaggacctcaacaacaagtttttcaaactggcttcacatctgctcaatgggctgaacttatggctgtaattatggtgttaaaaacctttaaacagccagttaatattgtttctgattcagcttatgtgatacaagccacacaaaatattgaatgtgctttaattcacaatgtgactgatgaacaacttaatcttttatttcattctttatagcaagcagtacaacaaagacactcccctttctatatcactcacataagagcacacgttaacctcccaggccctttgactaaacttaatcaaagggcagatgcactggtttctgcagcttttgatgatgcacagacaatccattctttagcccatcttaatgccacaggtctgagaaaaatatataatttatcatggaaacaggctaaggaaactgctctgcctgtcaagttttacatctaccacatcaaggagcaggagttaaccctaaaggtttatctccaaattccatctggcagatggatgtaacacatgttcccgcctttggaaaattgtcttttgttcatgtttcagtagatacttattcacattttatctgggccacatgtcaaacagggaagctacagctcatgttaaaagacatcttttatcttgctttgcagttatgggaattccaaaaaaaataaaaactgataatggcccagggtattgcagtaaagccatgactgcattttttcagcagtggaatattacttatactacaggaattccatgaaactcacaaggacaagcaatagttgaaagagctaatcataccttaaaaactcaaatacaaaaacaaaaagggggaaatcagacatataaatccccacatatgcaattacacttagctttattttaaatttacaaaaagatcaacccatgactgcagctgaacaacatctgacaggacaaaaggaaaataaaaggctggacaagatatatggtggagggatgcacatacaaaaaggtgggaaaaaggaaaaataattacatggggaagaggatttgcttttgtctctccaggtgacaatcagttgtctgtgggtgcccaccaaacatctgaagatccatcatgagccacaccaggaagagaggaccctgggaagagccagagctccctatacaagtgatggcatgaatgaacatctcagagacaaagaaaaagactgtgaatactcgccaggtggatcttccaacatggggccaagtcaaactggcacagatggcagaggccaacctcagagcacagaacaaaccaaaaaca

SEQ ID NO: 13 - DNA, 2910 bp, Artificial Sequence
  10qV3_y
cccactcagaaccaaagccaaaacaccctatccaatcaacattataggatacatctgcaggaaaaatgctttctctatgaaagctactccataaaataagagagcaaccattccatgaaatatgcagacaccaatgtagaaacatgaaaagacacacgaaaaagtaaggaaacatgacaccttaaaaggaacacaattctctctctagtagcagacccctaagaaaagaaaatctacaaaatgcatgaaaagtaattcaaaataatgatctcacagaaactcagcaagaaacaatgcaatataaagagacaattcaacaaaaccagaaaaacaattcctaatatggagaaattcaacaaagagatagatgtcataaagtgaacaaaagtctcataactgaagaattcaattaatgaaatagacatacaatcaagaacttcaacaatagactagactaagcatgagaaataattcctgactttgaagaagacagatcttttgaaataaccttctcagacaaaaaaataagttttaaaaatgaaaaaagcctatgggacttatggaacaagagtaggctaataaatattcactttatggaaggtcaagaagatgaagacatggaaaaagggatagaaaacccatttatggaaataataactgaaaacttctcaagtctagggagagatatgaacatccagatacaagaagctcaaatatctccaaatagattcaatacaaaaaggtcctctccaaggtacattatagtcacattgtcagaagtcaaagagaaatagaaacttctagaaacagcaaaagtattgagtaatatataaaggaatgtcaataagactaacagtagatttttcatcagaaactttgcagtccgggagagaatggcatgatatgttcaaagtgctgaaggaaaaaaacctggccagcaagaatattatagccagcaaatctgtccttcagaaatgaaggagaaatagtcttttctagacaagcaaaccttgatcatcatactagctcagccctacaagaaatgcttaagggagtcctacatctagaagtgaaaggatcttaaccaccatgataaaatctcacaaaagaataaaaatcactaggaaatcagggcttggtgatttattgaatgtgggacgagcaaaagaaagggtggaacaaaggactatggctggactactgccctggccaaagtggctggttctgagaaggagcacaggggaatgaggaagaaaggactgagaggggaaggatcatgttgacatgttaatatgagctgcccatggggggtggaaatgtccaggaggaagttagatggtggatgcacacgtctgaagctttaaggagagaaaaccttcctagaattctctttacttatcagaaaagaccccaaagctaacaacactttgtaatctcatgagaaatggccaaaagaattagagcttgaccccaattgtggctttcctcacaggacttctccccgttggttccagcagtttccatggtcccaggccagggctgggaccctgcacagccccctggcgaaataaggctgggaaagaacaatcactttgtgggaaacttggaggttcaggggactgctgtttgaaggtggtggagggtgatagagttaactcaacactgtgatcatgtgactgggttgtaatgtgactatggggaaaccacccaacctctctggtccatggtttccccatctgtgctgcagggcggctggcttgcaggatagttcctaagaagcatcctcctcatcagctctctaactctaagctttaactttgattagacttttgctggaatttctctggccctgccagggtgacagagtcaggggagattttgaagaacaaatcagtccagacaactgtgacatcttttattttctagatggcattgttagatatggggctttaataagttgatctgatatctggggtccaccaggccagctctctgaatggcatttttcactgtgttacttgatttttctttaattgaatgtggatcagaatatagataagggagctggatgcaggaagaaaccttcaaaggcaggggttttgcagtcagggaactatactctccaagagatgggagggtagggcatgtcggggaggacctgcattcatgccaagctgcagtgtcacctttattaatgatgacggagagggagtcagcaggcagctagtgtggactgcagaggatggtaaccgggaggtgttactacttttctaaaggacagggaagtgggacaaatgggaattttaagaggcgagaggtgaacagagggaggagagaaaggcaaaaaaaaaaaattgagtatgatgtcatctggaaaattgcaaacaaatgaatcaatgaagtatcattcccaaagcagggaggcttccagagaaggagccagcctggcggggagctggggtctgaaaggaagcacaagctgggttggctatgcagtgccctagccagagataagccacagccggcacgtgagccccaggaatggacttccactttcttctgtctctttctgcccagagccccccctttttttcccttgaaagacatcagacggaatgctgtatccaaaagggctttacaagagctcattaatgtgaaccattggagttagctttttaaacagttatctctgatttcagcttcataggatttttctcatcatggggatcagcttcataaaagccagagcatgatgattctgccaatttttgctgttaacagatgaaatagtactccctttataaaaatgaggtgcaatgatttcgggtagctttctttccaggcattcacggccatcctcatcatgatcattttcagtgttccaagctgtcctcctgaggccagcgtggt

SEQ ID NO: 14 - DNA, 3058 bp, Artificial Sequence
  10qV3_z
tggattcaaagttgtgtccagccaccttttatgttagcaataggaaatgttattcttgacataaatacccattctatcacatgccccgaatgtcacttgtttagttgcattaactcaacctttgataaaaatcaaactttcttgttaattagagccagagaaggagtttggatccctgtgtctctaaatagaccacgggatgcatcaccatccattcatattgtaactgagattcttaaaaacctgttatcccattcaaaaagattcacagttgctcttgtatttgctataattggcctcattgctgttaccactactgctgcagtagctggtgtggctttacactcatctgtgcagactgttgaatttgttgataaatggcaaaagaattctacgaaattatggaactctcaggcccaaatagatcaaaagatagtcaaatcagtgatctctgtcagacagtaatttggatgggggatcacatcctaagtttagaaactagaattcaaatgcgttgtgtttggaatatatccgatttttgcattactcctcatagttacaatgagacagaacaccaatgggaaagaattaaacgccatctagagggcagagaggaaaatctcaccttcaatattgtaaaactgaaagagcaggtttttgaagcctctcaggctcacttggccctgccccctggcactgatattttaaacaaggcagctgatgagttgtctgcaatctatcctcttaaatggattaagaccattggaaactctacacttgtaaactttgttctagtaactgtgtggttgtgctgtctccttttagtctgcagatgtggaagctgcctctggacaggaagcccctgtagagaacaagcaatgatagctgtggcagttttgcaaaaataaaagggagacatgttggtagaagagctgaggcaggactagcttgtctgtcataatgtaaaagagtcttggaacatgtcctgggtccagggtctaaaccccctcatggcctttggaacaccaagctctgtgccaaagggtggaaggctgccctgtggcaccacaatctaagctcagggcataaaaccccttgtggctttgatggaatccagggctcagaccataaaacccctcgtggccttttgaatgtgcaccgacttgctggctccttgcttcttgctctcccagaatcgtaaattgattgtatcttgagttggaagaacatgttctccattatctcacgtagcagagcatgttccatgtacttcaaagaaaatgctaaaccgtcacagctacgcttgatgcaccactacctttctacccccacatccgcacgtcctcaccacctgcttctttgtttgatcaccaataaatagtgtgggctcccagagctcagggccttcactgcctccatactagcgttggccccctggaccaaccttatgcactcttagccttttctcattcctttgactctgccagactttgtagccaccatggcctggtgttgggtctgatcacctgtagggaaaagaaaaagagatcagattgtcactgtgtctatgtagaaaggaaagccataagagactccattttgaaaaagagctgtactttaaacagttgctttgctgagatgttgttaatttgtagttttgtcccagccactttgccccagccactttgacccaaccactttgatccaatctggagctcacaaaaacatgtgttgtatgaaatcaaggtttaagggatctagggttgtgcaggacgtgccttattaacaaagtgtttactagcagtatacttggtaaaagtcattgctattctctagtctcaataaaccaggggcacaatacactgcggaaagccgcagggacctctgcccttgaaagttgggtattgtccaaagtttctctccatgtgatagtctgaaatatggcctcatgggatgagaaagacctgactgtcccccagcctgactcccataaagggtctgtgccgaggtggattagtaaaagaggaaagcctcttgcagttgagatagaggaaggccactgtctcctgcctgcccctgggaactgaatgtctcggtataaaacccgattgtacatttgttcaattctgagataggagaaaaactgtcctatggtgagaggtgagacatgtttgcagcaatgctgccttgttattctttactccgctgagatgtttgggtggagagaaacataaatctggcctacgtgcacatccaggcatagtaccttcccttgaacttgattatgacatagattcttttgctcacatgttttttgctgaccttctccttattatcaccctgctctcctactacatgcctttttgctgaaataatgaaaataataatcaataaaaactgagggaactcagagaccggtgctggtgcaggtccttggtgtgctgagtgccggtcccctgggcccactgttgtttctctatactttgtctctgtcttatttctttttattatcaccctgctctcctactacattcctttttgctgaaataatgaaaataataatcaataaaaactgagggaactcagagaccagtgctggtgcaggtccttggtgtgctgagtgccggtcccctgggcccactgttgtttctctatactttgtctctgtcttatttcttttctcagtctctcgtcctccccgacgagaaatacccacaggtgtggaggggcaggccaccccttcaatcaccccagcagatggcttcaccagatataggatttagcattgacagatcttttctttcagcagtaaaaactgttgttcctcattcacgcctgcagcagagctgtccagcatccacatgccccctccacctgggcctgataatgctggtgaccccacccattccagtggcagagccaccatgtgcctgcttgcccccttcccccttgtgctcaaggacttgcccacccagcagcctctgccccaagaaaagacatgccatatccccccaaaacacctacaatctaggccactcaggcatttgcagacactgct

SEQ ID NO: 15 - DNA, 3376 bp, Artificial Sequence
  qA1
ccccctccctgtggatcctatagaggatttgcatcttttgtgtgatcagtgcagagatatgtcacaatatcccctgtagaaaaagcctgaaattgatttacataacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctgcctgggctgatcagtgcagagatatctcacaaagcccctataagccaaaccttgacaagggttacatcacctgtttgatcagtggaaatatatatcacaaagccccctgtagacaaagcccagacaatttttacatctcctgagtgagcattggagagatctgtcacaatgcccctgtaggcagagctgagacaagtgttacatcatctgggtgatcagtgcagagatatgtcaaaacgctcctgtaggctgaacctagacaggagttacatcacctgggggatcagtgcagagatacgtgagaattcccttgtaggcagggcctagacaagtgttacatcacctaggttatcagtgcagagatatgtgagaattcccgtgtaggcagagcctagacaagtgttacatcacctagtttatcagtgtaattattagtcataaagcctcctgtaggcagagcgtagacaagagttccctcctcaggatgatcagtgcagagatgtgtcacaaagcccctgtaggcagagcctagacaagagtttcatcacttggttgatcagttcagagatgtgtcagaatgtccatgtaggcagatctaagacaagcgtccatcacctgggtgatcagtgcagagatatgtaccagtgtcccctgtaggcagtgcctagacaagagttgaatcacctcagagatcagtgcatagatatgtcacaaagccttctgtaggcaaagcccatacaaggtttacatcacctaggtgatcagtgcagtgatatgtcacaaaaatccctgtagacagagcctagacaagagttacttcacctgggtgatcagtgcagatatttgacacaatgcccccatagacagagcctaggcaagacttccatcacctgggtgatcagtgcagagatatgtcacaaatccccctctaggcagagtatagagaagagtcccatcacctgggtgatcagtgcagagatatttcacaatgccccctgtaggcagagagtggacaagagttacatcacctagatgatctgtgcagagctatgtcaaaacgcccctgtaggcagagcctagatgagtgttacatcacctgggtgatcattgcagagatacgtcacaataccccctgtaggtggggcctagacaagagttacatcacctgggtgatcagtacagaaatatgtcacaaagcccctgtaggcagagcctagacaagagttacatcacctgggttatcagtgcagaaatatgtcacaaagcccctgtaggtcgagtctagacaagagttacatctcctgggtgatcagtgcaaagatatgtcacaaagccccctgtagacaaatcccagaaaattgttacatcacctgggtgatcagtggagatatgtgtcacaattcccctttaggcacagcttagacaagcgttacatcacatgagtgatcattgcagagttatgtcactatgcccccataggcagatccaagacaagagtccatcacctgggtgatcagtgcagaaatatgccacaatgccgccagtaggcagatatagacaagagttacatcacctgcgtgatcactgcagagatatatcacaatgcccctgtaggcagagcctagacaagagtcccatcacctgggtgatcagtgcagagttatgtcacaatgcccctttttggcagagcctagacaagggttacatcacctgggtgatcagtgcagagatatgtcacaatgtccctgtagccatatccttgacaaaagtcccatcacctgggtgatcagtgcagagatatgtcacaaagcccctgtaggcagagcctagacaagagttacatcactttgttgatcagttcagagatgtgtcacaatgcccatgtaggcagagcctagacaagtgttccatcgcctgggtgatcagtgcagagatatgtgacaaggcccccatacacagagcctagacaacagtcccatcccctgggtgatcagtgcagaaatatgtcgcaatgcccccataggcagatccaacacaagagttacatcacctgggtgatcagtgtagagatatgtcacaatgccccaataggcagagcgtagacaaaagtcccatcaccaaggtgatcagtgcagagatatgtcacaaagcccccataggcagagcctagacaagagttacatcacttggttgatcagttcagagatgtgtcacaatgcccatgtaggcagagcctacaccagtgttacatcacttaggtgatcagtgcagagctatgtcacaatacccccgtaagcagagcctagacaagagttacatcacctggttgatcagtgcagagatatctcacaatgtccctgcaggcagagtatagacaagagttacatcacctagatgatcagtgcagagatatttcacaatgccccctgtaggcagagcctagataagaattatattacctggatgatcagtacggtgatatgtcactatgccccctgtgggcggagcctagacaagagttacatcacctgggtcataagggcagagatatgtcacaatgctccagtaggcagagcctagacaagagtcctatcacctgggtgatcagtgcagaaatatgtcacaatgctcccagtagacagagcctagacaagagttacatcacctgggtgatcagtgcagaaatatgttgcaatgcccccataggcagatccaacacaagagttacatcacctgggtgatcagtgcagagatatgcaacaatgcccccagtaggcagagcctagaggagagttacatcatctgggtgatctttgcagagatatgtcacaatcccccaagtaagcagagcctagacaaaagttacatcatctgggcgatcagtgcagagagaagtcacaaaacccacataggaaaagactagacaagagttacatcatctgggtcatcagtgcagacatatgtcaaagctgccgtagacagagtgtagacaattattacatcacttgggtgatcagtgcagagatctatcacagtgcccccataggcagagcctagacaagagttcc

SEQ ID NO: 16 - DNA, 3118 bp, Artificial Sequence
  qB1
cgcctgtaattcctgcattttgggaggccaaggagggtggatcacaacgtcaggagattgagaccatcctgactaacatggtgaaaccccgtctctactaaaaatacaaaaaattagccgggtgtggtggcaggcacctgtagtcccagctacttgggaggctgaggcaggagaatggcgtgaaccaaggaggcagagcttgcagttagctgagatcgcaccactgcactctggcctgggtgacagagcgagactcgggctaagaaaaaaaaaaaaaaaaaaggcaaacgtggcaaacaaacatatggaaaaaagctctagatcactgatcactagagaaatgctcatcaaaaccacagtgaggcaccatctcacaccagtcagaatggctattactaaaaactaaaaaactaacagaagttggcaagattgcagggaaaagtgaacatgtatacaccattggtgggagtgtaagttaattcaactgtggtggaaagcagtatggtgattcctcaaagagctaaaagcagaactacaacatttgacctagcaatcccattactggttatatacccagatgaatataaatcattctaccatgaagttacatgcatgcaaatgttcattgcagcactattcacaatagcaaagacatagaatcaacccaaatactcatcagtcacggattggataaagaaaatgtgctacatatacaccatggaataatatgcagccataaaaaagaatgagattatgtcttttgcattagtatggatgaagctgaagggtattatccttagcaaactaacgcaggaacagaaaaccaaatacagcatgttctaacttataagtgggagctaaatgataagaacttgtgaacacaaagaaggaaacaaaagacactggggtctacttgatggggggagggtgggaggagggagaggagcagaaaagataactattggatactgggcctaatacctggatgatgaaataacaggtacaacaaacccctgtgacacgtatttaactgtataacaaaccctcccgtgtaccccctaacctaaaataaaagttaaaaaaaaagaaacacagctttttattttttttctttttaatcccataaaggtcatgggccatttgccacctgaacagtcagtaacacatgagtggaaagaaactgaacacccagggacaccagagaccgttcactgtagaggaaggaggcaggtataactcataaggtgatcttcctccagtcccagagttgttcccctctccttaaatgtgggtcccatggaattcagactaggaagtaaagcaaatgagaaaggcctacaggggagcagttcaaatgtgtggaaaaggatagagcagccccaatgaggaaggaaggctggacaagcaatggatttgtagggaagacaatgtgcacccatcggagctctgattccttcattttcactaccctcccctgcccactaacataaaaaaagtattgatggcaagtgttgggccataagacatttccttgctttataatctggatttggggggttacatttcagagataatgaaaatctcctcctttagttaacttttaatttcataattgtaactttgtttttacagatttgtgaacatgtaaaacaaagaaaacagcatgggagtaatttataatcaacaaatatgtgttcactgagtgacactcacatggcatatggcatatgggcatcctgagagtaggatcgtggggcatccgatggtgattgtccttagagttggtcggagcccagccttccacggtctgcgcactgctgtgtgtacaatcggtgccttctttaagttcacagcaatgccacaaggcaaatagcactgtcttcactttctgtgtgagagaactaaggctgggtgagagtatgcagagctggatcatggatttgtttggctccttctagtttcaaaacctgtaaatattttgttgtctctaatgctaatcttagcactatatttggaataagcatataaacatataaccacaatccactcaggtaataggcaaccaaattataaataaatgctcaacaagattatgaaatgattgaatgagttatatataagaataataactggtggaagtgttattcataggataaagtagaaatcatttaaatggctaacagtataaggagggttaaatcaagcatccaggtgtggaaatagttgatttacatatatagtttatgccatgtaagacgtttatgatccaatattggatgaaaaaacagaaacattttatgtacagtataatctaaaatttctacatttcattaaaagtgcaggaatataattatcaaaatttgagtgataaaattatgaatttttctttttcttataactcattttatattcaccaaagatcctccaaagagaatatacacttttataattagaaaaaaagttatttgttaaagagatcataaatgacataatattttacagaagttaaaagctgagattgtgaggatttccagggactcctgccaccagcccatctcgcatcatcatttacatggaaatgttgggactgaggtgacttcatgcctctcaattcccagccagctttctctccctctggctcctagccagcctgttaaccagaaaataatcagtaagacaaaaaacaccctagtgttctaacaagttacacataattgcttgcttaccaattgcctacctcagaactctgtttccctcatagagtcaatataatcatttattttaaattcagcttctgccattttacagtctgagatgcaggactttgcatagggcatggatgggtttccacagtccacaggaggatgggaggagagcagggcagatccagccgcactcagtgagagttctggctggagttacttggctggacctcactccagagagaggagaccttcttctggaccagctaccaagacagcccctagttggaaaatcttttcttctaaggtgagatgcagcccccactggagaggaaaacaccagagagtatttaaaaaggaaaaaccaaggtaagctgaggagattcttgacaggagacacagcagagtccacccag

SEQ ID NO: 17 - DNA, 1351 bp, Artificial Sequence
  qB2
agttccccaacgggacagcaggtgggagccccaaatcactggcccctgcagcatgggagcagcatgagccatctgcagtgacattgtctcctccagcagcagcctccatccagaaatccatctcagggtctgtcctcacagtaacctgatgccagagctgtgcactcaaagtatttggacgcagatattttgtggaactgagcattttccaggtcttgctggagttcattccgcgtgaacttgatggagatctttctcatgggcttctttatttttttcactcttttttttattattatactttaagttctagggtacatgtgcacaacgtgcaggtttgttacatatgtatacatgtgccatgttggtgtgctacacccattacctcgtcatttacattaggtatatctcctaatgctttccctccccccttcctccaccccacaacaggccccggtgtgtgatgttccccttcctgtgtccaagtgttctcattgttcaattcccacctatgagtgagaacatgcagtgtttggtttcttgtgcttgtgatagtttgctgagaatgatggtttccagcttcatccatgcccctacaaaggaaatgaactcatccttttttatggctgcacagtattccatggtgtatatgtaccacattttcttaatccagtctatcattgatggacatttgggttggttccaagtctttgctgttgtgaatagcactgcgataaacatacgtgtgcatgtgtctttatagcagcatgatttataatcctttgggtatataccaagtaatgggatggctggatcaaatggtatttctagttctagatccttgaggaatcaccataccatcttctacaatggttgaactagtttacagtcccaccaacagtgtaaaagtgttcctatttctccacatcctctccagcacttgttgtttcctgactttttaatgatcgccattctaactggtgtgagatggtatctcattgtggttttgatttgcatttctctgatggccagtgatgagcattttttcacgtgtctgttggctgcacaaaatgccttcttttgagaagtgtctgttcatatcctttgcccacttcttgatgaggttgtttgttttatttcttataaatttgagttctttgtagattctggatattagtcctttgtcagatgggtagattgcaaaaattttctcccattctgtaggttgcctgttcactctgatggtagtttctttttctgtgcagaagctctttagtttaattagatcccatttgtgcattttggcttttgttgccattgctttcggtgttttaggcatgaagtccttgcc

SEQ ID NO: 18 - DNA, 131558 bp, Homo sapiens
  misc_feature (1)..(131558): CHROMOSOME 13q PROBE SEQUENCE
ctgtggtgatgtgtcacaattccatctgtgggctgggctcaggtaggagagtcaaattgctcaggtgcttggcagaggcttatgtcaaagtcaaacctgcaggaagttttggggatgagatttaacaatcccgcacatgtcttgtttctaggtatgagagtcagcacctcctatatgttgggtctaagtacacaagtcacaatctcactagtggactgaattcgtgcatgagagcctcccagagcctcccacctcttctgctggctttgcccttgtgaaatcacagccccacaggtgtgcttctcccagcctgtctgtgtctgcgacactccttctccagggatctcagtgcttttatccacattgtcacctccactgccctcctcccggcttgttttcagacatcattttattttttacttatttattttctgttatactttaagttctagggtacatgtgtacaatgtgcaggtttgttacatatctatatatgtgccatgttggtgtgcctcacccactaacttgtcatctacgttaggtatatctcctaatgctatccctccccactccccccaccccacgacaggccccggtgtgtgacgctaaccttcctgtgtccatgtgttctcattgttcaattcccacctatgaataagaacatgcggtgtttggttttttgaccttgcaatagtttgctgagaatgatggtttccagcttcatccatgtccctacaaaggacatgaactcatccttttttatggctgcatagtattccatggtgcatatgtgccacattttcttaatccagtctgtcattgatggacatttgggttggttccaagtctttgctattgggaatagtgctgcaataaacatacgtgtgcatgtgtctttatagcagcatgatttataatcctttgggtatatgcccagtaatgggatgactgggtcaaatggtatttctagttctagatccttgaggaattgccacactgtcttccacaatggttgaactagtttacagtcccaccaacagtgtaaaagtgttcctatttctccacatcttctccagcacctgttgtttcctgactttttaatgatggccattctaactggtgtgagatggtatctcattgtggttttgatttgcatttctctgatggccagtgatggtgagcattttttcatgtgtctgttggctgcataaatgtcttcttttgagaagtgtctgttcatatgctttgcccactttttgatggggttgaataaagaataaaagagagaagaatcaaatagatgcaataaaaaatgataaaggggatatcaccactgatcccacagaaatacaaactaccatcagagaatactataaacacctctacgcaaataaactagaaaatctagaagaaatggataaattcctggacacatacaccctcccaagactaaaccaggaagaagttgaatccctgaatacaccaataacaggttctgaaattgaggcaataatagcctaccatccaaaaaaagtccaggaccagatggattcacagccgaattctaccagaggtgcaaggaggagctgataccattctttctgaaactattccaatcaatagaaaaagtgggaatcctccctaactcattttatgaggccagcatcatcctgataccaaagcctggcagagacacaacaaagaaagagaattttagaccaatatccctggtgaacatcaatgcaaaaatcctcaataaaatactggcaaaccgaatccagcagcacatcaaaaagcttatccaccatgatcaagtaggcttcatccctgggatgcaagcctggttcaacatatgcaaatcaataaatgtaatccagcatataaacagaactaatgacaaaaaccacatgattatctcaatagatacagaaaaggcctttgacaaaattcaacagcccttcatgctaaaaactctcaataaattcggtattgataggacgtatctcaaaataataagagctatttatgacaaactcacagccaatatcacactgaatgggcaaaaactggaagcattccctttgaaaactggcacaagacagggatgccctctctcaccactcctattcaacatagtgttggaagttctggccagggcaatcaggcaggagaaagaaataaagggtattcaattaggaaaagaggaagtcaaattgtccctgtttgcagatgacgtgattgtatatttagaaaaccccatcatctcaccccaaaatctccttaagctgataagcaacttcagcaaagtctcaggatacaaaatcaatgtgcaaaaatcacaagcattcttatgcaccaatagcagacaaacagagagccaaatcatgagtgaactcccattcacaattgcttcaaagagaataaaatacctaggaatccaacttacaagggatgtgaaggacctcttcaaagagaactataaaccactgctcaacaaaataaaagaagacacaaacaaatggaagaacattccacgctcatggataggaagaatcaatatagtgaaaatggccatactgcccaaggtaatttatagattcaatgccatccccatcaagctaccaatgactttcttcacagaattggaaaaaactactttgaagttcatatggaaccaaaaaagagcccacattgcaaagacaatcctaagccaaaagaacaaagctggaggcatcatgttacctgacttcaaactatactacaaggctacagtaaccaaaacagcatgatactggtaccaaaacagagatatagaccaatggaacagaacagagccctcagaaacaataatacacatctacaaccatttgatctttgacaaacctgacaaaaacaagaaatggggaaaggattccctatttaataaatggtgctgggaaaactggctagccacatgtagaaagctgaaattggatcccttccttacaccttatacaaaaattaattcaagatggattgaagacttaaatgtaagacctaaaaccataaaaagcctagaagaaaacctaggcaataccattcaggacataggcatgggcaaggacttcatgactaaaacaccaaaagcaatggcaacaaaagccaaaattgacaaatgggatctaattaaactaaagaacttctgcacagcaaaagaaactaccatcagagtgaacaggcaacctacggaatgggagaaaatttttgcaatctactcatctgacaaagggctaatatccagaatctacaaagaactcaaacaaatttacatcattttatttttaatatctttgggtcattttattttatattgtctcataaacagaatagcataattttgcattttaacctagtcccagaggctttccccgctcagttttatccatttatatgtattgacataagtatattgtatttgttctcatttctggctcttttgttttcatttccttacttttcctcctcttatattaatactattgagtttttcctcagcaaatgtcaagacagtgcttgcaaccaatgtttcaaatttatacttcatttactttctttggctctcttattcttcctgctttatgtaacaggaaacagataaagcccaccacaaaagcaatgttaagataaagtcaaacaaaaaaagggaaaacagaagcactagaaagagtttcctgcagccacagaccagtcactgcactttattgaagtccccagctttggcaatatggttcaagtgtaggctccaagacagagctggggaggggcaggctcttgcaggacactggctccgaggcatgcctcccccactgccctgtacctggacaaaaacgggaaacatgggaaacgactcattgtctctgaacctcaagctggaattatggtctgtgtgtcccaagaagatggctttctcagcagcatctttaagctcctagggcctttggctcctgtgtgtgagggaggaaggaggcgatgggcccagggccatagggtacctcagtcttcagtccccttctctgccatggggtctgcagttttgaactactggaacgtgaatgcgtctctcggtggtgcttccccaccttggaaacagaccagccacgtaccaagctggcgacgagggagcacggcacacacgtcttctcattttagaaagaaaaacaaagttaatttatttgaaaactgtaggtttaggaaagaaaaatgctaagtgttatgagaccagaaacctgaaacacagccttcagaagaccctcctgggcccagcggccaccttgacctgctgttggaccacctccctatggttcggccagctgccagacctttgggatccgggatctttgctctgggatcttcgctggcctcgcatggatgggacacatcactttacctttatagtcccagaaattcatgacactggcagagaaccccttccctgctctccagagcccaggcccctctcctctgccccaaagcttcacctgacggaggctgtactgctagtctcctggatatctcaatctggtttaaatattaactcgcaaaatttctcaaacattttaaaggaaggatttcaattttccatcctccttaccccttcaagtcatttatccctaactccaaaatgagaagataataattttgtacctgtttctactgaaggatttatataagattatccaacaaaacccactggccatcctggcacttcctggagcccccagctaactggaagtatgcattgctctaatttagtgccccaaccccaataactcctgcacatgagaaagaagcaaaaattaaacaaacacaaatgatctccaggagcaaaattagctgtttaaataatcggctgactcagtgcaggttgacacaggacacacaatggatgtgagttggctttgagctccttggcctccaaccatcatctcggacaaaagctcagcctcggggagccccattctcgcttctaaagatgggacaggaaccacctcctggacctcacgtcagggttaagtgaataacatttgcaaaaatgactgtgaaagtgactcacacttggctgggcatggtggctcatgcctgtaatcccagcacttcgggaggccgagacaggcggatcacgatttcaggagatcgagaccatcctggccaacatggtgaaaccccgtctctactaaaaatacaaaaattagctgagtgtagtggcgtgcgcctgtagtcccagctactcaggaggctgaggcaggaggatcgcttgaaccagggaggcggaggttgcagtgagccgacatcgcgccactgcactccagcttaggcgacagagcgagactacgtctcaaaaaaacaaaaaaacaaaaaaagtgactcacacttacttggcaaaagataaatttgagtttctttcctttttcctcctgaaggccctttaccatccagcaacaagccaacatggctcagtttactgattccgggaaaagtactttgctgcatttcagaaagatgtttaacaacaaaactgacaaaaggttatctgattatttgaatagagatttctcccaacaggatatacaaatggccaataagtgcatgagaagatgttcaatgttattagtaaatgcaaatgaagaccacagtgagatgtcacttgcagccactaacttgtctgcaatcaaaaggtagattacaataaggatgtggagaaactggaaccctcataggctgctggtaggggtaaacagtggtatgtatacatatgtaacaaacctgcacattgtgcacatgtaccctagaacttaaagtgtaataaaaaaaaaaggaaatgaaataaaaggtacacaaacttaaaatgaaaagaaaaaaaaagagtggtgcagccactttggaaaacagtttggtagttcatcaaaaagttaaacagttaccaaatcacccagcaactccactcctaggtctatattcgagagacttgaaaacatacattcatgcaaaaacccatacactgatgttcatagcagcacagttcacaatgaccaagaggtgtaaacaacccaaatgtctatcaactgatgaacagataaaacgtggtatagccatagaatggaatattattctaccataaaaaggaatgatactagttacagaatggataaaccttaaagacattattatgttccgtgaaaaagctggatactaaatgccgcatgttgtctgatcccttttatatgaaatgtccagaataggcacattcaaagagacaaaaagtaatcagtggttgtcggggctgggggaagggaaattggggagtgatcccaaaggagtttgatttttggagggggaggtgatgaagatgttctgaaattatattgtgacaatagttgcacaactctgaatataccaaaacacactaaaatatacactataaaggggtatagaattgtatctaaataaagctgttattttaaaaattgtatgtttaattctaagtaaagacttgtcaatgagttgaaatattccatttcccaagtaagttgatagtttcttgttcttgcctaagaaagcgttagtttcaaatatgtattttaaatccagcaggaagtgaaactggattagcaagcgcacagccaagcctggaccctaaaagagaagtgaggaagggtaaggattcagaacacaaatccagaaagaagacaagtgtgatgagggagtcacaaggaagacccacaaggccagccatgagctcccccaggctaacacagccagcggtctcagggcagtgtggggaatcagccttgcccagggccggggccagggcagccccgctgtcttcctctcttcactgttatcacagccggagcagctgtgaagtgcccaggaggcagctcctcaaacggtgtttcaacaatgtgcccgagcagatgacgcagcaaaactattttctgtgcttcttggactctgataactggaaacaaactatgcagagtcatgttaggaggtgagggtttgaattagattgttttctctagctatgaagacttattctgttactgtattttgagctgtggtggatttgtctataattcctaggtggatttttaacttaccttaaaagctctgtgattgttcctttaacaaatatctaaataaatcagattaaaatttttgaacgtgatgaaactacagcttgactccagaatacataacctaagatataaaaaagacccagagggccaggcacgatggctcacgcctgtaatcccagcactttgaggggccgaggtgggcggatcacaaggtgaggagttcgagaccagcctgaccaacatggtgaaaccccatctctactaaaaatacaaagaagttagccaggcatggtggcgggcacctgtaatcccagctactcagaaggctgaggcaggagaatcgcttgaacctgggagatggaggttgcagtgagccgagaacgcgccactgcactccaaccggggcgacagagcaagactccatctcaaaaaaaaaaaaaaaaagaaagaaaaaagaaaagaaaagaaaagaaaaaaggacccagagagtctcggttggagacatcaagggtatgtctgagagcccttgggatctgcttagttctctatcagccccaggtaagatcccgcctaagctgttccacaacaatgaacatcagcccatgggcaatgccttttaaaaatgagttaaaagactcacacacaccttcattcattcaacagaactctgcatatgtagtctcagcctcatggagctgatagctagcccctcagaaatgtcacggtccctaactactctttgctatttatttatttattatttttattttatttatttattttttgagatggatgggattacaggcacatgcaccaccactcccagctaatttttgtatttttagtagagacggggtttcaccatgttgaccaggctggtcttgaatgcctgacctcaggtgatctgtctgccttggcctcccaaagtactctttgctatttttaacctatattatagatttaaatttttgttttgttttgtttgaatgaaaataagaagataatttcaggctgggcatggtatcgcacacctacattcccagcagtttgggaggccaaggtgggagggtcgcatgagcccaggaattcaagaccagcctgggccacatgatgagaccccgtctctaccaaaaaaaaaaaaaaaaaaaaaaagaagcagcagctaatttcattccttatttcattcctttatgcacaagcccctttaaaatacacttgacatgaatagcagagcttgcctccggctcctccatgcagaattcggcaatcccaggttcctcaccgctcctcagggtggaccttccattgacagtgtttaaaatcacagactgtggggtcagacaggcctggatgtggattctgggtctcctgctgatcccccgagtggacctcccgaagcctctcctgtgcagcaggtggtgctggagcctgagatgccggcatcgggccgggaggagctgggcctctgcccactgcagccatgacctctccaggcagccccgcccccctgtgtcgtgaagcgcaactgggcacaggacctccattaacagtccacgtctgtgctaaacttgatggaaaaaatggcctctccatagcgatttgtgtccttggtttgcaagcctcgaatttgcacctccttttcgaggtggcagtttagggttgactctggcagcattttccgaatgtgcctgcctgtgtgtccccaccttgttccaccccacaagggttgtgctgtaggagtcctggcctctgccagccctccctgatgggccctaatgcaatgaacgtcaaaggatctatcacaaaactagaaaggctgctgtctggtcctgagaacattgttctcttttcaatctctctttccagagagcaaacagtttataaagactgacaaattgctacacatagtcaagaagtagacaaaatcttgccattacacagaagagccataatgtcagctcttttcccttctggactttgctcagagagtcagtggacaaatgagcatcctgccagcagccttagtgtaaaaaccagccacccacaagctctgatgacctgaaaagtcaagtaccaggtgcattttcagatccagagatgtgtctgtcccatgttactctgggtatcaaattaattgaaacctatagaactttgaaatgctttaaaaccatcataaatataatggatggagcagtgaggacagaagcctgtctatcctgtgtgaggtgggcacacaggaaatacgtgttaaatatagaaaggaagaaaataaaggagggagagaagaagggagaagggaaaagaaggtagaaaagtagtcctatagtggaagaggaggggaagagacagaaggaggaggaggaggacagaggagggagggggagggggaagggctagggaggggaggagaagtgaggggggaaaggggaggggaggaaggggaggggggaggggggaggggaaggggagggagggggagggaaagggggaggggagggagggggagggaaagggggaggggagggagggggagggaaagggggaggggaaggagggggagggaaagggggaggggagggaggggggatggaaagggggaggggaggagaagcgaagccgggggagtggaaggggaggggagggagaagagaaggagggaggcaaaaaggggagtgggaggggactggggaggacggacaaggagaaagaaggaggaataagaattgcaggcgtggcccaggctggtactggggtgggggaagggagtgccccctgcagaacaggaggctgttgccctagggtatgcagggctcacctgaggagcctccttcattctgagccctgagcgcctcctctgcctcactccggtccccaccctgcagtggtgaagccaaagtcggtaaagcagtggctgctgtgtgttttctccaggtcagtgggacatggggctgctggacatctcagccctttggccgtggctctgccgagtttgatcgccacactcttccctgctgccaccagaagggtcacacaaggccactctcaggactcccagacctcatctgtctcagagaagaacgaggccactataaaagggtgatgtctttggccttagggggcgtgtaggcccctctggttgcgatgtggatagagttccagatgtgagggaatgttctggaaatgcaacctgggcagctgatggagtccaggcccttcgggtgctaacgctgtgaaacatctctgtttcagaaatgggtgccaagcctggagggcaggcttgcttagcaaagaaggcaaactctggtgagctacagctgggttcagcacatgggtctggtagggaaacacacattgaaacgttattaaaaccagcaagactgacacaggcggcagactgtgacgtctgtcccatgttacactgagcttgttttcaggtccggagatgtgccaaagccgtgccacactgggctggagccactcgtgaaatgtcattacgctcacacagctgcaaagttctcagtatttaccaactaggacttttcttgtattggtatttgtttaattgtcttctctcctcaacagtaactataatgtcctaaaactccgtagacaataaatagttctttaaagaccagatttagttttcatgtaacctacaggttggggaaggggaaccacaaattgcattgaccctcaaaacagtggctgaaatggaaagaactccttaaacggaacccttggggaatccatctgtaaagccccattgcctctcagattctgtgatctgggcctcgagacaaccttcagagtccctcgctgcatgagtgcatgtctggtcagagactttcaaaaactgaaatgccccaggccagcagccatccgctaacaaggaccgtggagaaaagcccatggatgcgtggagaaatggtgcccactagtggcgatctccagaatggacgtcctgtgaagtagatgccgctggccggcggtggctcacacctgccagcacctcaggaggctgatacggcgggggagtgtggggggcggggcgcattgcttgagtctggaaattcaagaccagcccgagcaacacagtgagaccctacctctacaaaacatacaaaaactagccggatgtggtggtgcctatggtcccagctactcaggaggctgaggtgggaagattgcttgagcctgggaggtttaggctgcagtgagcagagatcacaccaacgcactccagcctgggtgactgagggagatcttgtctcaaaaacaaacaaacaaaaaagatccggctgactgaagtacccaggcaaattaaccagacacttggagattttctcattttctacaaggctgaggagaaattttaaggagcccacaattccaacaccccatcactctttctcatgtttttcccaatattttcatcaaatcataaattgtggcaaaataatgtataattctcaatacatacaaagtaactcagaataaggaggactgatcaagaagaatgtaaccaatcaccaagtgccacattcactgaaatcagcaattcataagcatagttatatgtgtggtgttgggcaggggggctgtggtgtgtgtgtgtggtctgtgttgtgtacaccagtgtgtggtgtgtgacgtctgtggggtatgtgtgtggtatgttgtgtgtggactagtgtgcagtgtgtagtgtgtctgtgaggggtgtgcactagtgtgtggtgtgtgtgtctgtggggtgtgtgtgtggtatattgtgtggagtatgtctgttgggtttgcactagtatgtggggtgtggtatgttgtgtgtggtgtgtgtggactagtgtgcgatgtgtagtgtgtctgtgggggggtgtgcaccagtgtggggggtgtgtgtgtgtgtgtctatctccagattagatgatttcctgctttttttcagtgatatactgctgtcatttttaaaaattatcctcagcactatgtttcagacaataatgataacatgtcttggcataagcaagctgtgtgttaataaaatatttttgacactgagcaacacctgtaagcattgcaaactatccagaggcagttgtttttttcacacatcactgtaggaagcttttcctcattcatcttatgggccctcactcactgcgttccagccagactaggtttctttgcattccctgaatgccctgggccttctcccacctccactccctcccctatccactggttcaaccaaatccttcagttccatcttggcgtccgtttgaggaccgccactgtggcctccacaggcgtcaagcctgtgttcgcagggccctccacaatctgcactccgtcacccgctcctggcagaacactggcacaggagaccaaagtacacgtggatgagtcaatcagaagaacaacttgtactagaaagtgctttcccaaatttgacatcaaaattgagaccagtgaacactttcatggctcatgacaaagttcatatccacctaacacaaaacgagggcattattcagttaatttgatatactaaaaagataatcagataaccagctagagattatgacacttctgaattggctgttaccctcatgtcgacggggctggacccgtgtctgtggacagcagtctttgagaagctgcagcgggagcctgtgcaggatgactagtgttgcagtgtcctgggggtggtgggacaggatgctaggaagaaaaggcgtccggcacctcaggatgctctgtccgggtggctcagcttgcttcccgtagcctcccagtccttttactgaagtttcaggaggtgatgcttgggacgccaggcctggcagattcctctccaggcttcccttcaccccgcagagcttccccgcactggcagaactcgatcactgccacccagagggcattgtctctgaagaggcatggagagtggggaagctgcaaaccccaggctgatgttcttggtgtccccacagcctgcttttgccttaaaaccccaggtatactcacaaatgggtacaacacccaagaggccattgtcagctcagggacttggttttaggtgcggctttgggaacttgcttaggtaagttagccttgctgggtcttattttccacatcagtaagtgccacgaagcttaacatttataagccaacagaatgcctcaattttaattaacaaacattaattatcaatcattttttacctggatggaggttaattcaaaggaaaggtattaacccagagggtttttctttttaagagtgttattgtggtgcccatgtcccggcggggaagctctgagcttcctctgcccaactctgcaggcgacgcttatggccccgtctgtggcgtccaggtgcacctggacaggtctccaaggcagcgctggagcagatttgcagccacagggtgtggagaaatgcaagatgaattgagacttgatgtttggatagatgttaattccaaacttgagaactgtctttgtaaaatgacaacaaagacgtatctgccaatcattgacttcttaatcactgcctttgctctttcaaagcctcaacattttgcagtttcagccaacagaattctctaactttgtaaactgctctcctgtggcgacaataccacctcactctaatcagtgagaagggtgattgttctctcttcctttcagagtaatttaaattcttgagaaaccacttggcatgtttaatattctggtcatactgaaaatccaagtcttgttttcctgaccgtatttcaggaactagtttaaggacggaattatcttttaaaaccacaaaacaaagatttttacacactgaatttcacaaaactcagtattgcagaattgcaaacatgctctgtgactcagtctccagggcttaacttcccccttgtagtaactttagagggtcctgaaattttagcttctttaacaagggcaaatactgggagagaatgtaaggggaggaaaataaacagaatgtcttctgttagacaagccagcctctggcatgtacacatttggcagagttctaattttgtttgctaacctccgatcactacctgaaatgggtctctgcaggatatcaaggtgtcagcagcgctgtgctcctggagcatctaggtgagaatcccgtccctggcctttcccagcttcaggaggccacctgcactcccagctcactgttcttccttctttccttcctccatcttcaaagccagcggcgtagcgtcttccagtctctttgactctgaccccccggcctccctctttcacttgtaagaactcttgtgattacatcatacccacccacataacccatcttaacttaatcaccccttgcaaagtccattttgtcgtgtgaggtaatacacaatatttatgagccctatcagtttaaccaattagtaagtgtggtagaggaaatacaacgatggaaggtttaacctgaggaaagaaaaatagctcagagcagtctgaactatttgaaggatgcaaaatgtatcaggacatgagtgtgggactaacttcagtcactctgctccccatgccacaggggcaattgtttaaggcattttgttcctgactgctgtgtcacccattattttcatgtttctggagtctgtgagacaaagaaaaatgtatagtcaatcaatagcttacgttgttttaatgtaaatttttgataaacaactcaggaactgccctttctttcctccttaaaccccccgcccccaaccaggtacctgctgctggttgggtgcatactcagggaaattcaaatctgtgctcccaggtggtcatcctcagggcttgtgctcaaataaactccatgctaactcgtattttctgaatctttctatctaaggttgacaaacctaaggggcatcattctaaaatcagtaaacatgattttgctgtaggctattacaggtatagaagggaagaaaagcacaagaaatctaaccaaatatagagactccaggtcattctattagaatgtgatgtggtttggatctgtgtccccaccaaagctcatgtggaattataatccctgctgctggaggtggggctggtgggaggtggttggatcgtgggggtggagtgcccattgctgctggcggtggggctggtgggaggtggttggatcgtgtgggtggagtgcccattgctgctggaggtggggctggtgggaggtggttggatcgtgcgggtggagtgcccattgctgctggcggtggggctggtgggaggtggttggatcgtgggggtggagtgcccattgctgctggaggtggggctggtgggaggtggttggatcgtgggggtggagtgcccattgctgctggaggtggggctggtgggaggtggttggatcgtgggggtggagtgcccattgctgctggcggtggggctggtgggaggtggttggatcgtgcgggtggagtgcccattgctgctggaggtggggctggtgggaggtggttggatcgtgcgggtggagtgctcgtgaagggtttagcaccatcctctacttgggactgtatagtgagtgagttcttgtgaggcctggttgttgaaaagtgtgtggcacctgccccctctctccctttctcttgttctggccaggtaagttgtgtctgcttccccttcgcctttcgccatgattgtaagtttcctgagacctgcacagaagcagaagcagctatgcttcctgtacagcctgcagaacagtaaaccaattaaacctcttttcttataaattacccagtctcaggtgtttctttttatcttttttttttttgagacggagtttcactcttgttgcccaggctggagtgcagtggtgcaatcttggctcactgcaacctccgcctctggggttcaagtgactctcctgcctcagcctcccgagtagctgggactacgggcatctgccaccatacccggctaattttttgtagttttagtagagatggggtttcagtatgttgcccaggctggtcttgatctcctgacctcacatgatccagctgcctcggactcccaaagtgctgggattaaaggaatgagccaccgggcctggcctcaggtgtttctttacaggaatgtgagaacaggctaatacagaaaattggtactgaaaagtggggcattcgtatgaagatacctgaaaatgcggaaataactttggaactgggtaatgggcagaggttggaaaaatgtggggggctcagaagaagacaagaagatgggggaaaatttggaacttcctagagacttttaaattgttgtgacccaaatgctgatagtgatatggagagatgggcaggctgataaggtctcagatagagatgagaaacttactaggaactgaagcaaaggtcacttttgctgtgcattagcaaagaacctggcagcattgtgcccctgctctagggatctctggaactttgaacttgagagtgatgagttagggtatctggcagaggactcctgaaagtgatttctgttaaatctcgccctggcaatattaattacaaatttaccttccaggtacaaaacaaggacaagatgagattagtcactcttctgcctaccctgatatgggctacataactctttcctctactccctcttgtcagatgtttgcctcatcttatgtaaattgttggtttactgagcactaatcagagcatcactgtggacccctccctttttttcctcctgcttgccgtctcctcctaaacacccagtccccataaccctatttggatggcacagttgatgcttctgtggcttgcgtttttcccgggcccatcatcagacttctactgatggagacatttgcctcagtcactcatttttggttaactaggaatctcattaaatggatagaagtcgttgaaaatatggcttaaaacacacctcaccacatgcctgccccaagttaatgagcagagttgattactgcctgcttgcagaagtgggaaaggaggaggaaaaggaaatatacagggggacaaatttttaaacacaacttcaaaatattacccagtcatatatgtatacacacacatagaaaattacacggcagattacttacagtagcgtgagagaatttttgagtgggtgggattctttacagcaagtttttttttttttgcttctttgaattttttttactcttttaggtttttatatttataaaagttatatttactttttaaaagtgtcaaccttaatattgagattcagaaatatgattacatatagtgtgtatttgagcacagagcctgagaatggccacctggacaaatggggtcagcctttccaagtggagctcaggtctctcttaccaggcggagacagaagctccagcaggatcaccacagttctcatatgagaccaggggcacacgccgcagcggttccattggttagggatctctgtatttgaaggaagattgcgttataactccatgaggaggggtagtgatctgagggggtcttatctccggtgccatttggtctttattgtttacagggaaaaaggcagaagttacagctgggtgtggcatgacctaggccgcgtagccacattcccctcaaggctcaggtaattcaaagttccaacagcttcaagtttaaattattttaagtttgaatgatttaatttcacaaaaggtaatatatgcacattgactgaaattcaaaagcacaaaaagatatccaaggaagtacctctcccacttctaccatctctcatcttccagcactccttccagaggccttgcgctatgccggttttctcatttttccttcctgggacagtttagggatttttctacctatcattaaattcgtattttactcaaatgcaggcatacgataaacacgatcctatacttgctttttcccacatactatatcttgaagatcgttcaatgtccatgcaaagctgactgttcttttgaacagccagcgagtattccattacgtgagcgtatcatgacctatttactcagttccatacttcatgggtgtttaaattgtctgcaatcttttgctattacaaacgaaaccacaggtcattttgcacacatgcaaaaatgcaaatccatggtatatttcccaaaataaaaatcattaggtttaacagcacgaacacctttaaatttggaagatattccaattcgttcatcctagagagtataccaataccactcacaaaacttgagggtgcctgcttcccgacattcctgctagcagtgtcatcaacctttgtgatctttgttaacatatgaaaagtggtactctaattttaatttttattatgaattaagttggactttctttcgtatgttaaagtctatttgcatttctttccctgtgaactgttttttgcctgtctttctcctgagccattggtctcttcccactgacttattacatatatgtaaagggaatattggctctacattataaggaaattcgaattttgtgataggtttcaaatttttccctaggttgtctttgacttcatttgctgtttgtgcacacaattttttttttttgtaatgagagtgcgttgctcttatttttaaaatgttgactacattctggggaaagaaggctcagcagccacctgcttttttgcccgggtgggtggtccggccccgagccctcctgactctctcgccaatgcccagaggcgccgcagcgattccagggaggccgcgctctcgccccaaggcaaccagaagcccacgtgccaggagaggcatcggaaccaacaactcgggtccgttttcccgcgccagatgggattcgtttgctgtggacttccggggcggggcctgcctggagcggaagagcctgggcagtgcacggggcctgggtggggggtgcgggtgtgggtggggacctgcggccttcgagtccgcggccttcgagtcctggggcggcggcggcggctgcaggcacgggcacgggcacggggcggggtgcttagggtgcaggaggcgcgcgcctagcggcggagtgtggcgtgaggccgggcccgcgccgccatgaacctagagcggctgcggaagcgcgtccggcagtacctcgaccaggtgggcggccccgactcggggtgcggggcccaggcctcgccgggtctttttccgcgcggcgtggggctggggtgactgttgtcggggctcttggtggggaaggactgggccggccctgggccttcatctggggccagcagcgctgtctccgcctcgcccagaagggagcggtccctgcagctgggaggggcacagcccggggagtggaggcccccgagcgcacggggcgcctcagtcgggtctgagcctcgttctgcacctggcgcggaggaggtgcccaggaaacactgactactcagtgcaagttgaaggacaatttttggaggaaagttgcagggctagctaataaaacatttttgagaggtttagttgtaatttttactgggctgaatttaagtgaataaagatgtgaagaagcctgttgtctcaaatcaatactttaaaaattctcgttaactatgtctgtgttctctgcactacctttgaatagcagagagcgtttaaggcaagaagtgtcttcgagaatcagcagcatcagttttatctatattgttgtttattttttgttgaaatgcttgctctttcaggaaattggaaagaaagggaaagaaaagccatggtaatgcagagttttcggaattgggtgctaactgacgtggccaaatgtttgcagtccatttggagggctgtgtttttatttaccatgagtcagttgaattcagtgttttctgatcactttttcttcggtacggagacgtagtgaggaatatgacaggcccggtttccagccagagacaccggtattgagcatatttataactatcccgttggcattcctcctgcctcgggctagcagcagtggtcctggcttgtctccaggggaactgaggcagagactgtagaagactgaggactgaggagcagaacagggtctaaagctagccgtaggtcactgtgggtggccctcattgtcggctccttcatttttgttagacttggaggaggtgagttcttccccatgttcagtgaagcttagttaatttatgtgtgtgtgtttttaaacttgagtgttattaaaactgccactgtgttatataaggtatttgagtatagtgttctgtcttctaaatttcactttgggattcattaacagctcgttgaaagtgttatataaatcacacatgaattgttaatgactagcttttgctgtttcacttgcatttcacttgtatgtttcacgatacatttttacagtgtatgagttttaaaaaattaccttcataagttgcatatacaaagatattacaggaatgtttgtattgcgacactgaactaaaatttaccctacgtgagatgtaagatagagatgtaacaaattgttgtcttgagatgaagatttattacatagaattatattaatatattacatagaattataataatattagataactgtttaagactattaaaataacagggcagttaccacctttttttttttttggtatgcagcaacagtatcaaagtgctctattttgggcagataaagtagcttcactctctcgtggtaagtgacaaaatgctaactggttttctgattaatcttaaaattcgttttctatttcaccttgtacctctgaccacttatgtgaatttttccctccagaagaaccccaggacatctattggttggctcagtgtctttacctgacagcacaatatcacagagccgcccatgcacttcggtcacgaaaactggacaaagtaagtgatggtatgaactttaaagctcttcagagctttaaaaaaaatgtcattagtggccgggcgcggtggcttacacctgtaatcccagcgctttggaaagccaaggcaggtggatcatgaggtcaggagctcaagaccaagatggtgaaaccccatctccgctaaaaatacaaaaattagccggacgtggtggcaggcgcctgtaatcccagctactcgggaggctgaggcagagaattacttgaacctgggaggtggaggttgcagtgagctgagttcgtgcattgcactccagcctggacaacagagcaagactctgtctcaaaaaaaaaaaaagtcattagggttttaggtgtatattccatttctgttgaaattatttaacttgatgcaatttttctttgtttttgtttttttgaggtctctctgtgtcacccaggagtgcagtgggatgatcgtggctcactataacctcagactcctgagttcaagtgatccttctgcctcagcctccctaatagctgggactacaggtgcatgccaccacacccagctaatttttaaatttttttggtagagacagagcctcactatgttgcccaggctagtcttgaactcctggtctcaagcagtcctcccgccttggtctcccaaagtgctgggattatagatgtgagtcatcacagtaggcccagtttttttttagtgaaagtgaagggaaaatgtcttatacctaatctgtcaccttctttttcccccatctcccatctagtctctaaattctgaagactgttttctaaacagctctggaatcatggatattcctcttcctccccactcgtacgtgtcaggcctttgatttttttgtctgttctgttacagcctctcttcttgtctcttttccccagtaacagtttctctcttcttatcttccctgtcacaagttcatcttgtgaactattacaggagtgagtagtctttaaaacacggatgtggttatattcccacaaagtcattcagtgacttcccattagctgcaggataaagtcagatgcatcagtaggtctgtgaggccctccttgttttctctccactctgcgtctaatccaccaccatcaatacctggagcacgtgctccagtcacgtggtgctgctgtgagctccttggacacccagcagttattcacactcagggcctgaagtggagctgctgcgatctccttggacgcccagcagttattcacactcagtgcctgaagtggagctgctgtgagctcctcggacgcccagcagttattcacactgagggcctgaagtggagctgctgcgatctcctcggacgcccagcagttattcacactcagtgcctgaagtggagctgctgtgagctcctcggacgcccagcagttattcacactcagggcctgaagtggagctgctgcgatctcctcggatgcccagcagttattcacactcagtgcctgaagtggagctgctgcgagctcctcggacgcccagcagttattcacactcagggcctgaagtggagctgctgcgagctcctcggacgcccagcagttattcacactcagggcctgaagtggagctgctgcgatctcctcggacgcccagcagttattcacactcagtgcctgaagtggagctgctgcgatctccttggacccccagcagttattcacactcagtgcttgaagagctacccagtgctccactcctctgcctgaccacttactcgctgcctcacttactcgctctcctgcagatgtttacttctccacctggttgatgctttctgttgccccgtttcgtcatgcttacctgttgtgcttagttcacatctcccctactgtaacctcctggggcagggccttactgcttttgaatccttggtgcctagtaaacagtaggttcataataaatatggtttaacaggactggattttttttttaactgcaagagtaaaaggagaaattatgaaattagttctattttatgcttatggctttctttgaaagtgaagatacatattattttgaccactacttaaacaaattaatttctttcctagttgtatgaagcatgtcgttaccttgcagctaggtgccatgtaagtatgctcataatttcatttttatttggttgaataaaatgagtgttatgcatctcttaaatatgtgtgagaattttagtgatggagcttagcaggttagtaaagtatttcatgaggaagtgttcctttccttagtcatgtttgctataaaatactgattcttcgtttaccacatgctgacatgcatgaagaaaattctactttagacatttcacatttaaataatgggcatagtatatgttatcctttaaaaattcggatcatgtgtttcgtgttagaagtcgtgagtgatgagcggcacttctgttttccacgtagtatgctgcaaaagagcaccagcaggcccttgatgttcttgacatggaagagcccatcaataaaagattatttgaaaaatacttgaaggacgaaagtggcttcaaagatccttccagcgactgggaaatgtcacagtcttcagtaagtagtactgtgagcacagctcagtaacggcggcgagaattgccctcattacgtggcagaaacacattatcttcttttacttattactatattaaaacaattgtggttgccaatttatttaatttgcccaccaaaaccaaaaaagattctgagacatttctgagcccttagttgcaagctgtaattgtatccgcagatgcctgcatccaaaataggctggcttgaaacttccttctcatatttgatgccttaagctactccctaaaagtggaataatttttcttctcctttctgtgtttcagcagtgttttggacctacaccaagaaactttgattagcgtacattgtttctttccaggaaatggttttgtgttctgtttgcttcaaagtacattgctatgtaattgtgtatatttcagtttgtatacttcatatctccatgaaattgcacattccttgctggcagacatcatatagtaacttattaagtgacatttacagatcactaatctggatttaaaaaaaaaaataaaaacttattgaggtataatttatatgccatgcagttcccccatttaaactgtatggtttgattccttttagtgtattcacagagctttgcaaacctcaccatggtcaattttagaatatttttattattcagaaagaatccctgtaagccttacctgacctaccaatttctctttcttcccacatcttttttttttaaattaagacagggtctttgctttgttgtccaggctggagtgcagtggcacaatcacagctcactgcagcctcaacctcccaagctcaagtgattctcccacctcagcctcccgagtagctgggactacaggcgcccgccaccatgcccggctaattttttgctattttagtagagacggggtttcaccatgttagccaggatggtctcgatctcctgacctcgtggtttgcccacctcggcctcccaaagtgctgggattacaggtgtgagccactgtgcccggcacagttggctaaattttttaaaaaatgttttgtagagacagggtctcactgtgttgcccaggatggtctcaaactcctgggctcaagtgatcctcctgccttagtttcccaaagtgttgggattacagtcatgagccactgcacctggcccatttatttatatttttggtagaagtttattacttgacttttcaccttagatagttatttttaagcataaaagaatattacacattctaggtttttgtttttgtttttttttctggccactcattaatgtatgtgttgggaagcagattcttctgttaagaaatgcatttatgaaagtgtgtggtacagtcattattttgggacctcttatccattaacttatttgctatgaaatgtgtgatttatttgcaacgcattattatttattttgtgaaattacatttttaaaaaacaactcctaggatattgcttagttcgaagactctcataattcattattttttctaaaattttactttatgaatcttcttcatatccttaatgatactagctgcctgttgctggatcatctttaaattcagttatgtgttcgaggtccagagtctagaatgtgcatcagatttccacatcttcgtgtttatggtagtcgtgagatggctgttgaacgccttactatgtgctagcaccacagagatggtagcatctctagccctcacagacttctataagttagatagtaaagagttctataagttagatagtgtttttatctttttacagatgaggataggaaatggaggccaagagaggttcaatcacttgtccgaggttacaagtaattggcagagctggggtttgaactcaggtctgtgctgcctcagcaggccattgtgctgactgctgtgaccactctgctgcattgctgccagtttctgctcattaagatccttttgaggggtgtctgcctatcacactgtcaatcttggagttttatacacatgcacacacactctgccatctctaaatcattgcactgttttggacaaaacaggtaatgaacctagtagccgtgacatgtacttttgttataaagatgtgttggaacagagcagttgaagcccagggaaggtgaattgaagaagctttgctgtgcattttttgtgtgtttttatttatgccttgacagttgtgattgactaaatccttttaaatgcaataaaaacttctcagcattcaaatacacaattctctatcaaaagttaagtttaagggccaggcatggttgctcatgcctgtaatctcagcacttcgggaggccaaagtgggaggatcacttgaggccaggagtttgagagcagcctaggcaacatagcaagattgtctgtacaaaaaatttaaaaaattaactgtttgtggtattgtgcacctgtaatcctagctactcaggaggctgaggcaggagggtcatttgagatcataggagttcaaggctgcagtgagctatgatcgcaccactgaactccagcctgggagacagagtgagactctgcctctaaggagaaaaaaaaaaagttaagtttaaaagtactgacttaatatgtgactcatcatttttccaacagataaagagttctatctgtcttctacgcgggaaaatctatgatgctctagataaccgaaccctggctacctacagctacaaagaagctttgaagcttgatgtctactgttttgaagcgttcgatcttttaacatcacatcacatgctgacagcacaagaaggtttggaaactcaggcttttttgttttatgtttagcaaaattaatattgtttggattttttgcctctgaaatcttctaagtcaacaacaggccacatacaggtttaaatagacctactcactttcttaatgtcttcaaatgtaaagcacgttgcagaatgcagtattgtacggtgccttgtgctgtccgaatctagggattgtgataggaagaggccatctttttgagaagtgattgagatagtataaaccaggatttcagtatctcatgcagtgattctcaaccgtgttgctctttacatccttgggaagacagagaaacatccatgtccagttcccaactccagagcagatgctttaaagctcccaggtgatcctaatgtaccacaggcactgagaatcagtccaagtatgttggcagtggtgtgaggtgcgtcaacaagggaaacagaaaatagagtggttggattaatgggattctaacatattttagcctagtgcagtggttctcaaccaaccagagtgattttgccgccccctgccccgggaacagttggcaatgtctggagatgttgtttgtcacagcttaggggcagggagggaggttgagaggttgctgctggtatctagtggttagtggccagaggtcctgcttagcacctgcagtacacaggctcccctgttcccagcagagcaagatccacgcagtcaggagtgcggaggctggtggttcttaggtgacaagagccacctggggacctgcctcagccctgaaagagtagcctccacccaccagatgattcccgtcttcaggcctggagccactgtgtcagcttctgttgcacattttgatcttatgttctcttttgagtttgatttacttagaccaagaactgtagaatcatatttttagttgttggctttttcccctctataaatattatgaggatattcttttttttctcaccatttttaagaaaaagaacttcttgaatcactaccccttagcaagctgtgtaatgaagaacaggaattgctgcgttttctatttgagaacaaattgaaaaaagtaagtaaaaccaaagagttagcacttgctttatgtaaatatcttattccataaaatttggcatctagtcttatattaagttttcaaaattaaaactttttaaatttagcacatttttaagaatagagcgtttaagatacataagattccaaaaaaaaaaagctttcaaaaatacctttttcctctgaaagactccttatactaaaagattgaattaccaagtaaatgtaaatggaatggtgttgcatggaaatgaagatttttttgttatgcttcctgaatgtttactttgagaaatacgtcccgctgtggaattctgttctaaaattgagaagggaattaaacttactgagaggagtacttataaaacctaaagttataagttttcagaggctctcacatttgttcttgtaaatgttttaaccacttgggccaaacacaaattgaatccacattttattttgtttttgctcattgagtttatgtttacatttctatgtgtttcagtataataagcctagtgaaacggtcatccctgaatctgtagatggcttgcaagagaatctggatgtggtagtgtctttagctgagagacattattataactgtgattttaaaatgtgctacaagcttacttctgtgtaagtatatccatccatttttctgtaggaacatggagttcactccatcttacctaggtgattcacggacgtgctctctgaataatcttgaactagatgataatttaagttatctttaccaattgtagagcactgaacagggtgctgtcttaaaagagcaatttacagacatgaatttgtgcatatttgtagatttattacattaacttgggagggatatacatgttgctcttactgatcttgataatttgcaagtttgaaaattccattttggcacattgttttgtaaaatttattctcaattatgagactcacactgtagatactaggtgtttaatatgctgcacgcagtagctaagccatctcccacatgtatcagttttccagtgtacctaatttaaactttagctaaatgacatatgctattttttgtagatcttggttcggtttaaaatccttgaaattacaaaaattttaattggcaaatacacataacctatttattagtttggtgcaaaagtaattgcagtttttgctgttgaacgtagtggtaaaactgcagttatttttgcaccaacctaataggatttcctgttggttttgattcttttaaataagcggcctacttattttatgtattacaattctgttgacttaaagtaaatgtatgtgataatgtttatttttattcctttcaaataaaccagaattttttctttcatttgtaacttaattatggccttaactgaatcaacctgaagataatggagtgaagaaagtgaagtgttacaacccacagcatcatatgtgataccataatgactgtctacacatagccgatcgtcgtgagtgctgtcacctgctggtttgggggtagtaatgtgtcgctttaactttcagagtaatggagaaagatcctttccatgcaagttgtttacctgtacatatagggacgcttgtagagctgaataaagccaatggtaagacttttttttaaattaaagtaattcttagacataaaacaaatcttttctgtaacttgaaattttgtctctgaaattctggatagttgagattgcaggtaacaacataatggaaccctactataaaagtaacaactggggaggaaatgtttaggaaagtagcaaatggccaattttgttttcccatcagaacaattagttctttatcactgcttcctactaccttttctcaaatatcatttatgtttcaaggtttgttagctttaaattgttgggtgtcagttctgcctcccccccccttttttttttttgtaaattccactggaaatctgatatggtaagagttgataactacttagaaattggaaaggagcagattgtttgctcatgttttccgtatttttaacatttgacatttagttgtgtttggggatgcatttgtccttaaatttttctaaactgaatggtgagctctgtcaggaaggaaaaccctcaccgaatccctgctgctcagagtgtggttcgtggaccagcaacatcgcatttgtggaagctggttggaaatgcagcatctccagctgctctggtctactgaatccaagtctgcatgcaggaaacatcatcttgaaatgccaggatttacacatttctcaagattaaatagtatcattgatctgataaacttgaaagtttgttcagccaaattggttgtgggtgattatgatggtccataatgggcccttggtgtgacaggcatgagcttctaagagaggtgaggtgctttgctgtatcctctgcagtattcgggaagtgctgtcttagggggtgtctgttcccaccttgcccacagtgtgtccttcatctaactctccagttagagagttaattttttatctagaaggaaagacaagtatgaaatcattgctgtaagagcagaattatatgattgtgaagagttgtaatgaaatcatgtcttaaattacatgtgatacgaacaattgttctttttctgctttttcctgaacagaacttttctatctttctcataaactggtggatttatatcctagtaatcctgtaagtaatataacttttagtcttagtttttttttttttacctgtactttaagaaaatgcttaactcgtatttagacaatagcttaagttcaagcaaatgatgttttattaaagtgttgcaacagaatctcctagtgtttattgtacttacaaaagcagcattaacttttcaacccatgaaggtattttttaattataccaaagcaaaaaggtttttaaacccttttttctgattatagaagttatacatgcttattttaaaatacttagcgtatagcaatataaaatactgaccatgacagtcacgcctccctttcctaagaaatgtggataattgtttggcagctttctcacattgcacatgtaagacaacctgttgaattgtgtactgattccaaactgtcatggatcggaatggagaactaggaatggaaaatggaatcagggactgcgtctttaaggacaaaatcaggaatcagggtgtaagactcagacgacaagaatttgcaccctatcccagtgtgaaaccagagcaagagaaaaccagctaaatagtcagaactcggacagagagcaaggaagagccagggtcaggctgggggagatgagcagtgcaggggagtcaggactcacaggctggtttttgaaacagggactcctcaggggccttgtcttgtgtagacgtctccttgagaaagagccatggagtgtctgccctcttctcttggaccttagtagactgtattagagaaatgtctaacttaactagaatatgtgataaggaacatgtagtataccctctgtctaccctaatttgttgcagattccaattagatattccaattccaatctttgtttatggtaaattttgaactttttaggtgtcttggtttgcagtgggatgttactatctcatggtcggtcataaaaatgaacatgccagaagatatctcaggtatgaatttatttttttcctctctagttaacctgtagaattgatgtcttgctcataagctttcaagtagtaattagtgagtactttaaaagcaatgtgaaagaataaatgtttttttttttcttcctttcttttctttctctctttctttcttgccttttcttttccttctcacctcccctcccctctctctcgagggttctcactctgttgcccagactagagtgcagtggtgcaataatagctcacctcaaactcaaatgatcctcttgctcagcctcccgagtagctgggactacaggcaccaccatgcctagctgatttaaacatttttttttttttttagtagagacgaatctcactgtgttgcctaggctggtcttgaactcctggcctcaagcagtcctcttgcttcggcctccgaaagtgttgagatgacaggcatgagccaccatgcctggctgaataaatgttttcttcaagtggtttgttctttataagatgccatggggagagagagaaaaaaaaaatcagtgatgtagaccctattctcagggaacctacatattttatttattaatgagaataaatggccaggcatggtggctcacacctgttatgccagcactttgggaggtcaatgcaggtggatcacctgaggtcaggagttcaagaccagcctgactaacatggtgaaactctgtctactaaaaatacaaaaaattagccagatatagtagtgggcatctgcatcccagctactcgggaggctgagacaggagaatcgcttgaacctggaaagtggaggttgcagtgagctgatatcgcaccattgcactccagtctgggtgacaagagcaaaactctgtctcaaaaaaataataataataattaataaatgagaataaatatgaaacaatagagaataatgtagtttgaaagtttctctctttcgcttacagagatcctgttaaccttttacttttaggatttcttttattaatagttgtgaaattacacaaaactttatgtaaaattggggtcactaaatgtgactataaaacagaattcttagcagggtgaactgattcatgtaatgatttgctgtggttttaacttaaggaaagccatgcagcctagtgaagctatttaaccagtattattcaagtttagaaaaatgtcaagtatgtgaagaattaattacttaaactctgtaaatgttctaatttctgtggtttcttccgtgcaaataaaacagaattggtctctaacggaaacagcataaactttggagtcaaatttgaattctaatccaagttctgtgacttaccaactgaaattccttgggaaagttaatttctcctgctctctctttctttaaaaatggggatgtcagccaggcgtggtggctcacgcctgtaatcccaacactttgggaggccgagacaggcagattaccttaggtcaggagttcgagaccagcctggccaacatggtgaaagtctgtctctactaaaaataaaaaaatcagctggacatggtggcatgcacctgtaatccccgctacttgggaggctgagggaggagaattgtttgagtccaggaggtggaggttgtagtgagctgagattgcactactgcactccagcctcgccgacagagtgagactctgtctcaaaaaaaataaaagggagtgggggtgggggagatgtcatactcatatagcaggattgttaggaagactgatgccacgaaagtaccctgtactaggaagaaactagaatgttccctggttggttgtatgttcagcattttagaagaacttctacatagaaagatgattagtattgtaaaagttattttgagctatgctgctttgatctagcgtgcaattcagtgatggactcttatcagtggtccccaacatttttggcaccagggaccattttgtagaagccccccctcccagggtggggggactggtttcttgatgattcaagcgcattacatttatcgtgtgcttttttctattattattacattgtagcatataatgaaataattatacaactcactataatgaagaatcagtgggagccctgagcttgttttcctgcaactagatggtcccatctgggagagatgggagacagcgacaggtcatcaggcattagattctcgtaaggagtgcgccacctagatccctcacatgtgcagttaacaatagggtttgcgcacttacgagaacctaatgcctctgatctgacaggaggcagagctcaggcggaaatgcaagtgaaggggagtggctgtaaatacagattaagcttttttttttttcaaatggcaagttctttcttgacatatgaagctttactcacctgcctgccactcacctcctgctgtgccgcctggttcctaataggccagggactgataccagtctgtggcccaggggttgggacccctgctctacatgaaacaggtaacatttggataaaagaaaacttaaaatacaaggaagtagggtatcaccccctatgtcttccacgttttgtgatatgttctctttattccttgtgggttaattatacagtgttcactgtatacaattattcactgatatatatgagtccagtccctgtaaccagggtatatctgttgaccatcatctataggatatcgcctaatccctcaagccttggtatacctgtctttaaaatataaatgaatttatcttgcaagttattaggatgagaggatgtatctactaggttacaaaatggagctcctggattaagtaatatctgtaagttctatttagaatgctaatgggttttgctacagtgcataaaaagctgtggatctcctcaaatttcttaaaattttgctatactattaaacagttttagttcaaatgaatcaacacaaatgttacattagtgtttaatgtggtgacagcttgtttactaaatggatgttaatgtttttgaggccatctccttatataaaattaacaattcaagaataaatactagctgggcacagtggctcgtgcctgtaatctcagtgactcaagagcctgaggcagaaggattacttgagcccaggattttgaggctgcagtgagctatgatcataccactgcattccagcctccagcgacagagcaaaactctgtctctaaaaataaaaaataataaataggctgggcgcagtggctcacgcctgtaatcccagcactttgggaggccaaggtgggtggagcacctgaggtcaggagttcaagaccagcctgaccgacatggtgaaaccccatctctactgtgaatacaaaattagccgggcatggtggcgggtgcctgtaatcccagctactcgggaggttgaggtaggagaattgcttggtatctggaggcggaggctgcagagagccgagatcgcaccactgcactccagcctgagcgacaaagtgagactttgtctcaaaaaaaaaaaaaaaagaataaatactatatgacttaaattaactcttttgttttagcaaagccacaacacttgagaaaacctatggacctgcatggatagcctatggacattcatttgcggtggagagtgagcacgaccaagcgatggctgcttacttcacagcagcacagctgatgaaagggtacggcagagcaaactcatcaaactccatgaagggatgtttttcctaatagaaatgaaatttctattactattactgctatttggttgctaaactaaacacaaacattttacttgccttttaatctagttgcttgtgattgtaaaataactatgaaagtggaggggagagaagctggtgtatgcatttgcctagcagttgattccaaccagtgcaagaagcaaccaggaaattaggccagtggaggagaggagttctggaagcccttcctcacatgctcatgcaagaataaaaatattcacgagcaaaattattatatcttatctattaccaaaaggtatttgatgtggcttagtggtaggtgcaggactgtatttgccagtattctagatttatatcaatattaaatgtattgaaactaagaatgtgttactttgaggagtagagtattgctgttcacattgactgtttggtctcaccatcggttacaacttacagggttggtttgtgtggactgcttctattgactgatgactgcatccctgcctggagaggtagtgggagtgcagtggtccagaggcctggtctggagtcatacctcctggactcttcctggctcttccactcccagccatgtgacgttgggcaagttttttagcctcctgagaaataggaataatagcaaaatttactcactggcttattgtgagaagtaattgagataataccaattaaagactttgaagagtgtcttacacatagaaagcactcagtgaaagttagctacttgtgtttatttacttgtatgcaggggtggaatcatatatttatctgtattgaatgtcatcctgttaggtttggtctctttgcaaactgttaggctctttttgtcactcagttctgcgtttaaattagtacccacctcagtcagtagtcatagtaacttggtaagtatcatagctccttagctgtattagtcagtagggctgccataacaaaataccacagactgggtgacttaaaggacatttattttttcatagttgtggaggttgagaagtccaagatcagggtgctggccagtgcagttcctattgaggagtctcttcctggtttgtagataactgccttcctgctgtgtcctcacatggcagagagagctctggtgtctcttcctcttgtaaaaacactagccctattggatgagaaccttacacgaaccctattggataaaagccttacactagccctgttggataagagccctacactagccctgttggataagagccctacactagccctgttggataagagccctacactaaccctgttggataagagccttacactagccctgttggataagagccctacactaaccctattggataagagccttacactagccctgttggataagagccctacactagccctgttggataagagccctacactagccctgttggataagagccctacactagccctgttggataagagccctacactagccctgttggataagagccctacactagccctgttggataagagccttacactagccctgttggaaaggagccttacactagccctgttggataagagctttacactagccctgttgggtaaaagccatactcttaggacttcatgtaacctttattatcttttcataggccctatcttcaagtacaggcacagtaggggttagggatttaatgtatgaatttggggggcagaggggacacaaacagtccatagcactaatatattactagaaaataaccttccagagtagagttgcacacccagttatggatccacctaaatggtcctcatactcagtccaggtctctccatcctgttcaccaagatgagtgagaccttttccagttctcttctgaagctcagctccagtatctgcatatttcccctatgtatcaatatgataatttgttaccaaaaaaaaaatctcaataattcactatgagttggtttttatgagcatatgctacagtctggtaatttttatttgatattttgggttctcagaaacagaatagttattagttagttcctagctgggaatcagaatcaatgataattaatgacacaataccttcagtgtttccaaatctaacaaactttgtcattaaattctcacattaagctaggtgtggtagctcacacctgtaatcccagcactttgggaggctgaggtgggaggattgcttgaggccaggagtttgatactagccctggcaacatagtgagaccttatctttacaaaaaaaaaatttaaaattagctgggtttgatggtgcctgcctgtaatcccagctatgcgggaaactgaggcaggatggtcactgtgccaccacaatccagcctgggtgaccagttgagatacatctcaaaaacctcatgatagctcatcaagataagcattatttttctttcaagtttacttatctgtcagagcagctctgcattttcttaacttgtctgtgtttggataaatgtttgaaattggatgaatatttttacgtgtcctttatgatttcttttttgagtcattggttatttaagaatatgttgacatttaacttccacatatttaagaatttcccaaattttcttcttttgttgttttttaacttaattccattgtagttagagaacatacttcgtgtgattttagtccttttaaatttattgaggctggccgggcgcagtggctcacacctgtaatcccagcactttgggaggccagaggggtgtggatcacttgaggtcaggagttcaagaccagcctggccaatatggtgaaaccccatctctactaaaaatacaaaaattagccgggtgtggtggctcacgcctgtagtcccagctactcgggaggctgaggcaggagaagctcttgaacccagaggcagaggttccaatgagctgagatcgcaccactgcactccagcctgggcgacagagtgagactccatctcaaaaaaaaaaaaaagaaaaaaattattgaggcttttttatgagccattctatagattgttccagattatttgagaattctgctcttgttgggtgaagtgttccatagatacctgttacatctgattttatagtgttgttgaagtcttctctttcctcattgatcttctacttatttattctctgcattattgcaagtgaggtattaaagtctccagattttattgttgaattgtgtatttctccctccaattccggcacatttcacttcatatattctgagtctctgttgttaggtgtgtacatgtttataattgttgtattttcttgatggattgagccttttatcattataaaatatcactaattatctctaataatacttttttttataaagtctgtttcatctgatgttactgtaggcacaccagctctcttttgttactgtttatatggtgtggtttttttcattcttttaatttcaacttgtgttttgaatctaaagcatgtctcttgttgacaacatacagttagatccatttatgtttaattactaacaagataggatttatgtctggcgttttgttctttattgtctatacatcacatcttttgttcctgtatttcttcttttgtgttgagtaaatattttataatgtcccattttaatcccctttttttattatactatatcttttgagttattttcttaggttggttatctttaggattataattagcaccttaatttaaaacgacctactttgaattaataccaatttaattttaatagtacgcccaaaccttactccgatatagctgtttcctcttgcctccttgtgctgttagtgtcatatagaatacatcttacacattataagtccatcagcacagatctgttattattatttattctgttgtcttttaaatcagatagaagagttaaaaacaagcacattttatactgtctcataattaccaatgtagctatttttatcagactctcatttcttcgtgttgtaaaccaaaaataaaatttgaagaccctccccaaccatctaaatgggcttcctcctctaggccagggccgtctaaatttaacctgaaagactggttcaggccatgatgggaagtgggggtccaacactcctctttacgccctccagcattaacatcaaatggaccttcagtctgataagaagaaacacttaccatctatcctctctgaagcctgctacctggaggcttcatctgcatgataaaatttggtctctgcaacctcttatcataacccagacattgcttactattgataataactctttcaaccaatcagaaaaatcttaaatctacctgttacctggaagcctgcacttcgagttcttagagtttgactcttctgcaacaatgtggatttgaattcctatgtagtatccttctgttttagcataaaggactccctttagtatttcttactgggcagatgtactaatgacagattttctcaattatacattgttattatttatgtaattttgtttttcaaagaagctgtgaggaaagaaggtgtatatacttatatcatttgttatattaaccctgttatttaccattttcagttctctttgttcctgtgggttcaagttaacatccagtgttactttcctactgtaatacagctttgctgtcacccgcttcctattacactgttactggtaaaccttacatttctgtatatgataggcccaacaatataattgcacattgagtatccctaatctgaaaatccaaagttcaaaatgttccagaagcccaaactttttgagcactgtcatgacactcaaaaagaattgctcactggagtattttggattttcagaatagggatgcataaattccaaaattaaaaaaaaaaaacctgaaatcccagacacttctggtctcaagcatttctgatgaagaatactcaacctgtacatagagtctcactctcttgcccaggctggagtgcagtggcaccatcacagctcactgcattctcaaactcctgggctcaagggatccttccgcctcagcctctcaagtgtctgggactacaagcccataccaccacgcttggctaattttttattactttttagagatggggtctcactgtgtgtcataaactcctgggctcaagcattcctttcaccttagcctcccagagtactgggattacaggtatgagcccagggggctcttttaggagctaccagacaggtcaaatagcagcagttccctgtggggtgtttgaggaactctaaattaattctgctttctccaatggctgctaggctaatggctttacaacggctgttgttatgagactgcttgtttttaaggttaatgcaaagctagggaaagggagatgggaataagacaagttaaagcagcacaaagttgttcttactgagatttagccatcttttcttgaatatacactcctcagattgttttaagagcttggttaccttccaaagttctgaaaaaattggttttgataatttttgccagtgttgttgttgcttctatggatgaatagatttttggagtccttatgctgccattttgaaagtacttctaccaggtgtaaatatttttaaagaatttgtcatgaccttctgtagaggatataatcttaatttgagttatgactctagattagaaccaaagcacacaccaagcaatcagtatttattgatgttctgagatcatcatcaaggaaagattgtcttattacacttgctttaatgcaatcactgatcccaatcgtgtgaacagatgaaaattcaattaaaaagtagttctgactctgcctagaactgtctcagaaggtgggaaaattgaatcaatacacagggcattattcataactcttctgcatagaactgattataatgggaattcacagaagggagagcatttatgttagaaaagtcagaggtagcttcataaagggaagtagaagttgaacccaatcttgaaggataaatggtggtgagtaaataagaggaagaccagtgtcggctggggcatgacattagcatcaaggaggaagacagcaccacagagacctgcccttttgctttagagagtttttgtttttttactgtggaataataatgaaaattaaaggtaaataagttgcagatagattctagaagtggcagctgattagtccaaagcccattaggcattcattaaatatttgtaaattaagagaaaaaagtaaatgctgtgttttggctacctttaaattattttgcagataggatttgaagttgacagattaatttctgctaaatcacagtttttggtgttgaatatgtgaaattttccaattttttaggtgtcatttgcctatgctgtatattggattagaatatggtttgaccaataactcaaaactagctgaaaggttcttcagccaagctctgagcattgcaccggaagacccttttgttatgcatgaggtcggcgtggttgcatttcagaatggagagtaagtactggaagaccagaaacccttctgttaactagtgattgtaaatacagtaggtgatcactagagttcactgacaaaagcgactcttcagtagatttgtacttttaaatgttctattttaggagaaggagatagaaaaaaaagagagaaactgttatcttggatttccttaaaaattctaaacccaggttttggtctttttttgatctgagctaccacttcttttcttacatgattggacagttcagattacacataccccacagtgtggctgggggcactgtggaatccttagttatctctgtgttgataaggagttgaatttacgtatttgagaagaatttgacatggtttttgaaacaaaagtgttttctatttagttactagtaagtcagaaaatttaattcctggtcagtgtaggctttactgtactctagaatatttgtagaataagtgatttggtgttatactcaaagagaaaaatgtatgtaatcctttgcatttcatgatatacaaattttaaaatggtaaattagtttcagttttttttggtttttgtttttgtttttttgagatggagtctcgctgtgttgcccaggctagagtgcagtggcgcattttcagctcactgcattctctgcctcccaggttcaagcgattctcctgcctcagcctcctgagtagctgggattacagatgcccaccaccacgcccggctaatttttgtatttttagtagagacagggtttcaccatgttggtcaggctggtctcgaactcctgacctggtgatccgcctgcctcagcctcccaaagtgctgggattacaggtgtgagcctccacgctcggctgtaaattagtattaatactactacagtgtcatttatggcatgtatgtgttctaaaaaatgggttatctaggtattttttaaaagtttgaagctttaaaataaatagtaatcttttaatataatacagtaatcatcagtatttgcgaatttgcctactcattaaaatttatttgtaaccgccaaatcagttcctgaggtgctttctgagccattggcagacatgcccagggctgggagaaagctgggtctcctggcacacacacttccagctggagtctaagaaggcaacgctctgccttttttcccagttcaactataaaccagtgtacttcttgtggtctatttagtgccacagttttcacatttttgtgctttttgttgatgatttcactgttttgaaataatccccaagcttagtgaaatgctatctagtgttaattgtgatacacttttgtgttacagaggaaacacatgtattagagaagcttcattcaagcaggcatcagtatttatagtgctgctggccctgacttcaatattgatgaatcaacaatatgtatcaaataagacgtctttaaatagaaacacacataaaacaaggttacgcattgatcaattagagaaaatgtgaccagagaattgcaggaatctaactgtatttcccctggcagccgcggttcaatatttgctaactcagtgtacacagtgactttatagaacataaatactgtggttgggtgcggtggctcccgtctgtaatcccagcactttgggaggccgagatgggcatattgcttgaggccaggagtttgagaccagcatgtgcaacatggcgaaaccccatctctactaaaaatacagaaattagccagggatggtgttgtacacctgtaatcccagctgtgtcaggaggctgaggcacaagaatcacttaaacccgggaggcagaggctgcagtgagctgagatcgcaccactgcactccagcctaggtaacagagtgagactcttaaaaaaaaaaaaaaaaaaaaggaatataaatactgtatctcccaaaatcttgaaatcttgaactcttgatattaatgccgtaaaattaatcatttgtgacttaactttgaatttttataattatttccagcagtttaccccattgcattttagtactactagaaaggtaatcaaaaatttttttaaagtttatatttgctaatttcgaataatctgcaaccttttttcctttcattttagatggaaaacagccgaaaaatggtttcttgatgctttggaaaaaattaaagcaattgggaacgaggtattctttgtagtacctgtaaatatacacatatacacccctgagtgtttactgttaaatgaggaaaacaacacagaaatctcttgccttcgaagatagtaacctgatattttcataacaagcggctgttaatgtttgcattgtcagttaactgttttaccacatggtaagaaatgagttttttccctaagagggcaagtgttggcgtttgcttcttaaatgggctctagctttgaaacgtgagaggcatcagaatagattgggtgattatttgaagctaggaatagtcattgggtcacggaaacaattgggcactgtagcgtcagatgtaatattcctctagaacttcagagagcttctttgactgaagggtaaataaagtatagcagcaggtcccacttctgaaaggatagggatgggataggtattactgaaattcacaccttttgttgctgttactgcatactgagtgtgtatttgggcctttccattttaaaatatttgactgcttctgttgtatgaacgatcccgtgaattggtgcttatgagttaacctaacaattatcaaggatgtgtatatactgatattacttaaaagtagtaacagttttagataccataaacttgcatagtaagagatttgcatgatgctcttctgtttcattcgtgcctgttttgtttcattctattctgattttcacttctcagcttcctgctgttactgtttgtgcacacctgcccacagttttcttctcccttggctgttagctctctctgctgttacagacacagctttgcctccctgccaagcgatctgcctgtccccatagacacagctttgtttactttggtctctcatgtcatctgaacttataaacactatttctgtgtttgcacatgcgtgtatataaagcaggtgcctctgtcgcatagtcttgggtagttctggataatccaagtgttttgttaataggggattgccacttgccaccttgtcagttttctaagaagtgactgccttatgccccagacgggctcagcagcatcagcatcacctagaagcctgctagaagtgcagaatctcagaccaactgaatcaaaatctgcagttttagcgtgatgcccaggggattatcacgcacagaaaagttttgagaagcactttcctaaaccatacttaagttgagcctgtattctttgatgattaaggggcactaaatttagagtcttacagaaacttaatggttttaagtatgaaagcacaacactattgaaatgtcaaaatgctcattaaaaagttatacgaagagggggaaaatattgactttattagaaactgcagcaaatgtcaaagagaaacagtcatgcctaagagtcattaaatgatttgtttttcattcacatgggctgtgcctcctagaactggtctcaatagagttagagaatctgtggctcaggtctgttcattcatttagttgacatatatttaatgagtgtcagccatgccataggctctgagctaggccttgggatgcaaaggggaatgagtttactatttcctgacctcagatcaactggcgaatgtaagtacgtaaattgacagtgaaaatacggtgcaattgaggaactaaaggtgcccacagagaaaggcagttcacccagaaatgagccttttgcatacacctcagtcctgaaagtcatcagacatcagctaggcaaaaattagggcaagggtattccagtgtttgcaaaaattggagctaattcattatggcttggattttgagttcacagagaatgtcaaggagtgggtcaggagggttaagtgtggatcactaaaggcccagaatgaaagagtttgtgccttatcctgaggagaatgggaaaacaaaagggatttaagaaggaaacaacccggtcagacttttatgattggggcccagatttgactctgttaatgaggaaggtgaggactaaataggttggagttatgcctagcaattagaaggaaccagccctggtgtgcctcatggaagggtcagagggggtgggggtgccattcactgactggaggggcactcagaagcagtgaggtcaggggctgggatggccagggagagatgggtgtggtgtgtggtgcactaaacttaaagtgcttgtaacatttacaagcagaagtatttagcaggcagatggatagagaggtctggtgtttgagggaaagctctgagttgcagatactcatttgaactccagcagggataggctcttggataaacttgtcctaatgataacagagtcctcaggaagtctcctagtaggaaagaaagggagatagataaaacagcagttaccctggccttgtggtgtgctggcggcacacactgggagagagcccagcttgcaaggcgtgaaataattcagtgcaaacaaatcaggctgaacagtgacatatataactcgtgggcttgatgttgctatgttttaggtaacagttgacaaatgggaacctttgttgaacaacttggggcatgtctgcagaaaacttaagtaagtgaagtagagcattttcagaaatatactttgtgtctcaaagtttacttaattttgaatactttgtcatattcttatttcatgagatcaaccagctttcttgtacttggctgctcagataaaatcagcatcaaatcagtttttattatttatctgtgttggtgtaattttattttagaaacacattgagaattttacttttttatatgaaaatttagtgttcaaaattaattcagaagaattaggacagtaggatataattaaaatcctatccatagtctgttttctgctagaggttttgtggtgtgataaacaggcgacagttgaaaaatgggttttttggttaaatatgtctgggcaattttaggttattcaagatttaacaagtatttctattgcaggatttctttgagtctttactatgtattacgaacttatagcattttccaaagtttaattgagcataaaacatttttttcacaaaagactttacaagtcaagtattccagagaactttaaggagcacttttctaagtaaatcttttggaccttaaagcgatactccttgactgcttttatctacccctgatatgggacactgactgtcacctaagagggtggtgtgtggtaagggcacagccaggaggcatggattaggttctggttgcagtgtcccactgaacatcaaaaggaatacagcagacatccctgcaggagtgcccttggccggcggagggagtgggcctggaagatgcatttagagggcatattgagatgcatgagagtaggtgtggagaacattcaccagtctagactgctgttctcattgagacagcgtttcttgggtgttgatatggatggttgctcatcttagacttaggaatttagtgcttttactgtagccaataattgtgttctctcccacagaaagtatgctgaggccttggattaccaccgtcaggcactggtgttgattcctcagaacgcatccacctactctgctattggatatatccacagtctgatgggcaactttgaaaatgctgtggactacttccacacagtatgtcttttctttgtacctaattttaaatctggttaacattgaccacttccttttttgaaaatattttttctaggtaatattgacttactattttaatattctttaggcaaccttacgtgttattcttttctttgcagagaaaagcatgtgggtgttgtacagtattcatcaaattagaaacaaacatgaatcctgtccttagtgtttgcagtgaggtgcgcaggtgcccagtggcactcaccagtgatctgagtgctgagcacggggttatctgtccacagtagcccctttgggaactcgcagtctttctgttcatgactgtccccttagatcacattagtagatgaacaaaacagtctctgcagtcatcacaaggaaagaacctttcattaatccatgtaccatattccatcaaatttaagacaccatgagtaagcaggatgaccacaaagaaaaaagtgctgccaattaaactttaacataattttttcccggcacttaatttttttgtacttagagaaaatattcttgaacatatttaggcagatttgtattactctagtacatacatagaaatgtacatataagtgaaataaatggttacaatattcagcccagagggccagtatctgtgttttctcaaaatagtgccctggtgctagcacacacgcccagttcctgactctgggtttcctcccagccacagacacctgctttccaactctctctctctctcgtccctctcagtctgctaaagtgtcaacagaaggtttttagaaaaccacaattcccgcatctgtttaagtgttcctcgcatggtttgtgatctagtctgtgctgccaacataaccaccaagttcacgcttctcaggcaggttacccatatcacagtggtggctggtggatggcaataataggaaaccacaggtaaaagggaagcaatgtttgaatttgagaaaatgtgggtcttagaatcagtgaactaatggtatttggagaaagagataaaatatgagaaaatttatcaaagataaactcatgagcgcttcgtaactgattttcaagagttcattagagattttgaggttagatgaagtcatgaaggtaccatgatggtcaccagaacactgagtttgtcagaagtttggactaagcacattttctagtcctgtatgatcttttaaatatgtatatttgagtattgtataatttacctttcttctgagatctgattaatacagaaggctcagctgggcgcggtggctcacgcctgtaatcccagcacattgggaggccgaggtgggcagatcatgaggtcaagagatcgagaccatcctgaccaatatggtgaaaccccgtctctactaaaaatagaaaaattagctgggcatggtagcacatgcctgtagtcccagctactagccctgcgcccggtcccaggctgaggtaggagacttgcttgaacccgggaggcggaggttgcagtgagctgagatcgcgccactgcattccagcctggtgacagagcgagactctgtctcaaaaaaaaaggaaaaaaatacagaaggctcagagaactctgtatggagatagaacttcttttttttcttttgagatagtattttgctttgtcgcctaggctagagtgcagtggtacgatctcggctcactgcaacctccacctcccagcagttttcctgcctcagcctccagagtatctaggattacaggcacctgctaccatgcctggctaatttttgtattttttattagagacggggtttcaccatgttttggtcaggctggtctcgaactcctgagcccaggtgatccacccgtctaggcctcccaaagtgctgggattacaggcatgagccactgcgcccagtcgagatagaactttttaaaaaatcttttttggggtagatttccagcataagaaaatagaatagtgtgatgaacctccatgacccataaccaacttcaacaatctttggttcatggccagtcatgtagctatgtctggccacttcccccaccctggatgccctggaatattttgaaatgaatcagtgtggtaagagaaggaaatatgttttcttttaataaccatgctgaatcttttatggcaggcccttggtcttaggcgagatgatacattttctgttacaatgcttggtcattgcatcgaaatgtacattggtgattctgaagcttatattggtaagataatcgttattcttattggtattgtactccattttttaggttgtcatatgtctctgtttatgtttctcatattctcgtctgaggttccaagttcatctttctaaccattcgtgctgcattaaaaacaagaaactccatttattttattagaacaatgctgtgggggagttagtgactcaccaagggtgcgttcagtttaaagctgaatacttgctaagtgcaaaataccactatagaatatgtgtaaagcactttgttatagaaggactcatctgtttctataatgggaaaagaaatcttagcaacatttttctgataaacaattgaagaggctctgtcattattttataatgcagggtataacaatataaatctttacatttcaatagaatgttgagatgccatacatttccataacagtgaataagtaatagtataagtttggatggaattgctgtacttgggcactcgtttagttataaacattatgtgataaggaggatgtggtgaggttattgagatctaggataggaaagttgcaagcttgctaactcccgttcctactaaagtaattttttttttttttgagatggagcttcgctcttgttgcccaggctggagtgcaatggcgcaacctctgcccaccgcaacccctgcctccctggttcaagctattctcctacctcagcctcctgagtagcttggattacaggcatgtgctaccacgcctagctaattatgtatttttagtagagactggtcttctccatgttcaggccagactggtcttgaactcctgacctcaggtgatctgcctgcctcggcctctcaaagtgctgggataacaggcatgagccactgcgcccagcccctactaaagtaatttctgatgtaatttcccccaaagtcaaaaagatcaggtaatgtgatacaaaacagagcagagccttagattttgagagggatctgtctgcttatgtgtcttggggttccctgaggaaaacagaggtttctcctaaaacggggtctgtggcaccctctggttttcccaaggagcccgaggctgtcagaaattaccttgggtcctctcatgtggacatccagagtggcgagaagacagactggggaactaatctagccaactgagaagaaaaacaaaaaatgagtaatacccattgtaaagttggataaactgaggcacatcacagtttaaaaatgcatgttcccataggctccacagctcactctcttaaccatcctgtaattgtgcctgctctatgcccagtcactgatgcacttgtgtggtagctcatggcacacctagcaaacagtttccctgcccccttagaacctgggtttcgtttcctgctctacggcagtataaacagtttttctctgcatgtgttgggttctaaccctatatatctcaaattttattaatattactgaatctttttttttttttgagacggagtctcgctctgtcgcccagggtggagtgcagtggcaggatctctgctcactgcaagctccccctcaccggggttcatgccattctcctgcctcagcctcctgagtagctgggactacaggcacccatcaccacgcccggctaatttttttgtatttttagtagtgatggggtttcacagtgttcgccaggatgatcttgatctcctgaccttgtgatccgccctcctcggcctcccaaagtgctgggattacaggcgtgagccaccatgtctggccaatattactgaatcttacagggggctgtgatgtctttaatccttagaaatattaatctatagacaaaggaatacatgaggttaaacagtattcacatttctatatgctttaaaacattcaggcaatgtattaagaaacacacctaagaaactgcagtgaatctactctggatgaaaatttagagaatatctcttcaaaagaagctatctaggccgggcgtggtggcccacgcctgtaatcccagcactttgggaggctaaggcaggcagatcacctgaggtcaggagttcaagaccaacctggtcaacatggtgaaaccctgcccctactaaaaaacaaaacaatacaaaaaaaaactggccaggcatagtggcagacgcctgtagtcccagctactcaggaggctgaggcaggagaatcacttgaacccgggaggcagaggttgcagtgagccaagattgtgccactgcactccagcctgggtgacagccaatctcaaaacaaaaaaaaaagtgctacgtattggtagttatgcatattcttacatatattaacagtattttacatgcttaaaaccttaaaaataattgaaaatgtcaaaattatatgccatatacatttctgtgggcttgaaaatgatacaatataggttgtttctttaaagcagtcactaaaatgtgtaaacttctagatagagtaatcactaaaaaggaaagttcataccaaaagtagtaatactaggaataaaagagggctcaacactgcctttgggagtcctcctcccatgaaacatggctggatttttctccttgtcctgtgtaacctcccttactttatcatagtttactgccttagttactctctcattcctccaaggagagcctcaagaaatttaggtaacaccagagtgtggccccagccagttgccctcagttaccaaggagcaattaggtaggaaatggccactgaaagactgaaaacaagaaaaaaactcaggtccctcacccaaactgggcagtggtggtcaggcttccacacggaaacctttcactgtcaccagagtgtccccagccagagacctgcagttgcctctgtgcttagatgctgtccaccaagggtcccgagttggaaaagggaaaggggggggagttcccctatgtagagcagagggatccccacatggagatagctccagctcatgcctgcaaatgaaggctctctagagagggaaaagggaaaaagaaatgaaaaataaatcccaaaatttgggcttacctcccgactggctcaccaaaatatgttactggtgtggaaggtcttcactacaagttgtccaggttcttggcatggtgaacaaagattgaacaaaacaaagcaatggaacatgaaagcataggtgtattgaagtgaaagtatactccacagagtgagaggggcttgagcaagcagctgatgagccccagttggggtttaagtacccttaggggtttcgtgttggttacaccctatgcaaatgaaggattggtctgtggcagatcagaggctgaagtgaagttacaccctatgcaaatgaagactgtgaccaatcagaggctgaagtgaaggctccctgtctttagaccgtattctgcctcagttaagaagcaaataaaggccctcagggaaatgaaattaaaaccataatgatggtggtgtgtgcctctagtgccagctactcagggggctgaggcaggaggattactggagcccaggagatcaaagctgcagcgatcacgccactgcactccagcctaagcagcagagtgaaaaccctgtctcaaaaaagtaataataataaataaaaccttagtgagaaaccagtacacacccaccagaatggctgaaagtacaggatgtggtgttggtgtgatgtggagcaacttgaactctcatagattcactgctggtgggttataaaagggtaaagcctctttggaaaactgcttaatatcacaataaagttaagcctattatatcagctctctattgctgcctaaccactccaaaacctcaaccaacaacgtgttatttttacaattctgtgggttgagtgacccatttgtgggtcttgtctaggtttactcatgcagctgtggtcagcagatacttgactgggtagatggtctaaaatggtaccattctccagtagggagctcagcttgggtgctgagtctctctctgtggggctttcatcttaggtttctttgtagcacgatgttcccagggttctaaaggacaagaaccacagttcaggacctcttgaaaattcagagttgcacaacatcacttccgtcacgttctgttccatcagtacaagtctcataagctcagagtcaatgtgggagggggctgtaccagggtgagggtgctgggaggtgtgattcattggggtgggtattagggtgatgattgaccatactgattttgtgatgtagcagttctactcccaggtatgagcccagtggaagcaagtgcttctgtccactaaaggtcacaaacaagagtattcatagctttattcattacagcacctaactggaaatgactcaaacgtccaccagtaggagagaggagaaataaattgtggtatgttcatacaatagaatactatattatttgaaagactactgaatcatttagaaacatgcatcagtttcacagatttttttattttttattattttttctgagatggagtttcactcagtcacccaggctggagtgcaggggcgccatctcggcttactgcaacctctgcctcctgggttcaagcaattctcctgcctcagcctcctgagtagctgggattacaggcgcatgccaccacacctagctaattttttgtatttttagtagagatggggtttcaccatgttggccaggctggtctcgaactcctgacctcgggtgatccgcccgcctcagtctccgaaagtgctgggattataggcgtgagccaccacgcccaacaaatttcacagatttttgacaaaaatgtgaatatgtactaattacttccatttataggaagattagtgtattagttcattctcgcattgctgtaaagaaatacctgagcctgggtaatttataagaaaagaaatgtaattggctcacagttctgcaggctatataggaagtatgatgctggcatctgcttggcttccggggaggcctcaggaaacttgaaatcatggcagaaggctaagtggggagccagcacttgacatatccggaacaggagggattgagaaagggaggaggtgctacatactttaaacaaccagaactcactatcacgagaacagcaccaagaggatggtactaaaccattcaggagaaaccacccccatgatccagtcacctcctaccaggccccacctccaacattgaggattacggttcaccctgagatttggtgggacacagatccaaaccatgtcaattagtaatgaacaaaactaattggtagcgatagaaaagtccaagaagtgcttatctcaggcttgggaaagctgacagtattgaccaggagtgggcacagggtcttaaggagctgcatgtgttctgtgccattctggcggtagtcatctgggatgcatgtacatagggttcattcagccatgtgcttaaggggattcacacttgactcttggttttagctcaattttaaaaaacaaatttaagctctggtttctgagagatgctaggttgaagcctcagctctgcccctcggcagccctgtggcaagaaaggcaagtctgagccttagtttcctcatttgttcaagggtgggaagttcataggaagctaaataaaggctagagagttgataatgagtcagagggatgtctccttgacctcgcagaagagatttacctttttttttttttttttttttttttgagacacagtctctctctgtcacccaggctggagtgcagtggtgcaatcctggctcactgcaagctctgcctcccaggttcacgccattctcctgcctcagcctcccaagtagctgggactacaggcgcccgccactatgcccggctaattttttgtatttttagtagagacggggtttcaccacattagccaggatggtctcgatctcctgacctcgtgatccacccgcctcggcctcccaaagtgctgggattacaggcgtgagccatcgcgcccggccaagatttaccattcatgtcatttttatttttagaaagtttgcatgtcattatctgccaaactacctatttctgaaagctattggtttgaactgtgtaaaactcattttgtttatgtcgaatgattgtacctaatacatctgttcagatctgttcaaatctatattacccttaaggttttaatatatgggaaatggattttatttcatcgtggcttttttatgcataagattggtgactatacccaatgggcacagcacagttctttttttttgagatggaatctcactctgttgcccaggctggagtgcagtggcactatctcggctcactgcaagctccatctcctgggttcatgccattctcctgcctcagcctcctgagtagctgggactacaggcacccgccaccatgcccggctaatttttttgtatttttttttttagtagagactgggtttcaccgtgttagccaggatggtctcgatctcctgaccttgtgatctgcccgcctcggcctcccaaagtgctgggattataggtgtgagccaccgcgcctggccacacagtgcagttcttacctgtaacagctgaggagaaagttgtccacaggcctcatacgtatttaggaggccttggaaatgggcaactgttgaaacactggagctgcaactgcatgtgcttctgaaagtcttctgacacttcctttgcaaatggtggaaaacaaatgaaaaagaagattggaatgttgttttccttctgctctcaacttgtgctcttaaaagtataatttgacttaaatgatagagatggtgtataatgatggaaatgataaatgatggagttttgtattttattctaaatagtataatctgacttaaatgataagagatggtgttatataacaatagaaatgataagtgatggaatttcttattttattctaattatagtatttctttttaggagcagacattaaagacaaattaaaatgttatgactttgatgtgcatacaatgaagacactaaaaaacattatttcacctccgtgggatttcagggaatttgaagtagaaaaacagactgcagaagaaacggggcttacgccattggaaacctcaaggaaaactccagattccagaccttccttggaagaaacctttgaaattgaaatgaatgaaagtgacatgatgttagagacatctatgtcagaccacagcacgtgactccagtcagtggtcctggtcccactgtcccagtgtaggttagtattccttcacatcctctccatggcttaagaatgtcccacttcctaacgtgactccaaactgcatctctacatttaggaacagagacccgccttaagagactggatcgcacacctttgcaacagatgtgttctgattctctgaacctacaaaatagttatacatagtggaataaagaaggtaaaccatctgttatgtctttttttttcttttccaataaacttttattttggactaatttttgatttacagaaaaatggcagagatagtacagagagcttatgtgtgcccctcacctagtttcccccattggtaacctcttctcttcccatggtacatttgtcatgactaagaaaccaacattgttagtgtccattcactaaaatccagccttgatttggatttcaccagtttttccgtaatgtctgctttctgtcccagagtcccatccaggcacatactgcatttagtcatcccatctcctttgcgttctcttgcctgtgacagtttctcagacttcccttgtttctcatgaccttgacagttttgaggagtactggccaggtagtctgtagaatgtccctcaattggagtttgatgtgtttctcaagcacctttctcatcacatcctactgggtggttgtaaccaccacacgacctatcccaggcagtgctgaccttggccacctggtcacagtggtgtctgccagatttctccactgtcaagttcctgtttttccctttccatactctgtgggttgaggaggtttcagctccaacccctggagagagcagtatctgtgcatgttattcttcttgaagatttgtttcttctctcctgtttattcgatcatttatcagtatggactcatgtttacctacaggcatgccttggggacactgcagttttggttccaaactggcaagataaagcaaatatcacaataaaactagtttcacaaattggtttcccagtacatataaaagttatgtttatactgtagtctattaagtatgtaatagcattatatccaaaaaaacagtgtatataccttaatttaaaaatactttcaggagttcgagaccagcctggccaacatggcaaaaccctgtttctattaaaaatacaaaaattagccgggcgtggtggtgggtgcctgtaatcccagctacttgggaggctgaggcaggagaatcgcttgaacccaggaggcagaggttgcggtgagctgagagcacacccactgctgcactccagcctgggcaacagagcgagcctccatctcaaaaaaaaaaaagagaacactttattgttaaaaaaaaaaatgcttatgatcctctaagcctttagcaagttgtaatctttttgctgatggagggtcttgcctccatggggatggctgatgatgatggtgctgaaggctggggtgaccggtaacttcttaaaataagagtgaagtttgctgcattgattgactctttcatgaaagatttctctgtagcatatgatgctgtttgacagcatattacagtagaatgtctttcaaaattggggttaatcctctcaaaccctgatgctgctttatcaagtaaatttatggaatcttctaaatcctttgttgtttcaacagtgttcagagtatcttcaccaggagtagattccattttaagaaactattttctgtgctcatccatagagagcaactcctcatctattaaagttttatcgtg[450 bp omitted]ggatgttgtgttagcagcatgcaaacagcattcatcttgtacatcacccacggagctcatggatgaccaggtgcattgtcagtattgtcaatgagcagtaatactttgaaaggaatctttttctgaacagtaggtctcacgggtaggcttaaaatagtaaaccatactgtaaacagatgtgctgtcacccaaactttttctgtttttaaagcacaggcagagtagacttagctctcagattttcttcaaaatgtgaatgagcattggctttagtttaaagcctccagctgcattagcccctaacaagtcagcctatcgtttgaaacaagacattgactcctcttcagctatgaaagtgctagatggcatcatattccaatataaggctgttttgtctgcattgatcgttttctgtttaatgtagccatcttcattaattatctttgctagagcatctagatggcttctccatcagcacttgctgttccatcttgcactttttatgttgtggagatggcttcttcccttatacctcatgaaccaatctctgctagtttctaccttttcttttgcagctccctcgcctctctcagccttcatagaattgaggagagttagggtcttgctctggattaggctttggcttacaggaacgttgtatctcatttgatctcgtatccagatcactcaaaccttctccctatccgccgcaataaggctgtttcacttttttgtgtgtgtttaatggagaagctcttttaatttccttcaataacttttcctttgcattcacaacttggccaactgtttggtgcaagaggcctggcatttggcccattttggcttttgatgtgcttcctcatttcccggccattatttctttagggttttttgtttgtttttttgttttgttttgttttccctttttctctcttgttgggattcctatatgcgtgcattggtatttctgatggtgtcccatgggtcccgtaggttctgttcttttcttacttgtgctcctcagactggataagttcaactgccttctcttcacctttgctaattctgcctgctcaagtctgctgctgaatccttccagtgaatgctttaatttcagctattgtgtttttcagttcccgagtttctatttggttccttgttatcatttctatctctgttgacagtttctgtttgttcatacatcgctctcctagttcttttagtccgttttccgtggtttccgctagccctctgagcatattaatacagttgatgaacatctttgactagtaattctaatatctggggtccctcagtgacagtttctgtcatattcttttcttcccataagtcagccatattttccattttatttgtgtgctttgtaattttgtgttgaggactgtacagtttgaatatgacactgcagtagctctggaaatcagattatccctctcctcagggattgcttctgttacttgttgagggctgcagttcatttgtgtagtgaattttccaagttatttttgcgaagtctgtattcctgaggttcttggtctttttattgtctctggtcagcctgtaacctgacagaaatttccttaaatgtctggatcttttcttcttttttttgagatgaggtcacactctgtcacctaggctacagtgcagtggcatgatcatagctcactacggcctctaacgccttggctaaagggatcttctcgcctcagcctccgagtagctaggactataggaatgcaccaccatgcccggctaatttatttattttatttttgtagacacgcagtctcgctgtgttacccagggtggtcttaaactcgggctctaatgatcctctcgcctcagcctaccaaagtgctgggatttcaggcgtgcataactgcgcctggcccgatatctagatctttttttttaaaagaagtgtatgtctctaaattttctgatagatggtattggggaaattgctgcagcctgagggggtcgagacaaaggcacgcatcgacgctggtccctcagggcaccatcatgtgaccaaaatgcacagggtcctcgctgaggacaggctgcagcaggtaggttggctgtccatccacaccaccctcttaaccaaaaagctgcagcctctgcatcatcaagccttccccgggttgcagtaagcatctgatgaggtgacagagttagaaaacagttgattctgctaatttgttgtccagcttaatggtggttttggtggaagaaccaattaatccctgaagcttcctgccctgccgttttctgtgacgtcactgctgtcttaaaactataactaaatattagtcatattaaaaaaaaactataggctgggcgcagtggctcacgcctgtaatcctagcactatgggaggctgaggcgggcggatcacgaggtcaggagattgagaccctcctggctaacacggtgaaaccccgtctctactaaaaatacaaaaaattagccgggtgtagtggtgggtgcctgtagtcccagctactcaggaggctgagacaggagaatggtgtgaacctgggaggcggagcctgcagtgagctgagattgcgccactgcactccagcctgggtgacagagcgagactccatctcaaaaaaaaaaaaaaacaaaaaacacaactatagctaaataatagtcatataaaataaattgcagtacttatcaaatttcagtttaacttttgaaagtcatagtatttaaggtaacaaactcagctattaacacattttatgatgctttaaaaagctcaaagaattgaacagctattggtgctaagttataattgtgttctataaaataaatgtgattcactattctttgtaattgctacctcctggtatgtataagataaagaaattgaattttacctattttctcacatactaatttggtgacgtatccttttaactgttggcaaatgttataggctctcttggcctgttttttattccattattttaacatcttttgcagttttctcacataagattgaaaactgaatctatgtgaaataagcaggtgtctatcagtaatgatatttaaaatatgtcacagctggattccctgttttgtaggagcatctgaaatcagccatagcggaacagatggaaatgccccatttgttggggtcaagaaggaaaagttcttacatagttttacacatttggctggtgattgattttgtgaaaagatatgttaaaatttgtttgttttttgagacggaggctcactgtgttgccctggctggagtgcagtggcgtgatatcggctcactgcaacctctgcctctcggactcaagcaatcctcctgtctcagcctactgagtagctgggattacaggctcctgccaccacacccagctaatttttgtatttttagtagagacggggtttcaccatgttggccaggctgatctcaaactcctgacgtcaaatgatgcacctgcttcagcttctgaaagtgctgggattacaggcttgagccacagcgcccggtctgaaaagatacgtttaaaaattttattttaaaaaggtgctacaagacaattctactaaatctctaatctttaaacttttttttaacctcttcactgagattaggaaaagaatcttgatatgactcctaggtgataactgtataaacagacaacacccccactcttcatcattctaataaagagagaggattatatgcttcatttgtttaaagtcctttattcaaactctcctactactaacatcagaggacatgttactctactttttcataaaaacaaacttgtgtagttattagaaagcatctattcacggcatcattaacaaattaggcattatacagaagtgaattttccaggcaactgaggtgctgccttaaacacaaaactttttatcttaacctatctgaagtgtattccctgtatttctaccttctgaagcagaacaatgatagcattcatttcttgaggatccccaggtgttaactctagtaaatcttttaaggagggctttgttccccttcatactgatgagggtctagcactgttgtatggtgcattgaagactagcattcagaccccatcctgtctccattcatcatggtgaaagtcaggtatcgcgctggaagttaatgcagtaatacatttagagaccatgagttaccccacgctaaattagactcaaactaatgcgttttcatgttttcatttttaaatggcttttctgtctctctgtcaggcatcatgtatgcctcccctgtcttgagttggggctaaaaaatcaagtgactgaacttaaggtaattatttttaaaacaactaaaatgaaagtattatattaataaagtgctgagttttttacatttatttttcctgccagagcaaaaaaaaaaaaaaaaccatttagttttctcattgatcactgttagaatttttctggtttctgtaagtgaatgtcaaaaggttcagacacttgagtaagctgtaattcggatcagcagcagtgttaacaccagtgtttaggcctctgagagtaaaaacaatgtgtcgtactgccaagtattgttcctgttactacccaattcttcagtatctcaggacacagtttatcctaaaaaggtgtcctgtttgctaactacagtggctgtgttatcgttaaaggaaatactgggaagtgttacggtcaccagcttcaagtcactgtcatctaccaatgagataggggctaccaagtaaaatgtagcccagggccacattcacagatggaagtgttcagttaagcaaaagtaaagttacatactaaaagcccacatgaagaaggatttggagatatttcttcctgctttgctttggctgtaaagttctgtacatgtatcaaacagagcatttcaacaggctggctaatgtgtaggaagctagcttttctttgtgatataatttgtcatgtaaaatcataagctattttacaagtgtatttcattcactgtatcttaaaattcatatggttttatttaaagataatttcatcaaacagctgaattctaaaatcctacctttttttcaaaatcatttttcagaactttgcaataacaggaagtaaagaatcagaagggtgttaaaaaattgacttggggggaaaaacatttggacattttgctggctaggtttacgaaatatacacatccagcaaacatgtgagtccctttagggtacagtgcagtgaagggagaagtggaaatactcaaggttctggggaaggtcaaatgaagggtccctcacattggctgaggccaacctggcttcagaggtgtgcagaggcaccaggccgagaacagggctttgcagtggtgagggcaaaacatgaacacaagaaataccctaagttactgacctggttgtgcctggtgacagatgggaccagctcttttctccatttggtaattttggaaaatttggcagagttaataaagaaatccccttggtgcaagattgctacaataagaatctattatagatggcagatgtgaagttcacagtcctttttattatcctaacctagtaacatgactagaggtaaaattaaatattgagttagatgattgtttaatattttaaaaccagagggaagacatacaaatagattgagagcagtttctttctaaagataatcagaatatatctcctatattttaaacactgcaggaacagtcaaaattttaaaataaatggtttccacatttttttatccccatctctccccctgactatatagtaaaatattttatttggcaaccacgttttcctacttttaatcacacatcatcagagcttttcatcagcctaactagtcttcctactcactgagttgtgacgaaagcctggtctaggcacttttaccacggccatgtttttgttaagatccctgcagactgtgaactatgaaaaccagcccctggccccgcttgtgggtgctgcagagccgcgtgtgaactgctcctaggaaaccgcgtctctgggcagggtggctctcgggccgtggatccgcctaaaagcttttctgccagtgacgcccttggtctctggatgtgttcacgcctcagtgattttactctctggtttcttgtgtgtgcttgagattcttgagagaaagcgtgctgcagatgaaaaatgcagcctacatgaaaaagagaaggggaaagccattttaaaataacttccatgtaagttactgtaatcaaagtgctgctgcctgtgtgttcataaaaaaatatgtgcaagggcccggagcggtggctcatgcctgtaatcccagcactttgggaggccgaggtgggcggatcacttgaggtcaggagttaccgagaccatcctggccaacaaggtgaaaccccgtctctactttaaaaaaacaaaaaaattagccaggggtggtggcgcgtgcctgtaggtcccagccactcaggaggctgaggcagaggctgcagtgagccgagatcgcgccactgcactccggtctgggcgacagagcgagactgtctccaaaaaaattaagaattcaaatgtgaacccggctttcatggcgccccggccacggagagcctgggggagtgggataaaaacggtgctgggatgggagggcggcttgtccgcgaactcctcccaccacgcccggaccaaagccgccggcgccccgcttccggggccgccctaaaccgcggccgccgctccctgaggggccgcctgacccggcggaaagccaggctgtgtggccggaggtcaccaccccgaccccgactagtcccgcagcgcctgaccccttcgtgggcggccccgaccgccgcggctcgaggaggggcggggctggggcggccgagctctcgcgaggtttcgtcgggggctggcggctgcggctcggcggagagtgcggcatgcgctcggaaaaggagggggccggaggccttcgggcggccgttgccgcgcggggcccgagcgggagggagaagctgtcggccctagaagtgcagttccaccgcgactcgcagcagcaggaggctgagacgccgccaacttcgtcctccggttgcgggggcggtgcgggcaaacctcgcgaggagaagaggacggccctgagcaaggtggggacgggggcggggcgcgcaaacctcgcgaggagaggacggccctgagtggagggaggggagggaggggagggaggggcgggggccgggcctcccagcgcggtacgcggtgccttttgagctccttgtccacgctccgccccggtgggaacggccgcgcgctccccgcaggtggtcatccgccgcctgcctccgggcctcaccaaggagcagctggaggagcagctgcgcccgctgccagcacacgactacttcgagttcttcgccgccgacctgaggtgaggcccgccccgaggggaggaagagagggcggaacccagagctcggcggcggtggccgtctcgttgccttacgttccttaccctgatttctcctatatgggagtcgtgtgagctttttgaataaatacatttcagtaaaacgtgtccgttccgtcaaagaaaaataccaccaacaaagaaacacagatgtggtttacatgaaaaatgcggatgattttcagacggctaacgcttaggaaagcgggtgcctttgaaaggaccagcgttgcccgcccggcgcctcccgggcttccctgctcgtccgcggacggggcgctgcggggccggggggcgccggctcttcctgtggcctccacgctggtgccgcagccagtgcggttttaaataccggagaaggtccccaagtcaggagagtctctcggcgccacgggttcctctgggagtgcgccctggccttgccttagggtttcagcctcggaggaccggttctgggcagtggagaagggacctgagttctgccttgtaaagttaacgttttgcgtttgtttttgctaaagaatatccaagttgttacaattaactgagatgatttggcacaaaagttttatctaaagtagtttgttgtgcccagaaaaggaaaaagaggctaaattaatggactattgtatttttcactgaccattttcactgttatctcttatttcagtctttatcctcatctctactcaagagcatacattaattttaggaatcctgatgacatccttctttttagagatcgttttgatggatatatcttccttgacagcaaaggttggattattggtttttaaaaatctattataatctgtaggtatactaatgaagtattgctagaatattcgtgctttttaaattattattattttttgagacagggtcttgctctgttgcccaggctggagtgcagcagccgcaatcacggctcactgcagcctcacactgcggctcaagcaatcctccagcctcctaggtagcagggaccacaggcgtgtgccaccatgatcggctgatttaaaaaaaaaattgtgtagagacgggggtctcactgtgttgcccaggctggtcttgaactcctggcttcaagtgatcctcccacctcagcctcccaaagtgctgggattacaggcgtgagccacagcatccggtctatttgtgcattttaattaaaaagttctcagactaaaataagtttataaattttgattatgttactgtaattatgtgcagatattaaaaattgaaattatttaattttattatttaatccaaaaaaattccagaaattctgtgtaatagtgacagcctatttatgtattatcatcagagaatgaaggttttcatgagtgagtttgttgcatgagagaacttgatctttttaggttctaaagctaaactattttacctgataggaattttccaaaatgtacactgacttctatcttcctaaacagcatccttcatgactgtaccatttatgataatgtcaccaggaagtcactgctgttaaacagctttgcctgcggagacagcagtcttcaactctactctcaatagttcccttatcagtacttctagggttctgctttcatctttttccttctcttctcctgtcaggctgtctgctgttgtgttcctggctgtcaagtttgtatacctttcacagcaagttgttttctgccctcccctccccagccaccctcttccgactccacgtgttttgacactgatgagtgttttctttttctccaatgaggggtaaggactagaaagattgttaaaaaaataagcattagccaagctagttcctgtgactagaagcaaatgtgatgcttgaactgacctttgtgttcattgctcatttattcagtaacttactgagtttccctttttcacttaaaaaatattggtgggccgggcacagtggctcacacctgtaatcccagcactttaggaggccaaggcgggtggatcacgaggtcaggagttcgagaccggcctgagcaacaacgtgaaaccctgtctctactaaaaatacaaaaattagccaggcatggtggcaggcgcctgtagtcccagctactcggaagactgagacaggagaattgcttgaacctgggaggcggaggttgcagtgagccgagatcatgccactgcatcccagccggggcgacggagcaaaaaaatatgtatgtgtatgtacgtgtatgtatatgtgtgtgtgtgtgtaaaatatgtgtaacatggccaggtgtgatggctcactcctgtaatcccagcactttgggaggccgaggaatgtagatcagttgaggtcaggggttcgagaccagcctggccaagatggtgaaaccctgtctctactaaaaaaaaaaaaaaattagccaggcatcatgaggcatgcctgtaatctcagatacttgggagactgaggcatgagactcacttgaacccgggaggcagaggttgcagtgagccaagatcgtgttactacactgtagccttggtgacagagtgagactctgtctcaaaaaaaaaaaaagtcacaaaatttataccatactgttttccacagtgactgcgccattttaggttcccacctgtagtgagcaaggggccagtcgtccttgtttccggttatctttgttgtcgttgttttgttttttagagatggggtctcactatgttgcccaggctggtgtcaaactcctgagctcaagcgattccccccacattggcctcccaaagtgctgggctacaggcgtgagccgctgccctgccgttgtttttatggtctctggcctaagcctgtgcttttgatgtcatatgcaacccattgccaaatccattgccatggagcttttcccctgtgtttttttccaagtgttttatggtttcaggtcttatatttaggtttgatccatatcgagttactttttgtatatggtgttaggtaagggtccagcttcattcttctggctgtggatatccagttttcccagcaccagttgttgaaaagactttcttttccccattgaatggtctgggcacccttttcaaaaatcagttgaccaagtattacaaaggtttatttctgtgctctctattttattcccttggtggatgtgtctgtctacatggcagtaccacactgattactttcagctttggaatcagggagtatgtcatctgccattgaatgagtatccactgtgtgctaggccttgtgggtggagcggtgacttggacatcgtccctgctggtccagtgccctgccgtccccctgagtcttgactttattctggatagtggaggttggcacaaaaatatctcccagttaaaggaattataattcagtcacctgactattactgacaagtcaaaaaaaaatgactcagtgggtttagtaccaaggtagcagtgttccatttgatgattcagcatatagcaggttctcttagtgaacatttctctttgtgtatttgtttttcccccacatagcaacgaagttagtttctaatgacttccattctctacttttatcagaagcagatttcacctggaatattctataaaccctttgaaaccctctattttagccatggtgtcttctaagcaaagtaattttcttgaacttaaataacaaattgatagttgaattaaccttttaaaataaaatgtaaagtgtagctaagaaatcattatttaaaggtattccaacgataaattatttgggatggggctggggaggtcaggtatattgaggtgtaagttacatatggtaaaagtcacccttttaaagtgaacaatttgatgaattttgaacaacttcagttatgcaaccaccacaacatgatggattgttttagtaaatgttcttcttaccaggagttcatccttgtttaagtctggagtttgccgtgttaaggttgcaggtgcttgaaagtgtaataaaattgtaggttttttaatcttttttttaatctcttactggaaggatgaattatagtttaaatagtaataatgcattgtcgttgttacacttactctttaagtaagttaggtcattattttccgaaatgaatgtagtagaatttcagaatggcttctggaacatgtttcctgttaaaaggcctagaatatcctgcagtggtagagtttgctccattccagaagatagccaaaaagaagctgagaaaaaaagatgccaagactggaagcatcgaagatggtgagccctttccaagtgctacgttatgaagctgccaaattaagaacactgagcaaatgtaattctcccgtagttgggaaagattatatttattttcttcctactttttaatgtctagatccagaatataagaagtttttagaaacctactgtgtggaggaagagaagaccagtgccaaccctgagactctgctgggggagatggaggcgaagacaagagagctcattggtctgttttgctcatttcttctcttttctttattgagagatttactcgtagaggatatacgttttttctctttttcttgactgtgataacaagttgaaacttgttacaggcttgacagaaatgtagagtgatttccagttttgacaaaagaatggcaagatggcagcatgggaaaatatttctgagcgttggttgatggagagcacgtgatttttttagttaagatcattttaagttatgatgcttccgaatgagcagtgcagaagacacatgaatggtctgttcctgagcgtcagggaaggcatgttgtgtcacatagttgaagtcagtggtcacagacggatgcctcaagagcccataggaatgagatgttttgcttgcggtgaggtcatcttgctgcttcaacacaaatgccaggctccccgagccaggccaacttgtggataagcccagccagagcacagaatcgagtttaaatccagccttccaccccatgcccacaagtggtgtactttagaaatagttacttcccagtagcaccaacatacttcccagtagcaccaacatgaaaatcaggcaaactgttgttgtttagtcccagacacacctggtagtgtttctcttctctgtggatctcctttcctcaaagccctggttcttcacctctcatctctcccgcccaggctgtgccctgcccacccatcaagatggatgtactttgggaggccagggtgggcggatcacaaggtcaggacttcgagatcagcctggccgacatggtgaaaccccatctctactaaagatacaaaaaattagcagggcatggtggtgcgtgcctatagttccagctactgaggaggctgaggcaggagaatcgcttgaacccagaaggcggaggttgcagtgagctgagatcgcaccactgcactccagcctggtgacagagcgagactccatctcaaaaaaccaataaaattgatgtgacatgacagccacattgtttatcatttattttcctgtctgtctcccaccacataaactcccttcttgttttgtctccccagtggaactatattttcatcttcatggttgtagaaaagttcaggaatttagtagatgttgattgaattaataaaaaaagattaatattactgaaatagtccgttcactttcttcatttatctagtaataaagctttttttccccccactctgttgcccaggctggagtgcggtggcacaatctcggctcactgcaacctctgccttcccagttcaggccattcttctgcctcagcctcctgagtagctgggattacaggcacccgccatcatgcccagctaatttttatatttttgtggagatgggtttcaccatgttggccaggctggtcttgaactcctgatctcagatgatccgcctgcctcagcctcccaaagtactgggattacaggcgtgagccaccacacctagccgtaataaggcttttgatcactaaagtaaacaaaatatgctgaggctgcatgctgcctgggcactgactctgggcctcagcatggggccaagtggtaagcaggttgcacaaggcctctgcctaccatcgggatgacaggttggaagcaggagcaggaatagtccaaccagggcacctcagtgtgtaatcactgccactgatgtgacaaagtggaatatgggagggaaagaggttgggagactccctccaagatagtggtggagctggagctgagactggctgcgggagggaaagaggtcaggagatgagctgagcctggccgtgctgagcagtggaggaaggaagcgccaggggcagggccggctgctgtcgcttttcactgcaggcgcagagtgcaggcttgtggggttggcacacatgggtgagggcagcgtggttggaggttcagggcactgcaagacatttagattcattccagggcaactggaagccactgggggagtagcatggtctgattatttgtccccgtcactttggtggtgtcacatgggatagactgtggatgccaagagatgagcgtgtgttttggaggtgggctggacaggaccgacagacggatagggtgtgggaggtaaggggacaggaagaattaagactccagtgcttggcttgaacagatgggtggattgtggcaccattatacacaagggaacatgagggtaggacagtattgggggcaaagtcgagatttcacttaagaacttgataaattgaagaagagctagaagcagcaaattagacgttattacaggcctcatctccctgcccctcccatttccatctcactttgttttaaatgtccccttgcaaaggatcggtcactttagggctgttttgagacatacctttttggggcagacacgtggctcctgccggggtggggagaggggggcatgtcctggcacctccccgagagtctgctgcacagccaatgcttcacaaaagttggctgaatgaatgaatgaatgtggcttagaacccaccatccctgatgggagcgattaattcattccaggacttggctgtagagtcttccttgtttctgtgattgctgtttctatgtatgaaaaattgaaacttggccgggtgcggtggctcacacctgtaatcccagcattttaggaggctgaggcgggcagatcatgaggtcaagagattgagacctttctggccaacatggtgaaaccccgtttttactaaaaatacaaaaattagctgggcgtggtagcatgcgcctgtagtcccagctacttgggaggctgaggcaggagaatcgcttgaactcggcaggcggaggttacagtgagccaagaccacgccactgcactccagcctggcgacaaagtaagactccatctcaaaaaaaaaaaaaaaaaaaattttaggcaattcatggcagattacctttttcttttctaagcttttggcctccagtggattcttctgcacaggtctgagaagcaagtgttaaatggaaatactaataagctgtttctggcaaggactctggagttgttgagttagtctggtgcatcggcctttagcatcggcctttagagcagacaggcctgggttcagctctgagacttctgtttacttgtgttctgttgcatggttgtgcgtattctgtctgataaataacatacttttatgatgtgtagaacactttcttattcagggtctaacgtactttgcccgtgatccctgtgggtgatagggcagacattttataggaagaggaaggtgggcaagcggagtctggggtcagcctccccacacatccaggtggtctcgcccggctcctggctgacaagttcaggctttccacagagcagagcagaaccctggctgatttgagataatttgcccattacaaagagctgaccatttacctgagccagtgtgctgtactcaccctgagccagagttcatggtcagcccagggacccctgggggagtgttctctgtggcaactgtgtctgccacgaaatgtggcatttgtacctgcatcagctcttccctacaaccactgtgcctggggcagggctcctgggacaatcggctcttccctacaaccacagactgtccctggggaagggcttctgggacaggtgccatttcatctatgatgtcagggtgtcaggggttctcagttccaagtgatggatccctggccagggcttctcctatgattcctgattcattcctggtcctcagaattccagagatgatgtgtcagttttcaggtacatccagggattcttcccagccctgtttagtctgagatcttacagacccacagagtagaccccgtcttcccccttcccatttgtgtccttgtgttggtgggatgtggggcctgtccatgtcagcgcaagtccctctgaggtggggcctgtggggcctctagcttgcctgggcacccacatccaggtcagatctgagaggtcgggcatgaaagcgaaggaccaagatagagtctgtagaaatgttcattccctagtagttggtgccttttctgcaatcagcagcgccatatggagtcattttctttttcttttcttttttttcttttttttttttttttgaggcagagtttcgctgttgttgcccaggctggagtgcagtggcgcaatctcggctcactgcaacctcctcctcccgtgttcaagtagttttcctgcctcagcctcccgagtagctgggattataggcgcgtgccatcacgcccggctaattttgtattttttcagtagagacggggtttccttaggttggtcaggctggtctcgaattcccaatctaaggtgatctgcccgccttggcctcccaaagtgctgagattacatgtgtgagccaccacgcccggcccatgtggagtcattttctaaactacaatccagagttgtatgtcttatcattgggattttatggagtatatattgtaagacattacaagaatccatgttattttaaaactgttgcttggagaacggaaaacgaaaaagttctcagaatatgcgatgtatcaaaataaatatatgttatttaatgatttaaggcacctacaaaaatatcatcttggaatattttaagcctcccttaagaatcaggaattagatgtctactatgtatgaaattgttgatactttcaaattacttgacctgacagtaatggaataaaaactcccctggagacagtgtgtaaatttaaaaagtaatgatatggttcccgtatttattttaaagattacattcaaaacaagtatttaaaatacatctttctttaggagttaaatacaatattacaatttttgtgttttagctagaagaaccacacctcttttggaatatattaaaaatagaaaattagaaaagcaggtaggtctggcctttacctgatgaacaccctccctgcttctgccattctcatccaggcagtttatacacacgctcttccatgtctttagagaattcgagaagagaagcgagaagaacggaggaggagagagttagaaaagaaacgtttgcgggaagaggaaaaaagaagaagaagagaagaagaaagatgcaaaaaaaaagagacagataaacagaagaaaattgcagagaaagaagtaaggattaaggtaattctgaggaaacatttcctttttccaaaaatagctctgctatagtaattacactttttacatatcttagttctttagaattttgaaaacattctgaattaatctaatattgtgttgttatttttttccatcttttccatctttctttccatcctacagcagtggttatcacagtgcgtcctgagccctgggagtcctcaggctgctgttgggtgcctgtcctcctgggtgtgcagtggggagcacaccccacaggagccccacgctggcacacgttgggctgctgctgacgctcttttttttttttttttttttaaactttccaactttcattttcggctcaaggcgtacatgtgcaggtgtgttacatgttcattgtcagctcaacgcgtacacgtgcaggtgtgttacgtgttcattttcgactcaaggcgtacacgtgcagatgtgtcacatgttcattttcagctcaaggcgtacacgtgcaggtgtgttacgtgttcattttcgactcaaggcgtacacgtgcagatgtgtcacatgttcattttcggctcaaggcgtacacgtgcaggtgtgttacgtgttcattttcggctcaaggcttacacgtgcaggtgtgccacatgttcattttcggttcaaggcgtacacgtgcaggtgtgttacgtgttcattttcggctcaaggcgtacacgtgcaggtgtgccacatgtttattttcggttcaaggcgtacacgtgcaggtgtgttacgtgttcattttcggctcaaggcgtacacgtgcaggtgtgttacgtgttcattttcggttcaaggcgtacacgtgcaggtgtgttacgtgttcattttcggctcaaggcgtacacgtgcaggtgtgtcacatgggtaaatcaagtgtcactggggtttggtgtgcagataattttgttgcccaggtaatcagcacagtacctgatgtttttcagtcttcaccctcctcccattctccaccctctacattttcctttaaaaaaaagttttcctcccagcactttgggaggctgaggcgggcagatcacgaggtcaggagttcgagatcaccctgactaacatggtgaaaccctgtctctactaaaaatacaaaaattagccaggtgtggtggcggacgccttaatcccagctactcaggaggctgaggcaggagaatcgcttgaacccagggagcagaggttgcagtgagccgagatcgcgccattgcactccagcctgggcgacagagcaagactccctctcaaaaaaaaaaaaaaaaaaaaaaaatttcctggccgggtggggtggctgacacctataatctcagcactttgggagaccgaggcaggcggattacttgagttcaggagtttgagaccagcttggccaatatggggaaaccccatctctactaaaaacacaaaaatgagccggacgtggtggcgtgtgcctggaatcccagctactcaggaggctgaggcaggagaatcacttgaacccaggaggcggaggttgcagcgagccgggatcgcgccactgcactccagcctgggcaacagagcaagactctgtcttaaaaaaaaaaaagtttccctgattaaaaaatacacatttgaaaaccactggttttgcctttctgtgtgaaggctgactcagaaccgggttttatcatttctttggcagtagcactaatgagtttctgtatttcttgctgagttttttctgtgactgatacattcatttatgagggtggtttaatacatagagggaatttttctctgtgtgaaatgtgttggccagaattgggaccagccattatctcctcagtactaaacctagatttgaacctaaggtatcactcattacttattatttattgaataccttatattcaataatattgtacaatatgaggaaaaaaatgaaatgtcaggacttggggaaagaagatagcttaggaaagggtggggaagagatcattgaaccatagatttgtttctgatatggtcagcagtcaaaaacagaaaagttggctgggtatgatggctcattcctataatctcaggactttgggagaccagggcaggtggattgctctagcccaggaggtcaagaccagcctgggcaacagagagagaccctgtttctgttttttgtagagatggggttcccactatattgcccaggctggtctcgtactcttggactcaagtgatcttcctgcctcaccctcccaaagtttggggattacaggcgtgagccaccatgcctggcctggtttagcttttaataagtatctgtgctcagtatgggggtctttcacttctaaatcatgtggaaaattgaaattcttttaatgcctgaaaaatggaatctgtggagaaatgcaaaagaaggtgtatcaacagcttaaagaaagacagatggctcatggctattttgctatttttttgtttggttttggtggggggggggtttgagacagggtctcaatgtgtcacccaggctggagtgtagtggcacagtcacagctcactgcagcctctacctcccaggctcaagtgatcctcccgcctcagcctcccattacaggggtgcaacatcatacctgaatagctaatttaaaaaaaaatttgtagaagtgggggtctcactatgttgtccaggctggtcttgaactcctgggctgaagtgatcctcccactgctggggttagaggcatgagccaccgtgcgtagcactcatggctattcttaataaagagaaatatggtttgggaggccgaggcgggcgtatcacgaggtcaggagatcgagaccatcctggctaacacagtgaaaccccatgtctactaaaaatacaaaaaaatagctgggcgtggtggcacacgcctgtagtcccagctactcaggaggctgaggcaggagaatcgcttgaacctgggaggcagaggttgcagtgagctgagatcacgccactgcactccagcctgggcgacagagcgagactccgtctcaaaaaggctggaccgcggtggctcacacctgtaatcctggcactttgggaggccgaggtgggcggatcattaggtcaggagatcgagaccatcctggctaacacggtgaaaccctatctctactaaaaatacaaaaaattagctgggcgtggtggcgggagcctgtagtcccagctacttggaaggctgaggcaggagaatggcatgaacccgggaggcggagcttgcagtgagccaagattgcgccactgcactccagcctgggcgacagagcaagactccgtctcaaaaaaaaaaaaaaaaagaaaagaaatacggcacacagaacagctcagctgtttaatgttcctctgaggatgctaagcagtgagcttatgtgtggccacatgggtgaacacacacaggcgagggctgggagggagcacagctgcccatgcgctccgcttggcctccggaagccttgcagggcgtggcagagctggcctgctccccagctcgtctccagcagctctggcctgctccccagctcgtctccagcggctctggcgtgctccccagctcgtctccagcggctctggcgtgctccccagctcgtctccagcggctctggcctgctggtttctcatgaatgcaccagcggattccacctcaggactccgcacgtttccctccacagggacttccccagatgtgcccgtggctccttcttggcatctggtcttagttcccgtcttggctctgcagagagccctcctgggccctcctcttcctgcgtctctgatgctttactctcccttcatgtctgttcgtagcttttgatgctacctgaggttatcttattcctttgtttcctttttcatatttttgccactgttcacagcattcccccttgaccaccgttgcctcaggtggtgcatcaggcgcaagttccttcgaggcaggggcacctcttgtctcattcacagccattccctagtgcctagcacagggtcttaggccctggttcctgcacacagctcctcagacctcgggaatctgcagtaatatgagtgtcttatatgccaatgagaggactaggggctgggggccccaggcagcttcaggatggggactagttggccagaaagccaaggcaggactagagggttgggactttcagtcccaccctagcctccgagagggcagagggcctagagagaggtcaccggtggccagtgacttaatcagtcacacctacataatgaaaccttaataatgcctaaagacagggcttccaggggccaggcacagtggctcacgcctgtaatcccagctctttggggggccaaggcaggtggatcccgaggtcaggagtttgagaccagcctggctaatatggtgaaaccccgtctctactaaaaatacaaaaattagccaggcatggtggcgcatgcctgtagtcccagttactcaggaggctgaggcagaagaatcgcttgaacccaggaggcgaaggtacagtgagccgagatcgcgtcactgcagtctagcttgggtgacagagcaagactccatctcaaaaaaaataaaaaaaatagacgggccttccgggttggtgagcacatcaaggtgctgggagggttactgcatggaagctccaagtaccactacaccataccccatacccaccttgtgcattttttccgtttggctgttctgggttgtatcctttatgattagccaataatcgagaaaacagtgcctttctgagttctgtgagccatttcagcaacttaccaaatctgaggagaggccatcgggaacccctgaatttattgctgactggtcagaagtaccggtgacacactgggacttggaactgtcgtctgaaaggggggcagtcttgtgggactgagcccttgacccgtgggttctggtgctaactccaggtcgatgatctcagagttgagttgaatgtggagcacgcagctggtgtcggaggggtggtattgaaaaagacgtgcagtgtcagaaatgtaagaacagttcctaggcacacagaatatttgttgaatgggtggatggagatttgcacttcctatggttcacatttattacagacatgttactgttgcaatttttataaaagatgggcagttaccagaaaggaaatgtttaagttcccagattccacataggcagaataagaggatgttcacagctgacatggggcccacgcttgatgagaagcggcttccaatgctccaccttgcatgatgcgttgtgttcccaagtgaacacgtgtgattgaatgcatctgtgtatggtcttgctatgctgcaattatgagtgagaacagaattctcagatgtacaccttccatttctgttctacaaatgatctgtctgtggtgcttccgttttttttttttttctcccttttctcaagttagctgaggtgctttagtttcaaaaactatacagagagcagggagaaaggccaggggcatacagggcagatgaaatatgtcgtggcgacttgttcattaattctgtttcaggaaagagaataatcagtgttttgaaaactataaactattctaaaaatcatcagtaggaagcagccttttaaaaatttttttttccttattacaaaactaatacttgtctatgtcaggaaaactagaccctatagatgaaaggaaataaagtcatttcagttctgccatctagaaataatcactattggccgggcatggtggctcatgcctgtaatcccagcactttgggaggccaacgctgggggatcacaggtcaggagttcaagaccagcctggccaacatgatgaaatcccatctctactaaaaatacaaaaattagccaggcacggtggcaggagcctgtaatcccagctacttgagaggctgaggcaggagaattgcttgaaccagggtggcagaggttgcagttagctgagatcaagccactgcaccccagcctgggtgacagagtgagacgtcgtctcaaaaaaaacaacaaacaaaaactacaaaagttagccaggtgtggtggcacgcccctgtagtcctggctattcaggaggctgaggcagtagagtcgcttgaacccaggagacggaggttgcagtgagccgagactgcaccactgcactccaacctggacgacagagcgagactccatctcaaaaaaggaaataatcactattaatacagtgctgcagagccctttaggtggtggtgtgtgtgtgaacataaatagaagtgggggctgggcacggtggctcacgcccataatcccagcactttgggaggccagggtgggtggattgcctgaggtcaggggtttgagatcagcctggccgacatactgaaaccccgtctctactaaaaatacaaaaaattagctgagcgtggtggtgggcgcctgtaatcccagctacttgggaggctgagacaggagaatcacttgaacccgggaggaggaggttgcagtgagccaagatcgtgccattgcactccagcctgggcaacaagagtgaaactccatctcaaaaaaaaaaaaaaaaattagacgggtatggtggcgcatgcctgtcgtcccagctactcaggaggtggaggcaggagaatcacttgaaccttggaggcggaggctgcagtgagccgggagtgcgccacggcacttcagcctgggcgacagagcgagactctgtctcaaaagagaaaagtgtaagtgagattgtggtgcatgttctgttttttagccctttattcattacgtgaacatctttgcatcacattaacctaccttacttttcatgactaaaaaatatttcattcactgaatgttccaccacgtattaaaccaaaaacctcatttttggacctttagctttaattctttgctgtgcaaacatttggcttcaatattggggtatatttgtaattatttatttaacagcttcttttaaaatatgctctcaaggtgatactttactaacattgtaatttctcagcttcttaagaaaccagaaaagggagaggaaccaaccacagagaaaccaaaagaaagaggagaggagattgatactggaggtggcaagcaggaatcctgtgcccccggtgcagtcgtaaaagccaggcccatggaaggctcgctggaggagccccaggagacgtgagcgtgctttcattgttatgaccacgtcagctcccagtcatggtgacgtaggctttgtcagtatgagtatgcctatttcacactgttcttggaatccaagactttccagggtgtgcgagtacacgttagccttcatttcccatcagcatggcaggaacgtagtgaactcgtcttgtctgcagtctcaagtctgtcttcagatcgggagtttcttcctgttgtttgctttgcttttgcctccatgtcctccttttactcctgagccgcctcctggattcctgctgttgcatgtcaggtcttcaggatctgtcttcccagtctttctttttgttttttgttttttatttttctctgtgtttgtgatatcttgccctttgctcttttatcccccttgcaatttatctacagaattgcatatttgggaaccccctcttttaggtctggcagatcttttgtgatagagaaacagaattttaagtgctgtgatcctttggttgaagctgtccctggctcctctggctgtggatcttgaccagcagctttggctgtgcactgtgggtcctccctttttcttttcctttgctgcatatgacagtgcagctctatggagggagctcatctgcgaaagggacagacacagcattatctgcccggtgggccagagagaccagccagatgccagtcaggagggatcctgtccctcttgctcctggcatccaccatcctcagggccccaggcacctgggcttaccttcccctccgggactcctgcccactcactgcctctcgccacccacagtctcctggtgcccagggccacttggcccacaggggatgatggacgtggattttaggtgggaagactaatgcgattaggctgccggcttcccagagccatagtcgtaggtgtgatttctcactgcctttcgttcagagttgatgaagtattttttctacttcttgggagttttggtttattgctttgtttataaaatttatatatgattatcaaatccatgattgtgaagcactgtgatgatgttaccggtgttgttactgtgacagttgatgacacagcaggacctactgccacgtgtctgctgtgaattcatacttggttacagatccttatggtccccaacagccctggcaaatgagcgaagcagccatttatttactattcatttaatattctgataaatgcataaagataattaatctgccattattaaatgtcagttaattttactctgcacatcatgatggatgtgcacctgggctcccaggacagccactggaccactttagtctcagtcattttatctgtagaaaagaaggtttattttattttgttttattattttattttattttaattttatttttgagacaaagtctcgccctgtcgcccaggctggagtgcagtggcacgatctcggctcactgcaacctccgcctcccaggtttaagcaattctgcttcagcctcccaagtagctgggattacaggcacccgccaccatgcctggctaatttttgttttttttttgtttttttgaaacggagttttcctcttgcccaggttggagtgcaatggtgcgatctcagctcaccacaacctctgcctcctgggttcgtgcaattctcctgcctcagcctcctgagtagctggaattacaggcatgcccctccatgcctggctaattttgtattttaagtagagacagggtttctccatgttggtcaggctggtctcgaactcctgattttaggagatctgcctgccttggtctcccaaagtgctgggattacaggtgtgagccgctgtgcctggcctaatttttgtatttgtagtagagacaggatttcatcatgttggccaggctgctctcaatctcctgacctcaaatgatctgcctgcccctggcgtcccagagtgctgggattacaggcacatgccaccacacctggctaatttttgtattttcagtagagagggggcttcaccatgttgaccaggctggtctccaactcctggcctcaggtgatcccacccaccttggcgtcccaaagtgctgggatttacaggcgtgagacaccatgcctggtcaaaagaaggtatttactttggagggttattgagaattagaaaatatgtataaaaagtaccttagcatggccaggcacggtggctcacacctgtaatcccagcactttgggaggccaagatgggcatatcataaggtcaggagatcgagaccatcctaactaacacagtgaaaccccgtctctactaaaaatacacaaaaaattagccgggcgtggtcgcgggcacctgtagtcccagctactcgggaggctgaggcaggaggatggtgtgaacccaggaggcggagctttcagtgagctgagatcccgccattttactccagcctgggcaacagagcgagactccatctcagaaaaaaaaaaaaaaagtaccttagtataaggcatattgcctatctgtcactcaactgaaatgacagatactattactatgatttattcctctttgtagtggtttttatcaacgcctgctgtgtgcccatcgcagtgcagcctcagcagtgcagcgctgagcacttcgcatgggtccctgttgtctgggaacctgtggtctagaaaggagggtgggcagccaaccggcggtttactgcagttgctgatcatttgtgttgaatcataggtcacacagcggcagtgataaagagcacagggatgtggagagatctcaagaacaagaatctgaagcacaaagataccatgtggatgacggcaggaggcacagagctcaccacgagcctgaacggctttccagaaggagtgaggatgagcagagatgggggaaaggacctggccaagacagagggaagaaggggagccaggacagcggggctccgggggaggccatggagagactgggaagagcgcagaggtgtgacgacagtccagcacccagaaaagagcgactggcaaacaaggtttttattaaacccaaaaagaaaaatgtgtctggctgtcttaaggtccaggctgcatgctgaccatgtcacccccacttggccttgtgtcttggggaacgcagtgctttgagcattttcaagagcagtttttcctgaaagtcagatcccagagtgagactagtcatcatcttttctcagataatcaaattatttttcaccaggaaaaagaaagattttatttagtataaaactagcacgtttatatgattcacttgagaataagattattaaatttacccttgagacaggaaggaaagttttaatgatatttcatggaggtttcttccacattattaacaacattctgattattggtgaatattcccatggctcacaaacacctgtaagttagatctgcacggacggtgagcacaggactgtggttacccccttagccaagcaaacaacttttttttttcaggagctaatttttgttcaggttgcattttcccagcgcagcactacagatggcatcacctttctgacagcaccaggccccaccctggcctcctagcaaactgagggctgcctagggttccagttcccactcacctccctccccatgtctaacccctggcagccactgtctgttctctgcctctgtgattttgtcatttcaagaatgcttgataaatggaaacacacagtatgtgagctttgagagtggctttcttcactcagtctaattcctttcagatccttccaggttgttgtgtgtatgaacgatgttcactttgaaactgtttcagtgtagctgggcatggtggctcacacctataatcccagcactttgggaggctgaggcgggtggatcacctgaggtcaggagttcgagaccagcctggccaacatggcgaaaccctgtctctactaaaaatacaaaaaatagctgggcgtggtggcgcgcctgtaatcccagctactcaggaggctgaggcaggagaattgcttgaacctgggaggcggaggttgcagtgagccaagatgatgccattgcactccagcctaggcaagaaaagcaaaattacctctaaaaaaaaaaagtttcagtgaagtcaaggctgaaagccagaggagagccttcttgtcttcacccttgaatgaacacctctagaatactccagttctctggggttgcctggacctcggggtgcacaaagtacagccatgcgtctttttgatagggcgttgctcataggagtcctcagtcttcagttggcttattttagcagtggtgaatggcctggtgtttcccccgacctgaggtgggggagcatttgctcctgtgtttagcctcacagtgctgtggtgggaatgaggcgctgtgtgcaggagctactttcccgggagcctggcatttgtcatgtacttctaaggtcaggctccatgctccatctgtgtatacaggtattgccctggtaagaagcagaattgtcggcccggtgcggtggctcatgcctataatgccagcactttgggaggccaaggcgggtggaacacctgaggtcaggagttcgagaccagcctggccaacatagtgaaaccccatctctacggaaaatacaaaaattaagcgggcatggtgacacatgcctgtaatcccagctactcaggaggctgaggcagaagcatcacttgaacccaggaggcagaggttgcagcgagcccagattgcgccattgcactccagcctaggcaacaagagtgtaactttgtctcagaaaaaaaagaaaaacaaacagagctatgcgtgctggctccaggcagcctgtccaggggcctgctgagcctgttcccacccctcccattggtcaggttcataccaaggaaaagaaaattaagttaaaccccctggtggtaagcagatgtgggcttccacacacagtccaggaggaggtagaggtgagctggtgaatcctggagaaggagcgcatgagatgtacccacatggtcgtccttgcagcccacacttgggctcctggtggctgttggaggctaaggctgcagctctgcagggagaataggcagggagcgggaggcgcgtggagcaggaggctcacggaagctggaagccgccttcctcctctgccaccccttcagtgctggggcagggctggtggccccatcaggaggaattgtgctgctgtcgctgctgacactagccttcagggccactctatgcttccctggctgttgccttcttcaggccctcaacagctgcccttcatggacttcatgggtcatttttattgcttcacctccaggagtttaataaaatgatggggtgatgtcttctaaaaagggaccagttcagtctccctgctggtacatgctaggcagggataagccccatggaaattgcttactactcacgttaataagttttagttggcagatctgaaaccatctgtgagggtccacatctcattttgttcacttcccaactcagagggggacgtcatgattgtactgttcccacagattacatgttcttgtttgagcaataacatttgcccagataaccagtgtttacatggcttttttaattgccttagaatgcctggtttctgagatagtgctgtgaaaaaagggctgatttttagctgcctagttgattttctttggacaattcatatcatcaagttctagaaatatagggagatattgacccttctgtgttgctacctcttggaagagtaacatgccctcttatcctggcaggaccggccagccttgcagctgtatgatccaggagctcgcttccgagcgcgagagtgtggcggaaacaggaggatctgcaaggcagaaggttcggggactggtcctgagaagagggaagaggcagagtgagtcactgcacgcacctggcctccatggacgagcaagggcatcccagaaacgtgtaaatgaccccgagtgtgactgggaaggagaacttattccttaccaggaaactggaagctaaaaatacagagggtgacgtagaaacacgcagaaaccattctaaagaaagtagtgatcttgtattaaattgagcagaattctcacagattttaccattcctgttataaactagtatttgttgtttagccaaaacagaaaatgatttccactggacagtagaaaaatatgtgtaaaatagggaagaaagttagtattggatcagtgtgagtcctgaagcactttcagtgctgtgagaacgacatccactttgggtttcattcgtttgtaagcagaggagctgtcagtcactcgtgcttctcggtggcctctgagccatggtgtcgagtgaagagtagttcttgtttgttacaacctttgtgagtcagccatgcccgcaaagcgtgctgtgttttagtcctggtaggaatatttatcagagttcacactatataaaacccaacagcttcaactattgccctttcaacagttttgccactgaccggatagaaacggtttcagtctctggatggatgtgtttgtggtttgtaaccattacggtttaaaccatggtttaagaatttgcccaaataacagaaattttgttcgggaagggataaactagatatagcatacagagcctgtttttgagttttagatactttatttgtaaataacttaaaatagctttctgaaaccgtgcattctgtagtttcttcctttcagtgaaattgctaaatgtcaatgtatttttggcactgcgattttaaccatttattaaataaaaattttgttaaagaagtactgaaatattctttgaatctgtgtcagggaaattctgatttgttagttcaaatgcctgacagcataatcatacagatcttctcttgtcttggtttggatgacactgagcagctgtcagaaacgtgcaaaaaattccaaaaggtaaggccttaaaatattttactggtaaatattacacagactttcaaaaggacagagaatgacttggaaaatcttttttttttttcttggagatggagtctcgctctgtcacccacgctggagtgcagtggcgtgatctcagctcaccgaaactcacgcctcctgggttcaagcaattctcctacctcagcttccagagtagctgggattgcaggtgtgcgccaccacacctggctaatttttcttaaacaaacactgtctacatctgccctatcagagcaagagagaaagacaaaacactaggttatgacaagcaaactcacccacccactctttaaaaaaaaaaaaaattcagggccaggcatggtggttcacgcctgtaataccagcactttgggaggtcgaggagggcagatcacgaggtcaggagatcgagactatcctggctaacacggtgaaaccccgtctctactaaaaataaaaaagtaaaataaaattagccgggtgcggtggcgggcgcctgtagtcccagctactcaggaggctgaggcaggagaatgaacctgggaggcggagcttacagtgagccaagatcgtgtcactgcactccagcctgggcaaaagtgcaagactccgtctcaaaaaaaaaagaatactaaacacaattcagaagagccacccagagggagacaacaatgtcacaactacttgtgccacacacacagactgtgctcttgtctattggttctacttgacatcaaagggaggctagttgtttgtttcatcaatgaaccacagctgcaggtggatttccacaccaggatgaatgaagactcagacatcaccttccatttccaagtgttctttggccgttgtgtggtcatgaacagcgtgagaatggggcctggaagcctgagatgaaatccaaaaatatgccctttcagaatggcaaagaatttgacctgagcatcttggtgctggacagtaagtaccaggtaatggtcgatggccaatgcttttagagctttgaccatcgaatcccgcctgagtctgtgaagatggtgcaagtgtggagagatgtctccctgaccaaagtgagtgcctgcaattgagggtgataaccagacttcctgttgacaaaggaatccgtttctgtgtgaccatgggattcccagagccagctaacagcataattcctcctcacttcaacccttactcttgctcattaaaacttcgccaaacttaaaacaaacaaaaaacactggattttaaaaacacatacgcccatgcttcttcacaaggggaatatcttatctgtaaaataaagccatattcccactggaaagtcggtgatcattaagaatttctagattgcttaaataacagtaccttgcatttatgaagttctttatgtttctcaggcagaatattgatagctgataaaactgatgagagtccattatattaccctctcttccttgaatgtttaaaatttctagtaataatgcattaaaaagaaaattcatctcggatggggatgtattttcccagttggtttatctatcagtggatgagttgaataccttttactaatagaataagatagtgttggacaattctactccagtttttaattttattaatataactactgagaaatcttgctcacacagtaaaaaggaagagaggagttttgttaggttaacaccacttaattctttaatttctggccgggcgtgatggctcatgcctgtaatcccagcactttgggaggctgaggcaggctaatcacttgaggtcaggagttcaacaccagcctggtcaacatggtgaaaccctgtctctactaaaaatacaaaaagactatctgggcatggtggtacacgcctgtaatcccagctcctgggaaggctgaggcaggagaatcacttaaaccggggagacagaggttgcaatgagccgagattgtgccactgcacacctgcctgggtgacatagtgagactccatctcaaaaaaaaattgttttctttcaagttatattgcaaaatgttcagttatctataatcagtattacttctaatggtctaccatctcataattgtttcaattttattgaaagctaacgattgggaatcacttaacttttaaaaggatttattattcaatcactaaaatcatccagtttctctgtttctgagaagtaaatataaaaggtttcccaggacttaccagctaactattaattacacctgttgcttttccactcttggagagaaagagctcaatctgacatattcagaaaggtttgggggattagcagtgatttcagcacatctttgtcagtgcaatatttttataaagtgttcaatgcatatttgccaaagccttggattgcagatttaattcatccaatttctggaaaatgtagtaaagtcaagattaaaattatatagagcaaatagtctttctcattacctaatttaaataatacaatataatgtttagctacatgtatgagttattggtgaggccagccgtgtgataatgggagttctatttcttcagtaaatatgtgtatgaatatgtaaaactctagcatggggcttttgggaatattgaaaagattttttaaattataattttgttttacaacatgaccaagtgttttacatgtatttaagaaaacattaaagtatatgctgctgaaaactaattgcatttatcatttagtttataacttttctaactaatggaaggtggaaataaaattttttttttgagacagggtcttactctgttgcccaggctggagtgcagtggcacagtctcggctcactgcaacctccacctcccaggttcgaatgatcctcccgcctcagcctcctgagcagctgggactacaggcgtgcgccaccacgcctggctaaattttttgtatttttagtagagacggggtttccccatgttggccaggctggtcttgaactcctgaccttaggtgataacacccaccttggcctcccaaagtgctgggattaacaggcgtgagccactgtgcctggccagaaataaaatatttaattggataaagaatgactgcaactgggaaacatttggtagaaacaggttttaactatcttaatgattactgcacactttattaggatgaaaccacataaaaacttacccttaggttttataggatgtattttggaaacagccccattagcaaattgcttaaactttgatatatgttccttgttcattctgtgtctgagtcatgtttatggagctggggtcttgctgtgttgcccaggctggaggccattgacaatttacgggtgtgatcacagtgcactgcagccgcaaactcctggcctcaagcagtcctcccacctcagcctctcaagtgcatgtttttgcctttctgaaaatcatactttaagcccatccccttacctgtttccccaaactgtctctcaaatcttttattcttattgggtcctgatactcatcctatgaagtaagtattctctattaatggtcagagttcttgttactgtaattaaatcagtataaaataatggcatctaacattgattctgttcagtgtgctagcactaagtgctctgaacacttcatgctgtcttatgccaaaacaagcttataaggtgggtactatcgttccactgcatgcaaacatatgagaaagtagccttgcccaagttcacattcaagcatgtgcacgcgcacacagtaagttacagaggcaaattctaacataggttggtctgacttcacagccaaagctctcagcgactgtgcctgccattcccagaacaggagaaagtatgttttcaaccagcattaaagctgtaggtctctagtgttgccagtgccttacctcagctttagtgatggcaggtggtagaagtccttgctcggagagggagcctggctgcacacctggtacacaatgcggggcaggagagaccatgggctctctctgcagttctgcaaccacaagcagtatccagggccaggctggtttctatgctcaccactcctgcttttttttttcttcaaccagcagagcaggtgaccatagttcttgggcagcctgcttgaaacaaagtgccctctgctgctcttttcatgaagagtgtagcaggattgattgaggctggctttgcacttacacggtaaactggccatatgagaacttagctgctctgccggccagcaccaggctcccagttgcctttcactcgctcactggcgctcaactagcattgattgagtgcctgcaatgcagcagtcagtgcccatctgccacccctggcagctgtgtggacctagactgcccttcagcaaagtctgctgctctgcctgagaggaaaggaagagccccatgcccaccagcaaaggcttctggggcctgggcatcctctcctccctccaaaactggggctagtgaatggttaaaaaaaaaaattaagaaaatattaatggacttgattctaatttttatatttgatttttatttttcagtcctatttaaaaaaatgtctaaaactcggaaataaggcaaaaaagagtgattgcatggctgtgtcatgtctgacaagctccgattatttaacagttgacccacatttgtgcccacactgtgacatcacatgtttccttacggatttttgcccagtagagatgtgagtgatttctataacatggcctcaaaggtggcagttgtgcttccctgtaggatcctaaccacttttgtttctaactggccatttgactccactttttctagtggctgcacgtactcagctttatgaaccttctgaactagcttcatcctcctctggattccctcatgaatgagttaaataaatcctctgagattgagaccgtcctggccaacatggtgaaaccctatctctaccaaaaatacaaaaattagctgggcgtggtggcaagtgcctgtagtcccagctactcatgaggctgaggcaggagaatcacttgaacccgggaggtggaggttgcagtgagtagagatcgcaccactgcactccagcctgccgacagagcaagactccgtctcaaaaaaaaatacaaaataaaataaatcctctgatatgtgttcactttatattcgtgtgagtcatgtgtttaacttttgagaactatactttgacattccccaaagcccagcaacagagctagatactcaggaggaattccattttatcatacttcccattaagtaacttattttagttcccattattttgcattattataaattaagtagaaatagcagaataatttgtccttttttattataaagtagctaagttgattatattagatctaaattataagctggactgcttttattatttctcttacaggaagcacttaataatttatttctaatttttattatgttccttgtaatacatattaatgcagcaatttgaatggaaaaattaagtctgactttctgacagaaagccagtctgattttggctgcttcatttgacaacatgattctgtgtaggttttgaatatgtgcagatactcagcactgttattctttatacacagaagacaaatgacaaagtccagtgccatctcagctaatgagtttgcagctcctcaaaggctgtcaggctgccactttctactgcaattgtagttcacctgcctctacccattccctgattcaagtctggtccctggaacatagtataggttgagaaaacatcaaaataaactgttgagaagtatatgttcaatagatccttaagtgaaattttgaaagtccataagggtctggttttagtacaggattttatgagagttatttctgctttctttggttactttaaagtttagtgcaatatactgggaacctttcaaaaggccaaattgggagtttatttgcattattcatcttgaactatttaatattctataactacaatagcttttttaacatgatttcaattcagaataatagcagagggtgttttggtttttgttatttcgtcaccaagagttgccttctgctagagttttgttcagtgttcaattggaataggtctcttgggatcatcaactcattctgacacttcaggaaaaaacaactgaagcagccattcaaattaggtcctgcagccacaacgaacctgtttcatattaagagtcaggatctcaaaaagttctcaatcagtaaatctttgctattcagagaaaataaaagatcctctcatttctgcatttaacatttacagtttacaaactgttttcattggctcttttaaaagcttgtaactaggtggatactttttaaagaaatctattaaatttaatgaatcaattttgctgcaaaaacaaaacttagaaaaatcctaaaatatttaatattgatatttgaatcctagaaatcagaatcattccggaaattaacacaggatcctaaattttgcatgaaagggaggtagagccaggcgctgtggctcacgcctgtaatcccagcactttgggaggctgagacgggcggatcacgaggtcaggagatcgagaccatcctggctaacacggtgaaaccccgtctctactaaaaaaatacaaaaaaattagccgggcgtggtggcgggagcctgtagtcccagctactcgggagactgaggcaggagaatggcatgaacccgggaggcggagcttgcagtaagccgagatggcgccactgcactctagcctgggcgacagagcgagactctgtctcaaaaaaaaaaaaaaaaaaaaaagaaaagaaaagaaaagaaaaaaatgtaaacacaatatagcaacttttgtgatacagcaaaagcaatgctaaagggggagtttatagctataaatacatcaaaaaccaagaaaatctcaaattaacaacctaactttacaacttaatgaactataaaaagaaaaactaaatccaaagctggcaaaaagaaggaaataataaagattaaagccaagataaatgaaatagaaaatagaaaacgatagagaaaatcaatgaaaccaaaaattggttctttgaaaatgtcaaagcattattcacaatatcttgaatagggaagcaagccaagtgtccatcaacaggtgagtggataagcaaaatgtggcatatccatacaatgggatagtattcagtgttaaaaaggaaggaaattccacaatgtgctacaacatggatgaaccttgaggatgttctgctaagtaaaataagtcagtcacaagacaaatactgtatgattccactcttatgagtagtcagactcagagacacaggaagtaagatggtgactgccagaaagtagaaggtagaggggaaccgagagttaacatgtaatgcatagtttgtttttcaagatgaaagaggtacagagatggatggtggtgttatgaatatacttaccaccactgagctgtacacgtaaagatagttaagacggtaaattctatggattttatttttatttttttgagacagagtctcactctctcccccaggctggagtgcagtggcgcgatctcgctgtaacctccgcctccccggttctatgggattctcctgcctcagcctccctagtagctggggttacacgcgcccgccaccacgcctggctaatattgtatttttagcagagacaaggtttcaccaggttggacaggctggtctcgaactcctgacctcagatgattcacctgcctaggcctctcaaagtgctgggattacaagcgtgagccaccgcgcccggccattctatagttcattttcaccacaaaaacaaaaatgaaattcacaacaagcaaaacttcagaagggtacaaaggtagagagcagtgtgagctaattttacagtctttcaaagcatacagactgatggcatcagcattagaggtgcatgcattttaattagaattgtggtggtaacattcataaaactgaaaacaaaaaacgaagaagataacttctgggaatcgggactttcagtggaggcggggtgatgagaaaaggaatatttactgtcatccaacaactttgtcttgtacttttgtcataggtattatgccagtaatatccctgagaatgtatggggcaggtctagctctttttttcctaataaattcattcacacaacaatcaaaacagcaagaaaaggaagagatatccgcacggcggcggcaggccggggccgttccaagggcctggccggaggccacaggccagacgcggcggaaccctcccggagccgcgcccgggcgcgcgcgggaagcggaggcggagtgggcacgtgcggcacttccggggcggggcgggcacgcagcccttccgaaggcccgcgcgagccgctagttttgcccacgcacttttggcacagccgcgccacgcgatcggcgatctgattggcccgcgcgggagggcgcgcggcgccaaacttgcttcccgtcagcccccgcccgtcccgcgggagcgcgcacgctcgcgcacccggatcccggctcctgcatccagtcgccattcgggaggccgctgcgctgcagggcctcgcggagccgcccgcgaccgcgagccgggccctccgcgcggtccatcgcccactggacgccgcccgcggccggaccggtgaggagcgagagcgagcgggggaggggcgtggttggggcctgcatccccgagccccgtgtcggccgccgacctggcggggacggtcccgggaagccgcgggtccgagtgaagggggcctgcgctgcctcgcttcccacacggcccgagggaagggcccgcccccacaggccggggcgggagtgcgtctttgtgcagggagcggggaggcccggtgtcagtcgctgccgaggctggaaacattgcgtttgaaaccattgggaagttgagggcggcacaagacgaaagccattctttctgttttagttctagactgcctcctcttctgtcattattactgtttaaaaatccgctatacagcctccaaagtgggagaggacatttgtaaacctgataaagagttaatgtctagaatacaagaagagatgcaactgaccaacaaaaagcaacccagttcaagaatgggcaaggaatttgaatagatatttctccagagaagagagacaaatggccaataagcacgtgcagtgatgctcaacatcactaaccattaaggaaatgcagatgaaaaccacaatgagataccacctcacacctgttagggtggctattaaaaaaaagggaaattatgagtgttggggatgtggagaaattgaagctcgggtgcattgctggtgggaaagtagggtggtgcagcagctatggaaattggtagtcgttcctcagttctaggtttatacccccaaaaattgacagcagagactcagatacttgttcacaaatgttcatcgcagcattattcaaaatccagatgtctatcaacatgtggatgaaaggcaagctgtggtatatacatataatgaatggaccattgttcatctttagaaaggaatgaaattctgatacatgctgcaatataaacgaacacattatacagtataaacgagcacattgaacacattatgttgagtgaaataagacacaaaaaagacatactgtatgattccccctcaggcacagaattgagattaccagcactgaaagtggagagtggaagttactgtttcatgggtacagaatttctgtttgtggtgaagaagttctggaagtggatagtggtgatgtttgcacaacgttttagtactaaactaactaaattgtgcttttaaaaatgattaaatggcaagttttgtttttttttttttttttgagacgggagtttcgctctcgttgcccaggctggagtgcaatggcccgaccttggctcactgcaacctccgccccctgggttcaagggattcttttgcctcattctcccaagtagctggcattacaggcgcttgccaccacacctggctaattttgtatttttagtagagatagggtttctccatgttggtcaggctggtttcgaactcctgacctcaggtgatccacccgcctcggccttccaaagtgctgaaattacaggcatgaaccaccatgtccggcctaaatggcaagttttttgtttttatttttgtttttgttttttaatgagccagagtttcacttttgttgcctgggctggagtgcagttatgcgatcttggcccactgcagcctccgtctctcggatttaagcgattctcctgcctcagcctcccaagtagctgggattacaggcgcctgccaccacacccggctaattttgtatttttagtagagacgaggtttctccacgttggttaggctgctctctgactttcaaccttaggtgatccgcccaccttggcctcccaaagtgctgggattacaggcgtgagcctgtggtggcgcgcgcctgtagtcccagctactggggaggctgaggcaggagaatcgcttgaacctgggaggtggagattgcagtgagccgagattgcgccattacactccagcctgggtgacagagcgagactccctctcaaaaaaaaataaataaataaaaatgactccttgtgtaaactatagactacttaataaatcagtattagcttatcattgtagcagatatctcacactaatgcaagatgttaatacaggtgaagtatccttcattcgaaatgcttggaaccagaagtgctttggatttttttttttttttttggattttggaatagttgcattgtacttactggttaaacatccgtaatctgaaatctaaaatgctccttttggcactcaaaaagttttggatgttggagcataatcagatttcagattttcagattagggatgctcagcctgtagtaggaaaaactggggacaagaagataggtgggaattcttgtacattcccttcagtttttcttcaaacataaaactacttggaaaattagtttattaatttctttcttttttgggttgggggtgagtatggagtcttactctgttgcccagctggagtgcagtggctcagtcttgggtcactgcaacctccgtctcccgggttcaagcgattctcctgcctcagcctcccaggtagctgggattataggtatgcaccaccacgcccagctaatttttgtatttttagtagagacagggtttcaccatgttggctgttggccaggctggtcacgaactcctgtcctcaaatgatccgcctgccttggcctcccaaagtgttgggattacaggtgtgagccactgcgcccgcccagtttattaatttcttaaaggccaggcatggtagctcacacctgtaatcccagcactttgagaggccaaggcaacaagatctctggaggccaggagttcaggaccagcctggacaacatagtgagacctcatcgctacagaaaaaaaaaattagccagatgtggtggcaggtacctatggtcccagctacttaggaggctgaggcggggggatctctggagcccaggagtatgaggctgcagtgagttgtgatggcgtcactgcaccaaaaccttgggcaacagagcgagaccctgtctctaaaaaagaaaaaaagtccgctaagataaagaacaatgctagtgtgtatagactaatagaacccatttagctttgaacatgttctacttattgaattttaatttaaaaaactatgcatcagatgtgttttaaaagatgatcagtggtgttcgacttcaaagaagaaattgaatgggagatgggaaataggtatgatacaccatctggcataacattgcattgaaggatactgggtgatagactggcagagcaccaatgattagaatttaggttttttagagacgagatgtaatctgtctcaaagacaaaaagaaatttttgttaatctggacaattctgagttaatttttagttagggttaggagttcttaaatttaggaagaaattttgctagaacctaaaagctggaaggacgcccagctctctaggctttgcctaaaagttgaattctgggactaaattctgtaacatcttcgtggatcgttctgctactgtgggaaagacagcattttgttacagcagagaccagaattgagaaaaccagaataaaaaaactgttccctctgacctcagtgttgtgatctttgacaggtcacttctttccctcagcatcagtcttttcagttataaaatgaagaggttgaactcaagacctctgaagatccttctagtcctatgctgatcatcatttttttttttttttggttttcttttttgagacagggtctcactctgtcatctaggctggagtgtaatggtgcagtcttggttcactgcaactcctgcctctcaggctcaggcgatttctcttgcctcaggctcccgagtagctgggactataggtgcattctgctgcacccggctaatttttgtatgttttgtagagacggggtttcgccgtgttgcccagggtgttcttgaacccctgagctcaagggatccacctgcctcagccttccagagtgctgggattacaggcctgagccaccacacccggcctgatcatcagattctaattatggagtactcctacctgacaaaggaactcgttctagttcatgatagagtagattgtaaactgtgttaagccacaattcttttcctgctagcttctgattgtcacttctaggatagctcatatattatctataattcttccccttttgaagttaaatgtctttatttgtaaatgacttttttttttttaaagggccatctgccatcctggttgcctttttttttttttttttttttttttttttaatgttccctagtttgttagagactctcttaaagtgtccttcctgcccagagctggggacagtgatccactggaatagtgtactctgcccagtgcagaatatgggatatagttctctatagtcctgtatttatgcacttgatatgtggattctaagtgtttgtttcatcttgtttattttggactattgtacaggctttaaaagatcttactaaattccactgtattagtttgtgtatatctacaaagaacctggacagatagcagaactttattctctcatcagatactgatcatttgtctgatttgggcaagctgtttaatgtcaagcttgagtttccttaactataaattgaagttttaaagtagatgatccctgaggtcttttcctaccctcaaatcctatgactttgagactagatggtaacttaggtttcctctagttttaaaatcctgtgagtctaagattgagagaatgttaggattgaacagagcacatactgtgtgtcagggatgtgcaacggttccagaggtgaataaagggataatagtccctcctctccaggagctggcatcctgtttttggtatcaggctgtagtgccagagggtgacatgtacagctggggcttctaggaagattgggaagaagtggggaattgaccagtacagttgactcatgaataacagaggttagaacttcaaccaaatgccaataaaaaagagcgtttacagaatgtgaaacatttatatggagggcagatgtttcatatatgggaattccacaaggctgactgtaggacctgagtatgtgtggattttggcatatgggggaagtcctggaaccaggtccctgtgtttacagaaagacagctgacttaaaaagctgtcctgcctgaaggatgaaggagtttgcctgatggccgggtggcaagacagggagtgtaaaggagaggaaatagtttatacaaatgcacagacgctggaatagtcagggagttacatgtgggccagtattttgagaatgcagtctggtcatgggccagactgtgaagagctttatatgccatattcaaattgtatactgtaggtgtcttagttgacttggaggacatgtgaacaggcagatcacattaggcttctgtttggaagacaggtttggtagctgtacgatgaatgggttggaagaaaaggagactagtggcagggagttcagatgtcagaattaaagcagtgataggggtaggggatagagaaggggtagatgaatgtgctggagctaatcagaagcataatatgagctgttgtgtagcttgtgcatggccagtttatcacattctgttcctgtcagcagactcacccacaccatgatgctttatctttgaaatgcatgcaggaagagaagagggcagagcaatcactgaagcaggtccatgtgttcttccttggggtcaggacactgccatcctgctcctcccagagtcctttctttttgtgcctcttggtcctctttgttgtggtgtcccttcctccactgcctgttaatttggcactctgctgtgtaatatcaataattgacaaaagtcagccattgttatagtcagtagtcacaccagcagtcttctgagtgcttatcagagaagtgaagttaggtggtgggtttgagaaggatttgattacacacacgggcataattccatagtggatcattaaagagaaagtagacaaggatggtgtcaggagtggtatctactgtagtctttggtgtcatttcataaaagtaagtgttcgggctgggcgcggtggctcacacctgtaatcccagcactttgggaggccgaggcgggcggatcacgaggtcaggagatcgagaccatcctggctaacacggtgaaaccccgtctctactaaaaatacaaaaaactagccgggcgtggtggcgggcgcctgtagtcccagctactcaggaggctgaggcaggagaatggcatgaaccctggaggcggagcttgcagtgagccgagatcgcgccactgcactccagcctgggcgacagagcaagactctgtctcaaaaaaaaaaaaagtaagcaggcttagtggactggactcctctcactgttagggcaggatggtgagagaaatacacagaattccattacagggggcacacacgatcacagatatagttttgattacttttctttttttttttttttttttccaggttcaacttctcatctttgttcttcttcatatactataggctgtttgctgtggtttagtcaaaaagccatgtagaatgcctgccttttgaagaccacttttaaggtgtctagtaagacagcaggtaagtctagtagggaaagcatgcttaggagaatattttatgaggagaaaattaaaaaaaaatacctggagttcttgaggaaaatctctaaaagtgtacacgcccttatggatataatgtgtgttgtgtgctagatatacataatttgggatatgagaagtgaattctgtagcattctttgtagatttaagtatgagattggcctgaagagctggtggtaggcagcgggaagaggagggatggctgggaggaatggggaatggtaggtgagtcagactcaaacccagcacacctgagctcttagtctctgttgtaattacaatatgtaatatagtgctctttaatattagcttggctgttcattattgccagttgggctgttcattattgcctcatgggattattgagacatatcctacgttttcccctaaagtcttctagttttatttaacaatgggctctgagacggatggttaacccattatttttccatgcattcaattacgtttgtaagttaaagatcattttcagagatttttcatcagtacggaattatttatttttcttttgttacagtcctggccccaaaacagttttctaaaatacaggataactgctgggcaactcagaaagagtaaatagtttccgaagaaaagtatttgtttcctcaagtatctgtcacctttttagctttagatcaaaaaataaaaaaggcaaatgatataccttggataaaagtgttaggaagcagtattgccagtgagaacatctttaaaaacaaattatcaaagttagaaatgttaatttttttttttttttttttttttgagatggagtcttgctctgtcaccaggctggaatgcagcggcatgatctcagctctctgcaacctccgacttctggtccaagcgattggcctgcctcagcctcccaagtagctgggactacaggcacacgccaccacgcctggctaatttttgtatttttagtagagatggggtttcaccatgttggccatgatggtcttgatctcccgacctcgtgatctgcctgcccggcctcccaaagtgctgagattacaggcatgagccactgcgcccagcctatacatttttttaaaataatggttttgtaattaataaagggagagcctgcatttctaatctgtactgttctttgaggaggagtaaatatcattaccgtcaataagggaaggtgttcagtctttctcattaaagagaagcacattaacacaatagtgaaatactagctttcacctaggttgtagcaaggattaggaatttataggaaattgtctctcatatatacacgttgttatgcagtaatagaagtttttgagagtttggtaaaatgtatcaattttacacacaaatggccaagtaatccattgctaggaattttccctgcagaaataccctcacatgcacagataagtacagggaaagtcagtgcttattccatatgtaaaaatgtcttagagataaattctaaatgagagtaagttgccagataatatgtttaatatcccatttgtgtaaagcatgtgtgttgatcaatatatgcttaggacatttcttccaggacacatgagtacactggctacccctagggactgacactctgtatctcgtactgtgtgaatgtgtttttaactgtgagcttctattccctttactacttaagaagactggtttacaatttttttaaattcatgttttccggcaactgtgttccagctgctggggtgtcctctgatttgccctttggttggtatgcgccttcacatcattcaggatctctgctggaatgtcccatgcatagaggtcttttccaacggcctctacatcactctccaccttatctttccaactcatgaacgccatattgcaaagcactgctttaagcctaagtgcctagaacattgtaggacacaaagttatttatttattaaatgaattaatagaatactatattgggatagattaaacattacaaaatatgttgacatgaaatcaagccagcatcatgttttaaagtcatctttttatagctagatttaatttattaagtgctttctgcaaaagttttagtctcttgttcatccttaaatagccaagatttaattttgatattcactcatctttgtgtgctgaggacttttgtactcggagtttgagagtcaaagtataaagcacatgcaatagctaattctagagtcaggctcacatcactgatagtcctatagcctggaccagagaaagtcaggtaacttctctgaacctgtttcttcatctgtaagtgaaaataatagttgagtcatatcattaggattaaatgagatgatgtatgtgaaactcctagcagagtgcctgggactgttctatatctcctttttcttcaaattctccctagagatactatcaaataaagcacgcactgttctagacttaaaattatgcagttatagaatggactgacagcattttattcagaattacagttcatgaaaataatttattcctactttattgcagcagtattgaaagtttttaaagaatataaccgtgtgtgttggtaacagacagaagaatggaagcattccaggaacttcgtaaaccatcagcacgtttggagtgtgaccattgcagtttcagaggcacagactatgaaaatgtacaaatccatatgggtaccatccatccagaattttgtgatgaaatggatgctggtgggctaggcaaaatgatattttaccagaaaagtgcaaagttatttcactgccataaatgcttcttcaccagcaagatgtactctaatgtatactatcacatcacatccaaacatgcatccccagacaaatggaatgataaaccaaaaaatcagttgaacaaagaaacagatcctgtgaaaagccctcctcttcctgaacaccagaaaataccctgcaattcagcagaaccaaaatccatacctgccctttcaatggaaacacagaaacttggttcagttttgtctccagaatcgccaaaacctactcctcttactcccctggagcctcagaaacctggctctgttgtttctcctgagctacagacacctcttccttctcctgagccttcaaaacctgcctctgtttcttctcctgaacctccaaaatcagtccctgtttgtgagtctcagaaacttgcccctgttccttctccagaaccacagaaacctgcccctgtatctcctgagtcagtaaaggctactcttagtaatcccaaaccccagaagcagtctcatttcccggaaacattggggccaccttcagcctcatctccagagtcaccagttctagctgcttccccagaaccttggggaccatccccagctgcatctccagaatctcggaagtcagcccggactacctcccctgagccaaggaagccatccccttcagagtctcctgaaccttggaagccgttccctgctgtctccccagagcctaggagaccagcccccgctgtgtcaccaggctcttggaaaccagggccacctgggtcccctaggccttggaaatccaatccttcagcatcatcaggaccttggaagccagctaaacctgctccatctgtgtctcctggaccttggaaaccaattccttctgtatctcctggaccttggaaaccaactccatctgtgtcttctgcatcctggaaatcttcatcagtctcacccagctcctggaagtctccccctgcatctcctgagtcatggaagtctggcccaccagaactccgaaagacagctcccacgttgtctcctgaacattggaaggcagttcccccagtgtctccagagcttcgcaaacccggcccaccactatccccagagatccgtagtccagcaggatctccagagctcagaaaaccctcagggtcaccagatctttggaagctttctcctgatcagcggaaaacttctcctgcttcacttgatttccctgagtcccagaaaagttcccgtggtggttctcctgatctctggaagtcttccttttttattgagcctcagaaacctgtcttccctgagacccgaaaaccaggtccttctgggccatctgagtcccccaaagcagcctcagatatctggaagcctgttctctctatcgatactgagcctagaaaacctgccctgtttcccgagcctgccaaaacagcccctcctgcttctccagaagcacgcaaacgtgccctttttccagagccccggaagcatgcccttttccctgaactccccaaatctgctctattctcagaatcacagaaggctgttgagcttggtgatgaactacaaatagatgccatagatgatcaaaaatgtgatattttggttcaggaagaacttctagcttcacctaagaaactcttagaagatactttatttccttcctcaaagaagctcaagaaagacaaccaagagagctcagacgctgagcttagtagtagtgagtacataaaaacagatttggatgcgatggatattaagggccaggaatcaagcagtgatcaagagcaggttgatgtggaatccattgattttagcaaagagaacaaaatggacatgactagtccagagcagtctagaaatgtgctacagtttactgaagaaaaagaagcttttatctctgaagaggagattgcaaaatacatgaagcgtggaaaaggaaagtattattgcaaaatttgttgctgtcgtgctatgaaaaaaggtgctgttttgcatcatttggttaataagcataatgttcatagcccttacaaatgcacaatctgtggaaaggcttttcttttggaatctctccttaaaaatcatgtagcagcccatgggcaaagtttacttaaatgtccacgttgtaattttgaatcaaatttcccaagaggttttaagaaacatttaactcattgtcaaagccggcataatgaagaggcaaataaaaagctaatggaagctcttgaaccgccactggaggagcagcaaatttgataacacagtgtgaatatttgttctacaaaggtgtttgttggaaccattctttgtaagtatagcttatcagatagcatagttggatcagtagatgacatgtatggtgtaccgtgtttcactgtctcagttgtgttactaagaatgagcatttgatcatttttttctggtctctgtctatgtgactatcttgtaagtcaataaatttctgtatagtccagatggattaaacttctcatttcttttaaatatgtatgaataataatacaaggaagtaggcattccatttaataatcaagagcaagttgtactcaaagcattcagttaaagtgtatctgtgtgtggaactaatttcagacaatagaaaatattagttgaaatgtttaagaattaggcatgaaaaataaatttgagaaattttgtttccttacatgtatttttaaatcataagagttattttctatctgatgtaaaattagtttataaatcttaatcagcttctagatgtttattagcttttatgtcatgaaatgttggagtctcagggttgctgattttctgctaatgggaaaaattgactaagtctttaaaatagtttgcagccttctcccacaggagacaagtgaaagataagtgtgattttagatctttcttgtccatagttgttttcagtggagtcttccattctgtatcttaccctaagatctggttcttccctccccatccccaccccccacccaccgcctgccagctcacactaatagatgattcttaattgccaaatgtgttagagtttgtatatcctactcctgggccttacatgtcgcctgttggggcttaagaccaggttgataagtaggaactgaaagtcttccagattcacagtagaaaattttatagacatttctgttaaagaaatatatcgattttatgtttttcaattatgttactgtaaataccttgtacctgttcatggattattttattctaaaatattttgtcaaatgtgtatcaaccaaattaaaaagaaaggttttcatgtcagcaacatcttcatgtgtgttttcttttgtgtagttttcattgtatgatatacagatccattatttccatgttttcaacatagttttcttatctggagcagcattttcttagctgtgttttctccaagttctgtggtcaagtaagttaggggaatgcgtgtatagggaggactctgtgtcaaaaacctcatcctttcatatgaaaggttttttaattccagtggttgcctatcctgcctgcctctaaacagtgtgaggcatttaatctcaaagcattctgatgtgtagccaggattgagatgctgtactgggtttattttagctattcccagacatttttttaacaaggaacagttttgtcctagttccccacccgctggcttccctattaatatgcccgttgggatagcttaacaatatttagtaatatggtttggctctgtgtccacactcaaatctctctctttttatttttgttttttattttgagaccgatccttgctgtgtcacccaggttgctgtgccgtggtgtgatcttggctcactgcagtctctgcctttcagattcaagggagtctcgtgcctcagcctcctgagtagctgggactacatgtgtgtgccaccacatctagctaatttttgtagtttaatagagatggggttttgccatgttggccaggctggtctcgaactcttgggctcaagtgatccacctgcctcacttgagggagtgctgggattacagctgtgggccaccacacctgacccccacccaaatgtcatattgaattgtaatccccagtgttggaggaggggccagcctggtgagaagtgattggatcatgggggtggacttccccttgctgttctcgtgatagtgtgtgagttatttaaaagtgtttagcccctccccttcgctctgttcctcctgctccagccatgttggatatgccagcttccccttcaccttctgccgtgattgtaagtttccagagccgccctgcctcctgccccagctgtgtttcctctgcagcgtgtggaaccattagctaattaaacctcttttgtttatgaattacccggtctcaggtctttgcagcagtgtaagaacagagtaatacatttagataataccaagtaaggtcttagaagcctcagatctgttaacttcggtgaacacagcactgagtagctgctgctatcaatttacgtggcaccttgagaacgagcttccagatcaggagttcccttctaggcagtgatgccaacaaataggacaggtaagatatcttggatatactagttgtccttttcttgtcgatactgactgactgaagttgtttttgagaaacaaaaagggtctgcattacctagaaactctcctggctcctgcagtctcagaccaaccctgttacacagattcttttctaagacttcaccctgtctggtgcaaacctggcagttcatcctagacctttgctccaggtggcaccaactaagtcacatgacttgccatctgatttggggttttttataatagtgccataggataagagttaattgcctgggtaagtggtagtgatgcatgtgtagttttggcttttaaggactttatttggctaaacccctgtgttgttggtttaccttactggcttccactctttctttttttctgtttttgagactgagttttgctcttgtctcccaggctggagtgcagtggcgtgatctcagctcaccacaaactctgcctcccgggttcaagcgattctcctgtctcagcctcccaagtagctgagattacaggcgcctgccaccacacccagctaatttttgtatttttagtagagacagggttttactattttggccaggctgttcttgaactcctgacctcaggtgatccacccaccttggcctcccaaagtggaggatggagacgtatttaaaagcactgagcacaaaggaggcactcaatccatggtggtaattattattctaaccttattagccccctgccgattcaatgtataccattcactagtttgtttaaataatttgtgaatcagtttatattttcaagttgccatatgttttagggttctccggagagtcagaaccaataggatatatatatggtatagaatggtaaccagcactctacactctgtttttatgagtctgacttttctagattccacatacaagtgagattatgcagtatttgtctttctgtgtctggtttattttacttaacataatgtccttcaaattcatccctgttgccataagtggcaggattttcttcttttcaggctgaatagtatttcattgtgtgtgtgtgtatatatatacacacaatttgctaagcagggagatccgcagaccctcttggctcccagcatgctggggtcaggtttgcagtcttaggagatcggtgaactctttttggagaggatctgcaaataccaacagtttcagggtttcctcaggtccaggagtgccaggctcctgtgcactcttcctttggggaagcaaaggaagactccatcagtgcacacagaatgcattaattatactttcttattttctgttttcttcatgctctagcatctggggtttactgggaaaagattgccttttccagggccaggcaattaaactaccaatccagaatgcgtactcctaaccacctactttatctgtatatatattaccttcatatatatatacacacacatactacattgtctttatcagttcattcattgatggacacgggtagattccatatcttggttattgtgagtaatgcagcaataaacatgggaatgcagctatttctttggggtcccgatttcatttttttgtggataaacacccagaaatgagatggctcttcattactctgtgaaaggccgtaaaagccaccacacccctaagtgaaatcaccacattagtgaaaataagctgtatctctccacccaacgttctcttcccatgcttttagcactcctggtcccagtgccaggggttttctgtaactccagctttttgcatgctctattagcattagaaaattatttctctttaatcaggaaagccacgctcaggctattgcaaatcacccactttgaacaggcctgtgacaagcctagactagatacaagtaattaataagaggcaggtcgctgggcgcggtggctcaagcctggaatcccagcactttgggaggctgaggcgggtggatcacgaggtctggagttcaagaccagcctggccaatatggcaaaaccccgtctgtactgaaaatacaaaaattagccgggtgtggtggcacgtacctgtagtcccagctactcaggaggctgagacagaagaatcacttgaacccaggaggtggaggttgagccgagattgtgccacggcattccagcctgggtgacacagcaagactctgtcaaaaaataaataaataaaagcaggtaaaataattaagcaacaataaacacaattggcagtggtcatccagaggactatgacttccctgaggaggacgtgtgaaggttgaaaaccaggtaataagtggcctgaagacatcaaggagtgcaagtgaccctattattcattattccttaatctgttctccagccaagactgtggggttagtgcctcccactttcatggagataaccgactgtatacaaacagggtctgtgtgggagtgggatttcaaggactctcagcactttttctatatttgctactttactgagttggatacaggcaataaggtctccatttgacttgcgtggggctgaggcactgatattcacaaatacaagtatagtactgggtataatggaaccgatgggcattctgatttggttaaatttctgttggtagatggttgtaaataacagaacaaccacctgctctggtttgcccatgactgttacagcaacttgggatggctggtcacccagaagaacacagccattcttagagcacagggtgcccccagcctcactgtgtttgcaggtgccaaccttcgatccctacggctgtcattctcttttttttttttttttttttttgagatgaagtcttgctctgtcgcccaggctggagtacagtggcacgatctcagctcactgcaagctccgcctcccaggttcacacctttctcctgcctcagcttccccagtagctgggactacaggcgcccgccaccacacccaggtaatttttttgtatttttttaatagagacggggtttcaccatgttagccaggatggtctcgatttcttgatctcgtgatctgcctgcctcggtctcccaaagtgctgggattacaggtgtgagccactgcgcccggcccagctgttgttcttgagatggcatcttcctttcagtctcacctttcctggcaccagacccagcaaaaggcaaggcaacaggaagcagcccatggctccttttgagcctgaggccccagagcctgcatctggaaccttctaactgcagaggctgctcccaggggtctgcagaagggatggcatatctccccatggtctgtgtcatgggccccaccaacatagtctacaaggtctcaccggcaggtcttcaggcctaactctaaattcaggaagctccctacagacaacacaggttcctgaatgttgtcgtctaatctagcttcaaaatcttttcacttacatatggggtgcacattctttacttggaaaatgatttcacaggagaatgcctgactgaaatttactcatgagtaaacctttgcactctttcctctgaaaaataataacatgaggcccattatcagttagtgataatctaagaaaagtgatgaagacagaggcctagatctgttctgagttggggttgcccaaacctgcagggtgagtagcctctgatttgtaccttctggtgggtgaggtaaatgtgctcctgcaggtcttatactcattgcattatctccatgtagcatcacatatgactttaaagcactgtgcttggccgggcgtggtggctcacacctgtaatcccagcactttgggaggccgaggtgggcggatcacgaggttgggagatcgagaccatcctggctaacacggtgaaaccccgtctctactaaaaaatacaaaaaattagccgggcgtggtggcaggcgcctgtagaggcaggagaatggcgtgaacccgggaggcggagcttgcagagagccgagattgtgccaccgcactccagcctgggtgacacagcaagactccgtctccaaaaaaaaaaaaaaaaattctgagtacattttaaaacataaggcatccaatggaatttttaaaacccttatatggataattttaaacatttgtgtaaataaacaatgaatttaacaaactcctatatgtctataacccttcagaacaaccggaaacacatggccaattctgttctgtctatattcccaaataaaattcatacattgtgattagctgatgtgttttttagtctatataatttatacatttcccctatatctcctatatatcattttcttgtaatttatttcttgaagaagctgtgttgattttcctgattcatcttcagagtttaaaatagaatttcacaaaaagaaaaaaataaaagcagactgaaggaataaaaatgtcagaaaagaaatcaatgggatttaaatggtacagctgctttggaaaacagtctggcagttcctcaaaaagtaagacatagaattactctttgacccatcaattctactgaagtcttcatactgaagaggattaaaaacatgtccacacaaaaactgggacacagatgtccatagcagcattatccataatagccaaaaggtagaaacaaccgaaatgtccaccaactgatgagcggataaataatatgaagtatattcatacaatggactattactcagcctcaaaaaggaatggttgccagaggttggaggaggaaaaaatggaaaatacagtatttctttcttgggtgctgaaaatatgttagaattaaatagtggtgaagtttgtgatacttccagaaagtgctaaattgtacacttcaaatgaataaaatgtagtatgtgaattatacctcaacttaaaaatcaagaacattttccagaaataaagtttaagaattttcagatttaaaagatctattgagttcccttcaaaatgaatttcaaagttcttctacactaaagcacattattgtgaaatttgataacatcagagaaaaagatctgaaaagcttccagagaagtgaaagaaaaagatcacctgcaaagaatcaagaaccaaaatggcactgaagtttttcattgaaattttagatataataagaaaatgaaatttggccttcacattctgagagaaatacatatattttaaaccctactaagccgccaacccagtgtgagaatagtgctcaggcattttgggacatgcacagcatcaaagctttaccttcgattaggaagctacagtaaagtggcaacctaagaaatgggagaatgaattccaggagcaggtgatccatgcaggagagaggagcagcattgccgagagccaggggtgaagcaattcaagctggagacactgagacaaacacaggggcgcctggcattctctcagaggcagtacagtggttctcacaactcagtaaatgtggtaattatctcttatctggttacaatgcatcttcccaggcctgccagcagggattctaaatcctgatgcaaatgatgtggcagcacttgtggttatagacatgagtgtttgaatcacagatccaggtccaaacccaaatcccctcagtaattaactgtatgctgtcattcaattacttaatctctccagcacgccacttcctcaggccaattcatatgggaatataaattcataggagtgttataaagggtagtttgtacaatgagctcgtgactatgaaatgcttagcatggcatccaaaaagtcctcagtaagtggtgcaacaatacctttttattacagaaaacacactcaacctagaaaacagtgttcataggggattcagagaagtaagtacagttgggtgagcagagcaagccagcgtggagagggtctttcctaggtgaggcctctaccacatttgaaagtgggatgcagggcaagagctcaacatcaaccaggacgggcaggaatgaagtaaatagctcacacctgttccttcatgggtcagttcctttcattttcacttttgactctgatgcaaaattagaactctgctcaaatgtcactattcttaataaagtaaagtaaataagtaggatccaaaaacttcaattgcataaaatacgtgctcttatgacaaggcaaatgggaaatattttgcagttggtcattgccatcacaataacacaaattctaaaacttatactgtttacaactgaaccacaccctcaaataatcaaaaaatggtccctccctaaactctctccccaggaagagaaagtctttggctgccccaacctgagcaggaaagcaggtggagagggggagcccacagctacagtggcaagaagtctgagggctgagggctttgaccaaataatagggaattgggttgagttgtttgaatcattttccaataactgacgtataaaatagattcattgaaggagctggggaatgggagcccacccctcagtctccagcctcccctcagtctccagcccccccaaactacatgtttctccagagtaaccttgagcctctcccagcatggattaaaacccatggctcttccagatctaaaatctgaggctctacaatttaagtcatagttaggcacttccatacctgtgatggtaacattcttacatactcagtctatcattgtttaatattgaatgaaagacacacattttgtttacttattatgcagcagctagtctgtgttcacctgcgttgtctcagggtcagctctttatttccaagaatcctgtcccatttcctggagcctattgaccgggggaaagatgtggtcaaaagttaaaggtatgttgggcaaagccttagaaacagcttttctgacaagcaatggggaaaagaccccgttcaataaatggtgctgggagctgggcccctttcttacaccatacacaaaaatgaactcaggatggattaaagacttaagtgtaaaacccaaaaatataaaaaccctggaagacaacctaggcaataccatcctggacataagaacaggcaaagatttcgtgacaaagacaccaaaagcaatatcaacaaaagcaaaaattgacgagtgggatctaattaaactaaagagcttctacacagcaaaagaaactatcaacaaagtaaacaggcaacttacagattggaagaaagtatttacaaactatgcatctgacaaaggtctaatatccagcatctataaggaacttaaacaaatttacaagagaaagacaaccccattaaaaagtgggccaaggacatgaagagacacttctccaaagaagacaaacatgtgggccgggtgtggtggcttacgcctgtaattcctgcactttgggaggctgaggcaggtggatcatgacatcaggagattgagaccaacctggctaacatggtgaaaccccgtctctactaaaaatacaaaaaattagccgggtgtggtggtggtcgcctgtagtcccagctacttgggaggctgaggcaggagaatagcatgaacccaggaggcagagcttgcagttagctgaggtcgcgccactgcactctagcctgggtgacagagcaagactccggctcaaaaaaaaaaaaaaaaaagataaatgtggtaaacaagcttatagaaaaaagctctagatcactgatcactagagaaatgctcatcaaaaccacagtgaggcaccatctcacaccagttagaatgcctattactaaaaagtaaaaaaccaacagatgctggcaaggttacagggaaaagtgaacacttatacaatgttggtgggagtgtaagttaattcaaccatggtggaaagcagtatggtgattcctcaaagagctaaaagcagaactaccattcgacctagcaatcccattactggatatatacccagatgaatataaataattctaccatgaagatacatgcatgcaaatattcattgcagcactattcacaatagcaaagacatagaatcaacccaaatgcccatcaatcatggactggataaagaaaatgtgctacatatacaccatggaatactatgcagccataaaaaagaacaagattatgtcttttgcattagtatggatgaagctgaaggatattatccttagcaaactaatgcaggaacagaaaaccaaatacagcatgttctcacttataaatgggagctaaatgataagaacttgtgaacacaaagaaggaaacaaaagacactggggtctacttgaggggggagggtgggaggagggaggggagcagaaaagataactattgggcactgggcttaatacctggatgatgaaataacatgtacaacaaatccctgtgacacgtgtttacctgtataacaaaccctcccatataccccccaacctaaaataaaagttaaaaaaaaaaaaaaggaacacagctttttatttttttctctttaataccataaaggtcatgggccatttgccacctgaacagtcagtaacacatgagtgtaaagaaactgaacacccagggacaccagagaccgttcactgtagaggaaggaggcaggtataactcataaggtgaacttcctccagtcccaaagtggttcccctctccttaaatgcgggtcccatggaattcagatttagaagtgaagcaaatgagaatagctcacaggggagcagttcaaatgtgccgaaaaggatagagcagccccaataaggaaggaaggccagacaagcaatggatttgtagggaagacaatgtgcacccatcagagctctgattccttcacttccactaccctgccctgcccactaactttgaaaaaatatggatggcaagtgttgggccgtaagacatttccttgctttataatctggatttagtgggttaaatttcagagatagtgaaaacatccccctttagttaacttttaatttcataattgtaccttttttttttttttttacagatatttgaacatgtaaaacaaagaaaagagtacggaagtaatttataatcaacaaatatgtgttcactgagtaagtgatactgacatggcatatggcatacaagcatcctgagagtaggatcatggggcattcaatggtgattgtccctagagctggttagggcccagctttccactgtccttgtactgctgcgtgtacaatcggtgtcttctttaacttcacagagatgccacaaggcaaatagcattgtcttcactttctgtgtaagaaaactaaggctcggtgagagtatgcagagctggttcatggatttgtttgggtccttctagtttcaaaacccgtgaatattttcctgtctctaatgctaatcttagcaatatatttggaataagcatataaacatataaccacagtccactcaggtaataggcaactaaattataaataaatgctcaacgagattatgagtgattgaatgagctatatacaagaataataactggtggaagtgttattcataggataaagtagaaatcatttaaatggctaatagtataaggagggttaaatcaagcaggtgtggaaatagctgatttacatatatagtttatgccatgtaaaacgtttataatctaatattggatgaaaaacagatacaatattttatgtacagtataatctaaaatttctatgtttagttaaaaatgcaggaatataattatcaaaatttgagtgataaaattatgaataatttttttctttgataactcattttatatttaccaaagatcctccaaagagattatacactttttttaactagaaaaaagttatttgttaaagagatcataaatgacataatattttacagaagttaagagctgagattgtgagaatttccagggactcctgccatcagcccacctcgcatcattgtttacatggaaatgttgggactgaggtgacctcatgcctctcaattcccagccagccttctctccctctggctcttagctagcctgttaaacagaaaataatcagtgagacgaataaactacaccctagtgttctaacaagttacacataattgcttgcttaccaattgtctacctcagaactttcttttcctcatagagtcaacataatcatctattttaaattcggcttttgccattttacagtccaagatgctggactttgcatagggcatggatgggtttccacagtccacacgaggatgggaggagaacagggcagatccagccccactcagtgagagttctggctggagttagttggctggacttcactccagagagagaccttcctctgtaccagctaccaagacagcccctagttggaaaatcttttcttctaaggtgagatgcagcccccactggagagaaaaacaccagagagttttaaaaaggaaaaaccaaggtaagctgaggggcttcttgacaggggacacagcagagtccacccagctggggtgaatctccgcagttccccaacaggacagcgggtgggagccccaagtcactggcccctgcagtgttggagcagcgtgagccacctgtagtcacattgtctcctctggcagcagcctccatccagaaatgcatctcagggtctgtcgtcacaggaacctgatgccagagcagtgcgctcaaagtatttggccacaggtattttgtggaactgagcattttccacatcttgctggaggtcattgtgtgtgaacttgatggagctctttctcctgggctttttttctaagtgtgggagagcatggcctgcatgactgctggcttccagcacgtgtcgtgcaaacccatgccagggtgtgtaattcactgtagcccttgacagaatagcaggggataaagctcattttttaatcttctgtttattaccctcagccaattaacttctatctacagtagaagatattttgccaattttttttcaaaatggagaagtaccgtgctctttaaaaagtcttttagaggcacagacttctcaatttcattacattaaaatcactaaaagatagaaaaaagtgaaattcacgtatcaaagaaattttcttttagcaaaccactctattttgttttgaaatgtttgtgaattttccatgcatgtgtccagatacataaggagtttattcattcaattaagttaaaacattggataacatggtaaattcaaaaagtagtgaggacacgttttgtttctctctgtaactgagaaaactgactgatgcctcctgtgtgcgagcactgtgtagctgatgcagatgtggaaaccaaggttttcttacttgctgccgctggccctggcactggatgttggctcctggagggcagggtgtctgtttttattaccactcagcaagtgccacacaaatacacatgggtgtctctgccctctagtggatcctggcaggcactgggcgcaaatgtgactataaaaaaaatgaaagatttaaaaaatgcccattacctctagaggggatttaattataacacgaggcttttttttttctttttcaaatgggctgatgggtaaaaataatgcaagactggagatgcgtttgcctttcggcatctatgatgactgagtctttcctcagtgctgccacagggagcacattcacacatatgtcacacaaataccagggttaggtcaaaaaaccaagtgttacctggaggaagccacagaggctatgagatgaagccttcacctagaagacacccgacagttcagaaaccgagatcctgtgaggtgtcttggagagctcagctgtgaccccgcaccgtatgaaagggtgggcattggggttcaggcaggtgaagagaattgtccactgttgagcatcatcagcctaaaacccacatgcagtgctactgctgcaaaaccactgactgttgaagccacaaaccgcagaaacagaagccctttatggtcctcactgtgcaactggtgacttttcagaagatgaagattaaacatattaatcataaattcaaagtatcagaagaaatactaggagattttctaacctctgctgccatgactttttttttttttgcaagattcgtgaattattgtaaataagctcacagaggccacagtgctcccctcaagtcatccagaaagatgcaggctgctaatccccacatctgacacatctgtgttttcttatgataatacacaaattaaagataggggagagagagagggacagaaagagagagacagagagaattggagctaagaggaaacactttcctttttatttttatttatttttaatttatttatttttaagaggaaacactttcatgttttgccccccaaaattcccagtgaacattcccattgccagtgggtgaaatccaaggccctggctccaggatcctgctctttcctgtggtgactcctgtgcagatttggcctagaaacactatggaagttgttacacttctctggacaagtcattatactcgtgcttttggctcacaggctgcacttccccggacaacccttcgccctaagctccaagtaattcagcttcatccttcagagaagagctcaggtgtcacttcctgtaaggtccctccctagccgcctctccagcagaagggcccccaccttgctctttgcacctctgtccctcgaggtggtgctgtcctctttggagggtgggaccccgggaactagagcccctctcatgctggtgacactcggcactctgtaagtgtgtgctgagcggatacaagtgcggcgagcttgcacagccttacaatgtgtactctcatgtctggcatagagacccattttcattctttgtaaaatgagtaagtggggctgggtgacccatactctcttttcaactcagaagtccactatgagcctgacgtcagatagcttcccagccagtgccagagcaggacccaggtttttcaatgtgtctgtgtccccacagcacggggaaagcacggaggaatgggaccagctctgcttcctgctgcccagtgtcagccagcaactccaggctcacaaagccaaggcaaaggcacctgcaggagatcttggttatttagtagctgtgattgggctgggaggagagtgtgttcacttctgattttccagtgaatagcatgagacagtaaggtttgaaatgtgttttttttctcttgccaaaaacgaaaacaaaaaaggctttttaccttctcatccttggagccactggcattagctatgggcctgatagtttaagaaaacttgaaaatgaactaggtgaaccagtattctccaaattaatattttgataaattgtggagttctgtattgcagcaaagctgttgcttaagtgggcaaaactctagaggataggaaaacacactcttgaaatcaggtggtgatgccctgaaattcagcaaactgcaggggcacgtgttgttctaccccaggtcagagtgtgtcagtcatcacttgatgccactcacagaccaccaacttcagaatatctaggtgtaaagctctgtacaaaagtcgtaacataataatgtaaataattttaccttaataatattatttatattacaccattacataatgtaaggctattaaaacatgtttgtcttcaaagaatggccttggtttctgtgggcagtgtctccttatggaaaggtagtgcattcctgctaagtcctggacaaaacgggcctccaggagctccaggctgcagcagcagcttctcatctatgtccttcactgcatgatgtcgttgttgactttgaaagctcctttcagtctagttttatcaacagagctagtttttcatgaggatctactacataccaggttccagaaggctaaatgccttttatttgttattattcactaaatacaaataacagctctcttctcattactcacacaacaaaatttagctgaggatggctctaggagatgcagaaggggctgcatcataaagaatgaagaagggatttgtcatgagagatgggacaggaaattctctagaggcagagggagagcatgagaatgttgggaagggaggagagattctcacacatctgggaagctgacaatccatgggcttcccatctgtgtagtttgccttctccagtgtctcattaaatgaggtcacactgtgtgtattctcagactgttttcttccacttagcaatgtgcatgcaagattcactcatgtctttgtgtgagttgatagcttgttcctttctatggctaaatagtattccattatatgaatgtaccacaatttggttatgcattttggggagcaaaaccttcctcttctaactttgttccaggattggagaccttcaaattaactgacaatagatacattagtagaagagacaatacttggcttcttgttccacaagtatcattgcgggacaaaattcagcagatggcaggatccagtttacaaagaggtaaaaatagcccagaaacaagaaacaagactagaatcagataactcacaatggctatagttttcctttaaaaaaattttttttgagacagggtctggctctgtcacccaggctggagtgcaaggtgcaatctcagctcactgcaacctctacctcctgggtgcaaacgatcctccctcctcagcctcctgattagctgggactacaggcacatgccaccatgcccatctaatttttgtacttttggtagagacggggtttcaccacgttgcccaggctggtcttgaactcctggcctcccaaagtgctggaattataggtgtgtgctgccatgaccagccatgttatagttttccattgaaacataaaatttctctctgtagtaaccatcatttttgatcataatcaaagtaagactattcttgtttcaaaaataagtctagttttattagattttgcttgattatttacgtaagtgcatcaagaacaggagatgaccacgtaggtgctttcaagcttctttgctggaagttttcatacagaatctcagatttgacttttaaaggccttattcaggctaaaagccaagctaagaacttactatcatatttcagctgcagtccttatagctttgtgtgaattcctctcttcttgaggcccccaaatatccctaaattcctgggcctaccaggaaatgaccttccttactaacctgtaaggctgtgaaccctgtaatctaggtatcaggctggcttttctcagagtgctgttgggaatgaagtttttggtgttccaaaataaaaaagaattaatgtgggaacaaatgatctcttagcaaggcaagctttactttctgcagaaagggtgctactcaatagctgtccagccacaagagcacaccaaacaaaggagacagagttacttataatctgatgtgtctaccctagtgctgtgtccagtttccattggctggaataggacctcacattttacactttacccgattggctgttagtttaaaactttcttaattaggtaaggggaatagaagaaagaaagaaaaggaagttgcccagggatagttaaggaaggatctccaaataaggaatggcatgcactatgggctggggcttgtctagttctgtccaggcatgctggagcaagctaggacaagtgatttggaatacacacacacacacacacacacacacataaatagtggatagcaatcttatagtaagaaattgtgactttataatctttgaagaagaactttcccatttctcacagtgctttgtaagcattgtttccataaaagtcaaccttacttccttaaaattgctggtcataaatgatcttaggtacacttcctaaatatgatattccagtaaaaaccttgataatataaccaaaatttccaattatgtccgttataaggtgaacagattcttattggacttttgctaacaactatatcatcgtgaaaatatgagtattcagtaaggatttcaaaattctggaaaaatcaggcagggaaaaaaagataaatgcctcatttctatttataaaagtataatctactaaattgttgtaagttacagttagattaagagaaagagatttcttaaatccagaaactagaacattaaagaaccagtaatgctccaaaaagctataaagttacaatcaattttcatcagttcattcagtgccatgtaatcaattccagtctttttggatcttgggttagcagtgtcatgaacccatcagtttctcaaccagacttctggagaccttcactgagtcaagtgtatggtcttaaagttatttaagcaatatcatcagaagcctataaccagagtacctgtcatagtcttttccgtgagtctcagagggagtcctgtgttggagacgaacattctgacttgtagctgattgcaggagctttcaggaaagcatcaggggaaataatatctaaatgacaaagagtatgaaatggctgtgatgaaagatctgatgagagttcattataccacagctgacaaggatattcaattttctgtgtggcatacaacatttatttacttatttagagacagagtctcactctgtcgcccaggcttgggtgcagtggcgtgatcttggctcactgcaagctccatctcccgggttcacgccattctcctgcctcagcctcccgagtacctggggctacaggcacctgccaccacgcctggctaattttttgtatttttagtacagacggggattcaccaggttagccaggatggtctggatgtcctgaccttgtgatccacccgcctcagcctcccaaagtgctgggattacaggcgtgagccactgcgcctggcacaacatttaaaataataattggaattatgactcattactctatactggcacatagcatggataaggaagacattgacaaatttccaggaattttatataatttctgaaaacataacattgtacccatacaaatataatacagggatggttaggtatctctttttatttgtatcttaagtatggttttccttataaaaaatacatcctactttacttgcaaaacatgccctacttttcttgcatgctttgcgtagagttgtttctagttattctattatttctagtagttttctttacatatattgattataattttaacacttagtaatcttttattttacagagaaaactaggaagtagagagttataaactgtcatatattaccattctatagtaggttagaaaatgtatgaatataccatctcccaacatctagagggatgtgtttcctcatagtacaatttctcagtgtggcagaaaaaaagacgtttattaatgggccaaaatatctttagtctctctgtaaaagtagcaaggcaaaattatataaacttgaatcatttatgctcagtaataaatgttttagtattgtatcttatttataaatggtctagatatttaatgcaaatcttttacttagcttaactttaaggttaaaaattaccaaaagtactttggaaactattcttaggcagatttactgtaaacaaattatttttgaaataatgtttttcgcttttcacaagatggcatcgaaagtgaagaaggaagctcctgcccgtcctaaagctgaagccaaagcgaaggttttgaaggtcaagaaggcagtgttgaaaggtgtccataaccacatgcaaaaagaagatccgcatgtcacccaccttcaggcggcccaagacactgcgactccagaggcagcccagatatcctcagaagagcacccccaggagaaacaagcttggccactatgctatcatcaagtttccgctgaccgctgagtcggccgtgaagaagacagaagaaaacaacacgcttgtgttcactgtggatgttaaagccaagaagcaccagatcagacaggctgtgaagaagctctatgacagtgatgtggccaaggtcaccaccctgatttgtcctgataaagagaagaaggcatatgttcgactggctcctgattatgatgctttggatgttgcaacaaaattgggatcatctaaactgagtccagctggctaactctaaatatatgtgtatcttttcaccataaaaaaatgatgtttttcataagaatgacaaattaattagaaccaaatctataagctttaagattttacgtttttagtaagtataatattagcttatttgactagaactcaagcagaataggaatttatgtttgttttatattcaatagtgataattttgaagacagttgttttattacaccaaaaatattatattaatcttatttaactaagttttatccaaatcgtgttaacttaaaaaacatttgatcagttcctatatttctaggagtttggtgaatatttacttataaatgcttatttttttccaagccaagttagaatagagcacttttagaggatttcataaatgaattttgcaatgctatccggagttaagaaaatatcacatatatgttacatacattaatagatatacaaacacaaatagagatttcatagctttcatcctgaaatttcagccatgaatcaggcataaatattctgatggttaattttagacatctacttgactggactaagagacacacatagctggtcaaacacaatttcagccatgaatcaggcataaatattctgatggttaagtttagacatctacttgattggattaagagacacacatagctggtcaaacacaatttctgggcatatctgtgagggtgtttctggaagacactgagataaccatgacccagtgtagatgggaactgatatggtttgcctgtttccccacccagatc

SEQ ID NO: 19 - DNA, 18 bp, Artificial Sequence
  Probe
  misc_feature (1)..(18): Telomeric sequence from PANAGENE PNA probes Catalog no F2003.Name TelC-Alexa647.  Sequence may repeat (CCC TAA CCC TAA CCCTAA)n.
ccctaaccctaaccctaa

(19) United States
(12) Patent Application Publication
(10) Pub. No.: US 2022/0162708 A1
(43) Pub. Date: May 26, 2022

(54) PHYSICAL CHARACTERIZATION OF TELOMERE (PCT)

(71) Applicant: GENOMIC VISION SA, BAGNEUX (FR)

(72) Inventors: Prakhar BISHT, Montrouge (FR); Mario Davide Maria AVARELLO, Montrouge (FR); Engin ALTUNLU, Metz (FR); Andrii KULAKOVSKYI, Cannes (FR); Aaron BENSIMON, Antony (FR)

(73) Assignee: GENOMIC VISION SA, BAGNEUX (FR)

(21) Appl. No.: 17/534,082

(22) Filed: Nov. 23, 2021

Related U.S. Application Data
(60) Provisional application No. 63/118,314, filed on Nov. 25, 2020.

(51) Int. Cl. C12Q 1/6886; G16B 25/00; C12N 15/10; C12Q 1/6809; G01N 33/50

(52) U.S. Cl. CPC C12Q 1/6886; G16B 25/00; C12N 15/1006; C12Q 2600/112; G01N 33/5091; C12Q 2600/106; C12Q 1/6809

(57) ABSTRACT

The present invention, called Physical Characterization of Telomere (PCT), provides and advantageous, accurate and convenient new methods for the visualization, characterization and measurements of telomere sequences. It employs probes and dyes to create a pattern of physical images, classifies the images, and determines the lengths of telomere sequences. PCT brings to a deeper understanding of telomere modifications that occur either genome wide manner or in a chromosome specific way.

### CROSS-REFERENCE TO RELATED APPLICATION

The present application claims priority to U.S. 63/118,314, filed on Nov. 25, 2020, the contents of which are incorporated herein by reference in its entirety.

The patent or application file contains at least one drawing executed in color. Copies of this patent or patent application publication with color drawing(s) will be provided by the Office upon request and payment of the necessary fee.

### BACKGROUND OF THE INVENTION

### Field of the Invention

The invention pertains to the fields of molecular genetics and medicine and involves the accurate and deep characterization of chromosomal telomeres.

### Description of Related Art

Telomeres are regions of repetitive nucleotide sequences at each end of a vertebrate nonlinear chromosome. In humans and other vertebrates, the telomeres typically comprise the repetitive non-coding hexanucleotide (TTAGGG)n¹. Human telomeres usually span 5-15 kb of polynucleotide sequences with heterogeneous lengths depending on the age of an individual and the tissue and cell type.

During chromosome replication, the enzymes that duplicate DNA cannot continue their duplication to the end of a chromosome, so in each duplication the end of the chromosome is shortened (this is because the synthesis of Okazaki fragments requires RNA primers attaching ahead on the lagging strand). The telomeres are disposable buffers at the ends of chromosomes which are truncated during cell division; their presence protects the genes before them on the chromosome from being truncated instead. The telomeres themselves are protected by a complex of shelterin proteins, as well as by the RNA that telomeric DNA encodes (TERRA). However, over time, due to each cell division, the telomere ends become shorter. Thus, the length of the telomeres declines as an individual ages, for example, on average from about 11 kb at birth to fewer than 4 kb in old age.

Telomeres protect the ends of a chromosome from being recognized as DNA double-strand breaks by binding to shelterin proteins and forming a specialized telomeric structure called T-loop. However, during each cell division, telomeres are vulnerable to gradual shortening by semi-conservative DNA replication. Cells that reach extremely short telomeres become senescence and subject to apoptosis.

Shortening of telomere length has been implicated in numerous age-associated diseases including arthritis, diabetes, infertility, cardiovascular and neurodegenerative diseases². Rare syndromes like pulmonary fibrosis, bone marrow failure, aplastic anemia, and acute myeloid leukemia among others have also been are linked to severe telomere length shortening³. In contrast to problems associated with shortened telomeres, cancer or neoplastic cells (as well as embryonic stem cells) often maintain or increase telomere length thus overcoming senescence or apoptosis and becoming immortalized.

Given the significance of telomere length and other characteristics to health and disease robust, accurate and reproducible measurements of telomere length and characteristics may be crucial to predicting onset of certain genetic and age-related pathologies in humans and other animal species.

A number of conventional methods, each with its problems or limitations, are available to score for telomere lengths including TeSLA, STELA, FISH, qPCR, TRF and TCA.

TeSLA stands for Telomere Shortest Length Assay. This is a classical method having good sensitivity having a lower resolution at 1 kb telomere measurements and a maximum resolution of only 18 kb. This technique is typically applied to human samples, has low throughput, and is extremely labor-intensive. It is not suitable for model systems beyond 18 kb of telomere lengths detection e.g. for in-bred strains of mice. Due to this limitation, firstly, TeSLA cannot detect interstitial telomere sequences (ITSs) longer than 18 kb, and secondly, it is impossible to distinguish ITSs from telomere signals even when below 18 kb⁴.

TeSLA requires about 1 μg of DNA and is used in combination with Southern blot analysis. It is adequate for short telomeres, but it cannot work for long telomere length identification. TeSLA cannot be exploited for diseases associated with telomere elongation or loss, due to its narrow 1-18 kb range. TeSLA also requires a week of complicated lab work to generate results during which time bias can be introduced by each required individual step and technique. Lastly, TeSLA analysis typically takes fifteen hours of interpretation to provide valuable results⁴.

Another method is called Single telomere length analysis (STELA) and a modified version known as Universal STELA (U-STELA). The amount of DNA required is about 2 μg and the assay uses ligation and PCR-based methods in combination with Southern blot analysis. STELA can provide detailed information about the abundance of the shortest telomeres. The Universal STELA (U-STELA) method was reported to detect telomeres from each chromosome using a suppression PCR strategy to prevent the amplification of the intra-genomic DNA fragments. STELA is limited because it can only work on a specific subset of chromosome ends. While U-STELA was designed to identify DNA with a low molecular weight of less than about 500 bp. However, these methods are inadequate to sufficiently suppress the amplification of larger genomic DNA fragments and U-STELA does not efficiently detect telomere lengths over 8 kb. These methods are also laborious requiring two weeks of bench work and subsequent analysis of the results is complex and takes about 48 hours.⁵.

Another technique for telomere detection is fluorescence in-situ hybridization (FISH) which was developed in the 1980s. It is a cytogenetic technique using fluorescent probes to bind to the chromosome with a high degree of complementarity. It is an easy method for the detection of RNA or DNA sequences in cells including those in various tissues and tumors. This technique is useful for identifying chromosomal abnormalities, gene mapping, characterizing somatic cell hybrids, checking amplified genes, and studying the mechanism of rearrangements.

RNA FISH is used to measure and localize mRNAs and other transcripts within tissue sections or whole mounts. It measures the length by the intensity of the probe.

Quantitative-FISH (Q-FISH) is an approach for the quantitative measurement of the length of DNA fragments hybridize with the probe. The resolution of Q-FISH was estimated to be 200 bp and the mean fluorescence intensity of telomeres measured by Q-FISH is correlated with the mean size of telomere restriction fragments. It measures the length by the intensity of the probe and telomere lengths can be measured by using live or fixed cells. Q-FISH can quantify each telomere signal in each nucleus, but the percentage of the shortest telomeres can be underestimated. For metaphase Q-FISH can detect telomeres from each chromosome, however, this method does not permit analyses on non-dividing cells, such as senescent cells or resting lymphocytes. Using resting or interphase cells on flow-FISH and HT Q-FISH are adapted for large scale studies to typically estimate the mean telomere length for interphase cells. While these approaches are an improvement over Q-PCR, one disadvantage of these techniques is the probe not only binds to telomeric repeats but also interacts with non-specific components in the cytoplasm. Probe hybridization kinetics do not permit robust quantitation of the shortest telomeres (<2-3 kb and it is impossible to distinguish interstitial telomere sequences (ITSs). Moreover, the wet lab work takes about five days, and the analyses about twelve hours.

An alternative method for visualizing telomeres is quantitative polymerization reaction or qPCR. Of the several major methods utilized to determine telomere length, qPCR remains a suitable method for large-scale epidemiological and population studies. However, inconsistencies in utilizing the qPCR method have been reported and highlight the need for a careful methodological analysis of each step of this process. This method provides relative quantification of telomere signals compared to single-copy gene signals. However, qPCR only measures the relative telomere proportional to the average telomere length from the reference sample. Besides, the qPCR method is not suitable to quantify telomere length for cancer studies since most cancer cells are aneuploid. Additionally, it is impossible to distinguish interstitial telomere sequences (ITSs) from telomere sequences and the lab work takes about 5 days and the analysis of the results about 32 hours.

Another method involves Southern blotting of a Terminal Restriction Fragment (TRF). It estimates telomere length by intensity and size distribution of a “telomeric smear” on an agar gel. This method requires a large amount of genomic DNA due to the lower hybridization signals of the shortest telomeres and TRF underestimates information about the abundance of the shortest telomeres. It is also impossible to distinguish interstitial telomere sequences (ITSs) and the lab work takes about a week and analysis of results is complex and takes about 48 hours.

Several commercial companies including Life Length (hypertext transfer protocol secure://lifelength.com/), Repeat Diagnostics (hypertext transfer protocol secure://repeatdx.com/), and Teloyears (hypertext transfer protocol secure://worldwide web.teloyears.com/home/) will measure telomere length. However, a major limitation of all these commercial techniques is that they provide the ‘relative/average’ telomere lengths and not a real ‘physical’ measure of telomere lengths.

One of the latest developed methods is the Telomere length Combing Assay (TCA) which is also known as Telomere Fiber-FISH (TFF)^{8,9}. TCA requires about 1 μg DNA, the lab work takes about 5 days, and the analysis of the results about 5 hours for automatic analysis and about 10 hours for manual analysis. A comparative analysis with other existing techniques including TRF, Q-FISH, flow-FISH, and qPCR was performed and demonstrated that TCA was more sensitive and accurate for telomere length measurements⁸. TCA provides a measure of telomere lengths by measuring the stretch of telomere signal obtained by hybridization with PANAGENE PNA probes. However, this technique has several limitations which make its use for detailed genome-wide investigations and a chromosome-specific (CS) detection impossible. TCA is unable to screen-out sequences that consist of telomeric repeats located away from the chromosome ends which are also known as interstitial telomeric sequences (ITSs)¹⁰. It lacks the specificity for performing measurements of genome-wide arm-specific telomere lengths for disease-related clinical diagnosis because TCA output consists of a very superficial analysis of the telomere length only. It cannot provide an exhaustive explanation of the causes such as genome rearrangement or identify the specific chromosome arm and/or the biomarker/loci of interest. Furthermore, this method can only distinguish telomere shortening but not terminal elongation making its use impractical for precise diagnostic and/or clinical studies/treatments, research purposes, or for drug design/screening/testing. This makes its results non-conclusive.

Given the above problems with conventional methods for visualizing or characterizing chromosomal telomeres, the inventors sought to develop an easier and more accurate method. As disclosed herein, the physical characterization of telomeres (hereinafter referred to as “PCT”) method was designed to overcome the limitations innate in the methods described above and other existing methods. As disclosed herein, the PCT method permits deep analysis of genome wide telomere modifications at the p and q chromosomal arms by SubTA; as well as a deep analysis of a specific chromosomal locus by DisTA. The PCT method permits detailed, unified, and convenient analysis of telomere modifications or events associated with many diseases including aging, cancer and other rare diseases. The results, which can be analyzed by computer programs, provide valuable prognosis of many telomere associated disease, disorders or conditions and is a valuable tool in scientific, diagnostic and therapeutic applications including evaluation of drugs or other agents targeting telomeres.

### BRIEF SUMMARY OF THE INVENTION

The physical characterization of telomeres (PCT) as disclosed herein comprises several new methods for taking physical measurements of telomeres. These measurements may be genome-wide or chromosome-specific.

Genome-wide methods include sub-telomere applications SubTAS, SubTAL, and SubTAE.

SubTAS (Sub Telomere Application for Shortening) is used to show which arm (p or q) of a chromosome is affected by a disease or a treatment. It also characterizes and quantifies genome rearrangements due to the presence of ITSs (interstitial telomeric or telomere-like sequences) versus the true telomere sequences or signals therefrom. SubTAS can identify true telomere sequences and signals at the ends of chromosomes.

SubTAL (Sub Telomere Application for Loss) identifies chromosome losses at the p or q arms of a chromosome.

SubTAE (Telomere Application for Elongation) distinguishes and quantifies the true telomere elongation signals. SubTAE can be used to test the efficacy of anti-aging or anti-cancer compounds/treatments.

Chromosome-specific procedures include, DisTAS, DisTAL and DisTAE.

DisTAS (Disease specific Telomere Application for Shortening) is used to characterize, quantify, and measure effects associated with shortening/shrinking of telomeres and at a chromosome specific region, especially shortening associated with a particular disease or chromosomal locus. DisTAS can identify true telomere sequences and signals at the ends of selected chromosomes.

DisTAL (Disease specific Telomere Application for Loss) is used to detect when a specific loss of telomere sequences or signals after a disease specific sub-telomeric signals.

DisTAE (Disease specific Telomere Application for Elongation) characterizes, quantifies, and measures effects of replication kinetics associated with telomere elongation, for example in embryonic stem cells or neoplastic cells. It is typically used in combination with incorporation of dNTPs analogs to characterize, quantify, and distinguish terminal telomere lengthening from other DNA replication signals.

These PCT methods represent a significant improvement over conventional telomere measurement or detection methods and permit the visualization, characterization and analysis of telomere modifications including telomere shortenings, losses, and elongations as well as distinguishing between true telomere chromosomal termination sequences and interstitial telomeric sequences.

These methods and analysis of the data they generate may be automated, semi-automated, or manually performed. The software disclosed herein provides automated or semi-automated detection of physical characteristics of telomeres that permits predictive interpretation of the analyses of the PCT data. The analyzed PCT data permits practitioners or researchers to improve prognosis and treatment of patients having diseases, disorders or conditions associated with alterations or irregularities in their telomeres.

The methods disclosed herein provide detailed, accurate and convenient tools for developing or assessing clinical/diagnostic treatments, drug discovery/screening/testing, gene editing control, cell stratification and for treatments based on modified cells.

The foregoing paragraphs have been provided by way of general introduction, and are not intended to limit the scope of the following embodiment/claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1: Synopsis of the physical characterization of telomeres or “PCT”.

FIG. 2: Schema I: the many levels of PCT.

FIG. 3A: Genome-wide identification of ‘p’ arm of chromosomes where the telomeric regions are identified in red at far right (e.g. AlexaFlour647 (PANAGENE)) and the physical lengths are annotated by ‘γ’ in kilobases. The specific sub-telomeric regions are identified in green (e.g. FITC (CytoCell)) and physical lengths are annotated by ‘α’ in kilobases. The DNA fibers are counterstained with blue fluorescent dye e.g. PO-PRO1 (thin line). The distance between the sub-telomeric and telomeric regions is annotated by ‘β’ in kilobases.

Similarly, FIG. 3B: Genome wide identification of ‘q’ arm of chromosomes where the telomeric and specific sub-telomeric region are identified in red (far right) and blue (thick interior line) fluorescence respectively (e.g. AlexaFlour647 (PANAGENE) & TexasRed (CytoCell)) and the DNA fibers are counterstained with green fluorescent dye e.g. YOYO1 (thin line). The physical lengths and the distances are annotated as ‘α’, ‘β’ & ‘γ’ in kilobases.

FIG. 4A-FIG. 4C. Schematic representation of the SubTA signals for the various telomere modifications.

FIG. 4A: Wild type genome-wide (GW) signal of sub-telomeric p/q arm of chromosomes (green/blue) where the telomeric regions are identified in red at far right (e.g. AlexaFlour647 (PANAGENE)). The physical lengths for the different regions are annotated by ‘α’, ‘P’ and ‘γ’ in kilobases. The specific sub-telomeric regions are identified in green (e.g. FITC (CytoCell)) or blue (thick interior lines) and physical lengths are annotated by ‘α’ in kilobases. The DNA fibers are counterstained with blue fluorescent dye in the e.g. PO-PRO1 (thin lines). The distance between the sub-telomeric and telomeric regions is annotated by ‘β’ in kilobases.

FIG. 4B: Representation of the telomere shortening GW by the identification of sub-telomeric p/q arm of chromosomes where the telomeric and specific sub-telomeric region are identified in red (far right) and blue (thick interior lines) fluorescence respectively (e.g. AlexaFlour647 (PANAGENE) & TexasRed (CytoCell)) and the DNA fibers are counterstained with green fluorescent dye e.g. PO-PRO1 (thin lines). The variation of the physical lengths and the distances are annotated as ‘Δα’, ‘Δβ’ and/or ‘Δγ’ in kilobases.

FIG. 4C: Representation of the telomere loss genome-wide by the identification of sub-telomeric p/q arm of chromosomes in green and blue fluorescence respectively, and the DNA fibers are counterstained with green fluorescent dye e.g. PO-PRO1. The variation of the physical lengths and the distances are annotated as ‘Δα’ and ‘Δβ’ in kilobases. The loss of the telomere is defined by the loss of the red signal at far right in FIG. 4A and FIG. 4B.

FIG. 5A-FIG. 5B: SubTAE signals from the Terminal Telomere Elongation. The light green dot/bar represents the incorporation of dNTPs: FIG. 5A: signal for Terminal Telomere Elongation: the replication of the ssDNA at the end of telomere and represent the elongation from this side; FIG. 5B: signals for Non-Terminal Telomere Elongation: replication is between sub-telomere and telomere at the beginning of telomere region.

FIG. 6A-FIG. 6C: Schematic representation of the DisTA signals for the various telomere modifications:

FIG. 6A: depicts the wild-type chromosome specific signal for the FSHD. Telomeric regions are identified in red at far right (e.g. AlexaFlour647 (PANAGENE)) and the physical lengths are annotated by ‘γ’ in kilobases. The FSHD specific sub-telomeric regions for D4Z4 tandem repeats are identified in magenta in longer, thick interior lines (blue and red fluorophores probes) and physical lengths are annotated by ‘α’ in kilobases. The chromosome 4 qA arm specific sub-telomeric regions are annotated in green in short, thick interior lines (Cy3 fluorophore) with i) 4 q 16 kb (adjacent to the D4Z4) ii) 4 qA1 2 kb (between D4Z4 and telomere). The DNA fibers are counterstained with blue fluorescent dye e.g. PO-PRO1 (thin lines). The distance between the D4Z4 and telomeric regions are annotated by ‘β’ in kilobases;

FIG. 6B: Representation of chromosome-specific telomere shortening. The signal from FSHD affected person carrying shorter D4Z4 and telomere: pattern of colors is the same as explained in FIG. 6A: the length variations are: ‘Δα’ for length variation of D4Z4 region, ‘Δβ’ length variation of the link DNA, ‘Δγ’ length variation of the telomere.

FIG. 6C: Representation of the telomere loss chromosome specific. The signal from FSHD affected person carrying shorter D4Z4 and telomere: pattern of colors is the same as explained in FIG. 6A: the length variations are: ‘Δα’ for length variation of D4Z4 region, ‘Δβ’ length variation of the link DNA. The variation of the physical lengths and the distances are annotated as ‘Δα’ and ‘Δβ’ in kilobases. The loss of the telomere is defined by the loss of the red signal shown in parts FIG. 6A and FIG. 6B.

FIG. 7A-FIG. 7B: DisTAE signals from the Terminal Telomere Elongation. The light green dot/bar represents the incorporation of dNTPs: FIG. 7A: signal for Terminal Telomere Elongation: the replication of the ssDNA at the end of telomere and represent the elongation from this side; FIG. 7B: signals for Non-Terminal Telomere Elongation: replication is between sub telomere and telomere at the beginning of telomere region.

FIG. 8: The detection and measurements of telomeric and sub-telomeric signal on the q arm of chromosome 13 in U2OS cell line. The ‘R’ signal defines the telomeric region (178 kb; PANAGENE probes), the ‘B’ signals defines the chromosome 13 q arm (CytoCell probes; 132 kb) and the ‘G’ signals validates the predicted distance between the telomeric and sub-telomeric region (17 kb; documented by CytoCell Ltd). Segments from left to right: red (R), green (G), and blue (B).

FIG. 9: The detection of replication events at telomeric and sub-telomeric regions via IdU incorporation. The red color signals define the telomeric region (PANAGENE probes) while the green color signal determine the IdU incorporation (mouse anti-BrdU; BD Biosciences). The overlap of telomere and IdU incorporation can be seen in yellow color (merging of red and green color) which shows replication within telomere and consequently allows to measure the length of the telomere. Likewise, the IdU incorporation within the sub-telomeric region shows the possible origin of replication (shown with yellow arrows). The DNA fiber is detected in blue color via use of ssDNA antibody (mouse antihuman ssDNA; Merck).

FIG. 10: The detection and measurements of disease specific region and telomere lengths in FSHD disease on chromosome 4 q arm. The ‘R’ signal defines the telomeric region (79 kb; PANAGENE probes), the ‘B’ signals defines the disease specific D4Z4 region (Genomic Vision; FSHD GMC probes; 190 kb) and the ‘G’ signals recognizes the chromosome 4 qA arm specific two sub-telomeric regions i.e. 4 q 16 kb and 4 qA1 5 kb (Genomic Vision; FSHD GMC probes). Segments from left to right: red (R,79 kb)), green (G, 5 kb), blue (B, 190 kb) and green (G, 16 kb).

FIG. 11A and FIG. 11B: Flow-chart of Classical FiberStudio®® Detection process. For each type of signal a specific algorithm with specific filters and processing operations is developed.

FIG. 12A-FIG. 12C: Representation of the Kernel method. FIG. 12A: A kernel, 3×3 convolution matrix. Numbers in the matrix are the weights will be applied while doing a convolution. FIG. 12B: Rectangular kernel used for telomere signals detection. Y and X are the dimensions that are defined by the developers as 15×5 or 150×10. FIG. 12C: Rectangular kernels for two different probes. For the detection, it's defined two types of kernels, one for telomere region, one for sub-telomere region. It's also possible to define one big kernel that can cover both of them.

FIG. 13: Flow of the detection steps in classical FiberStudio® software. First, on an image, all convolutions are applied with defined kernels, then follows a normalized correlation, dilation and erosion to obtain an object zone.

FIG. 14: An artificial neural network (ANN) structure. ANN has layers made by nodes, and each node has a ‘weight’ (a coefficient which is applied to the data coming from the layer just before) that is readjusted in the learning (also called training) process. At the end of every iteration in learning process, on output layer, predictions are made and according to the errors of predictions, weights are readjusted, this operation is called “back propagation”.

FIG. 15: AI based FiberStudio® software's steps. Software fist applies the detection to obtain the Telomere signal's position. Segmentation process sorts the correct colors and their lengths of the detected signal. As the last step, classification process assigns the correct signal category for the signal.

FIG. 16: Flow-chart of AI Based FiberStudio® @® detection process. One single algorithm for learning and generating Neural Network models and one single algorithm that calls these built models for a given image type.

FIG. 17: Architecture of neural network used for PCT's signals detection.

FIG. 18: Example of a segmentation process. The image on the left is the original signal from a scanned coverslip. The image on the right is the prediction which is generated by LinkNet Neural network model. As explained previously, the model learned and could handle the eventual gaps (holes) happened on the DNA fiber and by ignoring them, it could predict an incredibly close interpretation to the reviewers.

FIG. 19: An example of a signal's vector creation.

FIG. 20: Reporting Module's communication flow with Classical FiberStudio® and AI based FiberStudio®.

FIG. 21: Example of genome-wide identification of all sub-telomeric regions on all 8 chromosomes for all p & q arms by ‘Soup’ of 13 probes/sequences. The representation of the unique combinations of 13 probes for each p and q arms of different chromosomes, which localize physically near the telomere end site (T). The panel of 13 probes is demonstrated on the top of the image where each duplication box number and unique probes size (kb) is mentioned. The bottom left of the image shows the scale of representation lengths in kb.

FIG. 22: Example of chromosome specific identification of sub-telomeric regions on 8 chromosomes for all p & q arms by 16 probes. Represents the unique probes/sequences of distinct sizes and distances from the telomere end site (T) for each p and q arms of different chromosomes. The orange box shows the unique probes (size in kb). The bottom left of the image shows the scale of representation lengths in kb.

FIG. 23: Schematic representation of DisTA application for TERF1 gene characterization of telomere lengths alterations on chromosome 8. The green color probe identifies the 8p arm. This probe is 9 kb in size and is 176 kb away from the telomere end site. The blue color probes are for i) 8 q arm probe which is 7.2 kb in size and 13 kb distance from the telomere end site; ii) TERF1 gene probe is 30 kb long and 72 Mb(megabases) away from the telomere end site on q arm. The red color probes are for i) the telomere signals at each end of the chromosome 8 arm i.e. p/q arms; ii) Adjacent probe to the TERF1 gene which is 4 kb in size and 2.4 kb distance from the TERF1 gene towards the telomeric side.

FIG. 24: The identification of Gene of Interest (GOI) and the arm specific probes/sequences for detection and measurements of telomere lengths. The TERF1 gene (30 kb in blue) and the adjacent region (4 kb in red color) are identified. The chromosome 8 p arm is detected with a green color (9 kb) which is 176 kb from the telomere end site (in red color). Likewise, chromosome 8 q arm is detected with a blue color (7.2 kb) which is 13 kb from the telomere end site (in red color).

FIG. 25: The co-ordinates for the SubTA genome-wide ‘Soup’ of 13 probes. The accession numbers for the genome, the chromosomes arms, and the specific probes (identified by sequence coordinates within the target accession number sequence) are as provided and are accessible at Ensembl Rest API—Ensembl REST API Endpoints. [online] (hypertext transfer protocol secure://rest.ensembl.org/ [last Accessed 31 Aug. 2021]).

FIG. 26: The co-ordinates for the DisTA chromosome specific 46 probes. The accession numbers for the genome, the chromosomes arms, and the specific probes (identified by sequence coordinates within the target accession number sequence) are as provided and are accessible at Ensembl Rest API—Ensembl REST API Endpoints. [online] (hypertext transfer protocol secure://rest.ensembl.org/ [last Accessed 31 Aug. 2021]).

### DETAILED DESCRIPTION OF INVENTION

Background on Telomeres.

Cellular fate is driven by instructions written in the form of a code in polynucleotides having a double helix structure called the Deoxyribonucleic Acid (DNA). The DNA is composed of sequences that are known as genes, regulatory elements where repetitive DNA are interspersed at the chromosome level, in pieces of condensed and open regions¹¹.

DNA is a long molecule that can reach the length of few meters, but it goes to high-order chromatin organization in order to gain the length in the micrometers order. This high chromatin condensation is possible because of the existence of histones proteins (H2A, H2B, H3, H4 and their histone variants) and the formation of extra super secondary, ternary and quaternary structures. The different grades of condensations allow some part of DNA to be read, translated and traduced which leads to the formation of the euchromatin; an open form of DNA that can accessed by proteins. Likewise, the structures that are not accessible and inactive for transcription are called heterochromatin¹². Gene expression is influenced by the vicinity of a gene to the eu- or heterochromatins regions¹³. In addition, the gene's vicinity to these regions can change or lead to Position-effect variegation (PEV), or the chromosomal position effect (CPE) is reference to chromosomal structure¹⁴.

Two of the most known heterochromatin regions are centromeres and telomeres. Among them, telomeres are tandem repeats that protect the chromosomes from shrinking by forming a cap structure. Genes located in the proximity of telomere are triggered to be silenced by the effect of what is known as Telomeric Proximity Effect (TPE)¹³. These sequences of DNA that are subjected to TPE are called sub-telomeres, and are defined as the segments of DNA that lie between telomeric caps and chromatin. Specifically, sub-telomeres are immediately adjacent to telomeres and they are unique regions that contain long stretches of DNA but do not contain genes¹⁶. The structure of the sub-telomeres is similar between related spices and are composed by repeated units, but their sequences and the extent of these elements are totally not analogous¹⁷. Consequently, uncontrolled events on telomere, such as elongation shortening or loss, can cause unfortunate consequences for the cells fate. Normally, these cells arrest most of the vital biological processes and activate the pathways to bring to senescence and death. Sometimes, some of these cells, with affected telomere and/or sub-telomeric regions, escape to senescence and are the bases for developing diseases.

Rearrangements in the sub-telomeric regions appear to be responsible for 5% to 10% of cases of moderate and severe mental retardation. Most cases of sub-telomeric rearrangement are associated with novel or unnamed syndromes of disability. However, also telomere shortening has been related to the onset of sever pathologies, that collectively are known as ‘telomeropathies’, and they are the basis of the onset of aging related diseases and cancer. With these implications in mind, it has been extremely crucial, for us, to develop a high-throughput technique that characterizes and measures physical telomere & sub-telomeric lengths to establish the critical link between disease onset and early diagnosis. In particular, it has been very important to be able to analyze the telomere modification in genome wide (SubTA) and/or chromosome specific (DisTA) manner.

The Physical Characterization of Telomere or PCT provides several new methods for the visualization, characterization and measurements of telomere sequences. It is based on the use probes and dyes to create a pattern for the physical imaging, classification and sizes of telomere sequences. PCT brings to a deeper understanding of telomere modifications that occur either 1) genome wide manner or 2) chromosome specific way.

Genome wide: PCT is used to identify characterize and measure the telomere modifications specifically at each side of the chromosome arms: p and/or q arm. Indeed, allows to identify and link telomeres with their own sub-telomeric regions by using a set of probes for the p arms, another set for the q arms, the telomere probes and the DNA fiber counterstaining. Henceforth, characterization and measurement of the telomere modifications can be carried out by connecting the telomere and the sub-telomeric regions. Specifically, PCT can distinguish at the p and or q arms of the chromosomes: the telomere loss by missing telomere signals, telomere shortening by measuring the length and the telomere elongation by identifying the incorporation of nucleotide analogs at the beginning, mid or end of the telomeres.

These applications were grouped under the classification called Sub Telomere Application (SubTA). The SubTA, is sub-divided into three distinct categories on the basis of their application:

(a) Sub Telomere Application for Shortening (SubTAS) the method to visualize, characterize and measure telomere shortenings. SubTAS allows one to gather pieces of evidence of which arm of chromosome is affected by a disease or a treatment, Finally, SubTAS allows one to characterize and quantify the genome rearrangements due to the presence of ITSs versus the true telomere signals;

(b) Sub Telomere Application for Loss (SubTAL) identifies the chromosome loss at the p or q arms of the chromosome; and

(c) Sub Telomere Application for Elongation (SubTAE) distinguishes and quantifies the true telomere elongation signals. SubTAE can be used to test the efficacy of anti-aging or anti-cancer compounds/treatments.

SubTA by PCT: A Novel Method to Measure Physical Length of Telomeres in an Arm Specific Manner Genome Wide.

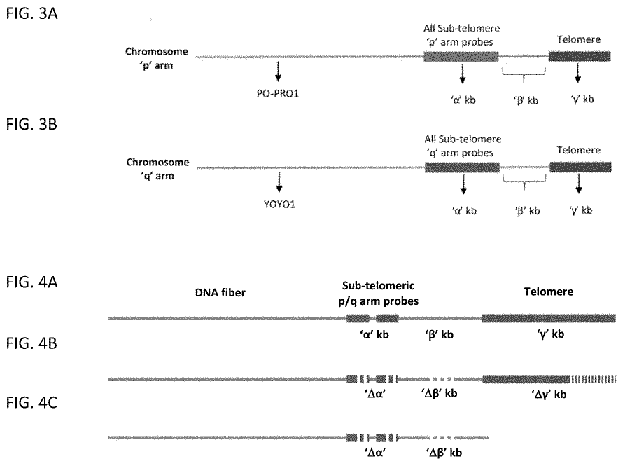

The first set of applications of the Physical Characterization of Telomere (PCT) are called SubTA, which stands for Sub Telomere Application. It is a state-of-the-art application that utilizes Genomic Vision proprietary technology to identify the telomere lengths on p and/or q chromosome arms and rearrangements genome wide. It identifies the physical lengths of telomeres, for example, measurements of at least 0.8 to 250 kb and more, on ends of chromosomes genome wide and on rearrangement of telomere sequences, FIG. 3A and FIG. 3B right end (red); (ii) it identifies sub-telomeric regions specific to each chromosomep and q arm (green for p arm & blue for q arm; FIG. 3A and FIG. 3B, respectively, with known length and distance (in kilobases) from the telomeric repeats; and (iii) identifies intact DNA fibers by double stranded counterstaining dyes (e.g. p arm with blue by PO-PRO1 & q arm with green by YOYO1; FIG. 3A and FIG. 3B, respectively (thin blue or green lines).

SubTA is a specific, very sensitive and precise tool that allows one to identify and separate ITSs (interstitial telomeric sequences) signals from the true telomeric signals genome wide. The sub-telomeric signals act as anchoring regions adjacent to telomeric signals to allow isolation of ITSs regions observed as a consequence of genomic rearrangements. Additionally, rearrangements within the sub-telomeric regions; a potential biomarker for pathologies e.g. severe mental retardation can also be scored with SubTA via the measurements of sub-telomeric lengths shortening/rearrangement events in an arm specific manner; see FIG. 8 where the R (red) signal defines the chromosome 13 q arm.

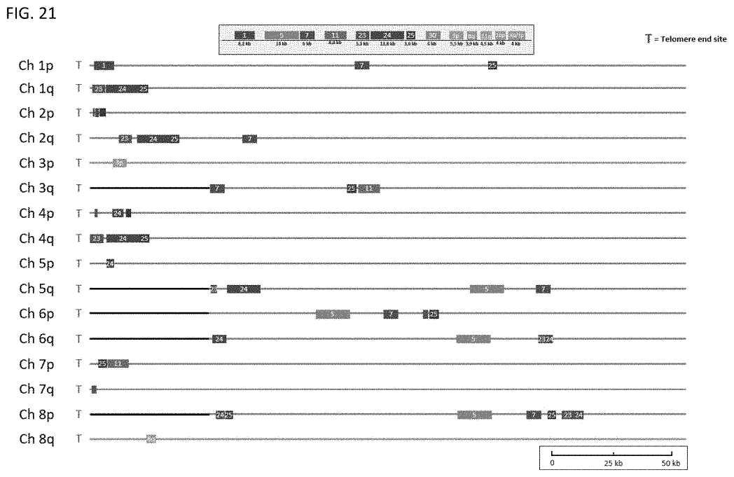

Since the sub-telomeric regions for all chromosomes accounts for highly polymorphic regions, scoring for p and q arms genome-wide by the usage of singular probes is extremely challenging. For such application a unique ‘Soup’ of 13 probes/sequences that can identify the sub-telomeric regions for entire genome in close proximity of few kilobases is provided. This includes the physical characterization of 41 p and q arms of chromosomes (excluding acrocentric chromosomes i.e. 13p, 14p, 15p, 21p & 22p) adjacent to telomere. The ‘Soup’ of 13 probes/sequences consists of 8 duplication boxes⁵⁰ and 5 unique probes (FIG. 21) which hybridize at physical distance of 1 kb to 200 kb from the telomere end site (as represented schematic in figure T=Telomere end site) for all p and q chromosome arms. With this panel of varied but unique combinations of 13 probes, not only is it possible to cover each p and q arms of all chromosomes to identify the TTS (true telomere signals) but it is also possible to discard the ITS (Interstitial telomere sequences).

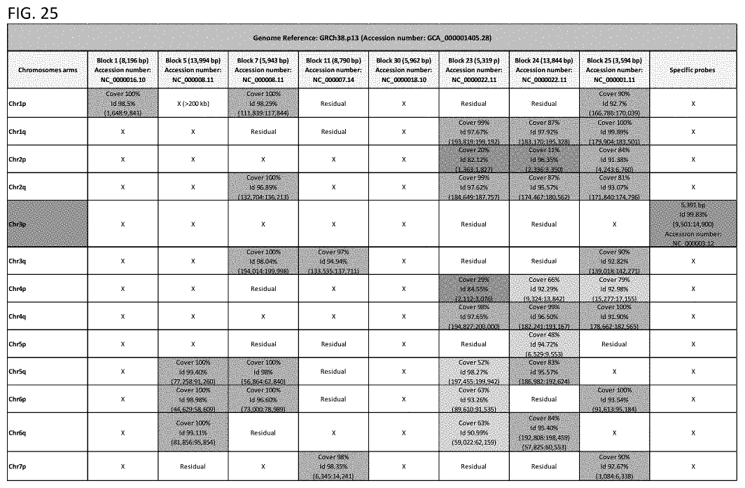

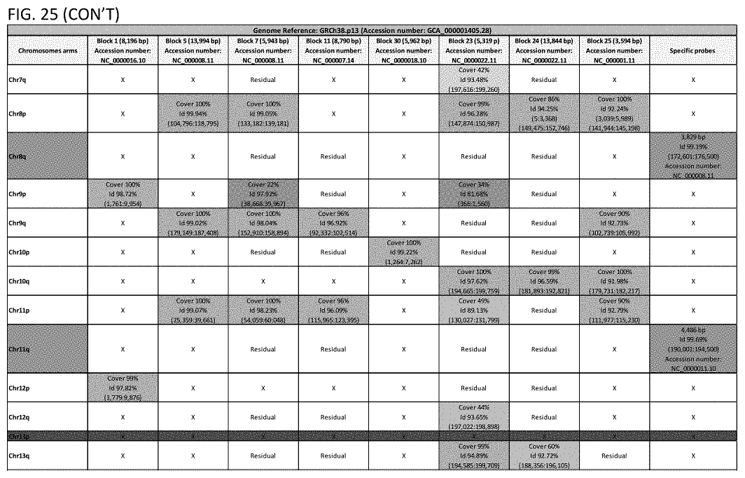

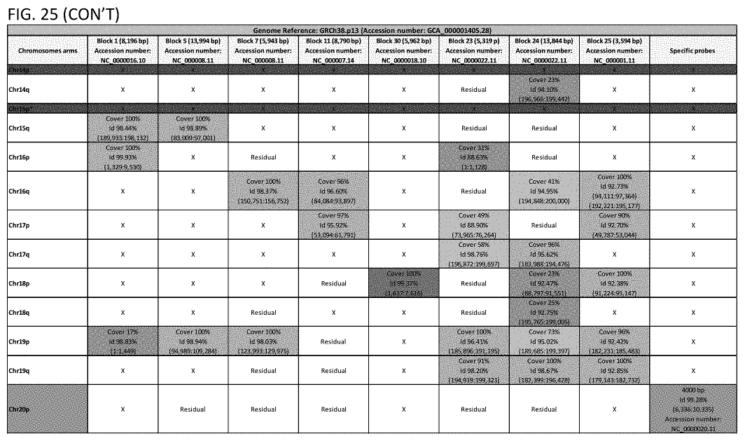

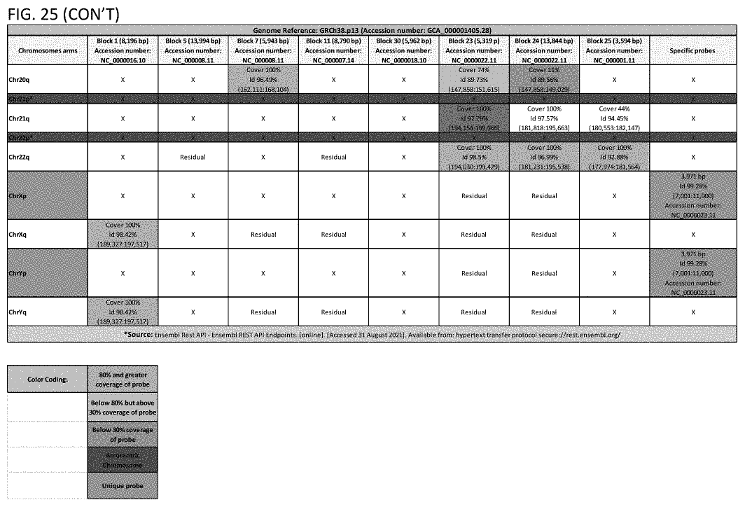

FIG. 25 provides an example of the SubTA approach and primer design for use in the present invention. The accession numbers for the genome, the chromosomes arms, and the specific probes (identified by sequence coordinates within the target accession number sequence) are as provided and are accessible at Ensembl Rest API—Ensembl REST API Endpoints. [online](hypertext transfer protocol secure://rest.ensembl.org/ [last Accessed 31 Aug. 2021]). The exemplary embodiment provides benchmark sequences; however, it is understood that the present invention is not bound to the specific defined sequences as it is well-known in the art that with sequences of the length of the probes permit localized mismatch while preserving global binding. Further, when considering the length of the sequences of the respective probes, the reduced degree of overall sequence identity is tolerated. Thus, an embodiment of the present invention are probes that are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 25.

Another use of SubTA, by PCT, is an application to uncover the role of replication kinetics into the Terminal Telomere Elongation (TTE). Generally, extended telomere is a benchmark for a predisposition to live a longer life than people with shorter telomere, even when they are suffering from some diseases¹⁸. Short telomere brings, indeed, to a predisposition of Alzheimer's¹⁹, to dementia and the early death in twin⁷. Telomere elongation in mice has proved to increase their life span, to ameliorate the aging disorders, the insulin levels, neurological conditions²⁰. In humans, the extension of telomere is connected to rescue of liver disease and pulmonary fibrosis²¹. Then, characterize and quantify the telomere elongation could help to contribute to find better compound and better treatment for a specific patient. SubTA exploit the incorporation of nucleotide triphosphate during the replication process to mark how the DNA duplication can impact the telomere size. Replication is the cellular process to copy the DNA molecule, in semi-conservative manner, before this nucleic acid is transferred to the daughter cell¹².

SubTAS.

The SubTAS is an application that identifies and scores for telomere shortening events in reference to sub-telomeric regions, see FIG. 3A-FIG. 3B. Specifically, it refers to length variation (shortening) of the telomere, sub-telomeric (p and/or q arm) sequences as well as for the possible variation between these regions. As shown in FIG. 3A-FIG. 3B, the wild type signal for the DNA fiber (light blue thin line), sub-telomeric regions at the p and/or q (green and blue thick interior lines, respectively), telomere region at far right (red).

A shortening event is illustrated by FIG. 4A AND FIG. 4B using dashboard signals for the respective sub-telomeric, telomeric and DNA link regions.

Chromosomal shortening (SubTAS) is distinguishable from chromosomal loss (SubTAL). In SubTAS, there can be events which include SubTAL, however, in SubTAL there cannot be any events which are in SubTAS. FIG. 4A, FIG. 4B, AND FIG. 4C demonstrate the events of SubTAS and SubTAL and exemplify the pattern of signal identification observable on a genome-wide scale.

SubTAL.

SubTAL is an application that scores for only total loss of telomere repeats in reference to sub-telomeric regions. It, indeed, refers to the complete loss of the telomere. In this case the signal as depicted in the FIG. 4C for SubTAL is identified.

The DNA fiber (in light blue), sub-telomeric p and/or q sequences (in green and/or dark blue) and the following known size of DNA fiber after it (in light blue thin line) with loss of telomere signal (absence of red signal in FIG. 4C that is present in FIG. 4A and FIG. 4B).

Sub-TAE.

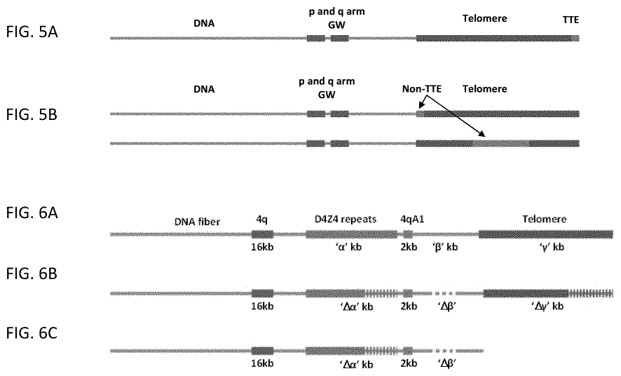

To verify telomere elongation events, a specific application called SubTAE was developed. The procedure includes the pulsing of a sample with dNTPs analogs prior to isolation for a specific time, according the model organism. Then, DNA is extracted, combed and step of hybridization and immunostaining are performed according the protocol in the Materials and Methods disclosed herein. FIG. 5A and FIG. 5B show the possible signals originated by SubTAE. The variety of these signals depends whether the replication is before the sub-telomeric region, between sub-telomeric and telomere, at the beginning of telomere or at the end. SubTAE leads then, to the characterization of the telomere elongation according the replication kinetics, quantification of the different patterns and the measurements of telomere elongation or sub-telomere elongation. Telomere length may be compared to a control value, for example, a value prior to a treatment or after a baseline telomere length is determined at a time zero.

SubTAE can identify, quantify and measure the Terminal Telomere Elongation events. SubTAE is used to understand the effects of a treatment/compound specific to be tested for its ability to elongate telomeres. The replication and maintenance of telomeres are two connected topics that have been investigated to uncover details of their connection with cancer, genetic diseases and/or aging^{23,24}. The average of telomere length in human is 5-15 kb, most of which is double stranded DNA. Though, there at the very end, a single stranded DNA sequence that is 30-200 nucleotides long and GT-rich 3′ overhang²⁵. TERT is the enzyme that elongate telomeres, it is a holoenzyeme composed by the catalytic domain and a small RNA. Recently, there is an increase interest to develop molecule that allow TERT to elongate telomeres, i.e. by delivery of nucleoside-modified TERT mRNA²⁶.

DisTA/Chromosome Specific:

The PCT is also used to visualize, characterize and measure sub-telomeric and telomere signals in a chromosome specific manner. In this case, the chromosome specific approach of the PCT, can already identify specific sub-telomeric p or q arms, according the selected biomarker. In this case, DNA sequences are hybridized for a specific biomarker and the telomere regions. Afterwards, the DNA is counterstained by specific dye in order to link the specific sub-telomeric biomarker probes with the respective telomeres. By this approach, many telomere modifications can be seen and quantified.

These modifications are grouped under the classification called Disease specific Telomere Application (DisTA).

DisTA by PCT: A Novel Method to Correlate Physical Telomere Length Measurements to Diseases Specific Biomarkers.

DisTA.

The second application derived from PCT is called DisTA, which stands for “Disease specific telomere length Combing Assay”. It is an application that implies identification of disease specific chromosome related ‘region of interest’ and its consequences on telomere length shortening/rearrangement events. DisTA can uniquely score for each specific chromosome and determine the following aspects: a) Detect and measure physical length (in kilobases) of the region of interest (disease related) for each specific chromosome. b) Detect and measure physical length (in kilobases) of the telomeres associated with region of interest (disease related) for each specific chromosome. c) Detect p or q arm of the region of interest (disease related) and telomeres for each specific chromosome. d) Detect intact DNA fibers by double stranded counterstaining dyes (e.g. YOYO1 & PO-PRO1).

DisTA is a novel assay for studying the telomereopathies, since none of the existing techniques/methods are able to correlate the physical telomere lengths with a ‘biomarker’ for the specific diseases. The biomarker can be a gene of interest or a sub-telomeric region on a defined chromosome.

For identification of target locus, all existing techniques/methods that use similar FISH probes are based on mathematical derivative of signal intensity¹⁷. None in comparison to DisTA, are able to measure pure lengths and thus are prone to high degree of bias due to mathematical derivations of signal strength quantification. Additionally, FISH based assays lose the ability of the picturing close proximity signals as the dot signals are almost overlapping to each other. Whereas, DisTA provides the ability to distinguish signals distinctly within a span of less than 2 kb on combed DNA fibers. DisTA is the perfect assay to identify biomarkers to correlate for a specific disease²². It can also be used to understand how the genetic background and the telomeres lead to the onset of a disease. DisTA is good to define the screening and efficacy of specific compounds that target telomere with the intent to block the disease progression, or ameliorate the symptoms for a patient. In addition, is the perfect system to help in the diagnosis of diseases generally (with large sample of the population) or for a specific patient to define a better course of action (precision medicine).

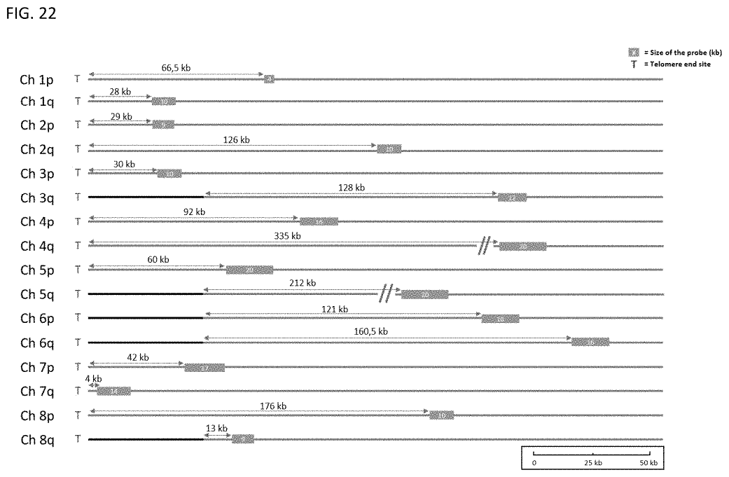

A panel of 46 distinct probes/sequences specific for each p and q arms of all chromosomes has been developed. These probes have unique sequences and precise physical distances from the telomere end site to identify each arm of all the chromosomes. The range of physical distances from telomere end site ranges from 1 kb to 200 kb (FIG. 22).

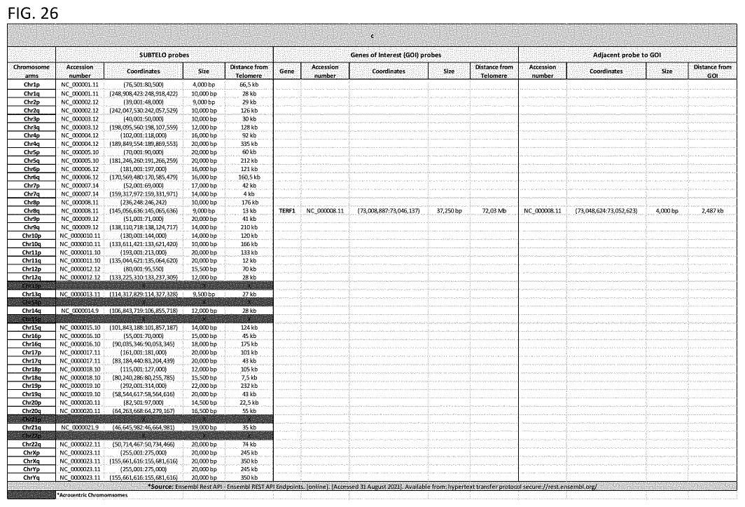

FIG. 26 provides an example of the DisTA approach and primer design for use in the present invention. The accession numbers for the genome, the chromosomes arms, and the specific probes (identified by sequence coordinates within the target accession number sequence) are as provided and are accessible at Ensembl Rest API—Ensembl REST API Endpoints. [online](hypertext transfer protocol secure://rest.ensembl.org/ [last Accessed 31 Aug. 2021]). The exemplary embodiment provides benchmark sequences; however, it is understood that the present invention is not bound to the specific defined sequences as it is well-known in the art that with sequences of the length of the probes permit localized mismatch while preserving global binding. Further, when considering the length of the sequences of the respective probes, the reduced degree of overall sequence identity is tolerated. Thus, an embodiment of the present invention are probes that are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 26.

DisTA can be further sub-divided into three distinct categories on the basis of their applications:

DisTAS.

Disease specific Telomere Application for Shortening (DisTAS) when there is shrinking of telomere at chromosome specific region for disease/locus specific manner. FIG. 6A and FIG. 6B describe chromosomal shortening events detectable using DisTAS.

DisTA/DisTAS for Evaluation of FSHD.

One example of such a disease is Facioscapulohumeral muscular dystrophy (FSHD). The onset of FSHD is considered to be due to the shortening of the sub-telomeric sequence on the chromosome 4 qA. Telomere rearrangements to the sub-telomeric region of interest i.e. double homebox protein 4 gene (DUX4) appear to be involved¹⁴. It was found that the severity of the disease is further aggravated due to telomere length shortening. Thus, precise determination of telomere lengths and D4Z4 tandemly repeated element will provide a more accurate diagnosis of the disease phenotype. DisTA is the only proprietary technique that can answer these questions. FIG. 6A and FIG. 6B provide schematically represent DisTAS for FSHD. Telomere are in the rightmost segment (red). The middle segments (magenta) are the sub-telomeres repeated units (D4Z4), short segments (green) depict chromosome 4 qA arm specific sub-telomeric regions i) 4 q adjacent to the D4Z4 ii) 4 qA1 between D4Z4 and telomere and counterstained DNA fibers (e.g. PO-PRO1)(thin segments in blue). This arrangement permits measurement of the physical length variation of DUX4 units and associated telomeres. In addition, these two entities can be associated to identify a correlation of telomere length with severity of the disease. At the same time, it is possible to identify rearrangements within the DUX4. Results indicate whether the telomere in the chromosome 10 qA is also contributing to FSHD and whether there are more rearrangements. Finally, DisTA can be used as an assay to better stratify the different patients affected from FSHD and identify the susceptibility of these patients to develop solid and/or liquid tumors.

DisTA/DisTAS for Evaluation of Gene of Interest (GOI).

Another scope of applicability with DisTA in telomere biology disorders (TBDs) involves identification of gene of interest (GOI) or biomarker which is not in close proximity of the telomere. Since, in most scenarios the causative effect of genetic modification which imply to telomere length degradation/maintenance, involves genes which are located elsewhere in the genome and not adjacent to telomere. In such cases, with the novel approach of combining DisTA chromosome arm specific probes and gene of interest (GOI) probes, telomere length alterations can also be characterized. For example, the gene of interest (GOI) TERF1 gene is scored, which is located on the chromosome 8 (q arm). TERF1 gene encodes for the protein Telomeric Repeat binding Factor-1 (TRF1). The gene encodes for this specific protein which is part of the telomere ‘shelterin’ complex; a nucleoprotein complex. The main role of this protein is to act and inhibit the telomerase activity throughout the cell cycle. Thus, it is involved in negative regulation of telomere maintenance. Over the past few years, it has been clinically postulated that the TRF1 protein corelates to telomere lengths in colorectal cancer^{51,52}. It has been shown that TRF1 was upregulated in tumor patients' samples in comparison to control samples. Thus, TRF1 levels are an important factor in tumor progression and could be used as a diagnostic parameter.

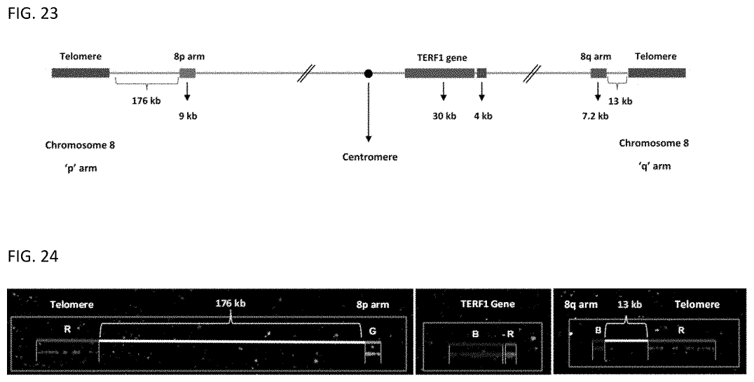

In FIG. 23 a schematic representation of DisTA with a panel of 3 color probes (red, green, and blue) for identification of each telomere w.r.t. to each arm as well as specific probes for TERF1 gene are demonstrated. The green color probe identifies the 8p arm of chromosome 8 with specific size and distance from telomere. The blue color probes are i) for identification of 8 q arm with a unique size and distance from telomere ii) for identification of the TERF1 gene with unique size. The red color probes are for i) the telomere ii) the adjacent short probe to the TERF1 gene identification.

Based on identifying signals for each p/q arm of chromosome 8 along with respective telomere and TERF1 gene signals, the measure of lengths and distances of each signal can be measured. Likewise, the number of events for i) TERF1 gene ii) chromosome 8p-arm and telomere iii) chromosome 8 q-arm and telomere can be counted, respectively. Thus, using Artificial Intelligence based FiberStudio® the statistical significance can be computed and depicted in co-relation to:
a) The shortening/loss of telomere lengths on 8 p arm.
b) The shortening/loss of telomere lengths on 8 q arm.
c) The number of intact TERF1 gene signals.
d) Statistical identification of arm specific shortening/loss of telomeres w.r.t TERF1 gene.
e) Statistical comparison of arm specific shortening/loss of telomeres w.r.t TERF1 gene in control vs patient samples.

DisTAL.

Disease specific Telomere Application for Loss when there is a specific loss of telomere signals after the specific sub-telomeric signals. FIG. 6C describes loss events detectable using DisTAL.

DisTAE.

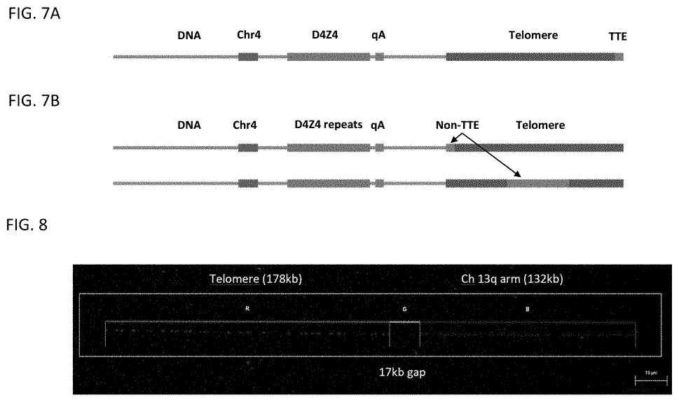

Disease specific Telomere Application for Elongation is used to characterize, quantify and measure the effect of the replication kinetics involved in the telomere elongation. DisTAE is used in combination with incorporation of dNTPs analogs to characterize and quantify the terminal telomere lengthening from the other replication signals. When the modified dNTPs analogs, e.g. IdU, are added during the cell division, a universal incorporation of these modified dNTPs is performed by the DNA polymerase complex on newly synthesized strands during DNA replication. One such scenario includes the incorporation of modified dNTPs while replicating through the telomeric ends as well. Likewise, events where drugs that aid in telomeric replication/elongation can also be scored by physically measuring the kilobases of newly synthesized telomere repeats. This pattern of identification of telomere elongation events is independent of cancerous or tumor cell types. It's a technique introduced to identify telomere elongation events in any cellular model. Cells are pulsed with dNTPs analogs, then DNA is stained. The specific signals from telomeres are depicted on the right (red). The signals of the chromosome specific locus D4Z4 repeats in the middle of the diagram (magenta) and the short segments (green) of the allele and the elongate dot in green FIG. 7A-FIG. 7B. DisTAE is a breakthrough method that can be used for aging related to diseases or cosmetics, when it is necessary to see the rescue of the telomere elongation and in oncology.

Software.

The PCT, as applied to either the genome wide or the chromosome specific applications, has been integrated into two software programs for automated or semi-automated analysis of the data obtained. The software programs are based on machine learning and artificial intelligence and classical block coding. Using these analytical software programs, PCT can provide a high-throughput for telomere analyses. In addition, these programs permit risk prediction of a specific treatment for a patient and assist in designing specific therapeutic compounds.

Software for SubTA.

In order to be a high-throughput and user-friendly technique SubTA is assisted with two software versions i.e. semi-automated; Classical FiberStudio® and automated; Artificial Intelligence based software programs for analysis of results obtained after scanning by the FiberVision® and/or FiberVision® S scanners. Both of the software versions provide multiple advantages for analysis to the user on a genome wide scale. These are; a) Holistic field of view of the coverslip scanned. b) Automated detection and measurements of telomere and ITSs signals. c) Automated detection and measurements of p and q chromosome arm specific sub-telomere signals. d) Visualization of DNA fibers counterstaining and determination of intact signals. e) Automated identification, statistical significance calculation and report generation of telomere lengths shortening w.r.t. sub-telomeric p and q chromosome arm specific signals. f) Automated identification, statistical significance calculation and report generation for the sub-telomeric rearrangements w.r.t. top and q chromosome arm specific signals. Thus, SubTA, in comparison to all existing techniques available commercially or research purposes, dominates in determining precise telomeric lengths measurements as well as offer additional information that none of the existing techniques can yet demonstrate w.r.t chromosome arm specific disease related instabilities.

Software for DisTA.

Similar to SubTA, DisTA is also assisted with two software versions, i.e. semi-automated; Classical FiberStudio® and automated; artificial Intelligence based FiberStudio® for analysis of results obtained after scanning by the FiberVision® and/or FiberVision® S scanners. Both of the software versions provide multiple advantages for analysis to the user on a genome wide scale. These are: a) holistic field of view of the coverslip scanned; b) automated detection and measurements of chromosome specific region of interest; c) automated detection and measurements of telomere. d) automated identification of p or q chromosome arm in reference to the region of interest and telomere. d) visualization of DNA fibers counterstaining and determination of intact signals. e) automated identification, statistical significance calculation and report generation of telomere lengths shortening w.r.t. p or q arm associated disease specific region of interest signals. f) automated identification, statistical significance calculation and report generation for the disease specific region of interest rearrangements w.r.t. p or q chromosome arm and telomere signals.

Both Classical FiberStudio® and AI Based FiberStudio® can work with both FiberVision® and FiberVision® S. Both Classical FiberStudio® and AI Based FiberStudio® communicate with scanners by obtaining scanned images and updating the software's database. To scan and analyze the coverslips, any combination of two scanners and two FiberStudio® software programs can be used. The only requirement is in order to use FiberVision® S, the software version of FiberStudio® must be at least 0.11, however at least the version 0.20.3 is preferred. The versions of classical FiberStudio® used for the analysis are 0.20.3 and 2.0. The AI based fiberstudio version is 3.0 Software inside FiberVision® S is “FiberVision® Scanner 2.0.0” developed by 3DHistech company for Genomic Vision. Information can be found on 3DHistech's website: hypertext transfer protocol secure://www.3dhistech.com/docs/common-scanner-information/fibervision-s/general-description/. FiberVision® product link: hypertext transfer protocol://www.genomicvision.com/products/molecular-combing-platform/scanner/. FiberVision® was developed in collaboration with the Franhaufer Institute and ITL. FiberVision® S product's page is not updated on the website yet. Classical FiberStudio® product Link: hypertext transfer protocol://www.genomicvision.com/products/molecular-combing-platform/software/. The content available at and through each of the above is incorporated by reference and was last accessed on Nov. 16, 2020.

Embodiments of the invention include, but are not limited to, the following.

A method for genome-wide or chromosome-specific detection of telomeres comprising isolating genomic DNA, hybridizing tagged telomere-specific, sub-telomeric-specific, or chromosome-specific probes to the DNA for a time and under conditions suitable for hybridization of the probes to the DNA, counterstaining genomic DNA sequences that are not hybridized to a probe, detecting the location of, or pattern of, the hybridized probes on the chromosomal DNA thereby providing data as to the location of the telomeric, sub-telomeric or chromosome-specific DNA on the chromosomes; and analyzing the data. Typically, and given the large amount of data collected, the data are analyzed using a computer program or algorithm.

This method may further comprise treating a subject from whom the genomic DNA was isolated for a disease, disorder, or condition associated with shortening, deletion, rearrangement, abnormality, or lengthening of telomeric sequences preferably as compared to one or more control values.

Treatments include reduction of risk or severity of a disease, disorder or condition associated with shortened, elongated or otherwise abnormal telomeres such as for the purpose to treat, prevent, cure, heal, alleviate, relieve, alter, remedy, ameliorate, improve or effect of these or at least one symptom thereof. Specifically, this method may further comprise treating a subject for a disease, disorder or condition associated with shortening or deletion of telomeres; further comprise treating a subject for a disease, disorder or condition associated with re-arrangement or other abnormality of telomeres; further comprise treating a subject for a disease, disorder or condition associated with elongation of telomeres, such as a neoplasm, tumor or cancer.

In some embodiments, this method can further comprise recording the locations of the probes on the chromosomal DNA, for example, by scanning, photography or other method.

Usually, said analyzing comprises computer analysis of the data as to the location of the telomeric, sub-telomeric, or chromosome-specific DNA on the chromosomes as manual analysis of such a large quantity of data would be impractical.

In some preferred embodiments, this method involves preparation DNA solution comprising the genomic DNA and may involve molecular combing of the chromosomal DNA.

Probes used in this method may be tagged with a color dye or other detectable indicator. In some embodiments, the probes will be color-tagged red, magenta, green and/or yellow-tagged probes and chromosomal DNA that is not hybridized to a probe will be counterstained blue. However, those skilled in the art may select one or more tags or counterstains depending on the particular PCT application.

In some embodiments, the probes for chromosome-specific, sub-telomeric, or telomeric DNA are labelled with haptens recognized by a color-labelled hapten-specific antibody or by a hapten-specific antibody and a color-labelled secondary antibody. In other embodiments, tertiary or quaternary antibodies may be used. Suitable haptens are commercially available and, along with labelling protocols are incorporated by reference to the suppliers and supplier reference numbers below. Haptens, such as those used herein, include the following.

| Product | Supplier | Reference |
|---|---|---|
| Fluorescein-12-dUTP | Sigma | 11373242910 |
| Digoxigenin-11-dUTP | Sigma | 11570013910 |
| BioPrime ™ DNA labeling System | ThermoFisher | 18094011 |
| dGTP | Sigma | 11051466001 |
| dTTP | Sigma | 11051482001 |
| dATP | Sigma | 11051440001 |
| dCTP | Sigma | 11051458001 |

This method may comprise manually detecting or visualizing the location of the hybridized probes on the chromosomal DNA. This method may comprise detecting or visualizing hybridization or the absence of hybridization to at least one region of interest on the chromosome using an image scanner such as a FiberVision® or FiberVision® S scanner.

Typically, the method also further comprises a computer or algorithmic analysis of the data. Such analysis or algorithms may use artificial intelligence methodologies to identify and/or correlate hybridization patterns to chromosomal DNA with particular conditions. Such programs may use machine learning based on providing the program with data showing known patterns or correlations (supervised learning), or may be designed to spot new, previously undiscovered patterns (unsupervised learning). Pattern recognition methods and algorithms are known and are incorporated by reference to hypertext transfer protocol secure://en.wikipedia.org/wiki/Pattern_recognition (last accessed Nov. 9, 2020).

For the Classical FiberStudio® detection system, the pattern recognition method is normalized correlation, in the help of OpenCV library's image processing operations. This method can be adjustable depending on the signal's features, by changing the kernels and the thresholds⁴⁶.

For AI based FiberStudio®, to detect a telomere signal, Deep Learning algorithms are used. Convolutional Neural Networks are used to learn automatically a signal's features and detect telomere signals on the coverslip. A supervised learning is applied (also called training) to obtain a convolutional neural network model⁴⁷.

For signal type recognition, Machine Learning classification algorithms are used. After feature extraction of the class patterns (for example q-arm telomere, p-arm telomere), which are defined as a probe's length, its repeat and its distance to other probes, supervised learning is applied to build a machine learning classifier in order to recognize a signal pattern⁴⁸.

In some embodiments of this method, one or more probes may be p or q arm specific and in other embodiments the one or more probes may be p or q are locus specific.

In one embodiment, the method involves genome-wide detection of telomere and sub-telomere sequences in genomic DNA, wherein the probes bind to telomeric and sub-telomeric sequences on the p and/or q arms of the chromosomes in the genomic DNA, and wherein said detecting comprises distinguishing telomeric and sub-telomeric sequences from interstitial telomeric sequences (ITSs). In some embodiments, this method is termed SubTA as disclosed elsewhere herein. In another embodiment, the method comprises genome-wide detection of telomere and sub-telomere sequences in genomic DNA, wherein said probes bind to telomeric and sub-telomeric sequences on the p and/or q arms of the chromosomes in the genomic DNA, and wherein said detecting comprises detecting a shortening of telomeres on the chromosomes of the genomic DNA compared to a control value. In some embodiments, this method is termed SubTAS as disclosed elsewhere herein.

In another embodiment, the method comprises genome-wide detection of telomere and sub-telomere sequences in genomic DNA, wherein said probes bind to telomeric and sub-telomeric sequences on the p and/or q arms of the chromosomes in the genomic DNA, and wherein said detecting comprises detecting a chromosome loss at the p or q arm of a chromosome compared to a control value. In some embodiments, this method is termed SubTAL as disclosed elsewhere herein.

In another embodiment, the method comprises genome-wide detection of telomere and sub-telomere sequences in genomic DNA, further comprising pulsing the genomic DNA with dNTP analogs prior to isolation; wherein said probes bind to telomeric and sub-telomeric sequences on the p and/or q arms of the chromosomes in the genomic DNA, and wherein said detecting comprises detecting an average elongation of telomeres on the arm or arms chromosomes in the genomic DNA compared to a control value. Such embodiments may comprise SubTAE or DisTAE applications.

Another set of embodiments are directed to chromosome-specific detection of telomeres and related sequences of interest.

In these embodiments, the method can comprise chromosome-specific detection of telomere and sub-telomere sequences in genomic DNA, wherein said probes bind chromosome-specific, telomeric and sub-telomeric sequences on the p and/or q arms of a chromosome in the genomic DNA, and wherein said detecting comprises distinguishing telomeric and sub-telomeric sequences on the chromosome from interstitial telomeric sequences (ITSs). Such embodiments may comprise SubTA or DisTA applications.

Such chromosome-specific methods may comprise chromosome-specific detection of telomere and sub-telomere sequences in a genomic DNA sample, wherein said probes bind to chromosome-specific, sub-telomeric, and telomeric sequences on the p and/or q arms of the chromosomes in the genomic DNA, and wherein said detecting comprises detecting a shortening of telomeres on the chromosomes of the genomic DNA compared to a control value. In some embodiments, this method is termed DisTAS as disclosed elsewhere herein.

This method may comprise chromosome-specific detection of telomere and sub-telomere sequences in genomic DNA, wherein said probes bind to chromosome-specific, sub-telomeric, and telomeric sequences on the p and/or q arms of the chromosomes in the genomic DNA, and wherein said detecting comprises detecting a chromosome loss at the p or q arm of a chromosome compared to a control value. In some embodiments, this method is termed DisTAL as disclosed elsewhere herein.

Such methods may also comprise target chromosome-specific detection of target chromosome-specific, sub-telomere, and telomere sequences in genomic DNA, further comprising pulsing the genomic DNA with dNTP analogs prior to isolation, wherein said probes bind target chromosome-specific, sub-telomeric, and telomeric sequences on the p and/or q arms of a chromosome in the genomic DNA, and wherein said detecting comprises detecting an average elongation of telomeres on the arm or arms of the target chromosome compared to a control value. In some embodiments, this method is termed DisTAE as disclosed elsewhere herein.

In some embodiments, PCT is used to evaluate effects of particular treatments on telomere length or telomere and sub-telomeric arrangement or rearrangement. Thus, the methods described herein may be performed on two or more samples taken from the same subject at different times, wherein said analyzing the data comprises comparing telomere lengths or configurations in the two or more samples.

The methods disclosed herein may practice using a kit reagents suitable for detecting or quantifying chromosome-specific, sub-telomeric, or telomeric sequences, such as oligonucleotide probes complementary to sequences of interest, haptens or anti-hapten antibodies may be provided in any suitable form, e.g. in liquid or lyophilized form. Kits may include reagents, supplies or equipment for molecular combing such as coverslips and molecular combing reagents. A kit or kit-of-parts may be a kit of two or more parts and typically comprises its components in suitable containers. For example, each container may be in the form of vials, bottles, squeeze bottles, jars, sealed sleeves, envelopes or pouches, tubes or blister packages or any other suitable form provided the container is configured so as to prevent premature mixing of components. Each of the different components may be provided separately, or some of the different components may be provided together (i.e. in the same container). A container may also be a compartment or a chamber within a vial, a tube, a jar, or an envelope, or a sleeve, or a blister package or a bottle, provided that the contents of one compartment are not able to associate physically with the contents of another compartment prior to their deliberate mixing by one skilled in the art. Kits may also be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device.

Other embodiments include a kit for detecting telomere shortening, rearrangement, loss or elongation.

Such kits may be used for detecting telomere shortening (SubTAS) and comprise at least one color-tagged probe that binds to a telomere and at least one probe that binds to a sub-telomeric sequence on a chromosome and optionally, immunostaining reagents, DNA extraction reagents, molecular combining supplies or equipment, and instructions for use of the kit to detecting telomere shortening; they may be used for detecting telomere loss (SubTAL) and comprise at least one color-tagged probe that binds to a telomere and at least one probe that binds to a sub-telomeric sequence on a chromosome and optionally, immunostaining reagents, DNA extraction reagents, molecular combining supplies or equipment, and instructions for use of the kit to detecting telomere loss; they may be used for detecting telomere shortening (SubTAE) and comprise at least one color-tagged probe that binds to a telomere and at least one probe that binds to a sub-telomeric sequence on a chromosome and optionally, dNTP analogs, immunostaining reagents, DNA extraction reagents, molecular combining supplies or equipment, and instructions for use of the kit to detecting telomere elongation; they may be used for distinguishing telomeres from interstitial telomere repeats, and comprise at least one color-tagged probe that binds to a telomere and optionally, at least one probe that binds to a sub-telomeric sequence on a chromosome, immunostaining reagents, DNA extraction reagents, molecular combining supplies or equipment, and instructions for use of the kit to distinguish telomeres from interstitial telomere repeats; they may be used for detecting telomere shortening (DisTAS) and comprise at least one color-tagged probe that binds to a telomere and at least one probe that binds to a sub-telomeric sequence on a chromosome, at least one probe that binds to a chromosome specific marker or locus and optionally, immunostaining reagents, DNA extraction reagents, molecular combining supplies or equipment, and instructions for use of the kit to detecting telomere shortening; they may be used for detecting telomere shortening (DisTAS) and comprise at least one color-tagged probe that binds to a telomere and at least one probe that binds to a sub-telomeric sequence on a chromosome, at least one probe that binds to a chromosome specific marker or locus and optionally, immunostaining reagents, DNA extraction reagents, molecular combining supplies or equipment, and instructions for use of the kit to detecting telomere shortening; wherein said chromosome specific probe(s) bind to 4 qA and 4 qB variants of the 4 qter subtelomere or other markers associated with FSHD interstitial telomere sequences; they may be used for detecting telomere loss (DisTAL) and comprise at least one color-tagged probe that binds to a telomere and at least one probe that binds to a sub-telomeric sequence on a chromosome, at least one probe that binds to a chromosome specific marker or locus and optionally, immunostaining reagents, DNA extraction reagents, molecular combining supplies or equipment, and instructions for use of the kit to detecting telomere loss; or they may be used for detecting telomere shortening (DisTAE) and comprise at least one color-tagged probe that binds to a telomere and at least one probe that binds to a sub-telomeric sequence on a chromosome, at least one probe that binds to a chromosome specific marker or locus, and optionally, dNTP analogs, immunostaining reagents, DNA extraction reagents, molecular combining supplies or equipment, and instructions for use of the kit to detecting telomere elongation.

Summary.

For each of the given chromosome specific or for the overall genome wide applications, the PCT gives the ability to detect the number of telomeres for a specific chromosome end, to understand the genome rearrangement due to the identification of ITSs, detect telomere events of shortening, elongation or loss, determine the physical telomere length for each event of shortening and elongation, identify the existence of a correlation between a telomere event (shortening, elongation or loss) respect to a specific chromosome region, determine the percentage telomere shortening and/or elongation compared to the given genome length. The PCT can be exploited by a variety of model systems (human, mouse, plant and/or human derived samples) collected by saliva, blood, organoid, xenograft, PDX, and adherent and suspension cell lines. Then, PCT, its applications and the derivative kits ready to use, can be easily used in research, in diagnostic, for drug screening/testing, for cells/samples stratifications, in quality control process for engineered cells/organisms.

PCT provides a breakthrough method to bring more details to support and help investigators to answer how telomere events (such as elongation, shortening and loss) occur. As consequence of the telomere modifications, researchers and/or physicians can use PCT to characterize what is involved and how to work with it.

The genome-wide and chromosome-specific applications of the PCT are explained in more detail in the sections below and in the Examples.

### EXAMPLES

### 1. Materials and Methods

### Materials

### Equipment Used.

| Equipment | Supplier | Reference |
|---|---|---|
| Incubator | Any | N/A |
| Thermo-block | Any | N/A |
| Water-bath | Any | N/A |
| Cell culture CO₂ incubator | Any | Any |
| LunaFL ™ Automated Cell Counter | Moka Science | L2001 |
| Hybridizer | DAKO | S245230-2 |
| Humid Hybridization Box | Dutscher | 068506 |
| (Glass box with cover) | | |

### Materials

| Materials | Supplier | Reference |
|---|---|---|
| Pipetman p10, p20, p100, p200, p1000 | Any | N/A |
| Tips for pipetman p10, p20, p100, | Any | N/A |
| p200, p1000 | | |
| Glass slide | Any | N/A |
| Gel plug mold | Bio-Rad | 170-3706 |
| Photo-resistant Eppendorf tube 1.5 ml | Any | N/A |
| 1.5 mL Protein LoBind ® tube | Eppendorf | 0030122356 |
| Luna-FL ™ Counting slide | Moka Science | L12001 |
| Vinylsilane Coated Coverslips | Genomic Vision | COV-002 |
| Sample Holders | Genomic Vision | HLD-001 |
| Humidity hybridizer control strip | DAKO | S2542 |

| Products, Reagents, Media | Supplier | Reference |
|---|---|---|
| DMEM cell culture media | FisherScientific | 11966025 |
| Trypsin/EDTA | Invitrogen | 25300-062 |
| AO/PI | Moka Science | F23001 |
| Trypan Blue Stain 0.4% | Life Technologies | 15250-061 |
| Nanobind CBB Big DNA Kit | CIRCULOMICS | SKU |
| | | NB-900-001-01 |
| DynaMag ™-2 | Invitrogen | 12321D |
| TelC-Alexa647, TelG-Cy3 | PANAGENE | F2003, F1006 |
| FITC, Texas Red | Cytocell | LPT13QR, |
| | | LPT21QG |
| IdU (5-iodo-2'-deoxyuridine) | Sigma-Aldrich | I-7125 |
| Mouse anti-BrdU (IdU) | BD Biosciences | 347580 |
| Goat anti-mouse Cy3.5 | Abcam | AB6946 |
| Mouse anti human ssDNA | Merck | MAB3034 |
| SAV-BV480 | FisherScientific | 564876 |
| Goat anti-mouse BV480 | Jackson Immunoresearch | 115-685-166 |
| YOYO1 | ThermoFisher Scientific | Y3601 |
| PO-PRO1 | ThermoFisher Scientific | P3581 |
| Na₂HPO₄•2H₂O bio ultra | Sigma Aldrich | 71636 |
| Tris base | — | Genomic |
| | | Vision |
| Formamide deionized ≥99% | VWR | 444472T |
| UltraPure™ SSC, 20X | Invitrogen | 15557036 |
| Salmon Sperm DNA, sheared | ThermoFischer Scientific | AM9680 |
| 10 mg/ml (ssDNA) | | |
| Tween ® 20 (polysorbate) | VWR | 28829.296 |
| technical | | |
| NaCl | Sigma-Aldrich | S-9888 |
| NaOH | VWR | 28 245.298 |
| Absolute Ethanol | Merck | 107017 |
| PBS 1X | Dutscher | 702594 |
| BlockAid ™Blocking | ThermoFischer Scientific | B10710 |
| solution | | |

Biological Materials.

Commercial human genomic DNA (TaKaRa Bio), Patient Blood samples (EFS: Etablissement Francais du Sang), and human cell lines HeLa & U-2OS were used to develop the assay. Hela & U2-OS cell lines were cultured in Dulbecco's Modified Eagle's Medium (DMEM; Gibco, Paisley, United Kingdom) supplemented with 10% fetal bovine serum (FBS) (Gibco™) with 1% Penicillin-Streptomycin (Gibco™) at 37° C. in 10% CO₂.

During the exponential growth phase of a cell culture, IdU (5-iodo-2′-deoxyuridine) was incorporated.

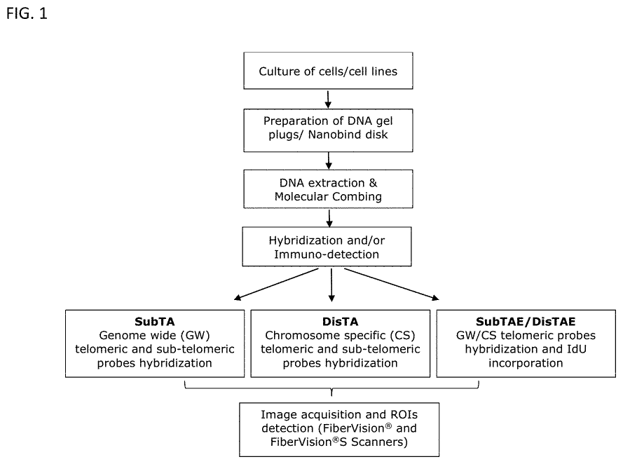

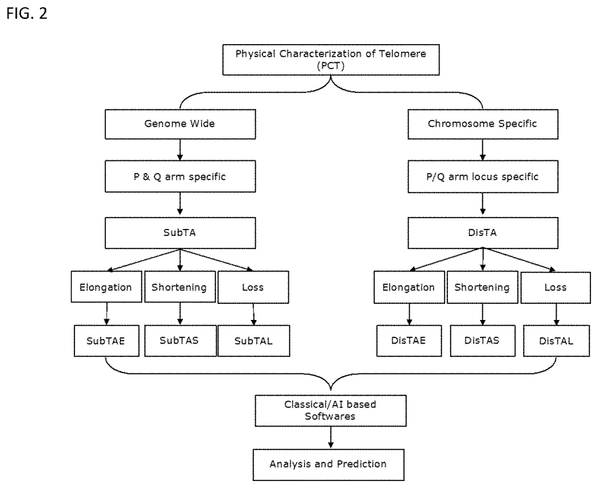

FIG. 1 provides a synopsis of features of PCT.

DNA Extraction: Genomic Vision Extraction Kit:

For the preparation of DNA solution cells were harvested by using trypsin, then complete DMEM was added to inhibit the activity of the trypsin. The counting of cells was carried out by Luna-FL™ Automated Cell Counter. In order to prepare 500,000 cells per gel plug (90 μL), cells were re-suspended in a volume of (45 μL per gel plug) PBS/Trypsin mixture (1:1) i.e. Buffer 1 (FiberPrep® Kit, Genomic Vision). Proportional volume (45 μL per gel plug) of 2% LMT agarose gel plugs (low melting agarose) i.e. Buffer 2 (FiberPrep® Kit, Genomic Vision) was added and gel plugs were casted (final volume of 90 μL) using Gel plug mold (BioRad Laboratories). These gel plugs were treated with 0.5M EDTA pH 8.0, 25 μl of 10% (w/v) of Sarcosyl/0.5M EDTA and 251 of 20 mg/ml Proteinase K (Buffer 4; FiberPrep® Kit, Genomic Vision) at 50° C. for 16-18 hours. The gel plugs were transferred in Reservoirs® (Genomic Vision) containing 0.5M MES solution pH 5.5 (Buffer 5; FiberPrep® Kit, Genomic Vision) for digestion using beta-agarase (Buffer 7; FiberPrep® Kit, Genomic Vision) for 16-18 hours at 42° C. From this DNA solution in Reservoir® (Genomic Vision) molecular combing was performed using the FiberComb® Molecular Combing System (Genomic Vision) with a constant stretching factor of 2 kb/μm using Vinylsilane coverslips (20×20 mm; Genomic Vision). To allow the complete attachment of DNA molecules, the combed coverslips were baked at 60° C. for 4 hours.

Nanobind CBB Big DNA Kit:

For the preparation of DNA solution cells were harvested by using trypsin, then complete DMEM was added to inhibit the activity of the trypsin. The counting of cells was carried out by Luna-FL™ Automated Cell Counter. In order to prepare 500,000 cells, centrifugation was done at 500×g for 3-5 min at 4° C. to pellet cells in a 1.5 mL Protein LoBind® tube (Eppendorf). After removal of the supernatant, added 20 μL of 1×PBS and pipetted mix 10 times with P200 pipette to re-suspend cells. Added 20 μL of Proteinase K and 20 μL of CLE3 (Nanobind CBB Big DNA Kit, CIRCULOMICS) mixed 5 times with the P200 pipette. Performed incubation at RT for 15 mins at 25° C. For RNA removal, added 20 μL of RNAase A and pipetted 5 times and incubated at RT for 3 mins. Added 200 μL of Buffer BL3 (Nanobind CBB Big DNA Kit, CIRCULOMICS) and pipette 10 times with P200 pipette. Carried out incubation at RT for 15 mins. Added Nanobind disk (Nanobind CBB Big DNA Kit, CIRCULOMICS) to cell lysate and added 300 μL of Isopropanol. Mixed 5 times by inversion and placed the tubes on rotator at 9 rpm at RT for 10 mins. The tubes were placed on the DynaMag™-2 magnet stand (Invitrogen) and discard the supernatant using P200 pipette. Add 700 μL of Buffer CW1 (Nanobind CBB Big DNA Kit, CIRCULOMICS) and mixed by inversion 4 times. Discard the supernatant and add 500 μL of Buffer CW2 (Nanobind CBB Big DNA Kit, CIRCULOMICS) and mix by inversion 4 times. The supernatant was discarded and placed the Nanobind disk (Nanobind CBB Big DNA Kit, CIRCULOMICS) in the Reservoir® (Genomic Vision) and add 200 μL of EB Buffer (Nanobind CBB Big DNA Kit, CIRCULOMICS). Incubated at RT for 20 mins and added 2 mL of 0.5M MES solution pH 5.5 (Buffer 5; FiberPrep® Kit, Genomic Vision) for 16 h-18 h at RT.

Molecular Combing from DNA Solution:

From this DNA solution in Reservoir® (Genomic Vision) molecular combing was performed using the FiberComb® Molecular Combing System (Genomic Vision) with a constant stretching factor of 2 kb/μm using Vinylsilane coverslips (20×20 mm; Genomic Vision). To allow the complete attachment of DNA molecules, the combed coverslips were baked at 60° C. for 4 hours.

Hybridization of Genome Wide Telomeric and Sub-Telomeric Region Probes (SubTA):

The Hybridization Buffer Mix (HBM) solution was prepared to facilitate the attachment of Telomere specific probes (PANAGENE; AlexaFlour 647) as well as the chromosome specific sub-telomeric probes (Cytocell; e.g. Ch-21 q with FITC, & Ch-13 q with TexasRed). This buffer was composed of Na₂HPO₄.2H₂O (0.1M) pH 7.4, Tris (1M) pH 7.4, 100% Formamide, 20×SSC, Salmon Sperm DNA (10 mg/ml) and DNAse-free H₂O. In the Hybridization Buffer Mix (HBM) the working concentration of 250 nM telomere probes (PANAGENE) and 13.3 ng/μl sub-telomeric probes (Cytocell) was adjusted per coverslip. On a glass slide (pre-heated at 80° C.) the combed coverslips were placed (engraved area facing downwards) on the drop of Hybridization Buffer Mix (HBM) with probes. These coverslips on glass sides were incubated in the humid hybridization glass box (Dutscher) for 10 mins at 85° C. for a denaturation step.

Post denaturation glass sides with coverslips were incubated in hybridizer (DAKO) at 30° C. for 20 hours. The coverslips were washed using the Wash Buffer (2×SSC+0.1% Tween) twice at 60° C. in water bath followed by one wash at room temperature. The coverslips were washed with 1×PBS and dehydrated with serial ethanol washes (70%-100%). Post dehydration the coverslips were counterstained with BA-YOYO1/BA-PO-PRO1 (ThermoFisher) by the use of FiberComb® Molecular Combing System (Genomic Vision). The coverslips were loaded in the specialized bar-coded Sample Holders® (Genomic Vision) to perform the automated scanning of coverslips by the use of FiberVision® scanner (Genomic Vision).

Hybridization of Telomeric and Disease Specific Sub-Telomeric Region Probes (DisTA) Applied for FSHD:

For the hybridization for Disease specific Telomere Combing Assay (DisTAS) the hybridization buffer was composed of 20×SSC, 4M NaCl, 10% SDS, 10% Sarcosyl and BlockAid. The hybridization buffer was complemented with 250 nM telomeric probes (PANAGENE) and 150-200 ng/μL of disease specific region of probes grouping labelled with different haptens respectively. For example, in FSHD (Facioscapulohumeral muscular dystrophy) the D4Z4 repeats for disease specific gene DUX4 was labelled with digoxigenin (Dig) and chromosome linked i.e. 4 q was labelled with fluorescein (Flu) (FSHD-Test®, Genomic Vision). An equal volume of 100% formamide (v/v) was supplemented to the probe hybridization mix (Hybridization Solution) and it was incubated for 30 mins at 37° C. 20 μL of Hybridization Solution was added on glass slide to which the combed coverslips (engraved area facing downward) was placed. These coverslips on glass sides were incubated in the humid hybridization glass box (Dutscher) for 5 mins at 85° C. for denaturation step. Post denaturation glass sides with coverslips were incubated in hybridizer (DAKO) at 37° C. for 20 hours.

Coverslips were washed using the 2×SSC thrice at 60° C. in water bath. Subsequently, they were incubated with the mixture of primary antibodies by adding a drop of the mixture directly on the surface (e.g. FSHD-Telomere; Mouse anti Dig-Alexa 647 & Mouse anti Flu-Cy3) with BlockAid for 20 mins at 37° C. in a moist box. The coverslips were washed with Wash Buffer (2×SSC+1% Tween) for 3 mins thrice at room temperature. The coverslips were rinsed with 1×PBS and dehydrated with serial ethanol washes (700%-100%). Post dehydration the coverslips were loaded in the specialized bar-coded Sample Holders® (Genomic Vision) to perform the automated scanning of coverslips by the use of FiberVision® scanner (Genomic Vision).

Hybridization of Telomeric and Disease Specific Sub-Telomeric Region Probes (DisTA) Applied for TERF1 Gene on Chromosome 8:

For the hybridization for Disease specific Telomere Combing Assay (DisTAS) the hybridization buffer is composed of 20×SSC, 4M NaCl, 10% SDS, 10% Sarcosyl and BlockAid. The hybridization buffer is complemented with 250 nM telomeric probes (PANAGENE) and 150-200 ng/μL of disease specific region of probes grouping labelled with different haptens respectively. For the TERF1 gene and the chromosome 8 q arm the probe are labelled with digoxigenin (Dig). The chromosome 8p arm is labelled with fluorescein (Flu) and the TERF1 adjacent probe is labelled with Biotin (Biot). An equal volume of 100% formamide (v/v) is supplemented to the probe hybridization mix (Hybridization Solution) and it is incubated for 30 mins at 37° C. 20 μL of Hybridization Solution is added on glass slide to which the combed coverslips (engraved area facing downward) is placed. These coverslips on glass sides are incubated in the humid hybridization glass box (Dutscher) for 5 mins at 85° C. for denaturation step. Post denaturation glass sides with coverslips are incubated in hybridizer (DAKO) at 37° C. for 20 hours. Coverslips are washed using the 2×SSC thrice at 60° C. in water bath. Subsequently, they are incubated with the mixture of primary antibodies by adding a drop of the mixture directly on the surface i.e. Mouse anti Dig-Alexa 647, Mouse anti Flu-Cy3 and SAV-BV480 with BlockAid for 20 mins at 37° C. in a moist box. The coverslips are washed with Wash Buffer (2×SSC+1% Tween) for 3 mins thrice at room temperature. The coverslips are rinsed with 1×PBS and dehydrated with serial ethanol washes (700%-100%). Post dehydration the coverslips are loaded in the specialized bar-coded Sample Holders® (Genomic Vision) to perform the automated scanning of coverslips by the use of FiberVision® scanner (Genomic Vision).

Hybridization of Telomeric Probes and Detection of Telomere Elongation by dNTP Incorporation (for SubTAE and DisTAE):

The Hybridization Buffer Mix (HBM) solution was prepared to facilitate the attachment of Telomere specific probes (PANAGENE; AlexaFlour 647). This buffer was composed of Na₂HPO₄.2H₂O (0.1M) pH 7.4, Tris (1M) pH 7.4, 100% Formamide, 20×SSC, Salmon Sperm DNA (10 mg/ml) and DNAse-free H₂O. In the Hybridization Buffer Mix (HBM) the working concentration of 250 nM telomere probes (PANAGENE) was adjusted per coverslip. Combed coverslips were denatured with 0.5 M NaOH/1M NaCl solution for 8 min at room temperature. The coverslips were washed with 1×PBS one time and dehydrated with serial ethanol washes (70%-90%-100%). Simultaneously, the PANAGENE probes were added in the Hybridization Buffer Mix (HBM) and heated for 10 mins at 90° C. On a glass side (pre-heated at 80° C.) the denatured coverslips were placed (engraved area facing downwards) on the drop of Hybridization Buffer Mix (HBM) with PANAGENE probes. These coverslips on glass sides were incubated in the moist box with humidity for 2 hrs at 37° C. Post hybridization, the coverslips were washed with Wash Buffer (2×SSC+0.1% Tween) twice at 60° C. in water bath followed by one wash at room temperature. The coverslips were washed with 1×PBS and dehydrated with serial ethanol washes (70%-90%-100%).

The coverslips were next treated with 1^{st} antibody solution i.e. mix of mouse anti-BrdU (IdU) in BlockAid. A droplet of 25 μL was added for each coverslip and was incubated in moist box with humidity for 1 hr at 37° C. Post incubation, the coverslips were washed with 1×PBS/Tween 20 (0.1%) 3 times and dehydrated with serial ethanol washes (70%-90%-100%).

The coverslips were treated with 1^{st} antibody solution i.e. goat anti-mouse Cy3.5 in BlockAid. A droplet of 25 μL was added for each coverslip and was incubated in moist box with humidity for 45 mins at 37° C. Post incubation, the coverslips were washed with 1×PBS/Tween 20 (0.1%) 3 times and dehydrated with serial ethanol washes (70%-90%-100%). The coverslips were treated with 3^{rd} antibody solution i.e. mouse anti human ssDNA in BlockAid. A droplet of 25 μL was added for each coverslip and was incubated in moist box with humidity for 2 hours at 37° C. Post incubation, the coverslips were washed with 1×PBS/Tween 20 (0.1%) 3 times and dehydrated with serial ethanol washes (70%-90%-100%). The coverslips were treated with 4¹ antibody solution i.e. Goat anti-mouse BV480 in BlockAid. A droplet of 25 μL was added for each coverslip and was incubated in moist box with humidity for 45 mins at 37° C. Post incubation, the coverslips were washed with 1×PBS/Tween 20 (0.1%) 3 times and dehydrated with serial ethanol washes (70%-90%-100%). The coverslips were loaded in the specialized bar-coded Sample Holders® (Genomic Vision) to perform the automated scanning of coverslips by the use of FiberVision® scanner (Genomic Vision).

Automated Detection of Telomere, Sub-Telomere and Disease Specific Regions by Genomic Vision Technology:

The FiberVision® and FiberVision® S scanners (Genomic Vision) have a high throughput multi-color channel image acquisition of entire combed coverslip. They acquire many pictures of the coverslip (25×25) by depicting the different channels of the fluorophores signals designed to represent telomeric and sub-telomeric regions (SubTA), disease specific regions (DisTA) and telomere elongation events (SubTAF/DisTAE) for hundreds of genome copies combed on an entire coverslip. The machines take one hour to acquire the images and stich all together in order to rebuild the digital version of the coverslip carrying the signals. After the scan, the entire coverslip image is transferred and stored at the workstation (the server) where the tiling and the analysis are performed by the FiberStudio® software. FiberStudio® software consist of individual custom designed algorithms that scores for telomeric and sub-telomeric detection for SubTA. While, telomeric and disease specific region along with identified chromosome detection for DisTA and similarly identifying telomere elongation events in SubTAE/DisTAE. Post detection, the user has access to the image of the coverslip, where the signals and the scoring can be reviewed and validated. In the end, a report describing the physical telomeric, sub-telomeric, disease specific region lengths measurements and genome wide telomere length elongation w.r.t. sub-telomeric, disease specific region and telomere elongation is generated for SubTA, DisTA and SubTAE/DisTAE respectively.

Novel Method for Physical Characterization of Telomere (PCT).

The PCT, and its derivate applications, brings the advantage to detect telomeres as well as specific region in the proximity of the telomeres, called sub-telomeric regions. It includes the idea to identify the chromosome specific or genome wide modifications of telomere & sub-telomeric regions in reference to the p & q chromosomal arms, depending on parameters of elongation, shortening and loss of telomere sequences. With this novel approach, the true physical lengths of telomere and sub-telomere regions are determined. Until now, it has not been possible to demonstrate the physical correlation of sub-telomeric and telomeric regions detection on intact DNA. The methods to visualize sub-telomeric and telomeric regions are based on Q-FISH, FISH and tangled DNA fibers using spreading methods using probes designed for Florescence in situ hybridization. However, these existing techniques are based on quantitative data of florescence signal detection that are non-conclusive with respect to physical telomere identification. In PCT the identification of telomeric and sub-telomeric regions is scored using FISH probes. In addition, visualization of region of interest can also be carried out using other substrates/molecules such as oligonucleotides, artificial chromosomes and enzyme-based nucleotide insertion methods. Thus, PCT is the only accurate way to identify physically the specific biomarkers for telomeropathies, cancer and aging related diseases, to understand diseases onset, severity or simply a genetic predisposition to have a specific disease^{27,12}.

Physical Characterization of Telomeres (PCT) Using Genomic Vision Technology.

With the use of Genomic Vision proprietary technology, the genomic DNA is collected and processed by using Genomic Vision DNA FiberPrep® kit. Then, DNA is combed on Engraved Coverslips® by using FiberComb® to stretch the single DNA molecules on the surface of coverslip that has previously coated with vinylsilane. After, the combed single DNA fibers are processed to hybridization protocol to allow the pairing of the telomere and sub-telomeric probes for genome wide or chromosome specific analyses along with immunodetection using thymidine analogs. Finally, the hybridized single DNA fibers are counterstained by using YOYO1, PO-PRO1, Syto40, Syto41, TOTO-1, JOJO-1, POPO-1, GelRed, SyberGreen, SyberSafe, ssDNA-BV480.

Additional features of PCT is the capability to take tracks for each of the events affecting telomere: elongation, shortening and loss, and to distinguish if these occurrences occur to which extent specifically on the short arms (p arm), long arm (q arm) or correlated to a modification of a chromosome specific region. PCT applications are very precise methods with accuracy between 0,8 kb up to 250 kb and more. This is possible by using the hybridization of sub-telomeric regions and by taking the physical measurements of the single DNA fibers, sub-telomeric regions and telomere.

The other available methods to take telomere measurements do not have the same accuracy for the measurement because they are based on a relative quantification of telomerase reaction (qPCR), or the length is derivate from the intensity of the signal (Q-FISH and FISH). Other methods measure telomere length by the molecular weight and the ability to migrate within a gel (TeSLA, STELA, TRF). Likewise, Telomere Combing Assay (TCA (or TFF)) utilizes the same principle of stretching DNA fibers and taking telomere length measurement. However, this assay has following shortcomings: a) it fails distinguish between true telomere signals and the interstitial telomere sequences (ITSs). b) It is incapable to identify, visualize and measure p and/or q arm specific sub-telomeric or disease specific chromosome sites. c) It is incapable to identify the terminal telomere elongation events. d) It fails to have high-throughput analyses approach and does not have the predictive tool to aid in clinical or diagnostic study/treatment. Thus, due to these inadequacies TCA (or TFF) fails to give a precise answer about what are true telomere signals and where in genome wide manner the telomere are affected, and if there is a correlation between telomere length and specific sequence on the DNA.

Thus, PCT has the incredible advantage to physically correlate together sub-telomeric and telomere regions independently from their distance. This correlation can be exploited then to perform studies for the comparison of telomere length genome wide and/or to identify new biomarker and correlate these to the telomere events of elongation, shortening or loss in chromosome specific manner. PCT has great accuracy because the coverslips carrying the single DNA fibers are hybridized with sub-telomeric and telomere probes which are acquired by Genomic Vision automated FiberVision® and FiberVision® S scanners at magnitude of 40× or 63× or 20×. After, the images are analyzed on GenomicVision software, i.e. Classical FiberStudio® software or Artificial Intelligence based FiberStudio®.

Physical Characterization of Telomeres (PCT) Using Sub Telomere Application (SubTA).

One feature of the PCT is its capacity to detect telomeres and study chromosomes in a genome-wide manner. This is classified under the name of Sub Telomere Application (SubTA) which is further subclassified into Sub Telomere Application for Shortening (SubTAS), Sub Telomere Application for Elongation (SubTAE) and Sub Telomere Application for Loss (SubTAL). Depending on the genome wide application of SubTA, the assay can be used to understand an overall and/or regional telomere modification, for example, it is possible to distinguish between the different signals from telomeres at the ends of chromosomes or telomere-like DNA such interstitial telomere sequences (ITSs).

SubTAS provides a well-defined identification and classification between true telomere signals and ITS. It further distinguishes between signals from the p arm or q arm of a chromosome in genome wide or chromosome specific manner (FIG. 3A-FIG. 3B AND FIG. 4A-FIG. 4C).

Likewise, for SubTAL is the application that highlights events of chromosomal loss occurring at a genome wide level, specific to each p or q arms of the chromosomes.

Similarly, another additional value of SubTA is the investigation, characterization, quantification and measurement of the telomere events such as elongation by the application called SubTAE (FIG. 5A-FIG. 5B). There is increasing evidence that due to physiological processes that telomere length diminishes during an organism's lifespan. Certain mechanisms involving telomerase, an enzyme which adds telomere sequence repeats, are responsible for slowing down telomere shortening. Telomeres cap the ends of eukaryotic chromosomes and protect them^{28,29} and telomere homeostasis is a key process for the determination of the replicative life span, cellular senescence, and cancer cell lifespan or immortalization³⁰.

The repeats of the telomere sequences are added by a specific enzyme called telomerase. Telomerase comprises a catalytic subunit, the telomerase reverse transcriptase (TERT), and RNA template that for human is known as human telomerase RNA (hTR). Normally, hTR is always expressed in all cells, while the TERT is restricted to stem cells^{31,32}; then telomere elongation happens only when cells carry fully active telomerase³³. Either TERT or hTR are limiting factors that could bring to haploinsufficiency of telomerase, which is associated to the development of pathological conditions due to telomere shortening³⁴⁻³⁵⁻³⁶.

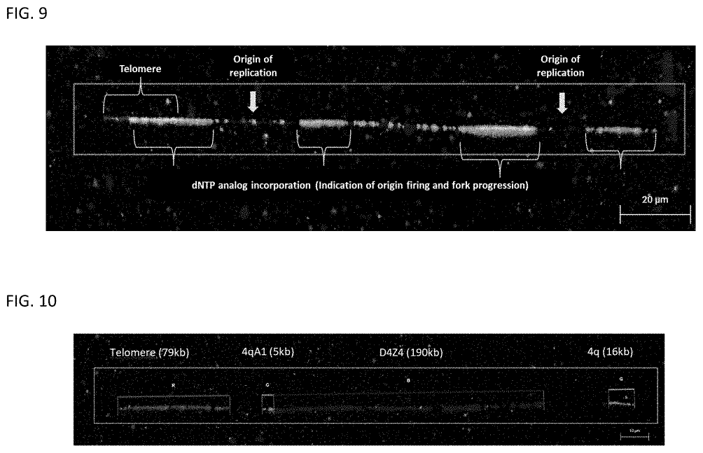

SubTAE, unlike other methods, gives an incredible output and resolution of the telomere elongation. In order to visualize the telomere elongation events, the inventors have developed a specific protocol that employs the use of thymidine analogs to depict elongated telomeres during replication. During this protocol, a sample is pulsed with a combination of one or two dNTPs analogs such as 5-ethynyl-2′-deoxyuridine (EdU), 5-chloro-2′-deoxyuridine (CldU), 5-iodo-2′-deoxyuridine (IdU), 5-bromo-2′-deoxyuridine (BrdU), 5-azidomethyl-2′-deoxyuridine (AmdU), 5-vinyl-2′-deoxyuridine (VdU). FIG. 9 depicts the IdU incorporation within the telomere as well as adjacent to telomere (sub-telomeric region) to illustrate the propagation of replication.

For high-throughput analyses and classification of results for each of the PCT applications, automated or semi-automated software, i.e. FiberStudio® Classical or the Artificial Intelligence based software programs are developed to strengthen the statistical significance to score for each event occurring within a genome.

Physical Characterization of Telomeres (PCT) Using Disease Specific Telomere Application (DisTA).

Another feature of PCT, named Disease specific Telomere Application (DisTA), involves identifying and characterizing events occurring in a chromosome-specific manner within a genome, for example, events associated with a presence or onset of a telomere-related disease, disorder or condition (telomeropathies). The impact of telomere shortening, elongation or loss on the sub-telomeric region of interest or vice versa within a specific chromosome causative to a disease, can be classified in PCT under the name of Disease specific Telomere Application (DisTA). The impact of telomere shortening, elongation or loss on the sub-telomeric region of interest or vice versa within a specific chromosome causative to a disease, can be classified in PCT under the name of Disease specific Telomere Application (DisTA).

DisTA based on application is further subclassified under three categories: Disease specific Telomere Application for Shortening (DisTAS), Disease specific Telomere Application for Loss (DisTAL) Disease specific Telomere Application for Elongation (DisTAE).

Depending on the application, DisTA can be used to score for the physical disease specific identification of region of interest as well as telomere length alterations associated by the use of DisTAS (FIG. 10 and FIG. 24).

Similarly, while identifying the events of loss of disease specific sub-telomeric region or the telomeric regions can be determined by the use of DisTAL.

Lastly, scenarios where the aim is to characterize, quantify and measure the elongation of telomere with respect to the chromosome of interest, DisTAE can be used.

To analyze the data acquired from the PCT methods, including identification of elongation, shortening or loss events from high-throughput analyses and classification of results for each respective application of PCT and determine their statistical significance, the inventors developed automated or semi-automated software such as FiberStudio® Classical or the Artificial Intelligence-based software.

High Throughput Automated/Semi-Automated Detection Algorithm to Analyze Data Obtained.

The inventors have developed two software programs to allow the PCT, and the derivative applications, to be high-throughput assays that can be used either in a research lab, pharma companies, biotech, clinical trials and hospitals.

The two software programs are based on our FiberStudio® and are based on classical image processing algorithms and/or on the machine learning and artificial intelligence.

The classical software has been coded to recognize all the signals coming from the different probes separately.

The detection process requires specific image processing operations for each probe. And uses specific filters defined by the developers for a given signal type.

After detection, signals are sorted according to priorities i.e. telomere signal first, then signals from sub-telomeric or disease-specific probes and finally the DNA fibers.

Patterns of signals are put beside each other the signals coming from the different probes are used to design a true validated region of interest (ROI) or object of interest.

Algorithms are applied that detect patterns down to a lower limit of resolution of 1 kb and an upper limit of 250 kb and more. The software is based on artificial intelligence comprising a convolutional neural network which is specific to object detection is previously trained to recognize a valid signal's features.

When an image is fed to the algorithm, by analyzing image's features, the neural network throws the predictions of the objects present on the scanned slide and filters the objects which are more likely to be validated as telomere signal (SubTA, DisTA). By the artificial intelligence-based software, in the same way like the detection process, an artificial neural network is previously trained by using the data of slides reviewed by researchers to detect and measure the length and characteristics of a signal (or dot). Each dot can be automatically measured with a very high accuracy.

A separate Reporting module was developed as the last step of the FiberStudio® software, which can use the detection data either from Classical software or AI based software to generate reports containing statistical analysis of detected signals and predictive analysis for diagnostics.

Classical FiberStudio® Software:

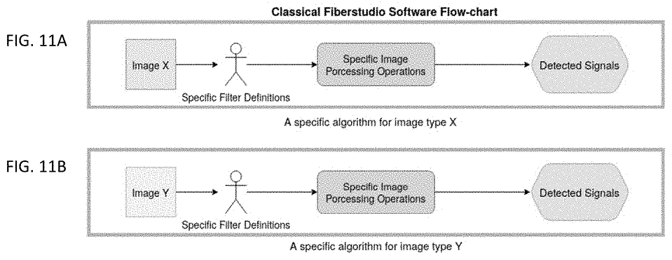

Classical FiberStudio®® is used to detect signals on a scanned image of a coverslip. The algorithm is developed specifically to help the investigator to answer each of the biological questions and parameters once the wet protocol has been performed (FIG. 11A-FIG. 11B show the flow-chart of classical FiberStudio® software). To detect the telomere signals, in classical FiberStudio®, a combination of some image processing methods and algorithms is used, by using OpenCV library. OpenCV stands for Open Source Computer Vision, which contains various function for image processing (hypertext transfer protocol secure://opencv.org/, last accessed Nov. 8, 2020, incorporated by reference).

Signals Detection.

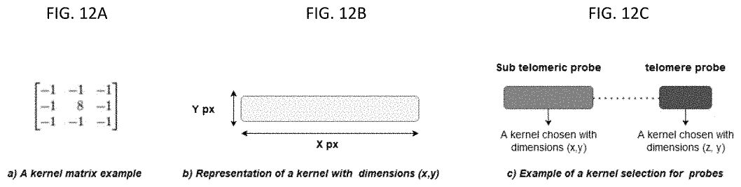

A detection algorithm uses predefined kernels to be applied on an image. A kernel is a 2-dimensional matrix (or it can be 3-dimensional for 3D image processing) containing weights, which applies convolutions on the images (FIG. 12A: is a general representation of a kernel). It is usually used for correction of the blur, sharpening or edge detection etc. It can be in various shapes like 3×3 or 4×4. Because the telomere signals are in long line shape, for this specific detection process rectangular and line-like shape kernel is used, like 15×5 or 150×10 (FIG. 12B: represents kernel designed for line like signals) and (FIG. 12C: represents the kernels designed for telomere probes).

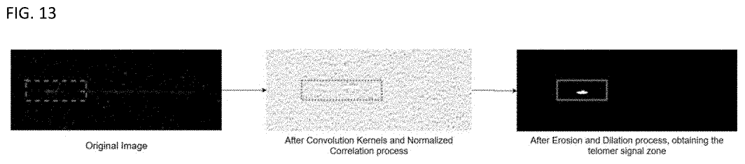

A convolution is an image processing operation of adding each pixel value of an image to its neighbor pixels by applying the weights in the kernel. After convolution process, normalized correlation, dilation and erosion is applied to generate zones, which are the objects that might be a signal. Normalized correlation is an operation to measure similarity of two patterns, it checks the correlation between two signals (for images signals are pixels values). Dilation and erosion are two morphological operations: dilation adds pixels to the boundaries of an object, while erosion removes pixels from boundaries. The combination of these two methods gives a combination of two actions: first, it distorts the pixels surrounding the objects, then it removes the noises around them to obtain a clear object zone (FIG. 13: shows the image processing flow of classical FiberStudio® software).

After obtaining this object zone, its surface is calculated and if it's above a given threshold the object is kept as a correct telomere signal. These zone thresholds are defined by developers and it can be changed any time depending on the expected length of the telomere. For example, mouse telomeres are longer than human telomeres, so the zone threshold is defined on the basis of expected telomere length of the species. To assign the colors of a detected signal, the values of the pixels' channel (Red, Blue and Green) are passed into the filters, which are basically predefined thresholds to assign a color on it.

Artificial Intelligent Based Software.

The new generation of software is based on artificial intelligence, using Deep Learning and Machine Learning methods to detect signals more precisely and faster than the classical software.

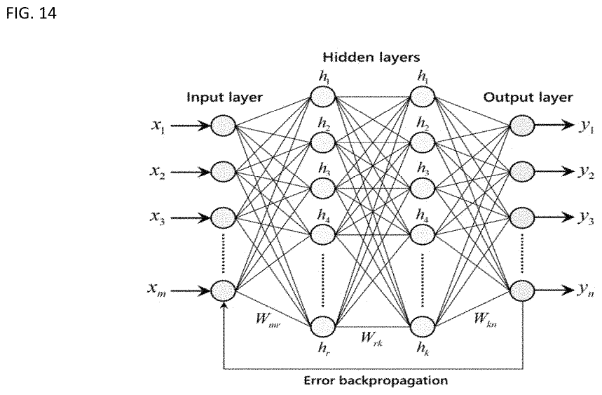

Machine learning is an ensemble of methods that computer algorithm can improve automatically through the given data. These methods build some mathematical models based on the given data to make predictions and decisions. While, Deep learning is a branch of machine learning that uses artificial neural networks and it does the learning based on supervised, semi-supervised or unsupervised data. A neural network (or artificial neural network) is a computing system inspired by biological neurons. It is constructed by connected units called “nodes”, which resemble neurons like function. Each connection and node have a number called “weights” which are adjusted in the learning/training process (FIG. 14: shows a general architecture of an artificial neural network). The automated software has separated modules that work together to give a high-quality output. The modules are: Detection module, Segmentation module, Classification/Clustering module and Reporting/Interpretation module.

Globally, the idea is to create neural networks and statistical models that learn features of signals from a large amount of data coming from validated signals by human investigators. Models are developed by the help of open source libraries Tensorflow, Keras, ScikitLearn and OpenCV. TensorFlow is an open source library for data processing and differentiable and parallel programming. It is used to make the calculations either in CPUs or in GPUs. Tensorflow is developed by Google Brain team and it was released as free library in 2015 (hypertext transfer protocol secure://www.tensorflow.org/).

CPU stands for Central Processing Unit, it is an electronic component in a computer that executes a computer program's instructions. GPU stands for graphic processing unit, it is an electronic circuit special for graphical systems and images. It's used in computer as a display unit. In Deep learning and artificial intelligence field, CPUs and GPUs are used for parallel and heavy calculations. Keras is an open source library for neural networks written in Python programming language. It is used to build and train neural networks and models (hypertext transfer protocol secure://keras.io/). ScikitLearn is an open source machine learning library, specific for python programming language containing classification, regression and clustering methods (hypertext transfer protocol secure://scikit-learn.org/stable/).

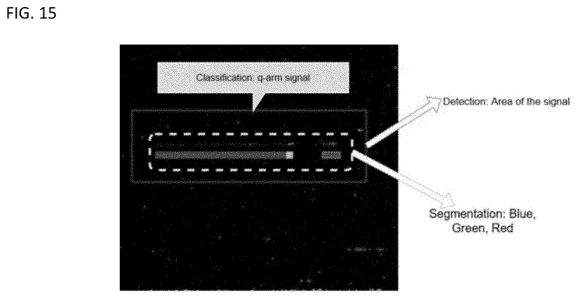

AI-based software, applies three main steps to obtain the correct Telomere signal. A first step is “Detection” process which involves finding an area that contains a telomere signal. A second step is “Segmentation” which to assign the correct color or colors on the detected signal. A third step is “Classification” which is to define the class of the signal for example if the signal is q-arm or the p-arm. FIG. 15 shows the implication of these three steps.

Detection Process in Automated Software Based on AI.

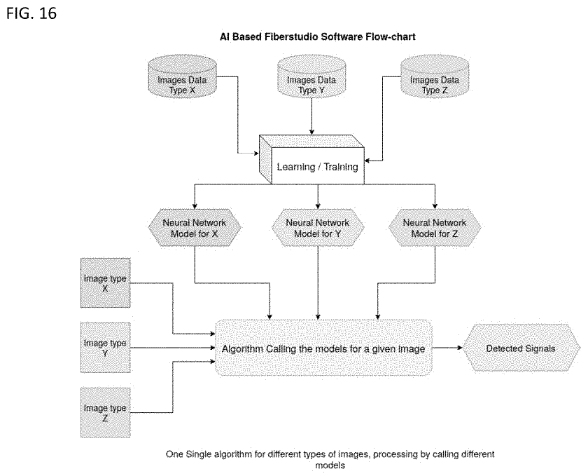

The AI based software uses convolutional neural networks (CNN). CNN is a type of artificial neural network, which has an architecture of multiple layers of nodes that can learn and extract features of an image. It's combined with two big parts: Convolutional Layers and Fully Connected Layers. Convolutional layers apply convolution operations on the image and learns the features of image by using dozens even hundreds of kernels. Fully Connected layers are an artificial neural network that learns based on these features extracted by convolutional layers, and makes predictions (FIG. 16 shows the flow-chart of AI based software).

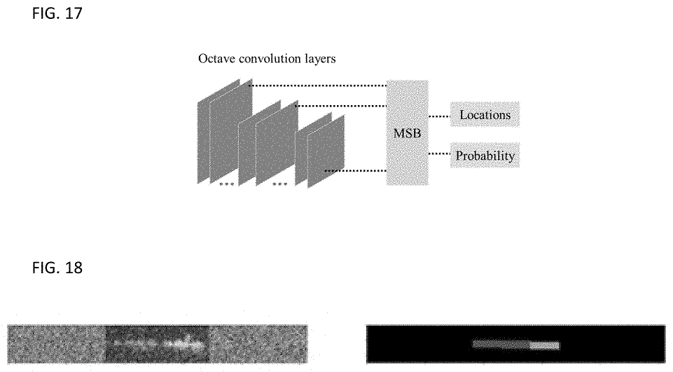

For the signal coming from the PCT applications (SubTA and/or DisTA), we're using octave convolutional layers in convolution operations, and multiscale detection block in making predictions. FIG. 17 shows the structure of the PCT's neural network for detection. Octave convolution layers apply average pooling operation for low frequency features and up sampling operations for high frequency features, after the convolution process. Low frequency signals of an image mean that pixel values changing slowly over the space (image zone), high frequency signals of an image mean that pixels which are changing values rapidly over the space. The average pooling is an operation executed to reduce the dimension of data, which combines the output of the convolutional layer into a one single neuron by using the convolution's outputs' average. Up sampling is an expansion process that is used to increase the dimension of the data, it generates more rich and distorted representation of the convolution output. Multiscale detection block (MDB) is a fully connected layers neural network, based on bounding boxes, which are fixed size zones that might contain objects. So MDB's job is to generate probabilities as predictions that if a zone contains an object and its location, which is adjusted to possible object's size.

The “training” phase means that the neural network is fed with all image data, the machine learns about telomere signals and becomes capable to detect them on a given coverslip. Several types of CNN models can be built and stocked for various signals detection. One model can be trained specifically for one type of signal or more global signal detection model can be created as well. More coverslip is scanned and reviewed/corrected by scientists, which means the model can be fed even more images to train and have more precise detection and prediction.

Segmentation Process.

Segmentation means that finding colors and their lengths of a detected signal. By using Linknet (a type of CNN), a deep learning model is built to define the colors of each pixel in order to obtain a correct segmentation of every color. FIG. 18 shows an example of a segmentation. LinkNet is an artificial neural network used for semantic segmentation, based on labeling each pixel of an image. For PCT's applications, it is used by labeling the color zones as the color interpreted by the user. So, in the training neural network can lead to assign some variation of colors to an interpreted color.

Segmentation's training process is quite similar to the detection's one. ROI images reviewed by technicians and scientists are given to the CNN by their colors and their starting/ending points, so that the network can run a learning process to understand which color may come after which one and which color can have more gaps (holes) or on which color gaps should be ignored. Various models can be created and added for different types of signals, if the gaps (holes) are important or if a combination of color should be seen as another color. For example, CNN can learn to interpret the Cyan color (equal amount of blue and green light) as blue or green.

Classification Process.

After segmentation, from the pattern of the signal, a numerical representation (a vector) is obtained. This vector contains very important information about the signal pattern such as a probe's length, its distance between other probes, its repeats in a signal and its position over the signal. FIG. 18 represents an example of a vector's creation process.

With this numerical representation or so called vector of each signal, by using machine learning methods a statistical model, called Gradient Boosting, is trained over the data to classify if the signal is a “p-arm telomere” or a “q-arm telomere”.

Gradient Boosting is a machine learning technique that forms of an ensemble of multiple learners, such as decision trees. For this gradient boosting model, an open source library XGBoost is used (hypertext transfer protocol secure://xgboost.readthedocs.io/en/latest/).

Classification's learning process is also similar to the previous steps. Signals' vectors are given to the machine learning model by their labels, as “q-arm telomere” and “p-arm telomere”. The algorithm re-adjusts its weights to make predictions.

For the signals that can't be identified, a clustering algorithm is applied and it may re-group and give some automatic labels over them.

Clustering is an unsupervised machine learning method to define similar signals and put them into groups.

Reporting.

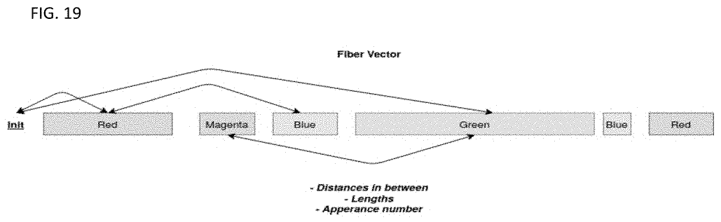

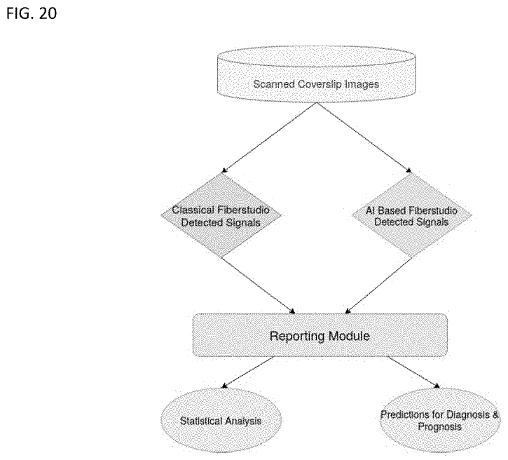

After detection and characterization of all signals, the separate reporting module can use the data coming from either the Classical FiberStudio® or AI based software to generate a report that contains descriptive statistics of all the signals to help scientists and technicians to analyze the data (FIG. 19). The module also provides risk scores and predictions made using machine learning models diseases linked to telomere length useful for diagnostics. When there are classified signals, the reporting module provides their percentage over the coverslip and their means and variances. Thus, providing statistics such as mean, median and variance of the lengths. All these pieces of information are depicted in a graph with histograms and/or heat maps.

The reporting module produces robust statistical results such as effectiveness of a treatment for telomere elongation or diagnosis-prognosis of a disease by sub-telomeric and telomere modifications, such as shortening, elongation or loss, with machine learning models trained over the clinical research data.

The applications derived using PCT are very precise methods to measure telomere for a kind of investigation that could not be possible before in a sole experiment. Indeed, telomeres are compared in genome wide manner, or distinguish generally between p arm and q arm of chromosomes or even to identify a specific region of the genome. Nevertheless, all the analyses can be done in a semi-automated or fully automated way by using FiberStudio® the Classical or the AI based software programs. Since by the aid of Molecular Combing System, the DNA fibers are stretched on coverslips, this allows the software programs: on one hand, to identify the combed DNA fibers, the sub-telomeric regions, the telomere signals and also distinguish the signals coming from the interstitial telomere sequences (ITSs).

Standardization Methods and the Mathematical Analysis Applicable with the Novel Methods.

PCT is the only method that allows a deep analysis of the telomere events like elongation, shortening and loss in genome wide as well as chromosome specific manner. detects telomere length distribution with great sensitivity.

To reach such precision and complexity, the inventors have identified a way to standardize and make quality control for each single experiment that is run with PCT application. In the first step, a cell system is embedded in 1%, 1.2%, 1.5%, 2% agarose plug. The number of cells used is 10,000, 100,000, 300,000, 500,000, 1,000,000. For each cell concentration the theoretical genome copy numbers (ptGCN) can be found:

ptGCN=n^{o} cells×2N

Then, the theoretical genome length into the plug (ptGL) is derived knowing that for male the length per cell is 6.2 Gb and for female is 6.3 Gb:

ptGL=ptGCN×6.2 (for male); ptGL=ptGCN×6.3 (for female)

To standardize the genome copy numbers (GCN) per coverslip the same number of cells were used, and the combed DNA for the gene sox5 (an identification gene once per genome) was hybridized. The theoretical GCN in the coverslip (ctGCN) is given by:

ctGCN=n^{o} sox5

Subsequently, this is traduced into DNA length: since the ctGCN per coverslip is known and so is the theoretical length (ctGL) for that specific coverslip:

ctGL=ctGCN×6.2 Gb (for male); ctGL=ctGCN×6.3 Gb (for female)

From these two equations, the number of the genome and hypothetical total length in either the plug or in the coverslip is understood. To estimate the actual theoretical genome copy number (θGCN), the ratio of ctGCN with ptGCN is calculated:

θGCN=ctGCN/ptGCN

Furthermore, the genome length (θGL) can be calculated. It is known that the length of genome is different if measured by crystallographic or molecular combed manner. To compare the two lengths, the stretching factor of the combed DNA is calculated, and the difference is of 1.6 Å (Ref). Finally, the θGL is calculated:

θGL=(ctGL*1.6)/ptGL

In order to measure the length of the combed DNA fibers, a specific algorithm designed was run to identify the combed DNA fibers and give the length per single fiber and/or the mean value to finally have the actual combed GCN length (aGCN). Whether the system is diploid or aneuploidy is determined by following the actual number of combed genomes (and the number of cells) for each specific coverslip.

aGCN=aGL/θGL

This new standardization method allows one to have a precise understanding of the exact numbers are used as reference within the experiment. It is possible to know how many cells and their genome length for each single coverslip. Furthermore, using a mathematical prediction model, this standardization method can also be applied to coverslips carrying a higher density of combed fibers.

Once the number of cells it is known, the theoretical number of telomeres signals per each coverslip can be derived as it is known that each cell has 92 telomeres (46 chromosomes*2 telomere per chromosome). The plug theoretical total number of telomeres (ptT) is calculated for the different cell concentration as follows:

ptT=n^{o} cells*92

This can be multiplied for the number of cells embedded in the plug to have the theoretical number of telomeres signals (ctT) that are expected in a coverslip by knowing the number of copies of sox5 gene:

ctT=sox5*92

Finally, the real theoretical telomere numbers (θT) can be found, including the variation introduced by the differential attachment of the DNA fibers to the coverslip surface at each combing procedure:

θT=ctT/ptT

Subsequently, the actual number of telomeres signals (aT) is counted, by comparing the aT with θT, and counting the ITSs, to find out whether there are more or less telomere signals for that coverslip:

TL=(aT+n^{o}ITSs)/θT

Furthermore, whether there is a loss of telomere in genome wide, chromosome arm specific and/or chromosome specific manner can be verified. It is also possible to follow if the telomere loss is due to a translocation event next to microsatellite regions by following the ratio of ITSs/Telomeres.

The standardization, provides an internal quality control for each single combed coverslip. Nevertheless, the exact number of signals for the model system by correlating with the absolute theoretical numbers, and/or the correlating the actual length with the absolute length can be validated.

In addition, by the use of these novel methods, it is possible to distinguish in genome wide p arm or q arm or region specific, the following parameters: mean of the length, absolute number of the telomere signals, correlation of the telomere length with the genome length within the same sample, genome wide distribution of the telomeres, number of true telomere signal and interstitial telomere sequences (ITSs), percentage of the ITSs events within the genome, telomere elongation, shortening or loss.

Multiple Model System and Collecting Strategies can be Used with PCT.

The methods disclosed herein can use a variety of samples. The FiberPrep® kit has been successfully used with samples originated from human, mice, plants, yeast and bacteria. In addition, PCT allows measuring signal from 1 kb up to 250 kb and more, and the possibility to use different model systems is still feasible to distinguish telomere length recognition between the different species/models.

The existing methods are mostly dedicated to one model system. They are unable to utilize multiple model systems for carrying out the telomere length analysis. Furthermore, their sensitivity, to distinguish the telomere lengths is only qualitative thus making the identification less accurate.

Thus, PCT allows collecting samples in multiple ways. Indeed, the DNA can be extracted from cell cultures, blood, tissue, organoids, PDX, saliva and small organisms. For all these samples sources, the plugs/Nanobind disks are generated and DNA are extracted.

Biomarker Identification and the Use for Diseases Stratification.

PCT is a powerful technique with the scope to uncover the genetic consequences of a disease from its onset, even when there are not significant diagnostic or physiological evidence of disease.

This new method can be performed on a sample to identify the genome rearrangements by the distinction of the ITSs and the telomere signals, and the telomere events (shortening, elongation, and loss). PCT can be used to understand whether there are telomere defects as a consequence of diseases and which arms of the chromosomes are affected.

First, by using PCT, the genome rearrangements within a sample can be estimated. This is achievable by correlating the number of ITSs upon the number of true telomere signals.

Secondly, it permits evaluation of telomere events (shortening, elongation and loss), and can measure telomere length thus providing a deeper understanding of the distribution of the telomere variations within a given genome. This step is crucial to comprehension of the range of telomere variations seen between healthy/sick or treated/non-treated samples including the comparisons between two or more drugs, agents or other therapies.

The invention concerns a process to follow the evolution of a disease linked to the modification of the telomere or sub telomeric physical lengths or size in the chromosomes of a patient treated or not by a drug or a therapeutic product/process, and to determine the efficiency of such drug or therapeutic by comparison with normal healthy subject/patient or with other control values, such as a pre-treatment assessment of telomere length or arrangement or with prior assessments taken during treatment, or assessments taken from untreated patients with a corresponding telomere-related disease, disorder or condition. A novel application of the present PCT invention concerns the follow up of the administration to the patients/subject of specific therapeutics or drugs in order to have an acute and specific measure of the efficiency of such therapeutics or drugs by using the present invention. The invention concerns also a process of following the evolution of diseases linked to the size of the telomeres in the chromosomes of a patient who is treated by drug or therapeutics. The evolution can be determined as well as the efficiency of the drug by applying the method according to the invention.

There are several methods that have been developed to change telomere stability or to prevent their shortening and loss. The wanted effect on the telomere is related to the kind of disease that is targeted. Specifically, there are few agents and treatments that can slow down the aging of human cells and mice, postulating, then, the possibility to cure the age-related diseases. Beside the nutrition supplement of vitamins, there are few treatments that show to be very efficient in elongating telomeres: 1) Hyperbaric Oxygen Therapy⁴⁹ (developed by Shai Efrati, Shamir Medical Center, ISR): the treatment consists to placed subjects in a pressurised chamber and given pure oxygen for 90 minutes a day, five days a week. After three months, the telomere of the subjects are elongated of 20% the telomere; 2) Nucleoside-modified TERT mRNA (developed by Rejuvenation Technologies, USA): this strategy is adopted by some of the pharma companies. The idea is to provide a new stamp to the TERT enzyme in order to elongate the telomere; 3) Gene editing of the telomerase: telomerase activity decreases with the aging. But for some time, there are stem cells at the periphery of a tissue/organ. These cells have fully active telomerase activity. The idea is to engineering cells to express enough levels of telomerase.

Beside the treatments to cure aging and genetic or rare-diseases, another set of compounds have the opposite effect: to block the telomerase activity. This is the case of cancer treatment, such as the myelodysplastic syndromes (MDS). Indeed, it is known that cancer cells have a higher telomerase activity, then the specific cancer can be affected by specific telomerase inhibitors: 1) Imetelstat® (developed by Geron, USA): is a drug in clinical phase 2. The Imetelstat® binds with high affinity to the template region of the RNA component of telomerase, resulting in direct, competitive inhibition of telomerase enzymatic activity, rather than elicit its effect through an antisense inhibition of protein translation. Imetelstat® is administered by intravenous infusion; 2) THIO(6-thio-dG) (developed by MayaBio, USA): it is a drug in preclinical studies. It is recognized by telomerase and incorporated into telomeres selectively in cancer cells. Once incorporated, it compromises telomere structure and function, leading to ‘uncapping’ of the chromosome ends resulting in rapid tumour cell death

Treatments that may increase telomere length include administration of particular foods, vitamins or nutriceuticals, vitamin C, vitamin E, nicotinamide riboside, antioxidants, oxygen, hyperbaric oxygen, steroid hormones, such as testosterone or estrogen, hGH, etc.

Thirdly, the observed events (shortening, elongation and loss) can be associated with the side of the genome. The telomere length distribution can be applied to the specific p and q arms to understand whether the telomere shortening is preferentially on charge of one or the other side of the chromosomes. In addition, variation of the telomere length between the p and the q arms of chromosomes can be assessed in a genome wide manner. PCT provides strong evidence of how telomeres are affected and what is the side of the chromosome that is preferentially affected in disease specific manner.

In addition, PCT can be applied to obtain stratification of diseases. It can be used, for example, to get the telomere length between kind of cancers and/or cells differing for the genetic background. In these cases, the telomere length distribution can be found, at p and q arms, which is peculiar for each of the considered systems. Thus, the telomere length represents the biomarker to stratify a disease like a type of cancer. This latest aspect of cell stratification of PCT opens a series of interesting scenarios for its clinical application. For example, clinical decisions in cancer treatment can be guided by detection of a specific type of cancer in a patient by performing PCT and comparing the data with the one in our dataset for the telomere length distribution.

In the PCT chromosome-specific applications, more detailed information about a specific disease and the identification of its biomarkers can be obtained and telomere events can be connected with a specific chromosome region by using probes for a specific sequence of the genome, such as the sub-telomeric regions of a chromosome.

This idea has been tested by using the telomere probes with some covering a specific sub-telomeric region of the chromosome 4 and/or the chromosome 10. In fact, these two sequences are known for a disease belonging to the muscular dystrophies and called facioscapulohumeral muscular dystrophy (FSHD)³⁷. It is accepted by clinicians that the disease is due to a shortening of repeated unit called D4Z4 on the Chr4 qA. More in details, the D4Z4 is located in the sub-telomeric regions of the chromosomes 4 and 10³⁸. The sub-telomeres are regions with a high recombination rate, and the sub-telomeric variations increases the genome variability and causes the onset of common or genetically inherited diseases³⁹. In this sense, FSHD patients might be prone to develop other disease that are related to metabolic and neurological disorders. In addition, there are hypothesis that bring new possible disease onset in response to FSHD; in particular the onset of cancers like melanomas, leukemia or lymphomas^{40,41}.

The PCT is set up for FSHD probes for both Chr4 qA/B and Chr10 qA/B and the telomere as well. The PCT correlates the severity of the FSHD with the telomere length of patients and could show that telomere events (shortening, elongation or loss) are additional biomarkers to predict the disease severity, for patients already suffering of FSHD, and as well to predictive biomarkers for development of other diseases such as cancer.

Similarly, for identification of gene of interest (GOI) or biomarker which is not in close proximity of the telomere can also be characterized by PCT. With the novel approach of combining chromosome arm specific probes and gene of interest (GOI) probes, telomere length alterations can be identified for gene of interest (GOI) which are located elsewhere in the genome and not adjacent to telomere. Application for the gene of interest (GOI) TERF1 gene, which is located on the chromosome 8 (q arm), is demonstrated. TERF1 gene encodes for a protein named TRF1 (Telomeric Repeat binding Factor-1) which has a role in negative regulation of telomere maintenance by inhibiting the telomerase activity. It has been clinically postulated that TRF1 corelates to telomere lengths in colorectal cancer^{51,52}. Thus, with the usage of PCT by identifying physical lengths alteration of the telomere with arm specific identification, in co-relation to the gene of interest (GOI), the prognostic/diagnostic significance can be developed for colorectal cancer patients.

The PCT can also be set up to use together the sub-telomeric regions for the chromosome 21 at the p and/or q arm (Chr21p/q). In this configuration, PCT have multiple advantages compared to the used methods. From one side, PCT can be easily used to screen patients carrying an extra copy of the Chr2l, to define Down Syndrome (DS) patients. On the other hand, the novel methods can uncover the function of telomere events in patients suffering from trisomy 21 syndrome. It has been found that telomere dysfunction is connected to DS. To such extent telomere length is considered as a biomarker of aging and dementia suffering patients, since replicative senescence could be accounted for aging of the immune system in DS patients. Lately, it has been seen that, in DS patients, telomeres shorten from age of 7 years and is more sever in female. However, in this study a wide range of aging is used for the elder patients (7-21 years).

Due to wide range of age sample size and lack of precision assays like qPCR and southern blot the relative quantification of the telomere length provided is imprecise and non-conclusive. In this case, PCT could give the advantage to have very precise measurements of the telomere dysfunction that might lead to stratify and refine better the ages of DS patients and telomere shortening. In addition, PCT can also give more precise information about the defects of T-lymphocytes in response to telomere dysfunction that are considered as biomarker for trisomy 21 and dementia such as Alzheimer disease².

Similarly, PCT can be used to determine the onset of myeloma in patients that show progressive degradation of the q arm of chromosome 13, starting indeed from the sub-telomeric region⁴³. Thus, comparative studies of the Chr13 q and the telomere length could finally define the telomere as biomarker for the clinical studies. Associated with breast cancer risks, i.e. the regions on the chromosomes 9p, 15p, 15 q and Xp^{44,45}. In this work, the telomere deficiencies are correlated in these four genomic regions with a potential risk to develop breast cancer. In this case, PCT can bring an absolute precision in the identification the actual biomarker between one or all, with very high precision and accuracy.

Biological samples comprising genomic DNA, chromosomal DNA, or RNA may be obtained from the fluids and tissues of a patient. These include blood, plasma, serum, urine, sweat, tears, breast milk, bile, interstitial fluid, cytosol, peritoneal fluid, pleural fluid, amniotic fluid, semen, synovial (joint) fluid, CSF (cerebrospinal fluid), lymph, mucous, saliva, or other bodily fluids, stool or fecal matter, or epithelium, hair follicles, or mucosal cells or secretions (such as from bronchial, nasal, buccal, or cheek swabs), or biopsy, such as a muscle biopsy. In some embodiments, samples may be further purified or isolated from other materials, for example, by removal of proteins, inactivation of nucleases, or by affinity purification of nucleic acids.

Molecular combing is known in the art and is incorporated by reference to Mahiet, et al., US 2016 0047006 A1, filed Mar. 4, 2015, entitled “Diagnosis of Viral Infections by Detection of Genomic and Infectious Viral DNA by Molecular Combing”; Lebofsky, et al., U.S. Pat. No. 7,985,542 B2, filed Sep. 7, 2006 entitled “Genomic Morse Code”; and Lebofsky, et al., U.S. Pat. No. 8,586,723 B2, filed Sep. 5, 2007 entitled “Genomic Morse Code”. Each of these documents is incorporated by reference in its entirety especially for description of supplies, such as detectable tags or indicators and method steps for molecular combing. Molecular combing also may be performed according to published methods (Lebofsky and Bensimon, MOL. CELL. BIOL., 2005, 25(15), 6789, incorporated by reference). Physical characterization of single genomes over large genomic regions is possible with molecular combing technology. An array of combed single DNA molecules is prepared by stretching molecules attached by their extremities to a silanised glass surface with a receding air-water meniscus. By performing fluorescent hybridization on combed DNA, genomic probe position can be directly visualized, providing a means to construct physical maps and for example to detect micro-rearrangements. Single-molecule DNA replication can also be monitored through fluorescent detection of incorporated nucleotide analogues on combed DNA molecules.

FISH (Fluorescent in situ hybridization) is a cytogenetic technique which can be used to detect and localize DNA sequences on chromosomes. It uses fluorescent probes which bind only to those parts of the chromosome with which they show a high degree of sequence similarity. Fluorescence microscopy can be used to find out where the fluorescent probe bound to the chromosome.

The inventors have developed specific features which can be combined with molecular combing procedures. These include development of the Nanobind CBB Big DNA Kit. This is a new technique which has been added to the existing molecular combing techniques to extract genomic DNA.

Another feature is an AI-based detection algorithm which is a novel detection algorithm developed for identification and classification for each individual application of the PCT.

Chromosome-Specific Probes and Sub-Telomeric Probes.

One skilled in the field may select probes that specifically bind to particular chromosomes or chromosome-specific sub-telomeric sequences. Nucleic acid sequences for the telomeric and sub-telomeric probes are based on the details shared by the supplier/vendor as they are commercially available products.

### Telomere-Specific Probes.

One skilled in the field may select probes that specifically bind to genomic or chromosome-specific telomeric sequences including those complementary to the hexanucleotide sequence TTAGGG.

These have been used to develop and test the PCT applications disclosed herein. Examples of such probes include:
Telomeric sequence: From PANAGENE PNA probes: Catalog no: F2003. Name: TelC-Alexa647.

Sequence:
(SEQ ID NO: 19)
(CCC TAA CCC TAA CCC TAA)ₙ

Length: 18 bp
Sub-telomeric sequence:
From CYTOCELL sub-telomeric probes. Catalog no: LPT13QR
Sequence co-ordinates details:
Database: UCSC hg38 (2013)
Region: q34
Coordinates: 114215870 to 114347428
Length: 131558 bp. (PDF of CHROMSOME 13 q PROBE SEQUENCE. SEQ ID NO: 18)
Disease specific Sub-telomeric sequence: FSHD probes. (DNA sequences for FSHD probes are shown in SEQ ID NOs: 1-17).
Sub-telomeric probes can be designed by using the Encode with the genome browser hg19 & hg38.
The co-ordinates for the SubTA genome-wide ‘Soup’ of 13 probes are detailed in FIG. 25.

The co-ordinates for the DisTA chromosome specific 46 probes are detailed in FIG. 26.

With reference to FIG. 25 and FIG. 26, the accession numbers for the genome, the chromosomes arms, and the specific probes (identified by sequence coordinates within the target accession number sequence) are as provided and are accessible at Ensembl Rest API—Ensembl REST API Endpoints. [online] (hypertext transfer protocol secure://rest.ensembl.org/ [last Accessed 31 Aug. 2021]).

In some embodiments, probes having sequences that are at least 95, 96, 97, 98, 99, 99.5, 99.9% identical to probe sequences disclosed herein or probes having deletions, substitutions, or insertions of 1, 5, 10, 20, 50 or more up to 1, 1.5 or 2% of total nucleotides in a probe sequence (or any intermediate value), may be used. BLASTN may be used to identify a polynucleotide sequence having at least 95%, 97.5%, 98%, 99% sequence identity to a reference polynucleotide. A representative BLASTN setting optimized to find highly similar sequences uses an Expect Threshold of 10 and a Wordsize of 28, max matches in query range of 0, match/mismatch scores of 1/−2, and linear gap cost. Low complexity regions may be filtered/masked. Default settings are described by and incorporated by reference to hypertext transfer protocol://blast.ncbi.nlm.nih.gov/Blast.cgi?PROGRAM=blastn&BLAST_PROGRAMS=megaBlast&PAGE_TYPE=BlastSearch&SHOW_DEFAULTS=on&LINK_LOC=blasthome (last accessed Nov. 17, 2020).

As stated above, with respect to the SubTA probes in FIG. 25 and the DisTA probes in FIG. 26, it is understood that these are exemplary embodiments of the primer design for use in the present invention. The accession numbers for the genome, the chromosomes arms, and the specific probes (identified by sequence coordinates within the target accession number sequence) are as provided and are accessible at Ensembl Rest API—Ensembl REST API Endpoints. [online](hypertext transfer protocol secure://rest.ensembl.org/ [last Accessed 31 Aug. 2021]). These exemplary embodiments provide benchmark sequences; however, it is understood that the present invention is not bound to the specific defined sequences as it is well-known in the art that with sequences of the length of the probes permit localized mismatch while preserving global binding. Further, when considering the length of the sequences of the respective probes, the reduced degree of overall sequence identity is tolerated. Thus, an embodiment of the present invention are probes that are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 25 and in FIG. 26.

Control Values.

Those skilled in the art may select a control or control value, such as a positive or negative control or control value based on the PCT technique being performed and on the type of subject or patient. Examples of control values include values from healthy or age (e.g., within 1, 2, 3, 4, or 5 years of age) and/or gender matched subjects or values from subjects having a particular telomere-related disease, disorder or condition. A control may be from untreated subject compared to a treated subject. Before and after values in the same patient or same patient cohort may be compared to assess efficacy of treatment with a particular drug or therapy. Control values may be obtained from an individual subject or be average values from a cohort of subjects.

Controls.

Some preferred controls were identified for PCT methodologies. In first instance a cell line derivate from hypertext transfer protocol secure://www.lgcstandards-atcc.org/products/all/HTB-96.aspx?geo_country=gb#documentation). This system is commonly used and accepted by the telomere community. By using U2OS, all the experimental work to prove the feasibility and work of SubTA and DisTA was performed. In addition, the repeatability was shown by using different model system such as: adenocarcinoma cell called HeLA (Ref: ATCC® CCL-2™, hypertext transfer protocol secure:://www.lgcstandards-atcc.org/products/all/CCL-2.aspx), and commercial human genomic DNA (TaKaRa Bio). After, to prove the sensitivity of SubTA and DisTA, blood samples from patients, healthy or affected by disease, can be used at different ages (i.e., 1, 5, 10, 20, 30, 50, 60, 70, 80 years old). The same blood samples and cell lines can be used also with drug treatment (treated vs non-treated) to prove the effects of selected drugs.

Diseases, disorders or conditions associated with telomere shortening include physical disease states associated with aging and stress exposure, including diabetes mellitus, obesity, heart disease, chronic obstructive pulmonary disease (COPD), asthma, as well as psychiatric illnesses, such as depression, anxiety, posttraumatic stress disorder (PTSD), bipolar disorder, and schizophrenia. The PCT methods disclosed herein may be used to evaluate telomere shortening, deletion, lengthening or other variation, and assess disease or health risks. Telomere length may be assessed after an infectious disease and correlated with recovery. PCT may also be applied to test the quality of embryonic stem cells, other stem cells, and other transplantable cells and tissues,

Diseases, disorders or conditions associated with telomere lengthening include neoplasms, tumors, and cancers, for example, glioma, serous low-malignant-potential ovarian cancer, lung adenocarcinoma, neuroblastoma, bladder cancer, melanoma, testicular cancer, kidney cancer and endometrial cancer, however telomere lengthening may decrease the risk for coronary heart disease, abdominal aortic aneurysm, coeliac disease and interstitial lung disease. The PCT methods disclosed herein may be used to evaluate telomere lengthening and assess disease or health risks.

Telomere modifications have a strong impact in the health of the somatic cells and then of the person. In the literature, there are many diseases that have been identified to be caused by the telomere modifications. These kinds of diseases due to telomere modifications more broadly cause: cardiovascular disease, stem cells cancer, stress, telomere shortening, metabolic diseases, diabetes, Alzheimer's, Parkinson's, infertility, menopause, arthritis, osteoporosis. It has been discovered in many studies the role of telomere in these diseases, and the list can become longer with the increasing of the technologies and the precision. In addition, there are already many diseases that are approved and recognized as clinical diseases to which PCT may be applied, as shown by the following table extracted by OMIM and Telomere Database websites.

These include those in the following links which are incorporated by reference (contents last accessed Nov. 24, 2020):

1) OMIM: hypertext transfer protocol
secure://omim.org/search?index=entry&search=telomere&start=1&limit=100&retrieve=geneMa
p&genemap_exists=true#;

2) Telomere Database: hypertext transfer protocol://telomerase.asu.edu/diseases.html

### EMBODIMENTS

1. A method for genome-wide or chromosome-specific detection of telomeres comprising:
   isolating or obtaining genomic DNA comprising chromosomal DNA,
   hybridizing tagged telomere-specific, sub-telomeric-specific, and/or chromosome-specific probes to the DNA for a time and under conditions suitable for hybridization of the probes to the DNA,
   counterstaining genomic DNA sequences that are not hybridized to a probe,
   detecting the location of, or pattern of, the hybridized tagged probes on the chromosomal DNA thereby providing data as to the location of the telomeric, sub-telomeric or chromosome-specific DNA on the chromosomes; and
   analyzing the data; and optionally,
   treating the subject when a correlation between a disease, disorder, or condition and the location of, or pattern, of hybridization in one or more chromosomes is detected.
2. The method of embodiment 1, further comprising treating a subject from whom the genomic DNA was isolated or obtained for a disease, disorder, or condition associated with shortening, deletion, rearrangement, abnormality, or lengthening of telomeric sequences compared to a control value.
3. The method of any of the foregoing embodiments, further comprising treating a subject for a disease, disorder or condition associated with shortening of telomeres.
4. The method of any of the foregoing embodiments, further comprising treating a subject for a disease, disorder or condition associated with deletion of telomeres.
5. The method of any of the foregoing embodiments, further comprising treating a subject for a disease, disorder or condition associated with lengthening of telomeres.
6. The method of any of the foregoing embodiments, further comprising treating a subject for a disease, disorder or condition associated with re-arrangement or other abnormality of telomeres.
7. The method of any of the foregoing embodiments, further comprising treating a subject from whom the genomic DNA was isolated or obtained for aging, stress exposure, including diabetes mellitus, obesity, heart disease, chronic obstructive pulmonary disease (COPD), asthma, psychiatric illnesses, such as depression, anxiety, posttraumatic stress disorder (PTSD), bipolar disorder, and schizophrenia when a correlation is detected.
8. The method of any of the foregoing embodiments, further comprising treating a subject for a disease, disorder or condition associated with shortening of telomeres wherein the disease is FSHD when a correlation is detected.
9. The method of any of the foregoing embodiments, further comprising treating a subject for a neoplasm, tumor, or cancer when a correlation is detected.
10. The method of any of the foregoing embodiments, further comprising treating a subject for glioma, serous low-malignant-potential ovarian cancer, lung adenocarcinoma, neuroblastoma, bladder cancer, melanoma, testicular cancer, kidney cancer or endometrial cancer when a correlation is detected.
11. The method of any of the foregoing embodiments, further comprising treating a subject for a breast cancer when a correlation is detected.
12. The method of any of the foregoing embodiments, wherein said detecting further comprises recording the locations of the probes on the p and/or q arms of chromosomal DNA.
13. The method of any of the foregoing embodiments, wherein said analyzing comprises computer analysis of the data as to a hybridization patterns of the telomeric, sub-telomeric, or chromosome-specific DNA on the a chromosome or chromosomes.
14. The method of any of the foregoing embodiments, wherein said analyzing comprises computer analysis of hybridization data as to the length of the telomeres on a chromosome or chromosomes.
15. The method of any of the foregoing embodiments, wherein said analyzing comprises computer correlation of the hybridization pattern with one or more symptoms.
16. The method of any of the foregoing embodiments, wherein said isolating further comprises molecular combing of the genomic DNA comprising chromosomal DNA.
17. The method of any of the foregoing embodiments, wherein said probes are tagged with a color or fluorescent dye.
18. The method of any of the foregoing embodiments, wherein said probes comprise red, green and yellow-tagged probes and wherein chromosomal DNA that is not hybridized to a probe is counterstained blue.
19. The method of any of the foregoing embodiments, wherein said probes are labelled with haptens recognized by a color-labelled hapten-specific antibody or by a hapten-specific antibody and a color-labelled secondary antibody.
20. The method of any of the foregoing embodiments, wherein said detecting comprises manually visualizing the location or pattern of the hybridized probes on the chromosomal DNA.
21. The method of any of the foregoing embodiments, wherein said detecting comprises scanning locations or patterns of the hybridized probes on a chromosome using an image scanner such as a FiberVision® or FiberVision® S scanner.
22. The method of any of the foregoing embodiments, further comprising computer analysis of the data describing the positions of, or pattern of, the hybridized probes.
23. The method of any of the foregoing embodiments, wherein the probes are p or q arm specific.
24. The method of any of the foregoing embodiments, wherein the probes are p or q are locus specific.
25. The method of any of the foregoing embodiments that comprises genome-wide detection of telomere and sub-telomere sequences in genomic DNA, wherein said probes bind to telomeric and sub-telomeric sequences on the p and/or q arms of the chromosomes in the genomic DNA, and wherein said detecting comprises distinguishing telomeric and sub-telomeric sequences from interstitial telomeric sequences (ITSs).
26. The method of any of the foregoing embodiments that comprises genome-wide detection of telomere and sub-telomere sequences in genomic DNA, further comprising pulsing the genomic DNA with dNTP analogs prior to isolation; wherein said probes bind to telomeric and sub-telomeric sequences on the p and/or q arms of the chromosomes in the genomic DNA, and wherein said detecting comprises detecting an average elongation of telomeres on the arm or arms chromosomes in the genomic DNA compared to a control value.
27. The method of any of the foregoing embodiments that comprises genome-wide detection of telomere and sub-telomere sequences in genomic DNA, wherein said probes bind to telomeric and sub-telomeric sequences on the p and/or q arms of the chromosomes in the genomic DNA, and wherein said detecting comprises detecting a shortening of telomeres on the chromosomes of the genomic DNA compared to a control value.
28. The method of any of the foregoing embodiments that comprises genome-wide detection of telomere and sub-telomere sequences in genomic DNA, wherein said probes bind to telomeric and sub-telomeric sequences on the p and/or q arms of the chromosomes in the genomic DNA, and wherein said detecting comprises detecting a chromosome loss at the p or q arm of a chromosome compared to a control value.
29. The method of any of the foregoing embodiments that comprises chromosome-specific detection of telomere and sub-telomere sequences in genomic DNA, wherein said probes bind chromosome-specific, telomeric and sub-telomeric sequences on the p and/or q arms of a chromosome in the genomic DNA, and wherein said detecting comprises distinguishing telomeric and sub-telomeric sequences on the chromosome from interstitial telomeric sequences (ITSs).
30. The method of any of the foregoing embodiments that comprises target chromosome-specific detection of target chromosome-specific, sub-telomere, and telomere sequences in genomic DNA, further comprising pulsing the genomic DNA with dNTP analogs prior to isolation, wherein said probes bind target chromosome-specific, sub-telomeric, and telomeric sequences on the p and/or q arms of a chromosome in the genomic DNA, and wherein said detecting comprises detecting an average elongation of telomeres on the arm or arms of the target chromosome compared to a control value.
31. The method of any of the foregoing embodiments that comprises chromosome-specific detection of telomere and sub-telomere sequences in genomic DNA, wherein said probes bind to chromosome-specific, sub-telomeric, and telomeric sequences on the p and/or q arms of the chromosomes in the genomic DNA, and wherein said detecting comprises detecting a shortening of telomeres on the chromosomes of the genomic DNA compared to a control value.
32. The method of any of the foregoing embodiments that comprises genome-wide detection of telomere and sub-telomere sequences in genomic DNA, wherein said probes bind to chromosome-specific, sub-telomeric, and telomeric sequences on the p and/or q arms of the chromosomes in the genomic DNA, and wherein said detecting comprises detecting a chromosome loss at the p or q arm of a chromosome compared to a control value.
33. The method of any of the foregoing embodiments, further comprising pulsing the genomic DNA with dNTP analogs prior to isolation, wherein the method comprises chromosome-specific detection of telomere and sub-telomere sequences in genomic DNA, wherein said probes bind chromosome-specific, sub-telomeric, and telomeric sequences on the p and/or q arms of a chromosome in the genomic DNA, and wherein said detecting comprises detecting an average elongation of telomeres on the arm or arms of the chromosome compared to a control value.
34. The method of any of the foregoing embodiments that is performed on two or more samples taken from the same subject at different times, wherein said analyzing the data comprises comparing telomere lengths or configurations in the two or more samples.
35. The method of any of the foregoing embodiments that is performed on two or more samples taken from the same subject at different times, wherein said analyzing the data comprises comparing telomere lengths or configurations in the two or more samples, and wherein the two or more samples comprise a control sample taken prior to treatment of the subject and a sample taken after treatment of a subject.
36. The method of any of the foregoing embodiments wherein the specific probes are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 25.
37. The method of any of the foregoing embodiments wherein the specific probes are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 26.
38. A kit for detecting telomere shortening (SubTAS) comprising at least one color-tagged probe that binds to a telomere and at least one probe that binds to a sub-telomeric sequence on a chromosome and optionally, immunostaining reagents, DNA extraction reagents, molecular combining supplies or equipment, and instructions for use of the kit to detecting telomere shortening.
39. A kit for detecting telomere loss (SubTAL) comprising at least one color-tagged probe that binds to a telomere and at least one probe that binds to a sub-telomeric sequence on a chromosome and optionally, immunostaining reagents, DNA extraction reagents, molecular combining supplies or equipment, and instructions for use of the kit to detecting telomere loss.
40. A kit for detecting telomere shortening (SubTAE) comprising at least one color-tagged probe that binds to a telomere and at least one probe that binds to a sub-telomeric sequence on a chromosome and optionally, dNTP analogs, immunostaining reagents, DNA extraction reagents, molecular combining supplies or equipment, and instructions for use of the kit to detecting telomere elongation.
41. The kit of any of embodiments 38 to 40 wherein the specific probes are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 25.
42. A kit for detecting distinguishing telomeres from interstitial telomere repeats, comprising at least one color-tagged probe that binds to a telomere and optionally, at least one probe that binds to a sub-telomeric sequence on a chromosome, immunostaining reagents, DNA extraction reagents, molecular combining supplies or equipment, and instructions for use of the kit to distinguish telomeres from interstitial telomere repeats.
43. The kit of embodiment 42 wherein the specific probes are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 25.
44. The kit of embodiment 42 wherein the specific probes are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 26.
45. A kit for detecting telomere shortening (DisTAS) comprising at least one color-tagged probe that binds to a telomere and at least one probe that binds to a sub-telomeric sequence on a chromosome, at least one probe that binds to a chromosome specific marker or locus and optionally, immunostaining reagents, DNA extraction reagents, molecular combining supplies or equipment, and instructions for use of the kit to detecting telomere shortening.
46. A kit for detecting telomere shortening (DisTAS) comprising at least one color-tagged probe that binds to a telomere and at least one probe that binds to a sub-telomeric sequence on a chromosome, at least one probe that binds to a chromosome specific marker or locus and optionally, immunostaining reagents, DNA extraction reagents, molecular combining supplies or equipment, and instructions for use of the kit to detecting telomere shortening; wherein said chromosome specific probe(s) bind to 4 qA and 4 qB variants of the 4qter subtelomere or other markers associated with FSHD interstitial telomere sequences.
47. A kit for detecting telomere loss (DisTAL) comprising at least one color-tagged probe that binds to a telomere and at least one probe that binds to a sub-telomeric sequence on a chromosome, at least one probe that binds to a chromosome specific marker or locus and optionally, immunostaining reagents, DNA extraction reagents, molecular combining supplies or equipment, and instructions for use of the kit to detecting telomere loss.
48. A kit for detecting telomere shortening (DisTAE) comprising at least one color-tagged probe that binds to a telomere and at least one probe that binds to a sub-telomeric sequence on a chromosome, at least one probe that binds to a chromosome specific marker or locus, and optionally, dNTP analogs, immunostaining reagents, DNA extraction reagents, molecular combining supplies or equipment, and instructions for use of the kit to detecting telomere elongation.
49. The kit of any of embodiments 43 to 46 wherein the specific probes are at least 60%, 65%, 70%, 75%, 80%, 85%, 90%, 92.5%, 95%, 96%, 97%, 98%, 99%, 99.5%, 99.9% identical to probe sequences corresponding to the coordinates defined in FIG. 26.
50. A process to follow evolution of a disease linked to the modification of the telomere or sub-telomeric physical lengths or size in the chromosomes of a patient treated or not by a drug or a therapeutic product/process, and to determine the efficiency of such drug or therapeutic by comparison with normal healthy subject/patient comprising: applying a PCT technique to genomic DNA of said patient to obtain an assessment of telomere length or configuration with respect to sub-telomeric sequences or other chromosomal sequences, and comparing said assessment to that of a control subject, and, optionally, continuing treatment, modifying treatment, or stopping treatment based on said comparison.
51. A composition for genome-wide or chromosome-specific detection of telomeres according to the method of claim 1 comprising DNA probes sequences corresponding to the coordinates defined in FIG. 25.
52. A composition according to claim 51 further comprising DNA probes sequences corresponding to the coordinates defined in FIG. 26.
53. A kit for detecting telomere elongation or telomere shortening (SubTAS) or (SubTAE) or (SubTAL) or (DisTAS) comprising at least one color-tagged probe that binds to a telomere and at least one probe that binds to a sub-telomeric sequence on a chromosome and optionally, immunostaining reagents, DNA extraction reagents, molecular combining supplies or equipment, and instructions for use of the kit to detecting telomere elongation, shortening or loss of telomere.

### Terminology

Terminology used herein is for the purpose of describing particular embodiments only and is not intended to be limiting of the invention.

As used herein, the singular forms “a”, “an” and “the” are intended to include the plural forms as well, unless the context clearly indicates otherwise.

As used herein, the term “and/or” includes any and all combinations of one or more of the associated listed items and may be abbreviated as “/”.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The terms “can” and “may” and their variants are intended to be non-limiting, such that recitation that an embodiment can or may comprise certain elements or features does not exclude other embodiments of the present invention that do not contain those elements or features.

Any numerical range recited herein is intended to include all sub-ranges subsumed therein.

A range encompasses its endpoints as well as values inside of an endpoint, for example, the range 0-5 includes 0, >0, 1, 2, 3, 4, <5 and 5.

Unless otherwise specified, all compositional percentages are by weight of the total composition, unless otherwise specified.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

### REFERENCES

1. Alnafakh, R. A. A., Adishesh, M., Button, L., Saretzki, G. & Hapangama, D. K. Telomerase and telomeres in endometrial cancer. Front. Oncol. 9, (2019).
2. Tsoukalas, D. et al. Association of nutraceutical supplements with longer telomere length. Int. J. Mol. Med. 44, 218-226 (2019).
3. Driscoll, M. O. The pathological consequences of impaired genome integrity in humans; disorders of the DNA replication machinery. 192-207 (2017) doi:10.1002/path.4828.
4. Lai, T. P., Wright, W. E. & Shay, J. W. Comparison of telomere length measurement methods. Philos. Trans. R. Soc. B Biol. Sci. 373, (2018).
5. Aubert, G., Hills, M. & Lansdorp, P. M. Telomere length measurement-Caveats and a critical assessment of the available technologies and tools. Mutat. Res.—Fundam. Mol. Mech. Mutagen. 730, 59-67 (2012).
6. Verma, P., Dilley, R. L., Gyparaki, M. T. & Greenberg, R. A. Direct Quantitative Monitoring of Homology-Directed DNA Repair of Damaged Telomeres. Methods in Enzymology vol. 600 (Elsevier Inc., 2018).
7. Kimura, M. et al. Telomere length and mortality: A study of leukocytes in elderly danish twins. Am. J. Fpidemiol. 167, 799-806 (2008).
8. Kahl, V. F. S. et al. Telomere Length Measurement by Molecular Combing. Front. Cell Dev. Biol. 8, 1-14 (2020).
9. Mazzucco, G. et al. Telomere damage induces internal loops that generate telomeric circles. (2020) doi:10.1101/2020.01.29.924951.
10. Kim, W. et al. Regulation of the Human Telomerase Gene TERT by Telomere Position Effect-Over Long Distances (TPE-OLD): Implications for Aging and Cancer. PLoS Biol. 14, 1-25 (2016).
11. Robin, J. D. & Magdinier, F. in Diseases: From Chromosomal Position Effect to Phenotype Variegation. Handbook of Epigenetics (Elsevier Inc.). doi:10.1016/B978-0-12-805388-1/00006-7.
12. Prioleau, M. & Macalpine, D. M. DNA replication origins—where do we begin ? 1683-1697 (2016) doi:10.1101/gad.285114.116.ical.
13. Schluth-Bolard, C., Ottaviani, A., Gilson, E. & Magdinier, F. A. A. Chromosomal position effects and gene variegation: Impact in pathologies. Handbook of Epigenetics (Elsevier Inc., 2011). doi:10.1016/B978-0-12-375709-8.00006-X.
14. Stadler, G. et al. Telomere position effect regulates DUX4 in human facioscapulohumeral muscular dystrophy. Nat. Struct. Mol. Biol. 20, 671-678 (2013).
15. Lundblad, V. Telomere maintenance without telomerase. Oncogene 21, 522-531 (2002).
16. Goldson, E. & Gardner, S. L. Developmental-behavioral aspects of chronic conditions. Dev. Pediatr. Evid. Pract. 301-404 (2008) doi:10.1016/B978-0-323-04025-9.50013-1.
17. Knight, S. J. L. et al. An optimized set of human telomere clones for studying telomere integrity and architecture. Am. J. Hum. Genet. 67, 320-32 (2000).
18. Rode, L., Nordestgaard, B. G. & Bojesen, S. E. Peripheral blood leukocyte telomere length and mortality among 64 637 individuals from the general population. J. Natl. Cancer Inst. 107, 1-8 (2015).
19. Jaskelioff M. et al. telomerase deficient mice. 469, 102-106 (2011).
20. Bernardes de Jesus, B. et al. Telomerase gene therapy in adult and old mice delays aging and increases longevity without increasing cancer. EMBO Mol. Med. 4, 691-704 (2012).
21. Povedano, J. M. et al. Therapeutic effects of telomerase in mice with pulmonary fibrosis induced by damage to the lungs and short telomeres. Ehfe 7, 1-24 (2018).
22. Riethman, H. et al. Mapping and initial analysis of human sub-telomeric sequence assemblies. Genome Res. 14, 18-28 (2004).
23. McLennan, D. et al. Telomere elongation during early development is independent of environmental temperatures in Atlantic salmon. J. Exp. Biol. 221, (2018).
24. Neumann, A. A. & Reddel, R. R. Telomere maintenance and cancer—Look, no telomerase. Nat. Rev. Cancer 2, 879-884 (2002).
25. Lee, M. et al. Telomere extension by telomerase and ALT generates variant repeats by mechanistically distinct processes. Nucleic Acids Res. 42, 1733-1746 (2014).
26. Ramunas, J. et al. Transient delivery of modified mRNA encoding TERT rapidly extends telomeres in human cells. FASEB J. 29, 1930-1939 (2015).
27. Martinez, P. & Blasco, M. A. Telomere-driven diseases and telomere-targeting therapies. 216, 875-887 (2017).
28. Blackburn, E. H. Walking the walk from genes through telomere maintenance to cancer risk. Cancer Prev. Res. 4, 473-475 (2011).
29. Shay, J. W. Role of telomeres and telomerase in aging and cancer. Cancer Discov. 6, 584-593 (2016).
30. Blackburn, E. H., Epel, E. S. & Lin, J. Human telomere biology: A contributory and interactive factor in aging, disease risks, and protection. Science (80-.). 350, 1193-1198 (2015).
31. Murnane, J., Sabatier, L., journal, B. M.-T. E. & 1994, undefined. Telomere dynamics in an immortal human cell line. embopress.org.
32. Srinivas, N., Rachakonda, S. & Kumar, R. Telomeres and telomere length: A general overview. Cancers (Basel). 12, 1-29 (2020).
33. Mendez-Bermudez, A. et al. Genome-wide Control of Heterochromatin Replication by the Telomere Capping Protein TRF2. Mol. Cell 70, 449-461.e5 (2018).
34. Assani, G., Xiong, Y., Zhou, F. & Zhou, Y. Effect of therapies-mediated modulation of telomere and/or telomerase on cancer cells radiosensitivity. Oncotarget 9, 35008-35025 (2018).
35. Boukamp, P. & Mirancea, N. Telomeres rather than telomerase a key target for anti-cancer therapy? Exp. Dermatol. 16, 71-79 (2007).
36. Armanios, M. & Blackburn, E. H. The telomere syndromes. Nat. Rev. Genet. 13, 693-704 (2012).
37. Lim, K. R. Q., Nguyen, Q. & Yokota, T. Dux4 signalling in the pathogenesis of facioscapulohumeral muscular dystrophy. Int. J. Mol. Sci. 21, (2020).
38. Nguyen, K. et al. Deciphering the complexity of the 4 q and 10 q subtelomeres by molecular combing in healthy individuals and patients with facioscapulohumeral dystrophy. J. Med. Genet. 56, 590-601 (2019).
39. Squassina, A., Pisanu, C. & Vanni, R. Mood Disorders, Accelerated Aging, and Inflammation: Is the Link Hidden in Telomeres? Cells 8, 52 (2019).
40. Gruszecka, A. et al. Telomere shortening in down syndrome patients—When does it start? DNA Cell Biol. 34, 412-417 (2015).
41. Armanios, M. & Armanios, M. Telomeres and age-related disease: how telomere biology informs clinical paradigms Find the latest version: Review series Telomeres and age-related disease: how telomere biology informs clinical paradigms. J. Clin. Invest. 123, 996-1002 (2013).
42. Jenkins, E. C. et al. Telomere shortening in T lymphocytes of older individuals with Down syndrome and dementia. Neurobiol. Aging 27, 941-945 (2006).
43. Li, J. et al. Telomere and 45S rDNA sequences are structurally linked on the chromosomes in Chrysanthemum segetum L. Protoplasma 249, 207-215 (2012).
44. Haycock, P. C. et al. Association between telomere length and risk of cancer and non-neoplastic diseases a mendelian randomization study. JAMA Oncol. 3, 636-651 (2017).
45. Zheng, Y. L., Zhou, X., Loffredo, C. A., Shields, P. G. & Sun, B. Telomere deficiencies on chromosomes 9p, 15p, 15 q and Xp: Potential biomarkers for breast cancer risk. Hum. Mol. Genet. 20, 378-386 (2011).
46. Nazanin Sadat Hashemi., Roya Babaei Aghdam., Atieh Sadat Bayat Ghiasi., Parastoo Fatemi., Template Matching Advances and Applications in Image Analysis. 2-3, (2016). (https://arxiv.org/pdf/1610.07231.pdf)
47. Zhong-Qiu, Zhao., Peng Zheng.,Shou-tao Xu., and Xindong Wu. Object Detection with Deep Learning: A Review, 2-10, (2018). (hypertext transfer protocol secure://arxiv.org/pdf/1807.05511.pdf)
48. Fabio Sigrist., Gradient and Newton Boosting for Classification and Regression, Expert Systems with Applications, (2018). (hypertext transfer protocol secure://arxiv.org/pdf/1808.03064v1.pdf)
49. Yafit Hachmo, Amir Hadanny, Ramzia Abu Hamed, Malka Daniel-Kotovsky, Merav Catalogna, Gregory Fishlev, Erez Lang, Nir Polak, Keren Doenyas, Mony Friedman, Yonatan Zemel, Yair Bechor, Shai Efrati. Hyperbaric oxygen therapy increases telomere length and decreases immunosenescence in isolated blood cells: a prospective trial. Aging, 2020; DOI: 10.18632/aging.202188
50. Stong N. et al. Subtelomeric CTCF and cohesin binding site organization using improved subtelomere assemblies and a novel annotation pipeline. Genome Res. 24, 1039-50 (2014).
51. Valls-Bautista C. et al. Telomeric repeat factor 1 protein levels correlates with telomere length in colorectal cancer. Rev Esp Enferm Dig. 104, 530-6 (2012).
52. Yin H. et al. Telomere Maintenance Variants and Survival after Colorectal Cancer: Smoking- and Sex-Specific Associations. Cancer Epidemiol Biomarkers Prev. 29, 1817-1824 (2020).

What we claim is:

1. A method for genome-wide or chromosome-specific detection of telomeres comprising:
isolating or obtaining genomic DNA comprising chromosomal DNA,
hybridizing tagged telomere-specific, sub-telomeric-specific, and/or chromosome-specific probes to the DNA for a time and under conditions suitable for hybridization of the probes to the DNA,
counterstaining genomic DNA sequences that are not hybridized to a probe,
detecting the location of, or pattern of, the hybridized tagged probes on the chromosomal DNA thereby providing data as to the location of the telomeric, sub-telomeric or chromosome-specific DNA on the chromosomes; and
analyzing the data; and optionally,
treating the subject when a correlation between a disease, disorder, or condition and the location of, or pattern, of hybridization in one or more chromosomes is detected.

2. The method of claim 1, further comprising treating a subject from whom the genomic DNA was isolated or obtained for a disease, disorder, or condition associated with shortening, deletion, rearrangement, abnormality, or lengthening of telomeric sequences compared to a control value.

3. The method of claim 1, further comprising treating a subject from whom the genomic DNA was isolated or obtained for aging, stress exposure, including diabetes mellitus, obesity, heart disease, chronic obstructive pulmonary disease (COPD), asthma, psychiatric illnesses, such as depression, anxiety, posttraumatic stress disorder (PTSD), bipolar disorder, and schizophrenia when a correlation is detected.

4. The method of claim 1, further comprising treating a subject for a disease, disorder or condition associated with shortening of telomeres wherein the disease is FSHD when a correlation is detected.

5. The method of claim 1, further comprising treating a subject for a neoplasm, tumor, or cancer when a correlation is detected.

6. The method of claim 1, further comprising treating a subject for glioma, serous low-malignant-potential ovarian cancer, lung adenocarcinoma, neuroblastoma, bladder cancer, breast cancer, melanoma, testicular cancer, kidney cancer or endometrial cancer when a correlation is detected.

7. The method of claim 1, wherein said analyzing comprises computer analysis of the data as to a hybridization patterns of the telomeric, sub-telomeric, or chromosome-specific DNA on a chromosome or chromosomes and/or of hybridization data as to the length of the telomeres on a chromosome or chromosomes.

8. The method of claim 1, wherein said isolating further comprises molecular combing of the genomic DNA comprising chromosomal DNA.

9. The method of claim 1, wherein said probes are tagged with a color or fluorescent dye.

10. The method of claim 1, wherein said probes are labelled with haptens recognized by a color-labelled hapten-specific antibody or by a hapten-specific antibody and a color-labelled secondary antibody.

11. The method of claim 1, wherein said detecting comprises visualizing and analyzing the location or pattern of the hybridized probes on the chromosomal DNA.

12. The method of claim 1 which comprises genome-wide detection of telomere and sub-telomere sequences in genomic DNA, wherein said probes bind to telomeric and sub-telomeric sequences on the p and/or q arms of the chromosomes in the genomic DNA, and wherein said detecting comprises:
distinguishing telomeric and sub-telomeric sequences from interstitial telomeric sequences (ITSs);
detecting an average elongation of telomeres on the arm or arms of chromosomes in the genomic DNA compared to a control value, wherein said method entails pulsing the genomic DNA with dNTP analogs prior to isolation;
detecting a shortening of telomeres on the chromosomes of the genomic DNA compared to a control value; or
detecting a chromosome loss at the p or q arm of a chromosome compared to a control value.

13. The method of claim 1 which comprises chromosome-specific detection of telomere and sub-telomere sequences in genomic DNA, wherein said probes bind chromosome-specific, telomeric and sub-telomeric sequences on the p and/or q arms of a chromosome in the genomic DNA, and wherein said detecting comprises distinguishing telomeric and sub-telomeric sequences on the chromosome from interstitial telomeric sequences (ITSs).

14. The method of claim 1 which comprises target chromosome-specific detection of target chromosome-specific, sub-telomere, and telomere sequences in genomic DNA, further comprising pulsing the genomic DNA with dNTP analogs prior to isolation, wherein said probes bind target chromosome-specific, sub-telomeric, and telomeric sequences on the p and/or q arms of a chromosome in the genomic DNA, and wherein said detecting comprises detecting an average elongation of telomeres on the arm or arms of the target chromosome compared to a control value.

15. The method of claim 1 which comprises chromosome-specific detection of telomere and sub-telomere sequences in genomic DNA, wherein said probes bind to chromosome-specific, sub-telomeric, and telomeric sequences on the p and/or q arms of the chromosomes in the genomic DNA, and wherein said detecting comprises detecting a shortening of telomeres on the chromosomes of the genomic DNA compared to a control value.

16. The method of claim 1 which comprises genome-wide detection of telomere and sub-telomere sequences in genomic DNA, wherein said probes bind to chromosome-specific, sub-telomeric, and telomeric sequences on the p and/or q arms of the chromosomes in the genomic DNA, and wherein said detecting comprises detecting a chromosome loss at the p or q arm of a chromosome compared to a control value.

17. The method of claim 1, further comprising pulsing the genomic DNA with dNTP analogs prior to isolation, wherein the method comprises chromosome-specific detection of telomere and sub-telomere sequences in genomic DNA, wherein said probes bind chromosome-specific, sub-telomeric, and telomeric sequences on the p and/or q arms of a chromosome in the genomic DNA, and wherein said detecting comprises detecting an average elongation of telomeres on the arm or arms of the chromosome compared to a control value.

18. The method of claim 1 which is performed on two or more samples taken from the same subject at different times, wherein said analyzing the data comprises comparing telomere lengths or configurations in the two or more samples.

19. The method of claim 1 which is performed on two or more samples taken from the same subject at different times, wherein said analyzing the data comprises comparing telomere lengths or configurations in the two or more samples, and wherein the two or more samples comprise a control sample taken prior to treatment of the subject and a sample taken after treatment of a subject.

20. A process to follow evolution of a disease linked to the modification of the telomere or sub-telomeric physical lengths or size in the chromosomes of a patient treated or not by a drug or a therapeutic product/process, and to determine the efficiency of such drug or therapeutic by comparison with normal healthy subject/patient comprising: applying a PCT technique to genomic DNA of said patient to obtain an assessment of telomere length or configuration with respect to sub-telomeric sequences or other chromosomal sequences, and comparing said assessment to that of a control subject, and, optionally, continuing treatment, modifying treatment, or stopping treatment based on said comparison.

21. A composition for genome-wide or chromosome-specific detection of telomeres according to the method of claim 1 comprising DNA probes sequences corresponding to the coordinates defined in FIG. 25.

22. A composition according to claim 21 further comprising DNA probes sequences corresponding to the coordinates defined in FIG. 26.

23. A kit for detecting telomere elongation or telomere shortening (SubTAS) or (SubTAE) or (SubTAL) or (DisTAS) comprising at least one color-tagged probe that binds to a telomere and at least one probe that binds to a sub-telomeric sequence on a chromosome and optionally, immunostaining reagents, DNA extraction reagents, molecular combining supplies or equipment, and instructions for use of the kit to detecting telomere elongation, shortening or loss of telomere.
